(19) United States
(12) Patent Application Publication
(10) Pub. No.: US 2026/0151350 A1
(43) Pub. Date: Jun. 4, 2026

(54) COMPOSITIONS AND METHODS FOR TARGETED DELIVERY TO CELLS

(71) Applicant: The Board of Regents of The University of Texas System, Austin, TX (US)

(72) Inventors: Daniel J. SIEGWART, Dallas, TX (US); Qiang CHENG, Dallas, TX (US); Sean DILLIARD, Dallas, TX (US)

(73) Assignee: The Board of Regents of The University of Texas System, Austin, TX (US)

(21) Appl. No.: 18/724,775

(22) PCT Filed: Dec. 27, 2022
(86) PCT No.: PCT/US2022/082411

§ 371 Date: Jun. 27, 2024

Related U.S. Application Data
(60) Provisional application No. 63/294,066, filed on Dec. 27, 2021.

(51) Int. Cl. A61K 9/51; A61K 9/00; A61K 31/7105; A61K 47/18; A61K 47/24; A61K 47/28

(52) U.S. Cl. CPC A61K 9/5123; A61K 9/0019; A61K 9/5146; A61K 9/5192; A61K 31/7105; A61K 47/186; A61K 47/24; A61K 47/28

(57) ABSTRACT

Described herein are compositions, kits, and methods for potent delivery to a cell of a subject. The cell can be of a particular cell type, such as a basal cell. In some cases, the cell can be a lung cell of a particular cell type. Also described herein are pharmaceutical compositions comprising a therapeutic or prophylactic agent assembled to a lipid composition. The lipid composition can comprise an ionizable cationic lipid, and a selective organ targeting lipid. The lipid composition can further comprise a phospholipid. Further described herein are high-potency intravenous dosage forms of a therapeutic or prophylactic agent formulated with a lipid composition.

### CROSS-REFERENCE

This application claims the benefit of U.S. Provisional Application No. 63/294,066, filed Dec. 27, 2021, which is hereby incorporated by reference in its entirety herein.

### STATEMENT AS TO FEDERALLY SPONSORED RESEARCH

This invention was made with government support under Grant No. R01 EB025192-01A1 by the National Institutes of Health. The government has certain rights in the invention.

### BACKGROUND

Nucleic acids that enable gene silencing, expression, and editing possess great potential for use as genetic medicines in multiple clinical settings including cancer, inherited genetic disorders, and infectious diseases. Due to the unfavorable pharmacokinetic properties of nucleic acids, viral and non-viral delivery approaches are used to facilitate nucleic acid delivery to target cells. Lipid nanoparticles (LNPs) represent the most clinically mature non-viral platform for the safe and efficacious delivery of genetic medicines. Indeed, LNPs were an enabling technology for the US FDA approval of the first siRNA drug, Onpattro, in 2018 and the mRNA vaccines currently being distributed for immunization against the SARS-CoV-2 virus, the causative agent of the COVID-19 pandemic. Despite this progress, intravenously (IV) administered LNPs typically accumulate in the liver and are internalized by liver hepatocytes, thereby greatly limiting the scope of their therapeutic applications.

### SUMMARY

The present disclosure provides, in some embodiments, a composition comprising a therapeutic agent assembled with a lipid composition. The lipid composition may comprise: an ionizable cationinc lipid; a polymer-conjugated lipid comprising one or more hydrocarbon chains that each comprise about 8 to about 20 (e.g., about 8 to about 18, about 8 to about 16, or about 8 to about 14) carbon atoms; and a selective organ targeting (SORT) lipid, e.g., separate from the ionizable cationic lipid and the polymer-conjugated lipid. The lipid composition may be characterized by an apparent ionization constant (pKa) from about 6 to about as determined by a 2-(p-toluidino)-6-naphthalenesulfonic acid (TNS) titration assay.

In some embodiments, the present disclosure provides a composition comprising a therapeutic agent assembled with a lipid composition. The lipid composition may comprise: an ionizable cationinc lipid; a polymer-conjugated lipid comprising one or more hydrocarbon chains that each comprise about 8 to about 20 (e.g., about 8 to about 18, about 8 to about 16, or about 8 to about 14) carbon atoms; and a selective organ targeting (SORT) lipid, e.g., separate from the ionizable cationic lipid and the polymer-conjugated lipid. The lipid composition may be characterized by an apparent ionization constant (pKa) outside a range of about 6 to about 7 as determined by a 2-(p-toluidino)-6-naphthalenesulfonic acid (TNS) titration assay. In some embodiments, the lipid composition is characterized by an apparent ionization constant (pKa) of about 6 or lower as determined by a 2-(p-toluidino)-6-naphthalenesulfonic acid (TNS) titration assay. In some embodiments, the lipid composition is characterized by an apparent ionization constant (pKa) of about 3 to about 6 as determined by a 2-(p-toluidino)-6-naphthalenesulfonic acid (TNS) titration assay.

In some embodiments, the present disclosure provides a composition comprising a therapeutic agent assembled with a lipid composition. The lipid composition may comprise: an ionizable cationinc lipid; a polymer-conjugated lipid comprising one or more hydrocarbon chains that each comprise about 8 to about 20 (e.g., about 8 to about 18, about 8 to about 16, or about 8 to about 14) carbon atoms; and a selective organ targeting (SORT) lipid, e.g., separate from the ionizable cationic lipid and the polymer-conjugated lipid. The lipid composition may be characterized by an apparent ionization constant (pKa) of about 8 or greater as determined by a 2-(p-toluidino)-6-naphthalenesulfonic acid (TNS) titration assay. In some embodiments, the lipid composition is characterized by an apparent ionization constant (pKa) of about 9 or greater as determined by a 2-(p-toluidino)-6-naphthalenesulfonic acid (TNS) titration assay. In some embodiments, the lipid composition is characterized by an apparent ionization constant (pKa) of about 8 to about 13 as determined by a 2-(p-toluidino)-6-naphthalenesulfonic acid (TNS) titration assay. In some embodiments, the lipid composition is characterized by an apparent ionization constant (pKa) of about 9 to about 13 as determined by a 2-(p-toluidino)-6-naphthalenesulfonic acid (TNS) titration assay. In some embodiments, the lipid composition is characterized by a zeta (ζ) potential of said lipid composition of about −10 millivolts (mV) to about 10 mV as determined by dynamic light scattering (DLS). In some embodiments, the lipid composition is characterized by a zeta (ζ) potential of said lipid composition of about 0 millivolt (mV) to about 10 mV as determined by dynamic light scattering (DLS). In some embodiments, the polymer-conjugated lipid is a polyethylene glycol (PEG)-conjugated lipid. In some embodiments, one or more hydrocarbon chains each comprise about 8 to about 18 carbon atoms. In other embodiments, one or more hydrocarbon chains each comprise about 8 to about 16 carbon atoms. In yet other embodiments, one or more hydrocarbon chains each comprise about 8 to about 14 carbon atoms. In some embodiments, a hydrocarbon chain of the one or more hydrocarbon chains of the polymer-conjugated lipid comprises no more than 3 unsaturated carbon-carbon bonds. In other embodiments, a hydrocarbon chain of the one or more hydrocarbon chains of the polymer-conjugated lipid comprises no more than 2 unsaturated carbon-carbon bonds. In some embodiments, the polymer-conjugated lipid comprises a polymer having a molecular weight of about 100 Daltons (Da) to about 100,000 Da. In other embodiments, the polymer-conjugated lipid comprises a polymer having a molecular weight of about 500 Da to about 100,000 Da. In some embodiments, the lipid composition comprises the polymer-conjugated lipid at a molar percentage from about 0.5% to about 20%. In other embodiments, the lipid composition comprises the polymer-conjugated lipid at a molar percentage from about 0.5% to about 15%. In yet other embodiments, the lipid composition comprises the polymer-conjugated lipid at a molar percentage from about 0.5% to about 10%.

In some embodiments, the present disclosure provides cationic ionizable lipids (e.g., ionizable cationic lipids). In some embodiments, the cationic ionizable lipid comprises a dendron or dendrimer comprising one or more branches, wherein said one or more branches each comprise two or more degradable functional groups. In some embodiments, said cationic ionizable lipid is a dendron or dendrimer that comprises one or more diacyl groups. In some embodiments, the ionizable cationic lipid is a dendrimer or dendron of a generation (g) having a structural formula:

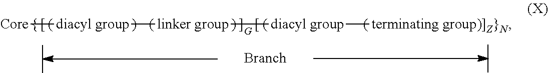

or a pharmaceutically acceptable salt thereof. In some embodiments, the core comprises a structural formula (X_{Core}):

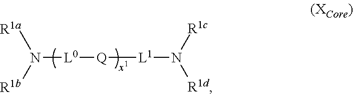

wherein Q is independently at each occurrence a covalent bond, —O—, —S—, —NR²—, or —CR^{3a}R^{3b}—, R² is independently at each occurrence R^{1g} or -L²-NR^{1e}R^{1f}, R^{3a} and R^{3b} are each independently at each occurrence hydrogen or an optionally substituted (e.g., C₁-C₆, such as C₁-C₃) alkyl, R^{1a}, R^{1b}, R^{1c}, R^{1d}, R^{1e}, R_{f}, and R_{1g} (if present) are each independently at each occurrence a point of connection to a branch, hydrogen, or an optionally substituted (e.g., C₁-C₁₂) alkyl, L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]—[(e.g., C₄-C₆) heterocycloalkyl]-[(e.g., C₁-C₆) alkylene], [(e.g., C₁-C₆) alkylene]-(arylene)-[(e.g., C₁-C₆) alkylene](e.g., [(e.g., C₁-C₆) alkylene]-phenylene-[(e.g., C₁-C₆) alkylene]), (e.g., C₄-C₆) heterocycloalkyl, and arylene (e.g., phenylene), or alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}, and x¹ is 0, 1, 2, 3, 4, 5, or 6. In some embodiments, each branch of the plurality (N) of branches independently comprises a structural formula (X_{Branch}):

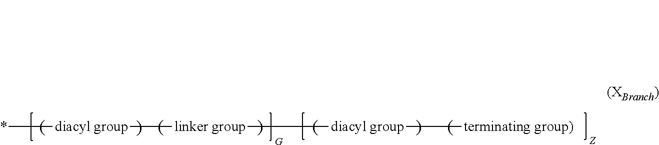

wherein * indicates a point of attachment of the branch to the core, g is 1, 2, 3, or 4, Z is 2^{(g-1)}, G is 0 when g is 1, or G is

$${\sum }_{i = 0}^{i = g - 2} {2}^{i} ,$$

when g≠1. In some embodiments, each diacyl group independently comprises a structural formula

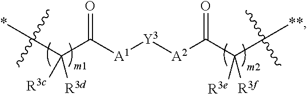

wherein * indicates a point of attachment of the diacyl group at the proximal end thereof, ** indicates a point of attachment of the diacyl group at the distal end thereof, Y³ is independently at each occurrence an optionally substituted (e.g., C₁-C₁₂): alkylene, an optionally substituted (e.g., C₁-C₁₂) alkenylene, or an optionally substituted (e.g., C₁-C₁₂) arenylene, A¹ and A² are each independently at each occurrence —O—, —S—, or —NR⁴—, wherein R⁴ is hydrogen or optionally substituted (e.g., C₁-C₆) alkyl, m¹ and m² are each independently at each occurrence 1, 2, or 3, and R^{3c}, R^{3d}, R^{3e}, and R^{3f} are each independently at each occurrence hydrogen or an optionally substituted (e.g., C₁-C₈) alkyl. In some embodiments, each linker group independently comprises a structural formula

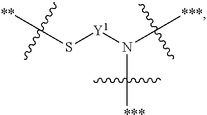

wherein ** indicates a point of attachment of the linker to a proximal diacyl group, *** indicates a point of attachment of the linker to a distal diacyl group, and Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene. In some embodiments, each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol. In some embodiments, x¹ is 0, 1, 2, or 3. In some embodiments, R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

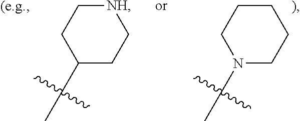

N—(C₁-C₃ alkyl)-piperidinyl

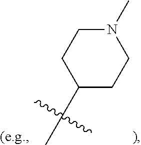 [SMILES: *C1CCN(C)CC1]

piperazinyl

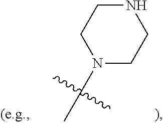 [SMILES: *N1CCNCC1]

N—(C₁-C₃ alkyl)-piperidinyl

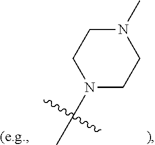 [SMILES: *N1CCN(C)CC1]

morpholinyl

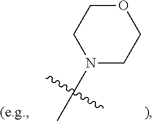 [SMILES: *N1CCOCC1]

### N-pyrrolidinyl

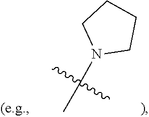 [SMILES: *N1CCCC1]

pyrrolidinyl

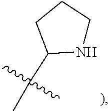 [SMILES: *C1CCCN1]

or N—(C₁-C₃ alkyl)-pyrrolidinyl

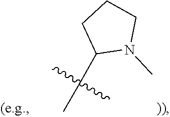 [SMILES: *C1CCCN1C]

(e.g., C₆-C₁₀) aryl, and C₃-C₅ heteroaryl (e.g., imidazolyl

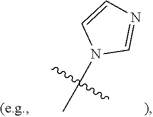 [SMILES: *n1ccnc1]

or pyridinyl

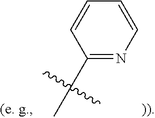 [SMILES: *c1ccccn1]

In some embodiments, R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH. In some embodiments, R^{3a} and R^{3b} are each independently at each occurrence hydrogen. In some embodiments, the plurality (N) of branches comprises at least 3 (e.g., at least 4, or at least 5) branches. In some embodiments, g=1; G=0; and Z=1. In some embodiments, each branch of the plurality of branches comprises a structural formula

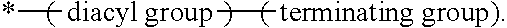

In some embodiments, g=2; G=1; and Z=2. In some embodiments, each branch of the plurality of branches comprises a structural formula

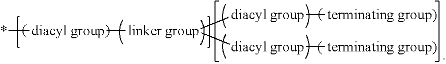

In some embodiments, g=3; G=3; and Z=4. In some embodiments, each branch of the plurality of branches comprises a structural formula

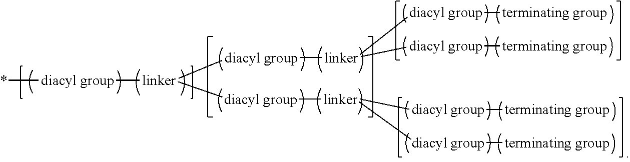

In some embodiments, g=4; G=7; and Z=8. In some embodiments, each branch of the plurality of branches comprises a structural formula

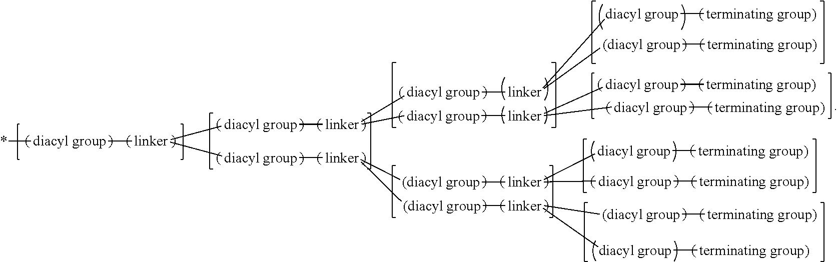

In some embodiments, the core comprises a structural formula:

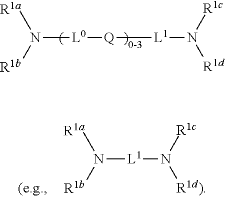

In some embodiments, the core comprises a structural formula:

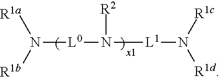

In some embodiments, the core comprises a structural formula:

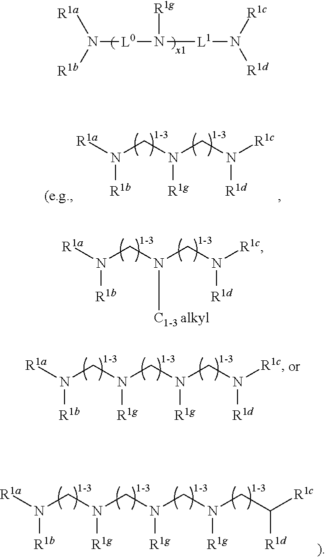

In other embodiments, the core comprises a structural formula:

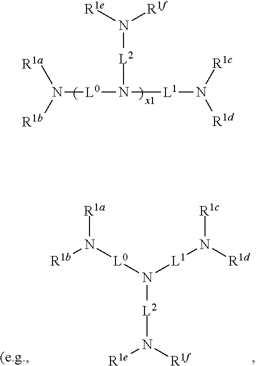

such as

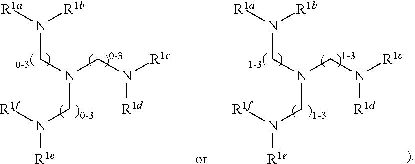

In yet other embodiments, the core comprises a structural formula:

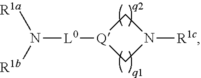

wherein Q′ is —NR²— or —CR^{3a}R^{3b}; q¹ and q² are each independently 1 or 2. In some embodiments, the core comprises a structural formula:

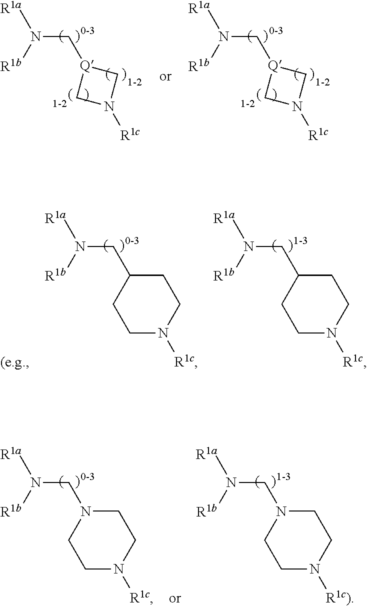

In some embodiments, the core comprises a structural formula

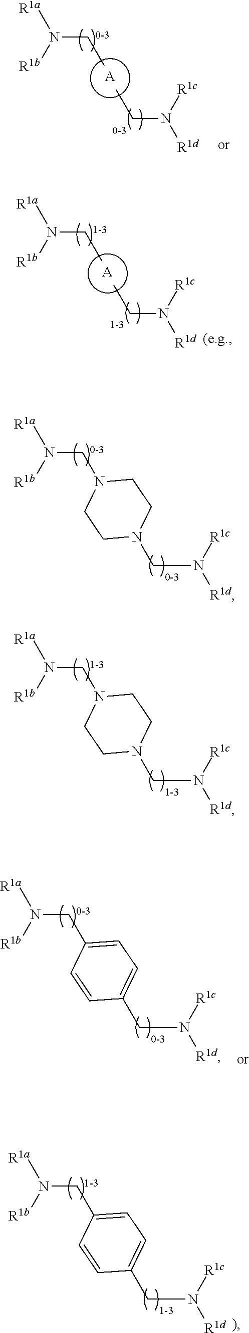

wherein ring A is an optionally substituted aryl or an optionally substituted (e.g., C₃-C₁₂, such as C₃-C₅) heteroaryl. In some embodiments, the core comprises has a structural formula

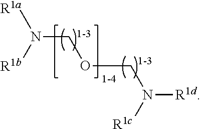

In some embodiments, the core comprises a structural formula selected from the group consisting of:

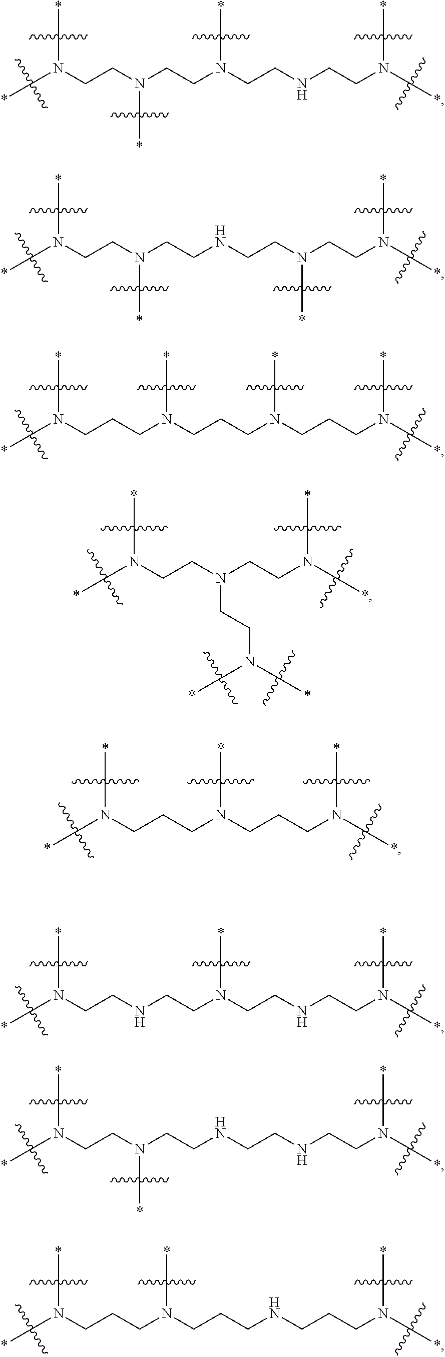

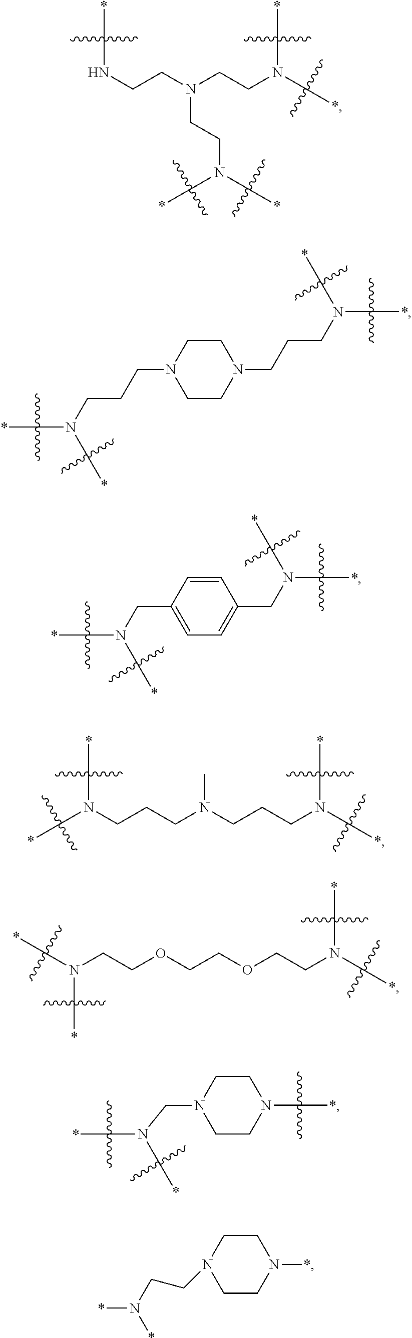

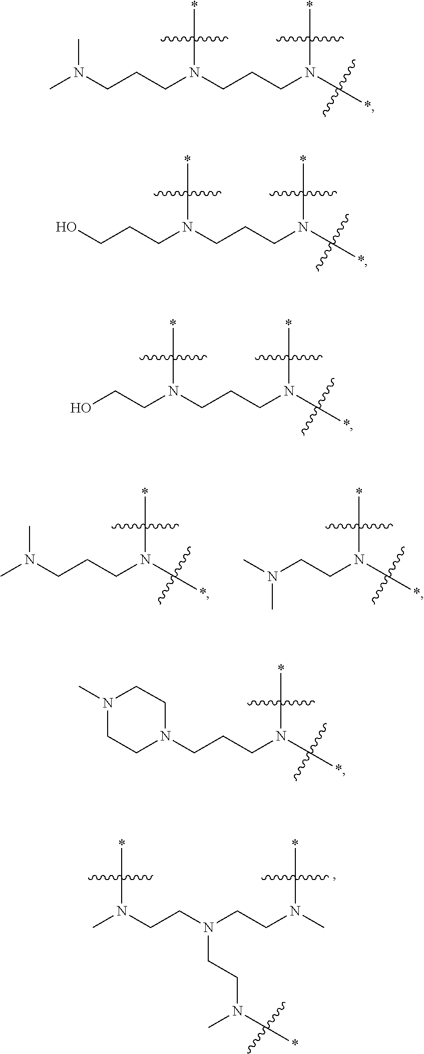

and pharmaceutically acceptable salts thereof, wherein * indicates a point of attachment of the core to a branch of the plurality of branches. In some embodiments, A¹ is —O— or —NH—. In some embodiments, A² is —O— or —NH—. In some embodiments, Y³ is C₁-C₁₂ (e.g., C₁-C₆, such as C₁-C₃) alkylene. In some embodiments, the diacyl group independently at each occurrence comprises a structural formula

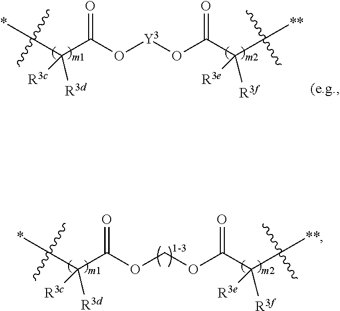

such as

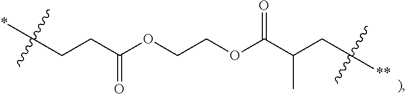

optionally wherein R^{3c}, R^{3d}, R^{3e}, and R^{3f} are each independently at each occurrence hydrogen or C₁-C₃ alkyl. In some embodiments, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

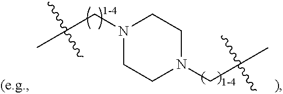

and [(C₁-C₄) alkylene]-phenylene-[(C₁-C₄) alkylene]

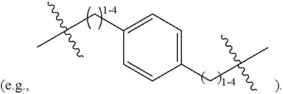

In some embodiments, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments, L⁰, L¹, and L² are each independently at each occurrence selected from [(C₁-C₄) alkylene]-[(C₄-C₆) heterocycloalkyl]-[(C₁-C₄) alkylene](e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene](e.g., —(C₁-C₃ alkylene)-piperazinyl-(C₁-C₃ alkylene)-).

In some embodiments, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

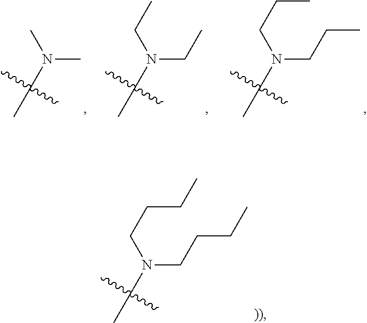

C₄-C₆ N-heterocycloalkyl

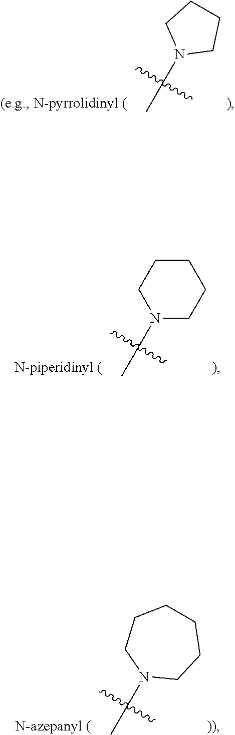 [SMILES: *N1CCCC1 ; *N1CCCCC1 ; *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

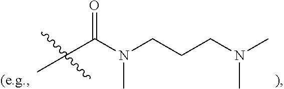 [SMILES: *C(=O)N(C)CCCN(C)C]

—C(O)N(C₁-C₃ alkyl)-(C₁-C₆ alkylene)-(C₄-C₆ N-heterocycloalkyl)

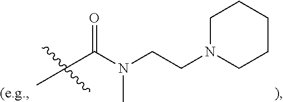 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

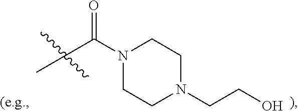 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl. In some embodiments, each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

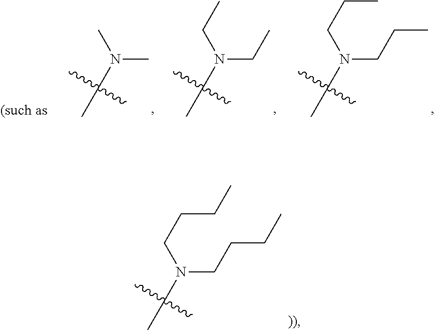

C₄-C₆ N-heterocycloalkyl

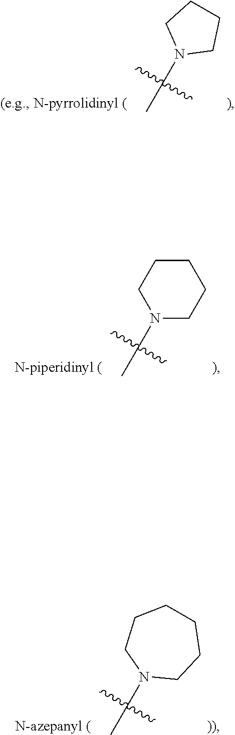 [SMILES: *N1CCCC1 ; *N1CCCCC1 ; *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

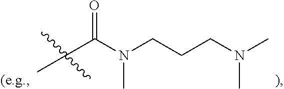 [SMILES: *C(=O)N(C)CCCN(C)C]

—C(O)N(C₁-C₃ alkyl)-(C₁-C₆ alkylene)-(C₄-C₆ N-heterocycloalkyl)

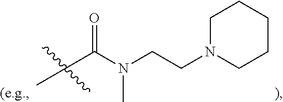 [SMILES: *C(=O)N(C)CCN1CCCCC1]

and —C(O)—(C₄-C₆ N-heterocycloalkyl)

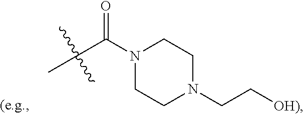

wherein the C₄-C₆ N-heterocycloalkyl moiety of any of the preceding substituents is optionally substituted with C₁-C₃ alkyl or C₁-C₃ hydroxyalkyl. In some embodiments, each terminating group is independently C₁-C₁₈ (e.g., C₄-C₁₈) alkylthiol, wherein the alkyl moiety is optionally substituted with one substituent —OH. In some embodiments, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

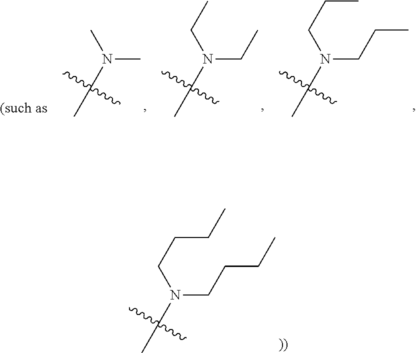

and C₄-C₆ N-heterocycloalkyl

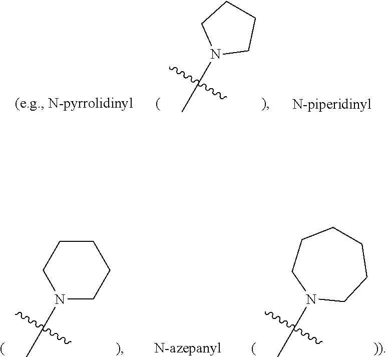 [SMILES: *N1CCCC1 ; *N1CCCCC1 ; *N1CCCCCC1]

In some embodiments, wherein each terminating group is independently C₁-C₁₈ (e.g., C₄-C₁₈) alkenylthiol or C₁-C₁₈ (e.g., C₄-C₁₈) alkylthiol. In some embodiments, each terminating group is independently C₁-C₁₈ (e.g., C₄-C₁₈) alkylthiol. In some embodiments, each terminating group is independently selected from the group consisting of:

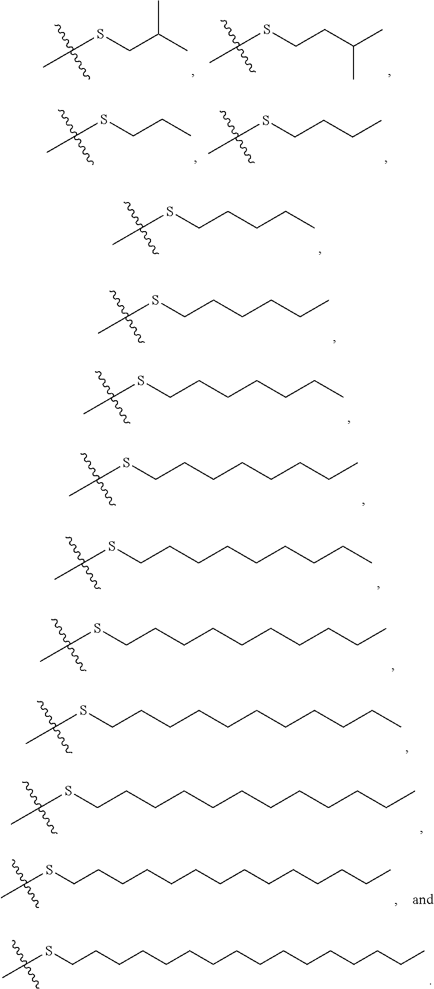

In some embodiments, the dendrimer or dendron is selected from the group consisting of

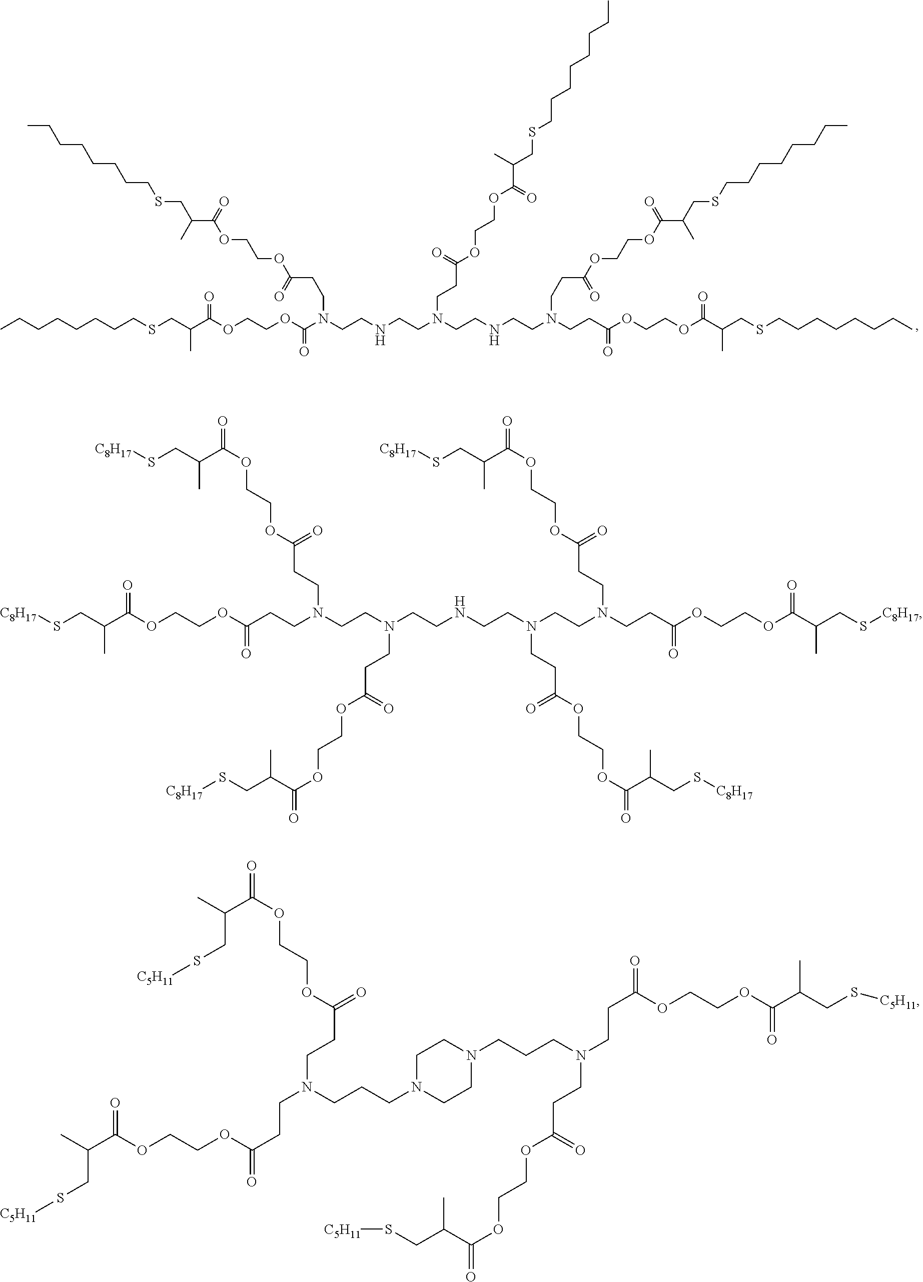

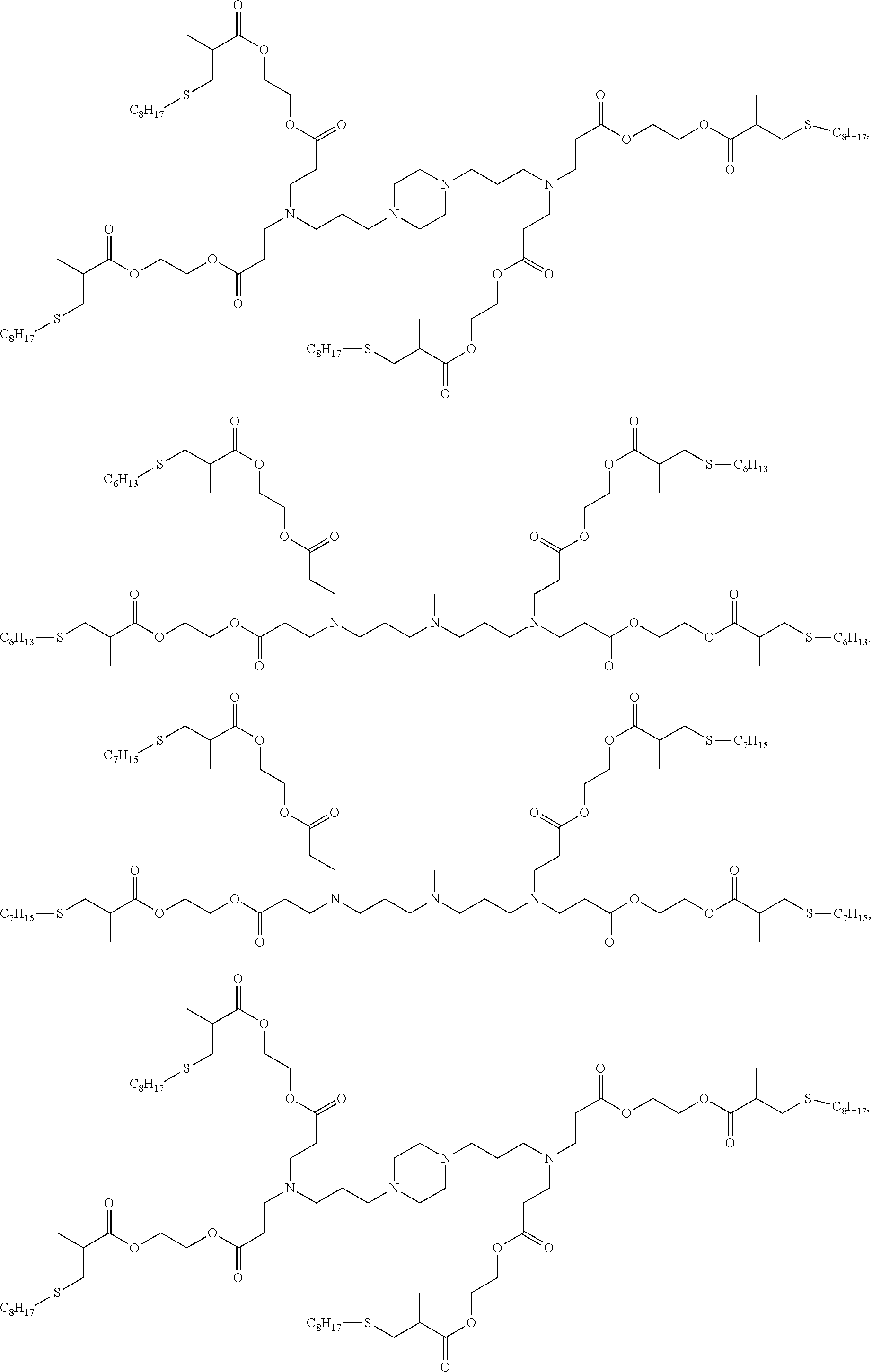

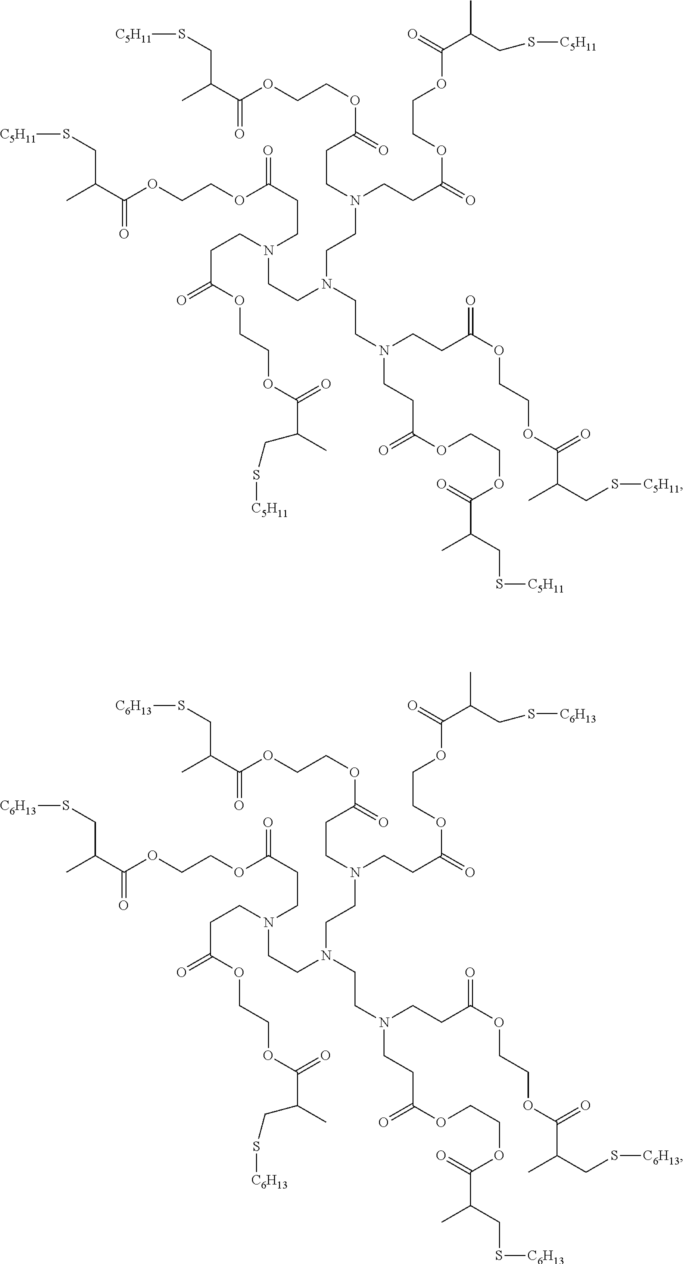

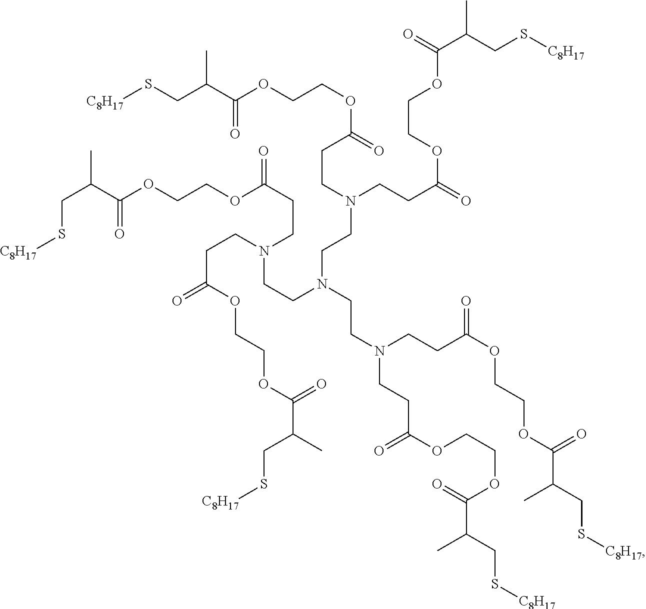

and pharmaceutically acceptable salts thereof. In some embodiments, said lipid composition comprises said ionizable cationic lipid at a molar percentage from about 5% to about 30%. In some embodiments, said lipid composition further comprises a phospholipid. In some embodiments, said lipid composition comprises said phospholipid at a molar percentage from about 5% to about 30%. In other embodiments, said lipid composition comprises said phospholipid at a molar percentage from about 8% to about 23%. In some embodiments, said phospholipid is not an ethylphosphocholine. In some embodiments, said lipid composition further comprises a steroid or steroid derivative. In some embodiments, said lipid composition comprises said steroid or steroid derivative at a molar percentage from about 15% to about 46%. In some embodiments, said steroid or steroid derivative is cholesterol. In some embodiments, said SORT lipid is cationic. In some embodiments, said SORT lipid comprises an ionizable cationic moiety (e.g., a tertiary amine moiety).

In some embodiments, the SORT lipid has a structural formula:

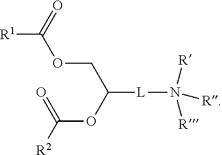

In some embodiments, L is a bond or a (e.g., biodegradable) linker. In some embodiments, R₁ and R₂ are each independently alkyl_{(C8-C24)}, alkenyl_{(C8-C24)}, or a substituted version of either group. In some embodiments, R′, R″ and R′″ are each independently alkyl_{(C≤6)} or substituted alkyl_{(C≤6)}. In some embodiments, the SORT lipid has a structural formula:

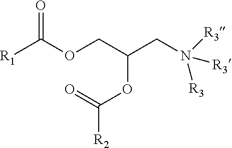

In some embodiments, R₁ and R₂ are each independently alkyl_{(C8-C24)}, alkenyl_{(C8-C24)}, or a substituted version of either group. In some embodiments, R₃, R₃′, and R₃″ are each independently alkyl_{(C≤6)} or substituted alkyl_{(C≤6)}. In some embodiments, said SORT lipid comprises a permanent cationic moiety (e.g., a quaternary ammonium ion). In some embodiments, said SORT lipid comprises a counterion to said permanent cationic moiety. In some embodiments, said SORT lipid is an alkylated phosphocholine (e.g., ethylphosphocholine). In some embodiments, said SORT lipid comprises a headgroup having a structural formula:

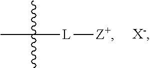

wherein L is a bond or a (e.g., biodegradable) linker; Z⁺ is positively charged moiety (e.g., a quaternary ammonium ion); and X⁻ is a counterion. In some embodiments, said SORT lipid has a structural formula:

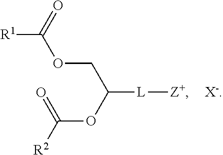

In some embodiments, R¹ and R² are each independently an optionally substituted C₆-C₂₄ alkyl, or an optionally substituted C₆-C₂₄ alkenyl. In some embodiment, said SORT lipid has a structural formula:

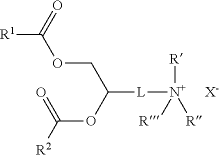

In some embodiments, R₁ and R₂ are each independently alkyl_{(C8-C24)}, alkenyl_{(C8-C24)}, or a substituted version of either group. In some embodiments, R′, R″ and R′″ are each independently alkyl_{(C≤6)} or substituted alkyl_{(C≤6)}. In some embodiments, X⁻ is a monovalent anion. In some embodiments, L is

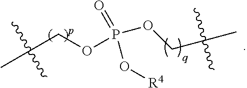

In some embodiments, p and q are each independently 1, 2, or 3. In some embodiments, R⁴ is an optionally substituted C₁-C₆ alkyl. In some embodiments, said SORT lipid has a structural formula:

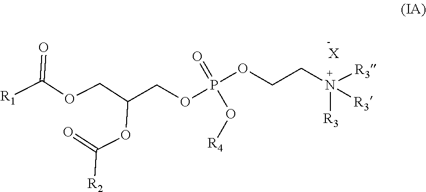

In some embodiments, R₁ and R₂ are each independently alkyl_{(C8-C24)}, alkenyl_{(C8-C24)}, or a substituted version of either group. In some embodiments, R₃, R₃′, and R₃″ are each independently alkyl_{(C≤6)} or substituted alkyl_{(C≤6)}. In some embodiments, R₄ is alkyl_{(C≤6)} or substituted alkyl_{(C≤6)}. In some embodiments, X⁻ is a monovalent anion. In some embodiments, said SORT lipid has a structural formula:

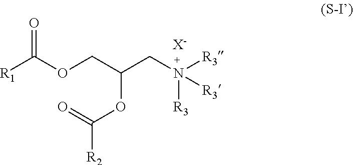

In some embodiments, R₁ and R₂ are each independently alkyl_{(C8-C24)}, alkenyl_{(C8-C24)}, or a substituted version of either group. In some embodiments, R₃, R₃′, and R₃″ are each independently alkyl_{(C≤6)} or substituted alkyl_{(C≤6)}. In some embodiments, X⁻ is a monovalent anion. In some embodiments, said SORT lipid has a structural formula:

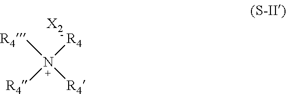

In some embodiments, R₄ and R₄′ are each independently alkyl_{(C6-C24)}, alkenyl_{(C6-C24)}, or a substituted version of either group. In some embodiments, R₄″ is alkyl_{(C≤24)}, alkenyl_{(C≤24)}, or a substituted version of either group. In some embodiments, R₄′″ is alkyl_{(C1-C8)}, alkenyl_{(C2-C8)}, or a substituted version of either group. In some embodiments, X₂ is a monovalent anion. In some embodiments, said lipid composition comprises said SORT lipid at a molar percentage from about 20% to about 65%. In some embodiments, said SORT lipid is zwitterionic. In some embodiments, said SORT lipid comprises a hydrophobically modified phosphate anion, a sulfonate anion, or a carboxylate anion. In some embodiments, said SORT lipid is anionic. In some embodiments, said SORT lipid has a structural formula:

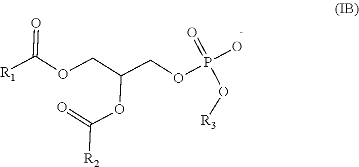

In some embodiments, R₁ and R₂ are each independently alkyl_{(C8-C24)}, alkenyl_{(C8-C24)}, or a substituted version of either group. In some embodiments, R₃ is hydrogen, alkyl_{(C≤6)}, or substituted alkyl_{(C≤6)}, or —Y₁—R₄. In some embodiments, Y₁ is alkanediyl_{(C≤6)} or substituted alkanediyl_{(C≤6)}. In some embodiments, R₄ is acyloxy_{(C≤8-24)} or substituted acyloxy_{(C≤8-24)}.

In some embodiments, said lipid composition is characterized by an average diameter of about 200 nanometers (nm) or less as determined by dynamic light scattering (DLS). In other embodiments, said lipid composition is characterized by an average diameter of about 150 nanometers (nm) or less as determined by dynamic light scattering (DLS). In yet other embodiments, said lipid composition is characterized by an average diameter of about 100 nanometers (nm) or less as determined by dynamic light scattering (DLS). In some embodiments, said lipid composition is characterized by a polydispersity index (PDI) of about 0.2 or less as determined by dynamic light scattering (DLS). In some embodiments, said lipid composition is characterized by a lipid fusion percentage of at least about 5%, 6%, 7%, 8%, 9%, or 10% as determined by a fluorescence resonance energy transfer (FRET)-based assay.

In some embodiments, said therapeutic agent comprises a compound, a polynucleotide, a polypeptide, a protein, or a combination thereof. In some embodiments, said therapeutic agent comprises a polypeptide or a protein. In some embodiments, said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof. In some embodiments, said therapeutic agent comprises a polynucleotide; and wherein a molar ratio of nitrogen in said lipid composition to phosphate in said polynucleotide (N/P ratio) is no more than about 20:1. In some embodiments, said N/P ratio is from about 5:1 to about 20:1. In some embodiments, said therapeutic agent comprises two or more polynucleotides that comprises said polynucleotide. In some embodiments, a molar ratio of said therapeutic agent to total lipids of said lipid composition is no more than about 1:1, 1:10, 1:50, or 1:100. In some embodiments, at least about 85% of said therapeutic agent is encapsulated in particles of said lipid compositions. In some embodiments, said SORT lipid is present in said composition in an amount sufficient to achieve a therapeutic effect at a dose of said therapeutic agent (e.g., at least about 1.1- or 10-fold) lower than that required with a reference lipid composition. In some embodiments, said therapeutic agent (e.g., heterologous polynucleotide) is present in said composition at a dose of no more than about 2 milligram per kilogram (mg/kg, or mpk) body weight. said therapeutic agent (e.g., heterologous polynucleotide) is present in said intravenous composition at a dose of no more than about 1.0, 0.5, 0.1, 0.05, or 0.01 mg/kg body weight. In some embodiments, the therapeutic agent is present in an aerosol composition at a dose of no more than 1.0, 0.5, 0.1, 0.05, or 0.01 mg/kg body weight. In some embodiments, wherein said therapeutic agent (e.g., heterologous polynucleotide) is present in said intravenous dosage form at a concentration of no more than about 5 or 2 milligram per milliliter (mg/mL).

The present disclosure also provides, in some embodiments, a method for targeted delivery of a therapeutic agent to an organ or a cell therein in a subject in need thereof. The method may comprise administering to the subject the therapeutic agent assembled with a lipid composition that comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition binds to a plurality of target proteins as determined by an incubation assay, which plurality of target proteins comprises a first target protein at a weight or mass ratio of no more than about 20:1, 15:1, or 10:1 to a second target protein that is different from the first target protein, thereby delivering the therapeutic agent to the target organ or the target cell in the subject. In some embodiments, the composition is according to any of the compositions described herein. In some embodiments, the method provides a (e.g., at least about 2-fold) greater amount, expression or activity of said therapeutic agent in said organ or said cell therein in said subject as compared to that achieved with a corresponding reference lipid composition (e.g., absent binding to said plurality of target proteins). In some embodiments, the method provides a (e.g., at least about 2-fold) greater amount, expression or activity of said therapeutic agent in said organ or said cell therein in said subject as compared to that achieved absent said polymer-conjugated lipid. In some embodiments, the method provides a (e.g., at least about 2-fold) greater amount, expression or activity of said therapeutic agent in said organ or said cell therein in said subject as compared to that achieved in a reference organ or a reference cell. In some embodiments, said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

In some embodiments, the present disclosure provides a method for targeted delivery of a therapeutic agent to an organ (e.g., liver) or a (e.g., liver) cell therein in a subject in need thereof, the method comprising administering to the subject the therapeutic agent assembled with a lipid composition that comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition binds to a plurality of target proteins as determined by an incubation assay, which plurality of target proteins comprises apolipoprotein E (Apo E) and serum albumin, thereby delivering the therapeutic agent to the target organ or the target cell in the subject. In some embodiments, the Apo E is present at a weight or mass ratio of no more than about 6:1, 5:1, 4:1, or 3:1 to the serum albumin the plurality of target proteins as determined by an incubation assay. In some embodiments, said plurality of target proteins further comprise complement C1q subcomponent subunit A, immunoglobulin heavy constant mu, complement C1q subcomponent subunit B, immunoglobulin kappa constant, immunoglobulin heavy constant gamma 2B, beta-globin, immunoglobulin (Ig) gamma-2A chain C region, complement C1q subcomponent subunit C, immunoglobulin heavy constant alpha, fibrinogen beta chain, fibrinogen gamma chain, immunoglobulin kappa variable 17-127, alpha globin 1, fibrinogen alpha chain, or any combination thereof as determined by an incubation assay. In some embodiments, said SORT lipid comprises an ionizable cationic moiety (e.g., a tertiary amine moiety). In some embodiments, said SORT lipid is an ionizable cationic lipid. In some embodiments, said lipid composition comprises said SORT lipid at a molar percentage from about 5% to about 65%. In some embodiments, said lipid composition is according to any lipid composition provided herein. In some embodiments, the method provides a (e.g., at least about 2-, 3-, 4-, 5-, or 6-fold) greater amount, expression or activity of said therapeutic agent in said liver or said liver cell in said subject as compared to that achieved with a corresponding reference lipid composition (e.g., absent said binding to said plurality of target proteins).

In some embodiments, the present disclosure provides a method for targeted delivery of a therapeutic agent to a non-liver organ or a non-liver cell therein in a subject in need thereof, the method comprising administering to the subject the therapeutic agent assembled with a lipid composition that comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition interacts with apolipoprotein E (Apo E) to a lesser degree than with an exogenous protein that is not Apo E in the subject as determined by an incubation assay, which endogenous protein is not Apor E is selected from beta-2-glycoprotein 1 (β2-GP1) or apolipoprotein H (Apo H), immunoglobulin kappa constant, complement C1q subcomponent subunit A, vitronectin, and serum paraoxonase/arylesterase 1, thereby delivering the therapeutic agent to the non-liver organ or the non-liver cell in the subject. In some embodiments, said non-liver organ comprises a lung, spleen, bone marrow, or a lymph node. In some embodiments, said non-liver cell comprises a lung cell, a spleen cell, or a macrophage. In some embodiments, apolipoprotein E (Apo E) is not the most abundant protein in said plurality of target proteins. In some embodiments, upon said administering, a surface of said lipid composition interacts with apolipoprotein C (Apo C) to a lesser degree than with apolipoprotein E (Apo E) in said subject as determined by an incubation assay. In some embodiments, the method provides a lesser amount or activity of said therapeutic agent in liver or a cell therein in said subject as compared to that achieved absent said polymer-conjugated lipid. In some embodiments, said SORT lipid is a permanent cationic lipid, an ionizable cationic lipid, a zwitterionic lipid, or an anionic lipid. In some embodiments, said lipid composition comprises said SORT lipid at a molar percentage from about 5% to about 65%. In some embodiments, said lipid composition is according to any of the lipid compositions described herein.

In some embodiments, the present disclosure provides a method for targeted delivery of a therapeutic agent to a lung or a lung cell therein in a subject in need thereof, the method comprising administering to the subject the therapeutic agent assembled with a lipid composition, which lipid composition comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition binds to a plurality of target proteins as determined by an incubation assay, which plurality of target proteins comprises vitronectin (Vtn) and clusterin, thereby delivering the therapeutic agent to the lung or the lung cell in the subject. In some cases, the vitronectin is present at a weight or mass ratio of no more than about 6:1 or 5:1 to the clusterin in the plurality of target proteins as determined by an incubation assay. In some embodiments, said plurality of target proteins further comprise serum paraoxonase/arylesterase 1, apolipoprotein E (Apo E), serum albumin, immunoglobulin kappa constant, prothrombin, complement C1q subcomponent subunit A, fibrinogen beta chain, beta-2 glycoprotein 1 (β2-GP1) or apolipoprotein H (Apo H), immunoglobulin (Ig) mu chain C region, alpha-S1-casein, immunoglobulin heavy constant gamma 2B, fibrinogen gamma chain, fibrinogen alpha chain, vitamin K-dependent protein Z, alpha-1-antitrypsin 1-3, plasminogen, apolipoprotein C-III, complement C1q subcomponent subunit B, thrombospondin-1, coagulation factor X, apolipoprotein A-I, immunoglobulin heavy constant alpha, immunoglobulin (Ig) gamma-2A chain C region, beta-globin, complement C1q subcomponent subunit C, protein Z-dependent protease inhibitor, or any combination thereof as determined by an incubation assay. In some embodiments, said SORT lipid is a cationic lipid. In some embodiments, said SORT lipid is a permanent cationic lipid. In some embodiments, said SORT lipid is an ionizable cationic lipid. In some embodiments, said lipid composition comprises said SORT lipid at a molar percentage from about 5% to about 65%. In some embodiments, said lipid composition is according to any lipid composition provided herein. In some embodiments, the method provides a (e.g., at least about 2-, 5-, 10-, 11-, 12-, 13-, 14-, 15-, 16-, 17-, 18-, 19-, or 20-fold) greater amount, expression or activity of said therapeutic agent in said lung or said lung cell in said subject as compared to that achieved with a corresponding reference lipid composition (e.g., absent binding to said plurality of target proteins). In some embodiments, said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

In some embodiments, the present application provides a method for targeted delivery of a therapeutic agent to spleen, bone marrow, or a lymph node or a cell therein in a subject in need thereof, the method comprising administering to the subject the therapeutic agent assembled with a lipid composition, which lipid composition comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition binds to a plurality of target proteins as determined by an incubation assay, which plurality of target proteins comprises beta-2 glycoprotein 1 (β2-GP1) or apolipoprotein H (Apo H) at a weight or mass ratio of no more than about 20:1, 15:1, or 10:1 to a second target protein that is different from the beta-2 glycoprotein 1 (β2-GP1) or apolipoprotein H (Apo H), thereby delivering the therapeutic agent to the spleen, bone marrow, or a lymph node or the cell in the subject. In some embodiments, In some embodiments, said cell comprises a spleen cell, or a macrophage. In some embodiments, said second target protein is selected from: immunoglobulin kappa constant, complement C1q subcomponent subunit A, apolipoprotein E (Apo E), immunoglobulin heavy constant gamma 2B, complement C1q subcomponent subunit B, vitronectin, complement C1q subcomponent subunit C, apolipoprotein C-I, immunoglobulin (Ig) gamma-2A chain C region, immunoglobulin (Ig) mu chain C region, serum albumin, serum paraoxonase/arylesterase 1, immunoglobulin heavy constant alpha, and immunoglobulin kappa variable 6-13. In some embodiments, said SORT lipid is a permanent cationic lipid, or an anionic lipid. In some embodiments, said SORT lipid is a permanent cationic lipid. In some embodiments, said SORT lipid is an anionic lipid. In some embodiments, said lipid composition comprises said SORT lipid at a molar percentage from about 5% to about 65%. In some embodiments, said lipid composition is according to any lipid composition provided herein. In some embodiments, the method provides a (e.g., at least about 2-fold) greater amount, expression or activity of said therapeutic agent in said lung or said lung cell in said subject as compared to that achieved with a corresponding reference lipid composition (e.g., absent binding to said plurality of target proteins).

In some embodiments, the present disclosure provides a method for targeted delivery of a therapeutic agent to a non-spleen organ or a non-spleen cell therein in a subject in need thereof, the method comprising administering to the subject the therapeutic agent assembled with a lipid composition, which lipid composition comprises: an ionizable cationic lipid; a polymer-conjugated lipid; and a selective organ targeting (SORT) lipid, e.g., separate from the ionizable cationic lipid and the polymer-conjugated lipid, wherein, upon the administering, a surface of the lipid composition binds to a plurality of target proteins as determined by an incubation assay, which plurality of target proteins comprises a first target protein at a weight or mass ratio of no more than about 20:1, 15:1, or 10:1 to a second target protein that is different from the first target protein, thereby delivering the therapeutic agent to the non-spleen organ or the non-spleen cell in the subject. In some embodiments, said non-spleen organ is not spleen, bone marrow, or a lymph node. In some embodiments, said non-spleen cell is not a spleen cell, or a macrophage. In some embodiments, beta-2 glycoprotein 1 (β2-GP1) or apolipoprotein H (Apo H) is not the most abundant protein in said plurality of target proteins. In some embodiments, said plurality of target proteins comprise clusterin. In some embodiments, said SORT lipid is a permanent cationic lipid, an ionizable cationic lipid, a zwitterionic lipid, or an anionic lipid. In some embodiments, said lipid composition comprises said SORT lipid at a molar percentage from about 5% to about 65%. In some embodiments, said lipid composition is according to any lipid composition provided herein. In some embodiments, said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

In some embodiments, said polymer-conjugated lipid is a polyethylene glycol (PEG)-conjugated lipid. In some embodiments, said one or more hydrocarbon chains each comprise about 8 to about 20 carbon atoms. In some embodiments, said one or more hydrocarbon chains each comprise about 8 to about 18 carbon atoms. In some embodiments, wherein said one or more hydrocarbon chains each comprise about 8 to about 16 carbon atoms. In some embodiments, said one or more hydrocarbon chains each comprise about 8 to about 14 carbon atoms. In some embodiments, a hydrocarbon chain of said one or more hydrocarbon chains of said polymer-conjugated lipid comprises no more than 3 unsaturated carbon-carbon bonds. In some embodiments, a hydrocarbon chain of said one or more hydrocarbon chains of said polymer-conjugated lipid comprises no more than 2 unsaturated carbon-carbon bonds. In some embodiments, said polymer-conjugated lipid comprises a polymer having a molecular weight of about 100 Daltons (Da) to about 100,000 Da. In some embodiments, said polymer-conjugated lipid comprises a polymer having a molecular weight of about 500 Da to about 100,000 Da. In some embodiments, said lipid composition comprises said polymer-conjugated lipid at a molar percentage from about 0.5% to about 20%. In some embodiments, said lipid composition comprises said polymer-conjugated lipid at a molar percentage from about 0.5% to about 15%. In some embodiments, said lipid composition comprises said polymer-conjugated lipid at a molar percentage from about 0.5% to about 10%. In some embodiments, said administering comprises intravenously administering. In some embodiments, a bodily fluid (e.g., plasma or serum) of said subject comprises said plurality of target proteins. In some embodiments, said plurality of target proteins are a plurality of endogenous proteins of said subject.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the disclosure are set forth with particularity in the appended claims. A better understanding of the features and advantages of the present invention will be obtained by reference to the following detailed description that sets forth illustrative embodiments, in which the principles of the disclosure are utilized, and the accompanying drawings (also “Figure” and “FIG.” herein), of which:

FIGS. 1A-1E illustrate selective organ targeting (SORT) lipid compositions (e.g., nanoparticles) for tissue-specific mRNA delivery including the unique biodistribution and ionization behavior thereof.

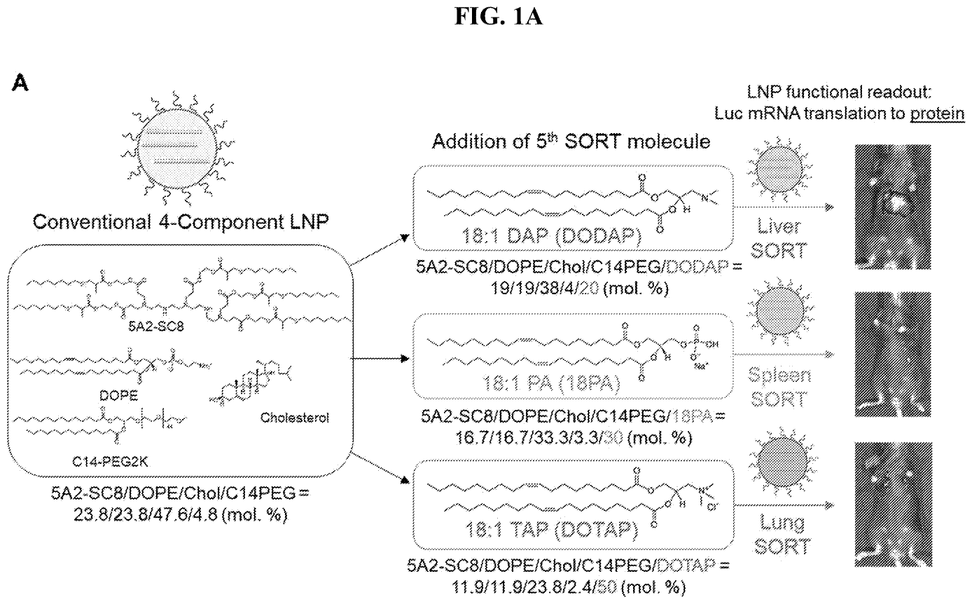

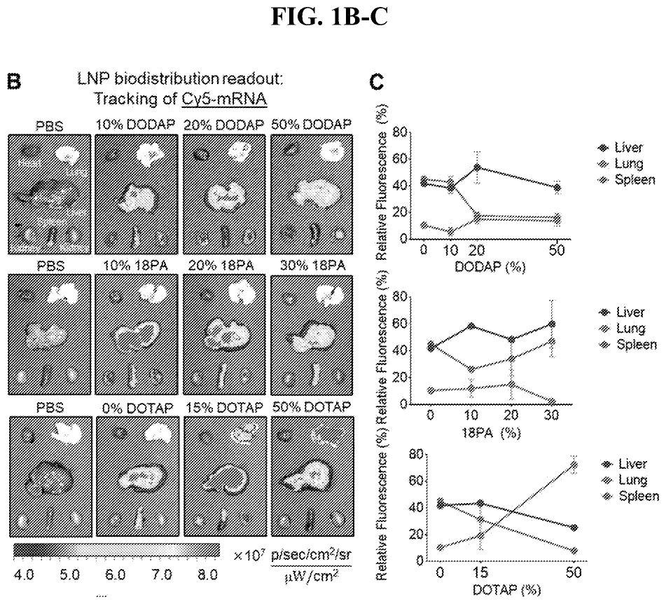

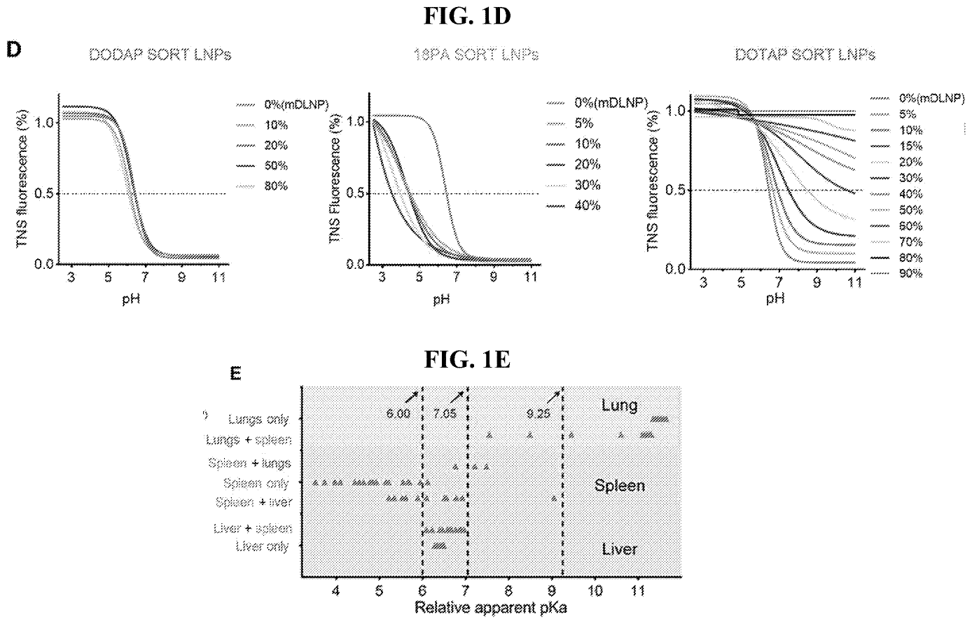

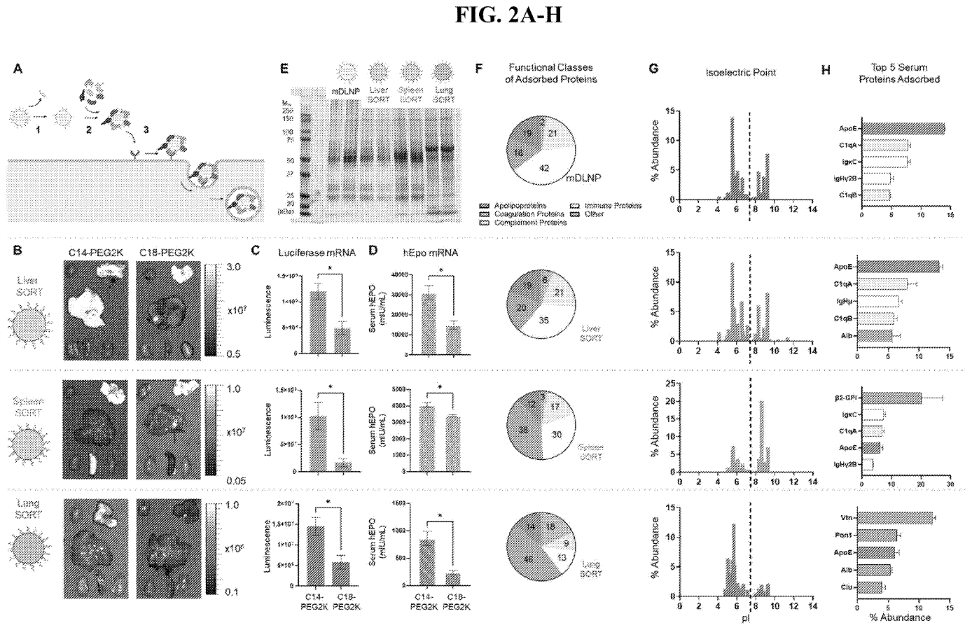

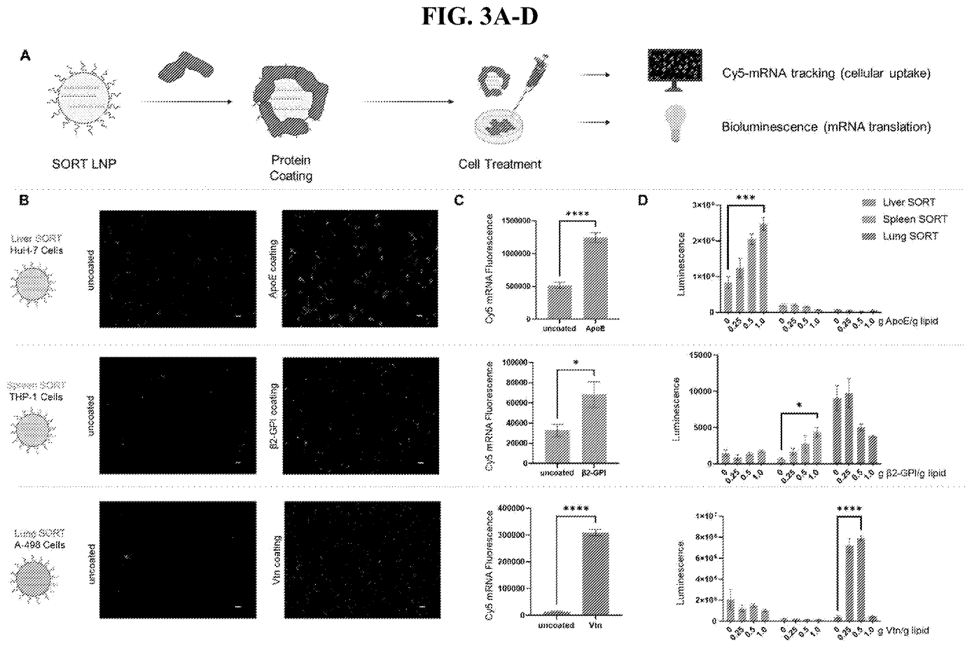

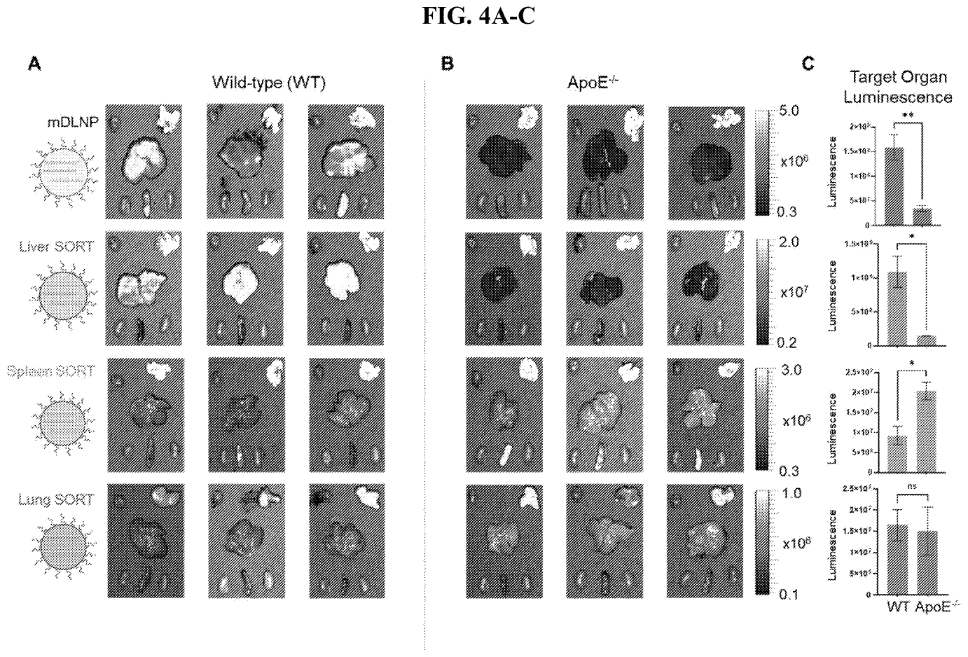

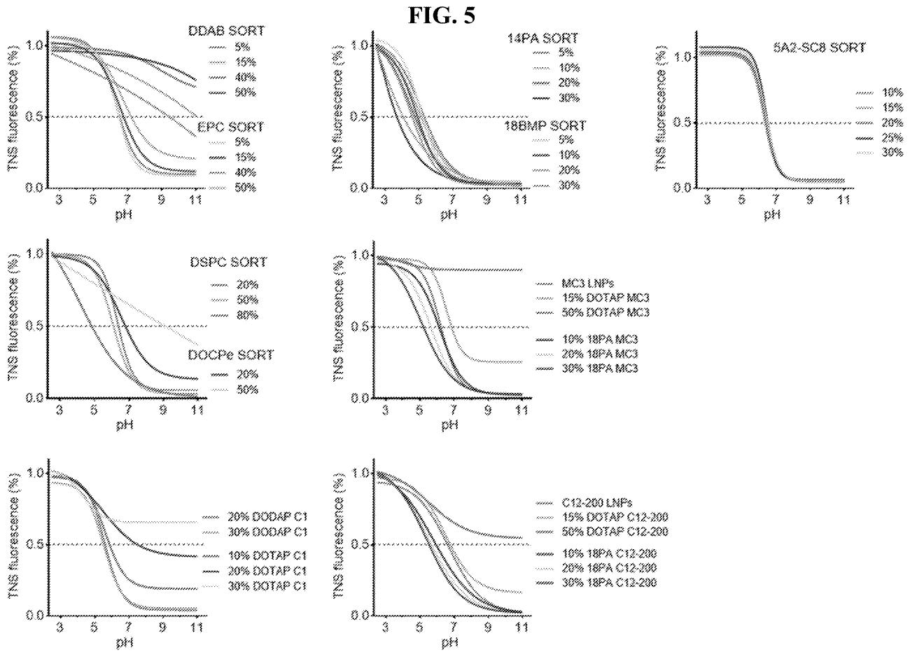

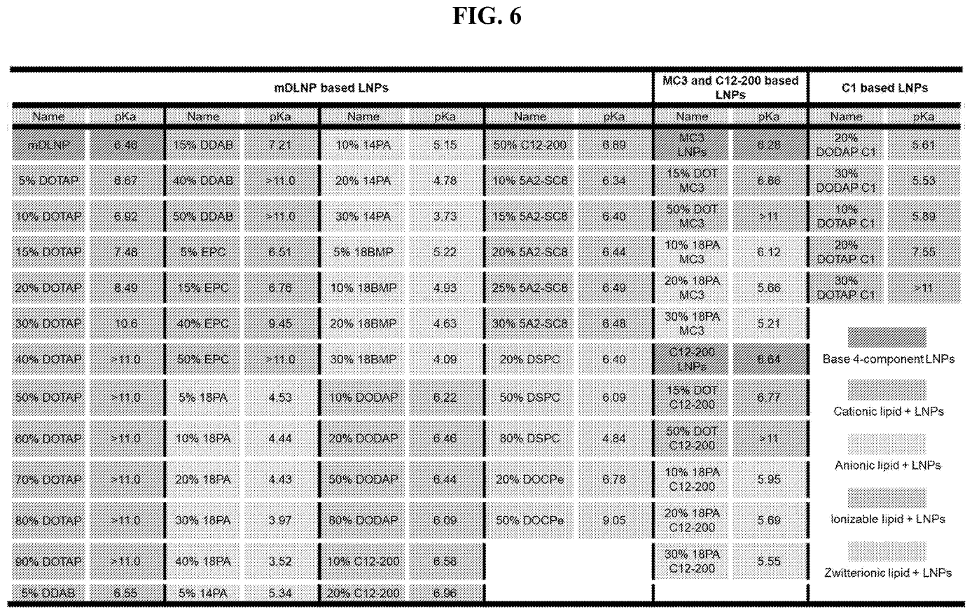

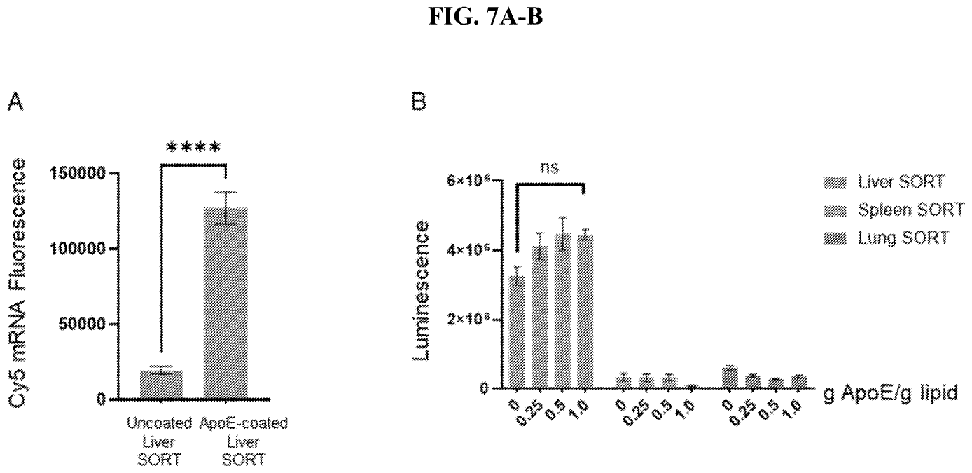

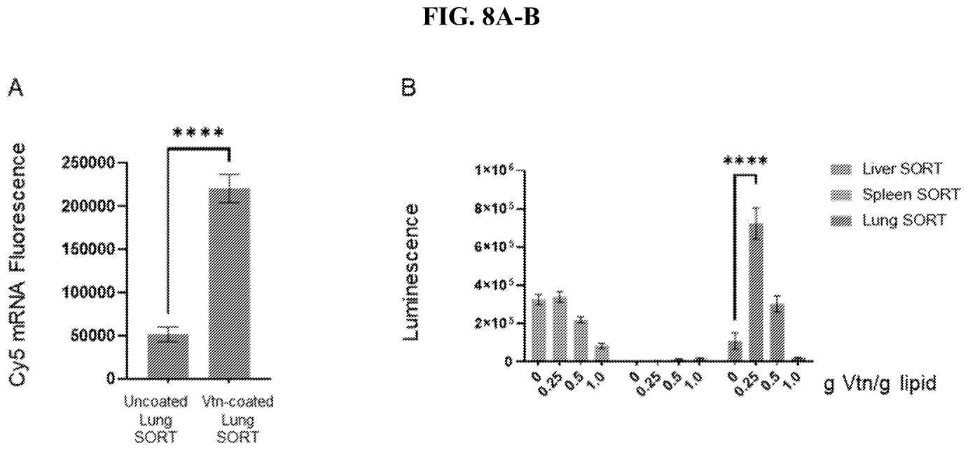

As illustrated in FIG. 1A, by adding a supplemental (e.g., 5^{th}) SORT molecule to a reference, e.g., four-component lipid composition (15:15:30:3 5A2-SC8:DOPE:Cholesterol:C14-PEG2K mol:mol), the tissue-specific activity of delivered mRNA changes based on the chemical structure of the included SORT molecule. For example, an ionizable cationic lipid (DODAP) enhances liver-specific mRNA translation (Liver SORT—15:15:30:3:15.75 5A2-SC8:DOPE:Cholesterol:C14-PEG2K:DODAP), an anionic lipid (18PA) results in spleen-specific mRNA translation (Spleen SORT-15:15:30:3:27 5A2-SC8:DOPE:Cholesterol:C14-PEG2K:18PA), and a cationic quaternary ammonium lipid (DOTAP) results in lung-specific mRNA translation (Lung SORT—15:15:30:3:63 5A2-SC8:DOPE:Cholesterol:C14-PEG2K:DOTAP).

FIG. 1B illustrates ex vivo fluorescence of Cy5-labelled mRNA in major organs extracted from C57BL/6 mice intravenously injected with example SORT LNPs (as described herein) that incorporate increasing percentages of different SORT molecules (0.5 mg/kg mRNA/body weight, 6 h).

FIG. 1C illustrates relative average Cy5 fluorescence measured in the liver, lung, and spleen as a function of SORT molecule percent inclusion (0.5 mg/kg mRNA/body weight, n=2). SORT molecules promote mRNA biodistribution to target organs. Data are shown as mean±s.e.m.

FIG. 1D illustrates representative TNS assay curves for determining the apparent pKa of example SORT LNPs (as described herein) incorporating increasing percentages of ionizable cationic, anionic, or cationic lipid SORT molecules. Apparent pKa was defined as the point at which 50% of TNS fluorescence was achieved.

As illustrated in FIG. 1E, LNPs were assigned a tissue specificity score based on the tissues in which functional luciferase mRNA was detected (liver expression=1.0, spleen expression=2.0, lung expression=3.0). For the 67 LNPs tested with the TNS assay, LNP apparent pKa was correlated with the specificity of luciferase mRNA tissue delivery.

FIGS. 2A-2H illustrate that multiple steps are involved in the mechanism of SORT LNP tissue targeting, including formation of unique protein coronas by way of example(s).

FIG. 2A illustrates a proposed three-step endogenous targeting mechanism for tissue-specific mRNA delivery by SORT LNPs, by way of example(s) only, where (1) PEG-lipid desorption (2) enables distinct serum proteins to bind SORT LNPs (3) resulting in cellular internalization in the target tissues by receptor-mediated uptake.

FIG. 2B illustrates ex vivo bioluminescence of major organs excised from C57BL/6 mice IV injected with example Liver, Spleen, and Lung SORT LNPs incorporating either sheddable PEG-lipid (C14-PEG2K) or less-sheddable PEG-lipid (C18-PEG2K) (0.1 mg FLuc mRNA/kg body weight, 6 h). Total luminescence produced by each organ is reduced when less-sheddable PEG-lipid is used, suggesting PEG-lipid desorption is a key process for efficacious mRNA delivery by the tested SORT LNPs.

FIG. 2C illustrates quantification of total luminescence produced by functional protein translated from FLuc mRNA in target organs of C57BL/6 mice IV injected with example Liver, Spleen, and Lung SORT LNPs incorporating either C14- or C18-PEG2K (0.1 mg FLuc mRNA/kg body weight, 6 h).

FIG. 2D illustrates ELISA quantification of serum hEPO in C57BL/6 mice treated with example Liver, Spleen, or Lung SORT LNPs encapsulating hEPO mRNA (0.1 mg hEPO mRNA/kg body weight, 6 h). Using a less-sheddable PEG reduces SORT LNP potency.

FIG. 2E illustrates SDS-PAGE of the serum proteins adsorbed to the surface of the reference mDLNP, an example Liver SORT LNP, an example Spleen SORT LNP, and an example Lung SORT LNP. LNPs with different organ-targeting properties bind distinct serum proteins.

FIG. 2F illustrates average abundance of proteins with distinct biological functions in the protein coronas of the reference mDLNP and an example Liver, Spleen, and Lung SORT LNPs. The choice of SORT molecule leads to large-scale differences in the functional ensemble of serum proteins which bind the LNP.

FIG. 2G illustrates isoelectric point distribution for the most enriched proteins which constitute 80% of the protein corona of the LNPs. A SORT molecule's headgroup structure influences the isoelectric point distribution of the protein corona.

FIG. 2H illustrates the top 5 most abundant serum proteins that bind different example SORT LNPs (n=3). The chemical structure of SORT molecule affects the number one serum protein that is most highly enriched on the surface of example SORT LNPs. Data are shown as mean±s.e.m. Statistical significance was determined using an unpaired two-tailed Student's t test (*, p<0.05).

FIGS. 3A-3D illustrates that distinct serum proteins regulate example SORT LNP uptake and efficacy in vitro.

As illustrated in FIG. 3A, example SORT LNPs were pre-incubated with either ApoE, β2-GPI, or Vtn prior to treating relevant cell lines to measure cellular uptake (Cy5-mRNA tracking) or functional mRNA delivery (bioluminescence).

FIG. 3B illustrates representative images of cellular uptake of uncoated and coated SORT LNPs (by way of example(s)) taken up by relevant cell types. Incubating an example SORT LNP with the protein it most avidly binds increases mRNA uptake in cell lines expressing the cognate receptor (250 ng mRNA per well, 1.5 h, scale bar=50 μm).

FIG. 3C illustrates quantification of Cy5-mRNA fluorescence in cells treated with uncoated or coated SORT LNPs (250 ng mRNA per well, 1.5 h, n=10) by way of example(s). Statistical significance was determined using an unpaired two-tailed Student's t test (****, p<0.0001, *, p<0.05).

FIG. 3D illustrates activity of functional luciferase protein translated from mRNA delivered by example SORT LNPs pre-incubated with respective proteins in relevant cell lines. (25 ng mRNA, 24 h, n=4). Statistical significance determined using one-way ANOVA with Brown-Forsythe test (****, p<0.0001,***, p<0.001, *, p<0.05). Individual proteins exclusively bind to specific SORT LNPs and enhance mRNA delivery only to cell lines expressing the cognate receptor. Data are shown as mean±s.e.m.

FIGS. 4A-4C illustrate that extrahepatic mRNA delivery occurs via an ApoE-independent mechanism.

FIG. 4A illustrates ex vivo bioluminescence produced by functional protein translated from FLuc mRNA in major organs excised from wild-type C57BL/6 mice IV injected with the reference mDLNP or an example Liver, Spleen or Lung SORT LNPs (0.1 mg/kg FLuc mRNA, 6 h). The role of ApoE on the SORT LNP efficacy varies based on the chemical structure of the included SORT molecule

FIG. 4B illustrates ex vivo bioluminescence produced by functional protein translated from FLuc mRNA in major organs excised from ApoE−/− mice IV injected with reference mDLNP or an example Liver, Spleen or Lung SORT LNPs (0.1 mg/kg FLuc mRNA, 6 h).

FIG. 4C illustrates quantification of total bioluminescence produced by target organs excised from wild-type and ApoE−/− mice treated with reference mDLNP or, an example Liver, Spleen, or Lung SORT LNPs. (0.1 mg/kg FLuc mRNA, 6 h, n=3). Data are shown as mean±s.e.m. Statistical significance was determined using an unpaired two-tailed Student's t test (**, p<0.01, *, p<0.05, ns, p>0.05). Elimination of ApoE from the serum using genetic knockout results in a marked reduction of hepatic mRNA delivery by reference mDLNP and example Liver SORT LNPs. In contrast, example Spleen SORT LNPs have enhanced spleen-targeting when ApoE is depleted from the serum while the efficacy of example Lung SORT LNPs is unaffected by ApoE elimination.

FIG. 5 illustrate TNS fluorescence curves for example SORT LNPs formulated with various mass fractions of cationic, anionic, ionizable, and zwitterionic lipids. The apparent pKa of an LNP is computed as the pH at which 50% of the TNS fluorescence is measured.

FIG. 6 illustrates apparent pKa values computed from example SORT LNPs incorporating cationic, anionic, ionizable, and zwitterionic lipids at various mass fractions.

FIGS. 7A-7B illustrate cellular uptake and functional mRNA delivery to low-density lipoprotein receptor (LDL-R) expressing Hep G2 cells by example SORT LNPs pre-incubated with ApoE.

FIG. 7A illustrates quantification of Cy5-mRNA fluorescence in Hep G2 cells treated with either uncoated or ApoE-coated Liver SORT LNPs (250 ng mRNA per well, 1.5 h, n=10) by way of example(s). Statistical significance was determined using an unpaired two-tailed Student's t test (****, p<0.0001).

FIG. 7B illustrates activity of functional luciferase protein translated from mRNA delivered by example SORT LNPs pre-incubated with increasing amounts of ApoE. (25 ng mRNA, 24 h, n=4). Statistical significance determined using one-way ANOVA with Brown-Forsythe test (ns, p>0.05).

FIGS. 8A-8B illustrate cellular uptake and functional mRNA delivery to αvβ3 expressing U87-MG cells by example SORT LNPs pre-incubated with Vtn.

FIG. 8A illustrates quantification of Cy5-mRNA fluorescence in U87-MG cells treated with either uncoated or Vtn-coated Lung SORT LNPs (250 ng mRNA per well, 1.5 h, n=10) by way of example(s). Statistical significance was determined using an unpaired two-tailed Student's t test (****, p<0.0001).

FIG. 8B illustrates activity of functional luciferase protein translated from mRNA delivered by example SORT LNPs pre-incubated with increasing amounts of Vtn. (25 ng mRNA, 24 h, n=4). Statistical significance determined using one-way ANOVA with Brown-Forsythe test (****, p<0.0001).

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “N-terminal end sequence,” as used herein with respect to a polypeptide or polynucleotide sequence of interest, generally means that no other amino acid or nucleotide residues precede the N-terminal end sequence in the polypeptide or polynucleotide sequence of interest at the N-terminal end. The term “C-terminal end sequence,” as used herein with respect to a polypeptide or polynucleotide sequence of interest, generally means that no other amino acid or nucleotide residues follows the C-terminal end sequence in the polypeptide or polynucleotide sequence of interest at the C-terminal end.

The terms “non-naturally occurring” and “non-natural” are used interchangeably herein. The term “non-naturally occurring” or “non-natural,” as used herein with respect to a therapeutic agent or prophylactic agent, generally means that the agent is not biologically derived in mammals (including but not limited to human). The term “non-naturally occurring” or “non-natural,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “equivalent molar dose” generally means that the amounts of materials administered to a subject have an equivalent amount of moles, based on the molecular weight of the material used in the dose.

The term “therapeutically effective and non-toxic dose,” as used herein, generally refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The terms “cancer” and “cancerous” refer to or describe the physiological condition in mammals that is typically characterized by unregulated cell growth/proliferation.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

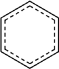

includes

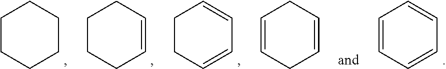

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

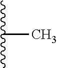

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

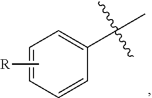

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

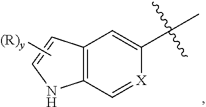

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl(C₂₋₁₀₎” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C₁. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

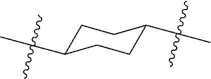 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

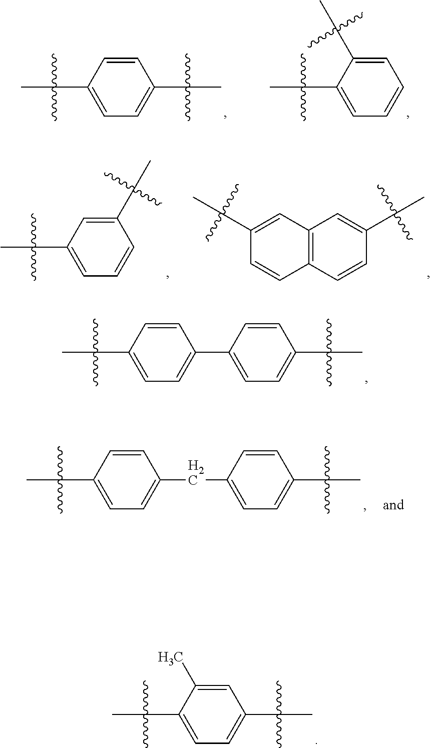

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

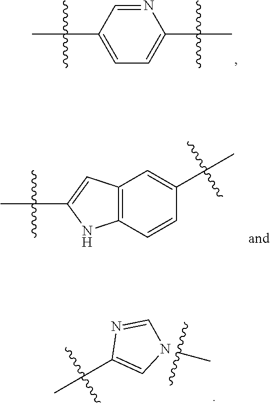

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

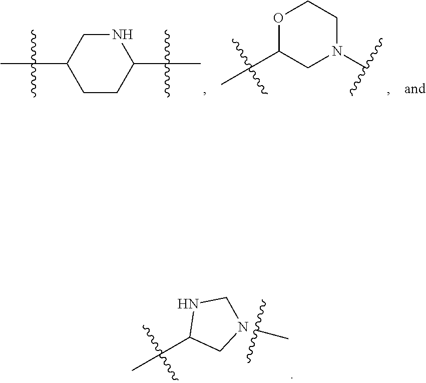

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

Throughout this application, the term “about” is used to indicate that a value includes the inherent variation of error for the device, the method being employed to determine the value, or the variation that exists among the study subjects.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Example of lipid compositions include suspensions, emulsions, and vesicular compositions.

As used herein, the term “detectable” refers to an occurrence of, or a change in, a signal that is directly or indirectly detectable either by observation or by instrumentation. Typically, a detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density

The term “potent” or “potency,” as used herein in connection with delivery of therapeutic agent(s), generally refers to a greater ability of a delivery system (e.g., a lipid composition) to achieve or bring about a desired amount, activity, or effect of a therapeutic agent or prophylactic agent (such as a desired level of translation, transcription, production, expression, or activity of a protein or gene) in cells (e.g., targeted cells) to any measurable extent, e.g., relative to a reference delivery system. For example, a lipid composition with a higher potency may achieve a desired therapeutic effect in a greater population of relevant cells, within a shorter response time, or that last a longer period of time.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer or dendron, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2^{a}, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### Compositions

### Lipid Compositions

In some embodiments, provided herein is a lipid composition comprising: an ionizable cationic lipid; a polymer-conjugated lipid; and a selective organ targeting (SORT) lipid, e.g., separate from the ionizable cationic lipid. The lipid composition may further comprise a phospholipid.

### Ionizable Cationic Lipids

In some embodiments of the lipid composition of the present application, the lipid composition comprises an ionizable cationic lipid. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some embodiments of the lipid composition of the present application, the ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g. molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments of the lipid composition of the present disclosure, the dendrimers or dendrons are assembled using the differential reactivity of the acrylate and methacrylate groups with amines and thiols. The dendrimers or dendrons may include secondary or tertiary amines and thioethers formed by the reaction of an acrylate group with a primary or secondary amine and a methacrylate with a mercapto group. Additionally, the repeating units of the dendrimers or dendrons may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer or dendron may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments of the lipid composition of the present application, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups.

### Dendrimers of Formula (I)

In some embodiments of the lipid composition, the ionizable cationic lipid comprises at least two C₈-C₂₄ alkyl groups. In some embodiments, the ionizable cationic lipid is a dendrimer or dendron further defined by the formula:

Core-Repeating Unit-Terminating Group (D-I)
wherein the core is linked to the repeating unit by removing one or more hydrogen atoms from the core and replacing the atom with the repeating unit and wherein:
   the core has the formula:

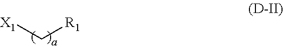

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

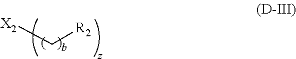

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

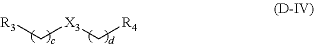

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

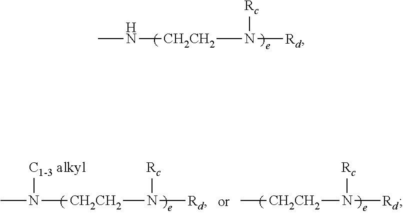

wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≥6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

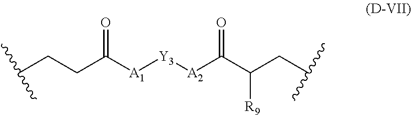

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

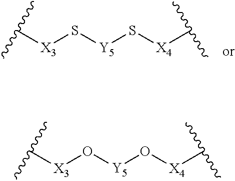

wherein:
X₃ and X₄ are alkanediyl_{(C≤12)}, alkenediyl_{(C≤12)}, arenediyl_{(C≤12)}, or a substituted version of any of these groups;
Y₅ is a covalent bond, alkanediyl_{(C≤12)}, alkenediyl_{(C≤12)}, arenediyl_{(C≤12)}, or a substituted version of any of these groups; and
R₉ is alkyl_{(C≤8)} or substituted alkyl_{(C≤8)};
the linker group has the formula:
wherein:

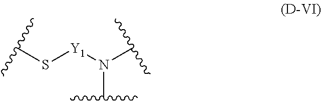

Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

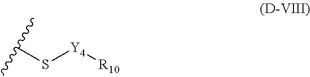

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkylamino_{(C≤12)}, —C(O)-dialkylamino_{(C≤12)}, —C(O)—N-heterocycloalkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
or a pharmaceutically acceptable salt thereof. In some embodiments, the terminating group is further defined by the formula:

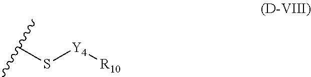

wherein:
   Y₄ is alkanediyl_{(C≤18)}; and
   R₁₀ is hydrogen. In some embodiments, A₁ and A₂ are each independently —O— or —NRₐ—.

In some embodiments of the dendrimer or dendron of formula (D-I), the core is further defined by the formula:

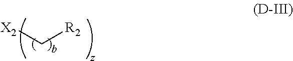

wherein:
   X₂ is N(R₅)_{y};
      R₅ is hydrogen or alkyl_{(C≤8)}, or substituted alkyl_{(C≤18)}; and
      y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3.

In some embodiments of the dendrimer or dendron of formula (D-I), the core is further defined by the formula:

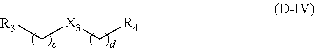

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

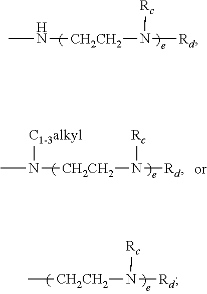

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments of the dendrimer or dendron of formula (I), the terminating group is represented by the formula:

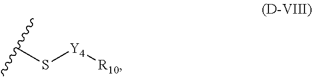

wherein:
Y₄ is alkanediyl_{(C≤18)}; and
R₁₀ is hydrogen.

In some embodiments of the dendrimer or dendron of formula (D-I), the core is further defined as:

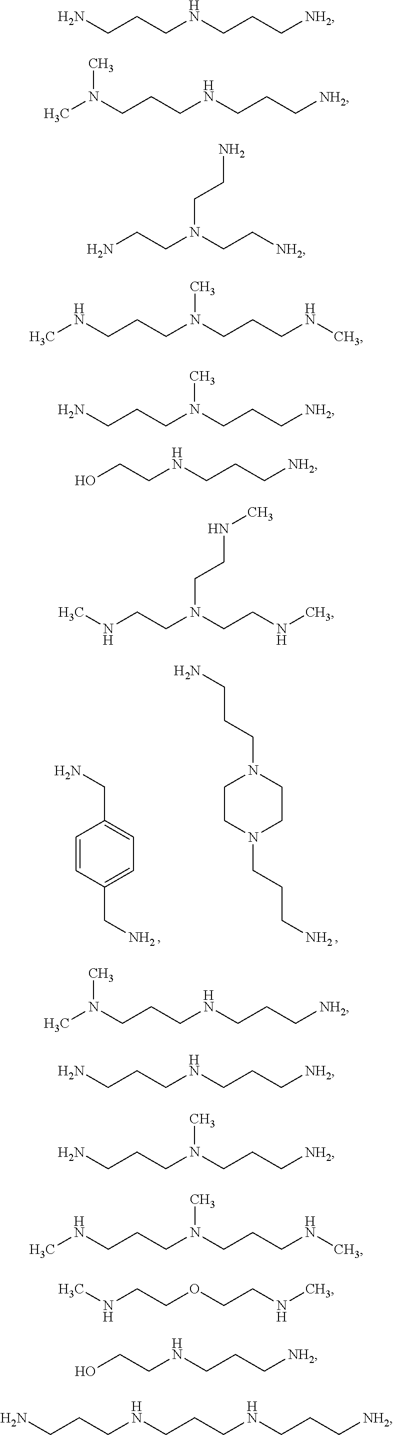

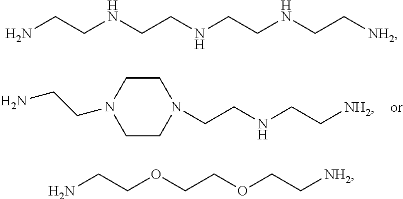

In some embodiments of the dendrimer or dendron of formula (D-I), the degradable diacyl is further defined as:

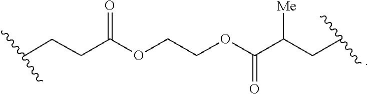 [SMILES: *CCC(=O)OCCOC(=O)C(C)C*]

In some embodiments of the dendrimer or dendron of formula (D-I), the linker is further defined as

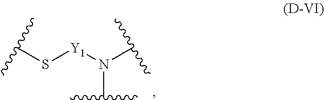

wherein Y₁ is alkanediyl_{(C≤8)} or substituted alkanediyl_{(C≤8)}.

In some embodiments of the dendrimer or dendron of formula (D-I), the dendrimer or dendron is selected from the group consisting of:

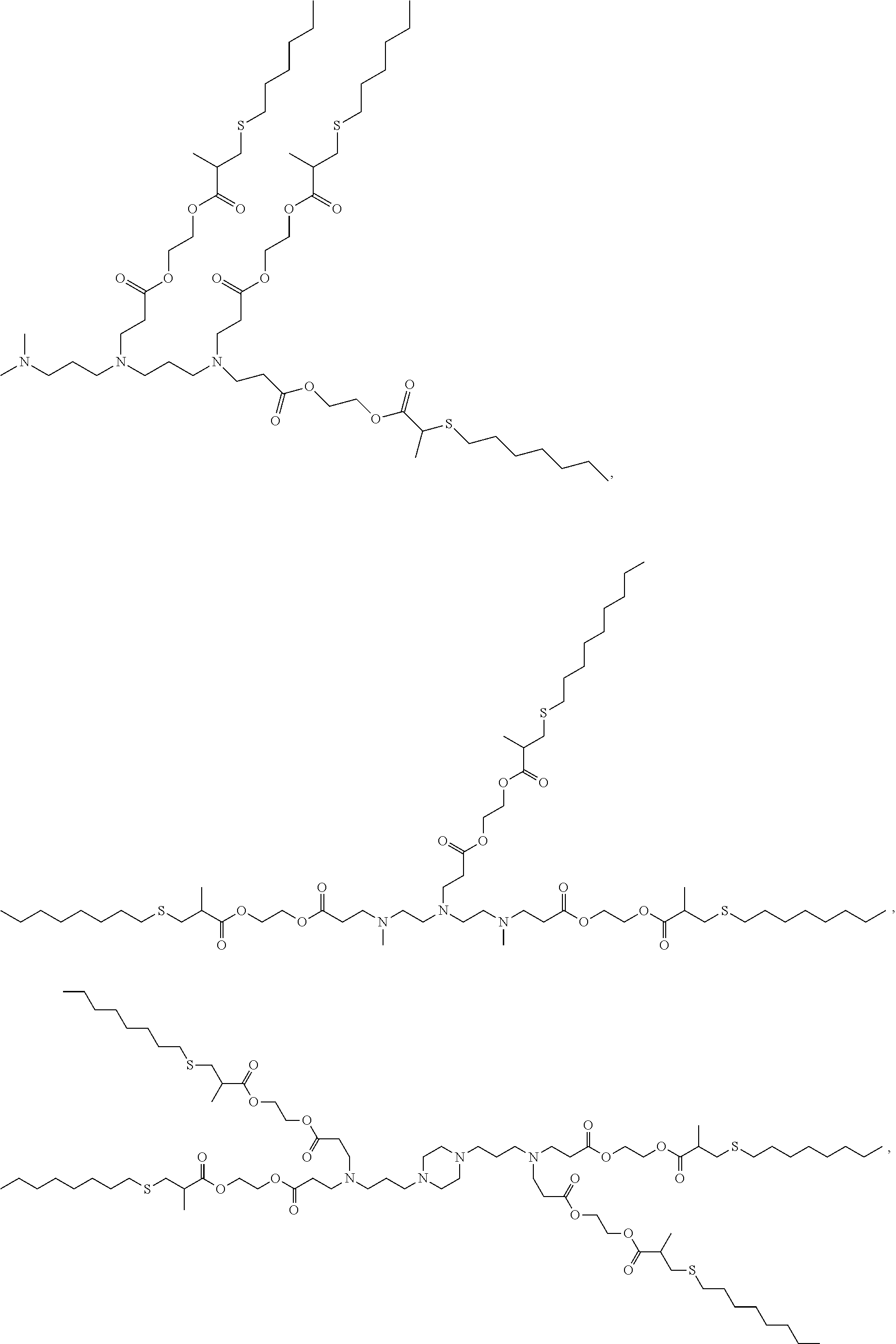

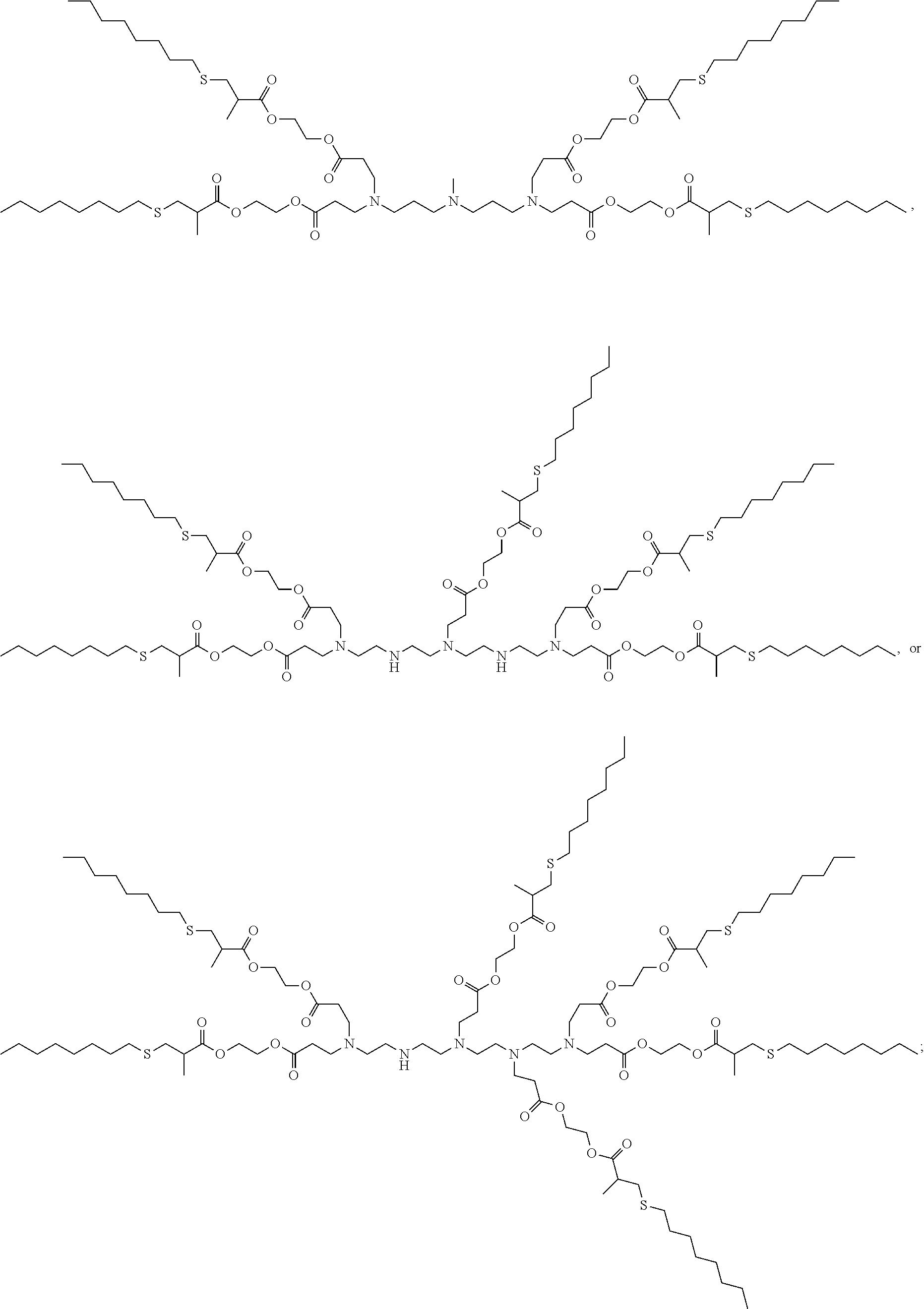

and pharmaceutically acceptable salts thereof.

### Dendrimers or Dendrons of Formula (X)

In some embodiments of the lipid composition, the ionizable cationic lipid is a dendrimer or dendron of the formula Core [symbol] Branch)_{N}. In some embodiments, the ionizable cationic lipid is a dendrimer or dendron of the formula

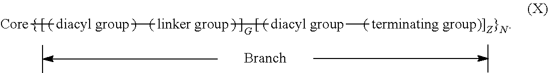

In some embodiments of the lipid composition, the ionizable cationic lipid is a dendrimer or dendron of a generation (g) having a structural formula:

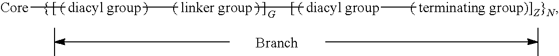

or a pharmaceutically acceptable salt thereof, wherein:
(a) the core comprises a structural formula (X_{core}):

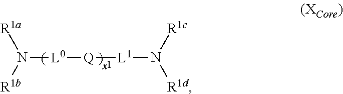

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

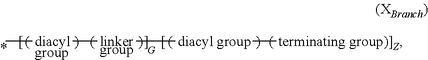

wherein:
   * indicates a point of attachment of the branch to the core;
   g is 1, 2, 3, or 4;
   Z=2^{(g-1)};
   G=0, when g=1; or

$$G = {\sum }_{i = 0}^{i = g - 2} {2}^{i} ,$$

when g≠1;
(c) each diacyl group independently comprises a structural formula

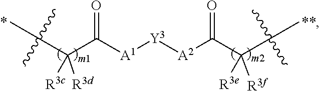

wherein
   * indicates a point of attachment of the diacyl group at the proximal end thereof;
   ** indicates a point of attachment of the diacyl group at the distal end thereof;
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—
      wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula,

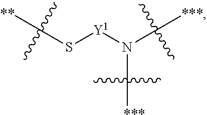

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R_{1g} or -L²-NR^{1e}R^{1f}. In some embodiments of X_{Core} Q is independently at each occurrence a —CR^{3a}R^{3b} R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene][(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene][(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene](e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

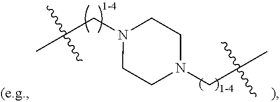

and [(C₁-C₄) alkylene]-phenylene-[(C₁-C₄) alkylene]

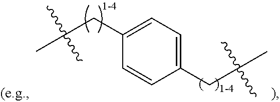

In some embodiments of X_{Core}, L⁰, L¹, and L² are each independently at each occurrence selected from C₁-C₆ alkylene (e.g., C₁-C₃ alkylene), —(C₁-C₃ alkylene-0)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene](e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene](e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core}, x¹ is 2. In some embodiments of X_{Core}, x¹ is 0, 3. In some embodiments of X_{Core} x¹ is 4. In some embodiments of X_{Core}. x¹ is 5. In some embodiments of X_{Core}, x¹ is 6.

In some embodiments of X_{Core}, the core comprises a structural formula:

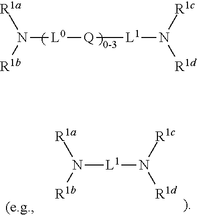

In some embodiments of X_{Core}, the core comprises a structural formula:

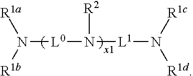

In some embodiments of X_{Core}, the core comprises a structural formula:

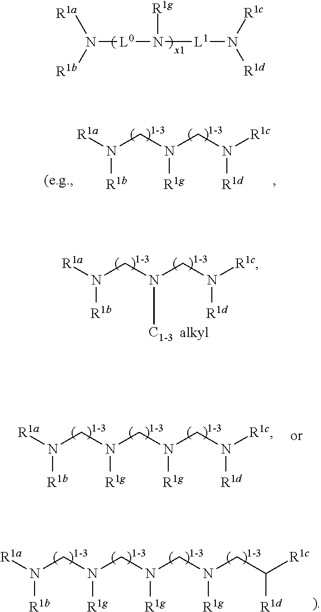

In some embodiments of X_{Core}, the core comprises a structural formula:

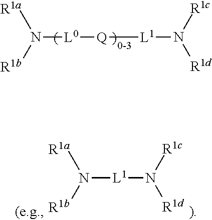

In some embodiments of X_{Core}, the core comprises a structural formula:

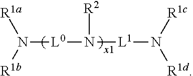

In some embodiments of X_{Core}, the core comprises a structural formula:

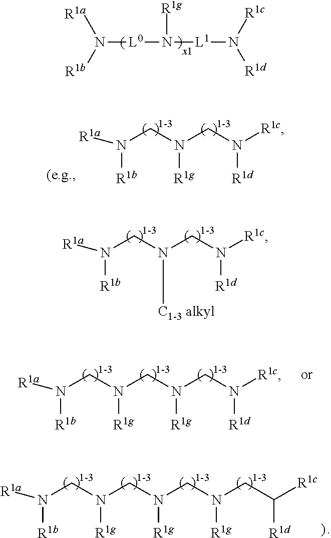

In some embodiments of X_{Core}, the core comprises a structural formula

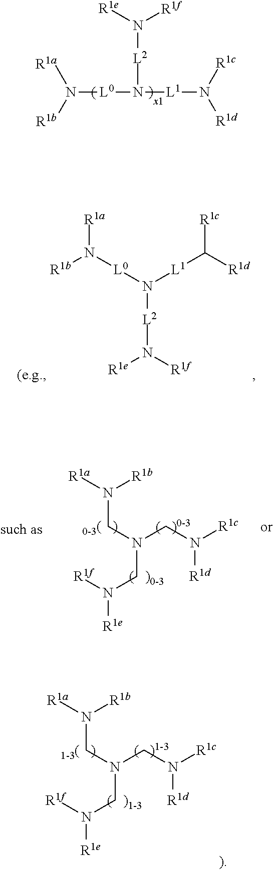

In some embodiments of X_{Core}, the core comprises a structural formula:

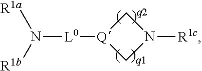

wherein Q′ is —NR²— or —CR^{3a}R^{3b}—; q¹ and q² are each independently 1 or 2. In some embodiments of X_{Core}, the core comprises a structural formula:

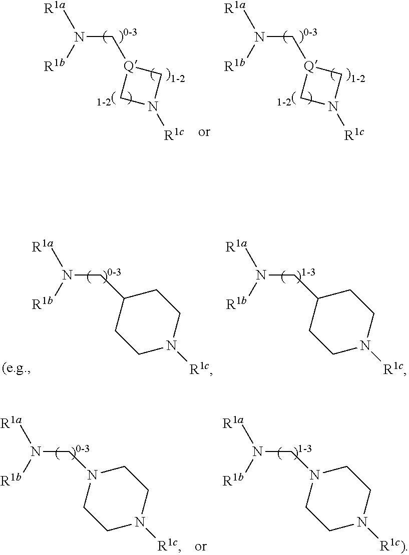

In some embodiments of X_{Core}, the core comprises a structural formula

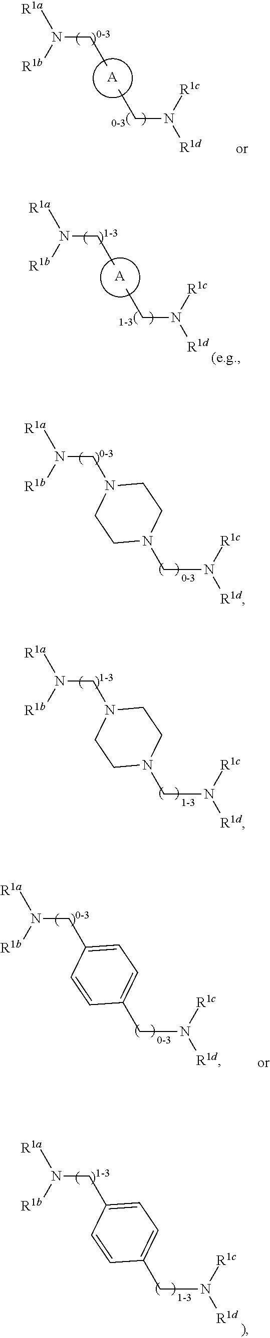

wherein ring A is an optionally substituted aryl or an optionally substituted (e.g., C₃-C₁₂, such as C₃-C₅) heteroaryl. In some embodiments of X_{Core}, the core comprises has a structural formula

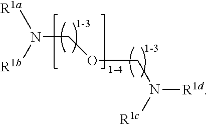

In some embodiments of X_{Core}, the core comprises a structural formula set forth in Table. 1 and pharmaceutically acceptable salts thereof, wherein * indicates a point of attachment of the core to a branch of the plurality of branches. In some embodiments, the example cores of Table. 1 are not limited to the stereoisomers (i.e. enantiomers, diastereomers) listed.

TABLE 1
Example core structures

| ID # | Structure |
|---|---|
| 1A1 | 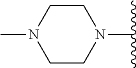 [SMILES: *N1CCN(C)CC1] |
| 1A2 | 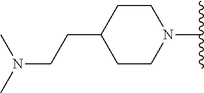 [SMILES: *N1CCC(CCN(C)C)CC1] |
| 1A3 | 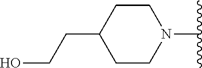 [SMILES: *N1CCC(CCO)CC1] |
| 1A4 | 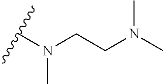 [SMILES: *N(C)CCN(C)C] |
| 1A5 | 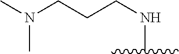 [SMILES: *NCCCN(C)C] |
| 2A1 | 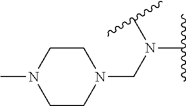 [SMILES: *N(*)CN1CCN(C)CC1] |
| 2A2 | 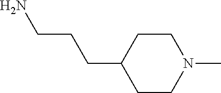 |
| 2A3 | 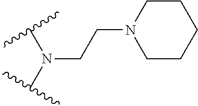 [SMILES: *N(*)CCN1CCCCC1] |
| 2A4 | 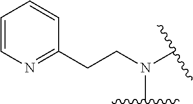 [SMILES: *N(*)CCc1ccccn1] |
| 2A5 | 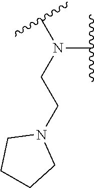 [SMILES: *N(*)CCN1CCCC1] |
| 2A6 | 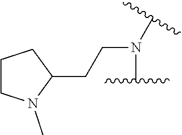 [SMILES: *N(*)CCC1CCCN1C] |
| 2A7 | 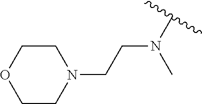 [SMILES: *N(C)CCN1CCOCC1] |
| 2A8 | 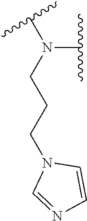 [SMILES: *N(*)CCCn1ccnc1] |
| 2A9 | 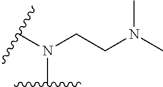 [SMILES: *N(*)CCN(C)C] |
| 2A10 | 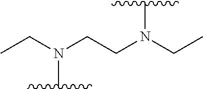 [SMILES: *N(CC)CCN(*)CC] |
| 2A11 | 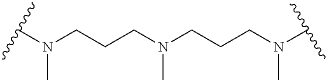 [SMILES: *N(C)CCCN(C)CCCN(*)C] |
| 2A12 | 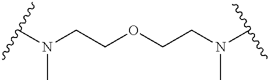 [SMILES: *N(C)CCOCCN(*)C] |
| 3A1 | 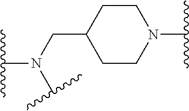 [SMILES: *N(*)CC1CCN(*)CC1] |
| 3A2 | 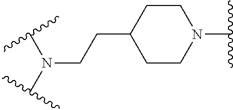 [SMILES: *N(*)CCC1CCN(*)CC1] |
| 3A3 | 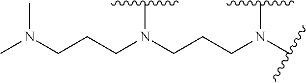 [SMILES: *N(*)CCCN(*)CCCN(C)C] |
| 3A4 | 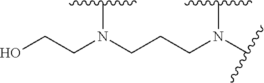 [SMILES: *N(*)CCCN(*)CCO] |
| 3A5 | 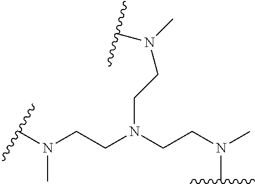 [SMILES: *N(C)CCN(CCN(*)C)CCN(*)C] |
| 4A1 | 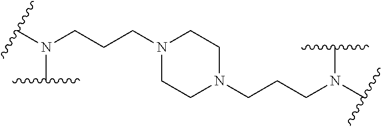 [SMILES: *N(*)CCCN1CCN(CCCN(*)*)CC1] |
| 4A2 | 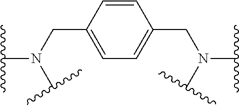 [SMILES: *N(*)Cc1ccc(CN(*)*)cc1] |
| 4A3 | 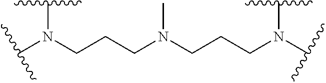 [SMILES: *N(*)CCCN(C)CCCN(*)*] |
| 4A4 | 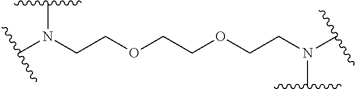 [SMILES: *N(*)CCOCCOCCN(*)*] |
| 5A1 | 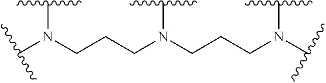 [SMILES: *N(*)CCCN(*)CCCN(*)*] |
| 5A2 | 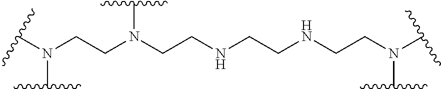 [SMILES: *N(*)CCNCCNCCN(*)CCN(*)*] |
| 5A3 | 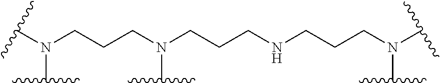 [SMILES: *N(*)CCCNCCCN(*)CCCN(*)*] |
| 5A4 | 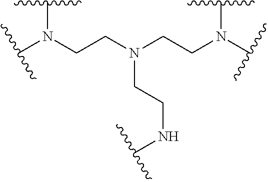 [SMILES: *NCCN(CCN(*)*)CCN(*)*] |
| 5A5 | 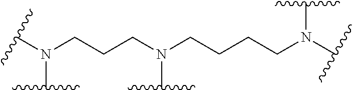 [SMILES: *N(*)CCCCN(*)CCCN(*)*] |
| 6A1 | 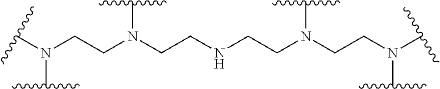 [SMILES: *N(*)CCN(*)CCNCCN(*)CCN(*)*] |
| 6A2 | 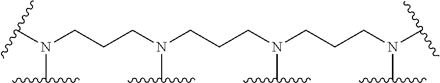 [SMILES: *N(*)CCCN(*)CCCN(*)CCCN(*)*] |
| 6A3 | 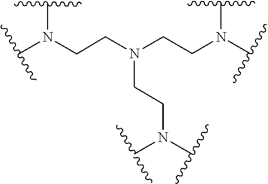 [SMILES: *N(*)CCN(CCN(*)*)CCN(*)*] |
| 6A4 | 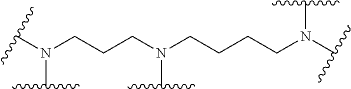 [SMILES: *N(*)CCCCN(*)CCCN(*)*] |
| 1H1 | 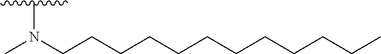 [SMILES: *N(C)CCCCCCCCCCCC] |
| 1H2 | 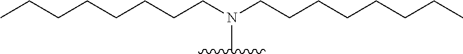 [SMILES: *N(CCCCCCCC)CCCCCCCC] |
| 1H3 | 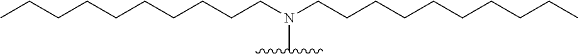 |
| 2H1 | 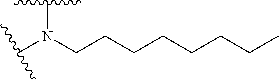 [SMILES: *N(*)CCCCCCCC] |
| 2H2 | 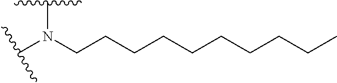 [SMILES: *N(*)CCCCCCCCCC] |
| 2H3 | 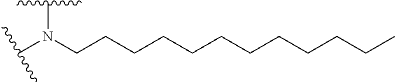 [SMILES: *N(*)CCCCCCCCCCCC] |
| 2H4 | 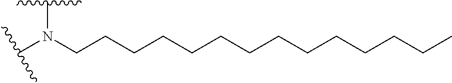 [SMILES: *N(*)CCCCCCCCCCCCCC] |
| 2H5 | 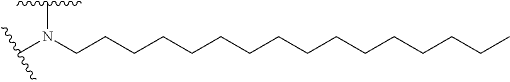 [SMILES: *N(*)CCCCCCCCCCCCCCCC] |
| 2H6 | 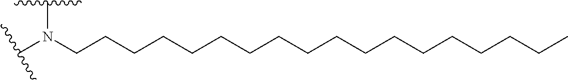 [SMILES: *N(*)CCCCCCCCCCCCCCCCCC] |

In some embodiments of X_{Core}, the core comprises a structural formula selected from the group consisting of:

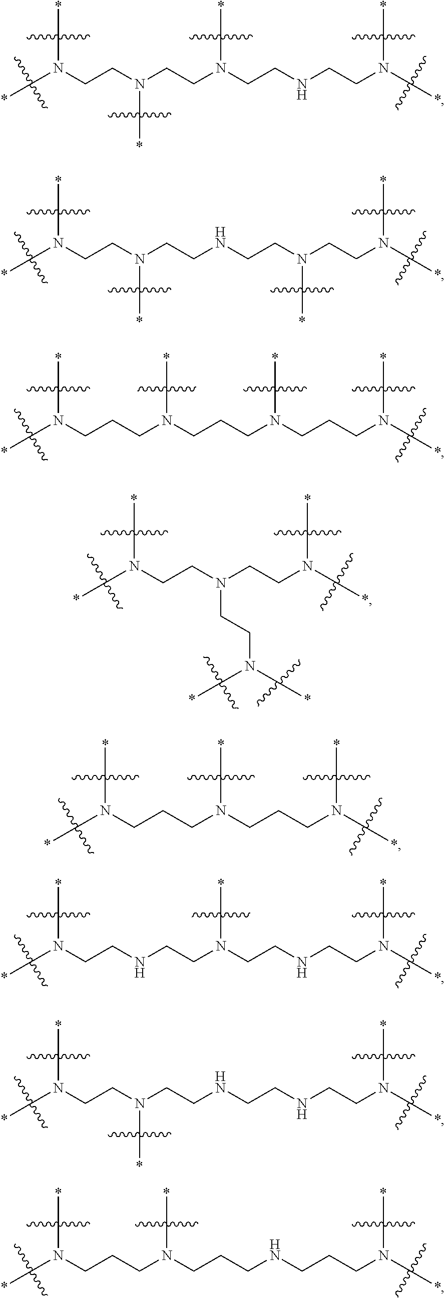

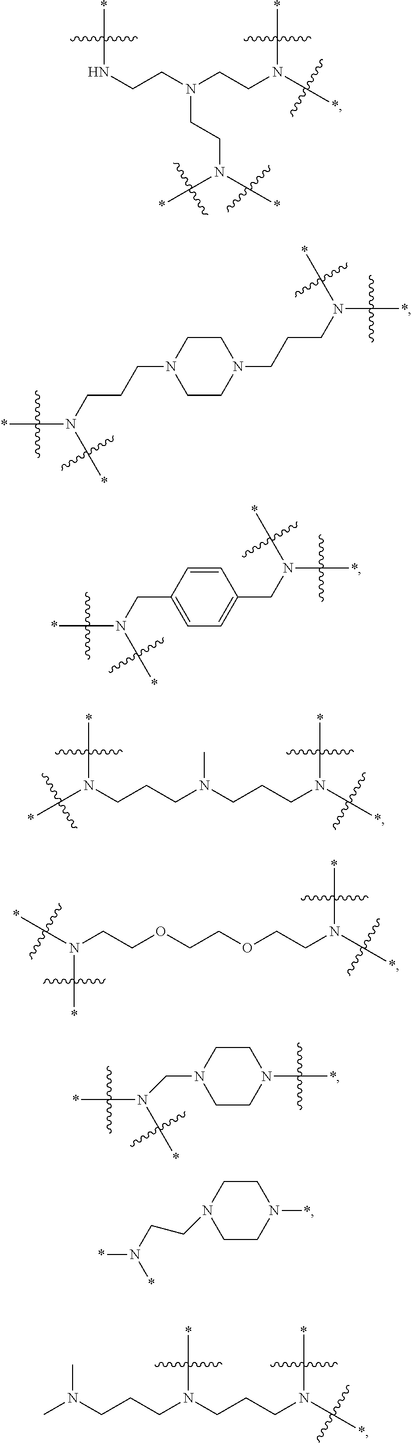

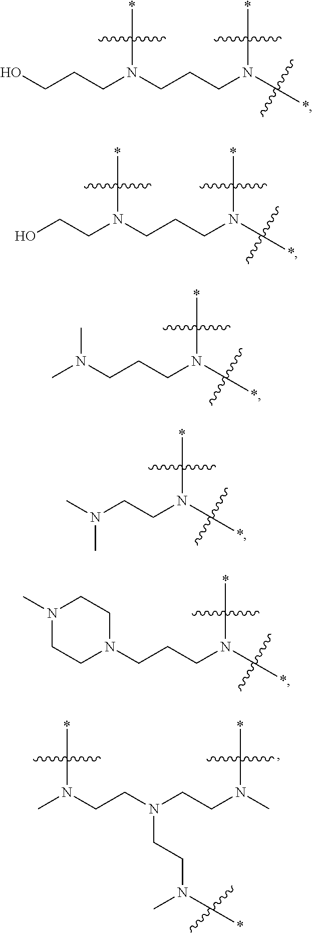

and pharmaceutically acceptable salts thereof, wherein * indicates a point of attachment of the core to a branch of the plurality of branches.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=₂^{(g-1)} and when g=1, G=0. In some embodiments of X_{Branch}, Z=2^{(g-1)} and

$$G = {\sum }_{i = 0}^{i = g - 2} {2}^{i} ,$$

when g≠1.

In some embodiments of X_{Branch}, g=1, G=0, Z=1, and each branch of the plurality of branches comprises a structural formula each branch of the plurality of branches comprises a structural formula

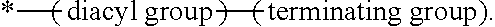

In some embodiments of X_{Branch}, g=2, G=1, Z=2, and each branch of the plurality of branches comprises a structural formula

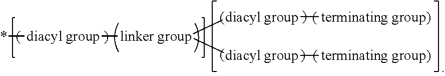

In some embodiments of X_{Branch}, g=3, G=3, Z=4, and each branch of the plurality of branches comprises a structural formula

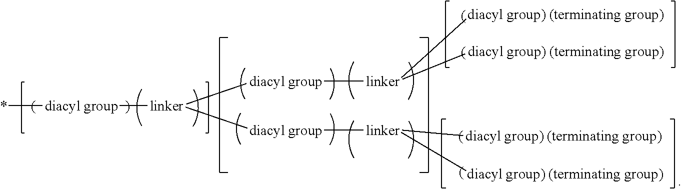

In some embodiments of X_{Branch}, g=4, G=7, Z=8, and each branch of the plurality of branches comprises a structural formula

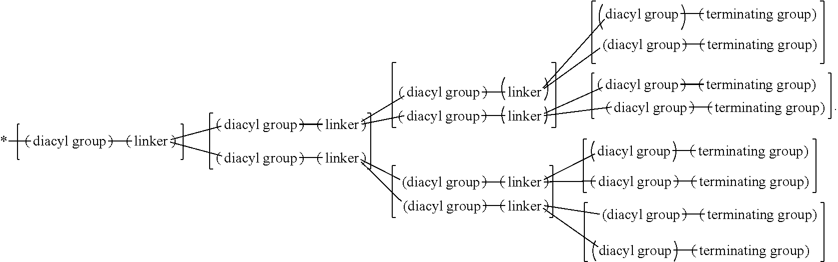

In some embodiments, the dendrimers or dendrons described herein with a generation (g)=1 has the structure:

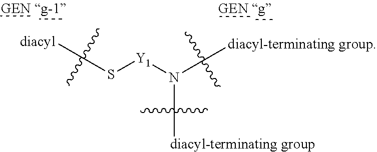

In some embodiments, the dendrimers or dendrons described herein with a generation (g)=1 has the structure:

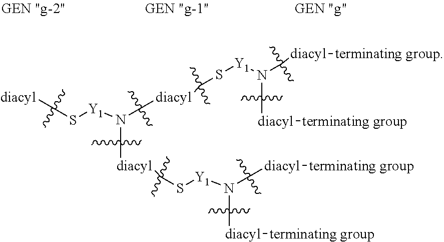

Example formulation of the dendrimers or dendrons described herein for generations 1-4 is shown in Table 2. The number of diacyl groups, linker groups, and terminating groups can be calculated based on g.

TABLE 2
Formulation of Dendrimer or Dendron Groups Based on Generation (g)

| | g = 1 | g = 2 | g = 3 | g = 4 | |
|---|---|---|---|---|---|
| # of diacyl grp | 1 | 1 + 2 = 3 | 1 + 2 + 2² = 7 | 1 + 2 + 2² + 2³ = 15 | 1 + 2 + . . . + 2^{g-1} |
| # of linker grp | 0 | 1 | 1 + 2 | 1 + 2 + 2² | 1 + 2 + . . . + 2^{g-2} |
| # of terminating | 1 | 2 | 2² | 2³ | 2^{(g-1)} |
| grp | | | | | |

In some embodiments, the diacyl group independently comprises a structural formula

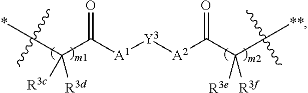

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

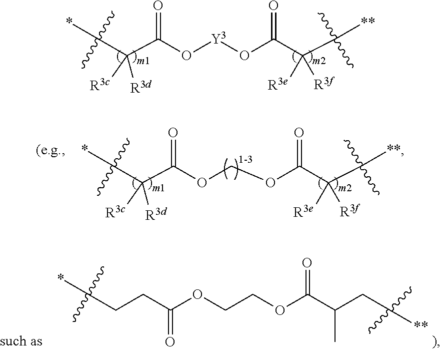

and optionally R^{3c}, R^{3d}, R^{3e}, and R^{3f} are each independently at each occurrence hydrogen or C₁-C₃ alkyl.

In some embodiments, linker group independently comprises a structural formula

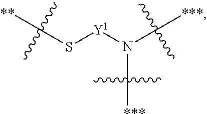

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

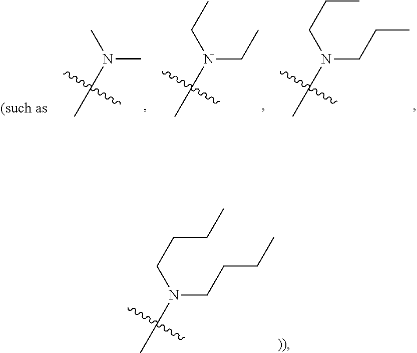

C₄-C₆ N-heterocycloalkyl

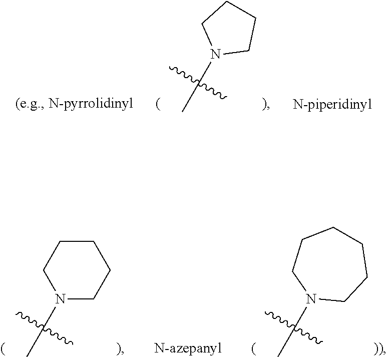 [SMILES: *N1CCCC1 ; *N1CCCCC1 ; *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

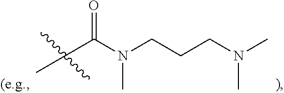 [SMILES: *C(=O)N(C)CCCN(C)C]

—C(O)N(C₁-C₃ alkyl)-(C₁-C₆ alkylene)-(C₄-C₆ N-heterocycloalkyl)

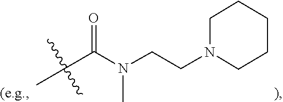 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

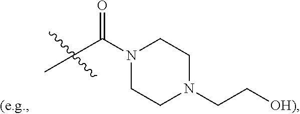

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

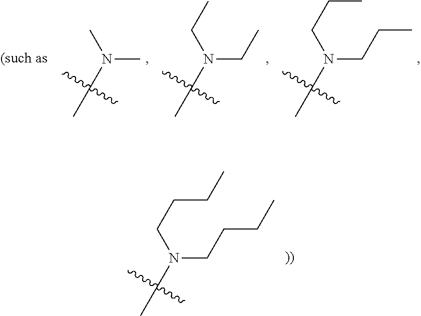

and C₄-C₆ N-heterocycloalkyl

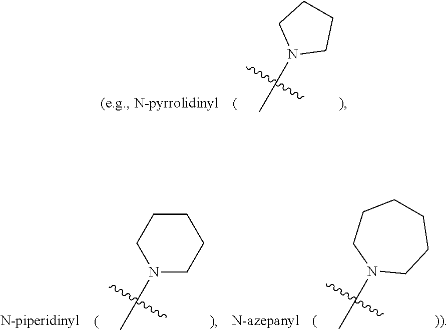 [SMILES: *N1CCCC1 ; *N1CCCCC1 ; *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table 3. In some embodiments, the dendrimers or dendrons described herein can comprise a terminating group or pharmaceutically acceptable salt, or thereof selected in Table 3. In some embodiments, the example terminating group of Table 3 are not limited to the stereoisomers (i.e. enantiomers, diastereomers) listed.

TABLE 3
Example terminating group/peripheries structures

| ID # | Structure |
|---|---|
| SC1 | 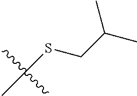 [SMILES: *SCC(C)C] |
| SC2 | 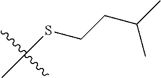 [SMILES: *SCCC(C)C] |
| SC3 | 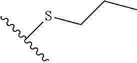 [SMILES: *SCCC] |
| SC4 | 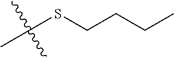 [SMILES: *SCCCC] |
| SC5 | 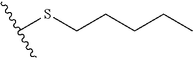 [SMILES: *SCCCCC] |
| SC6 | 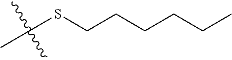 [SMILES: *SCCCCCC] |
| SC7 | 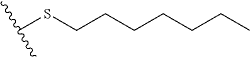 [SMILES: *SCCCCCCC] |
| SC8 | 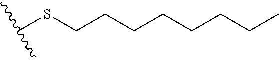 [SMILES: *SCCCCCCCC] |
| SC9 | 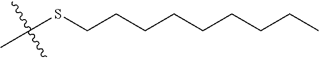 [SMILES: *SCCCCCCCCC] |
| SC10 | 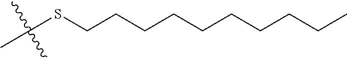 [SMILES: *SCCCCCCCCCC] |
| SC11 | 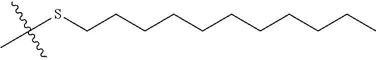 [SMILES: *SCCCCCCCCCCC] |
| SC12 | 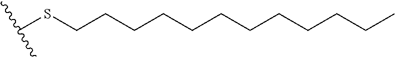 [SMILES: *SCCCCCCCCCCCC] |
| SC14 | 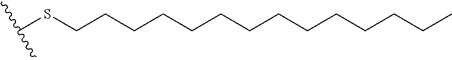 [SMILES: *SCCCCCCCCCCCCCC] |
| SC16 | 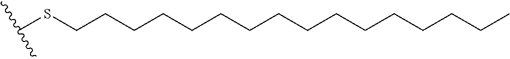 [SMILES: *SCCCCCCCCCCCCCCCC] |
| SC18 | 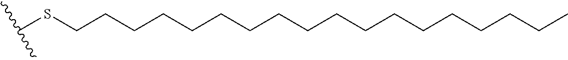 [SMILES: *SCCCCCCCCCCCCCCCCCC] |
| SC19 | 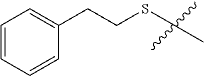 [SMILES: *SCCc1ccccc1] |
| SO1 | 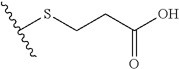 [SMILES: *SCCC(=O)O] |
| SO2 | 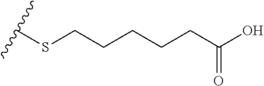 [SMILES: *SCCCCCC(=O)O] |
| SO3 | 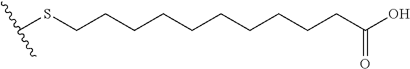 [SMILES: *SCCCCCCCCCCC(=O)O] |
| SO4 | 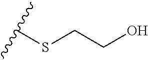 [SMILES: *SCCO] |
| SO5 | 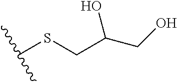 [SMILES: *SCC(O)CO] |
| SO6 | 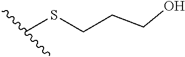 [SMILES: *SCCCO] |
| SO7 | 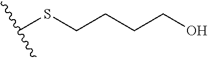 [SMILES: *SCCCCO] |
| SO8 | 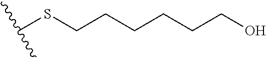 [SMILES: *SCCCCCCO] |
| SO9 | 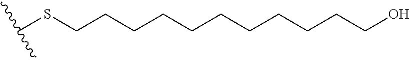 [SMILES: *SCCCCCCCCCCCO] |
| SN1 | 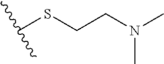 [SMILES: *SCCN(C)C] |
| SN2 | 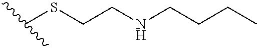 [SMILES: *SCCNCCCC] |
| SN3 | 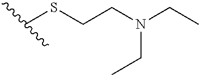 [SMILES: *SCCN(CC)CC] |
| SN4 | 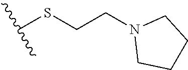 [SMILES: *SCCN1CCCC1] |
| SN5 | 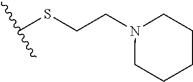 [SMILES: *SCCN1CCCCC1] |
| SN6 | 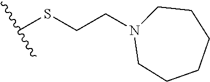 [SMILES: *SCCN1CCCCCC1] |
| SN7 | 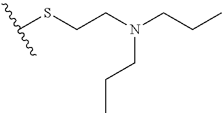 [SMILES: *SCCN(CCC)CCC] |
| SN8 | 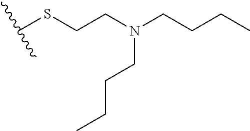 [SMILES: *SCCN(CCCC)CCCC] |
| SN9 | 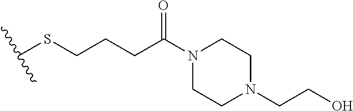 [SMILES: *SCCCC(=O)N1CCN(CCO)CC1] |
| SN10 | 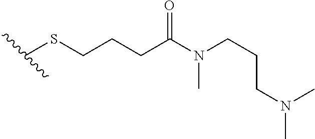 [SMILES: *SCCCC(=O)N(C)CCCN(C)C] |
| SN11 | 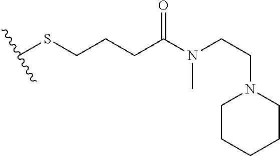 [SMILES: *SCCCC(=O)N(C)CCN1CCCCC1] |
| 2C | 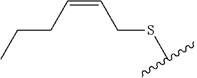 [SMILES: *SC/C=C\CCC] |
| 2T | 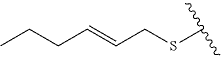 [SMILES: *SC/C=C/CCC] |
| 3C | 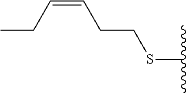 [SMILES: *SCC/C=C\CC] |
| 3T | 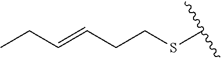 [SMILES: *SCC/C=C/CC] |
| 4C | 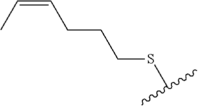 [SMILES: *SCCC/C=C\C] |
| 4T | 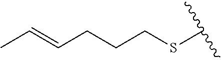 [SMILES: *SCCC/C=C/C] |
| 5 | 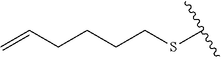 [SMILES: *SCCCCC=C] |
| 8/2 | 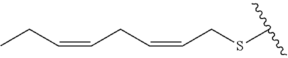 [SMILES: *SC/C=C\C/C=C\CC] |
| Citronellol (Cit) | 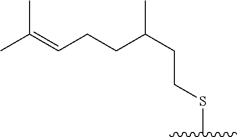 [SMILES: *SCCC(C)CCC=C(C)C] |
| Nerol (Nerol) | 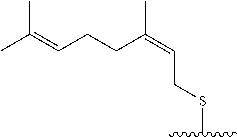 [SMILES: *SC/C=C(/C)CCC=C(C)C] |
| Farnesol (Far) | 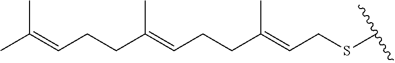 [SMILES: *SC/C=C(\C)CC/C=C(\C)CCC=C(C)C] |

In some embodiments, the dendrimer or dendrons of Formula (X) is selected from those set forth in Table 4 and pharmaceutically acceptable salts thereof.

TABLE 4
Example ionizable cationic lipo-dendrimers or lipo-dendrons

| ID # | Structure |
|---|---|
| 2A2- SC14 | 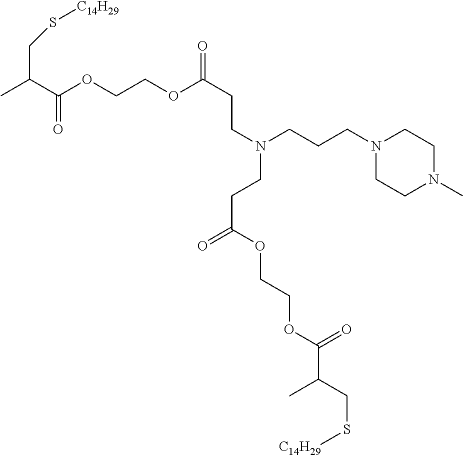 |
| 2A6- SC14 | 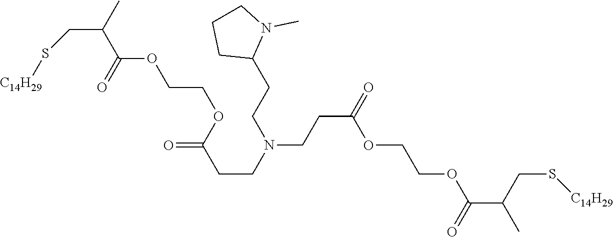 |
| 2A9- SC14 | 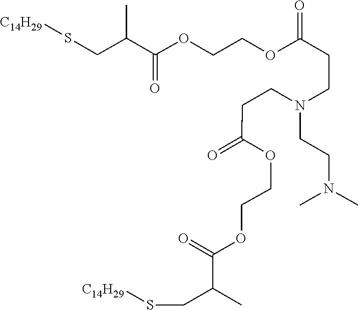 |
| 3A3- SC10 | 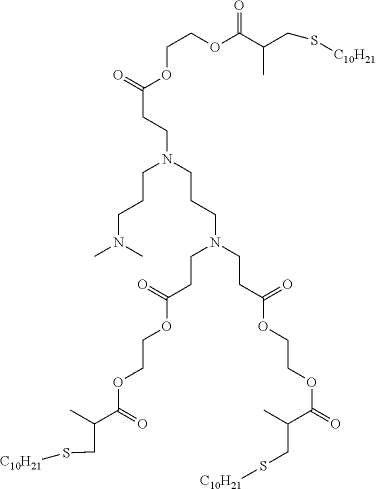 |
| 3A3- SC14 | 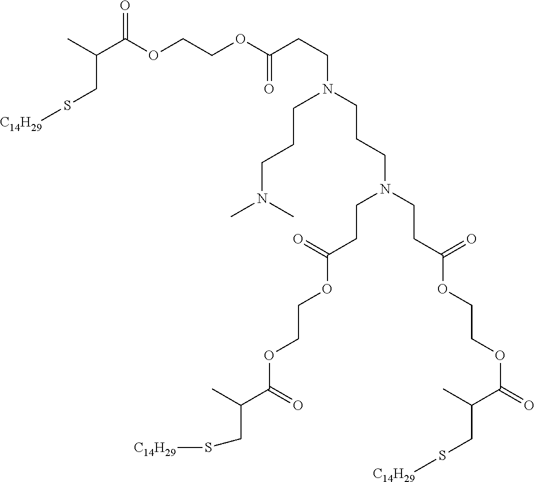 |
| 3A5- SC10 | 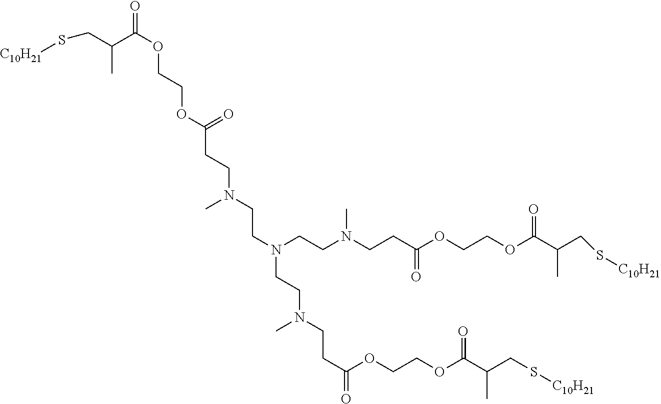 |
| 3A5- SC14 | 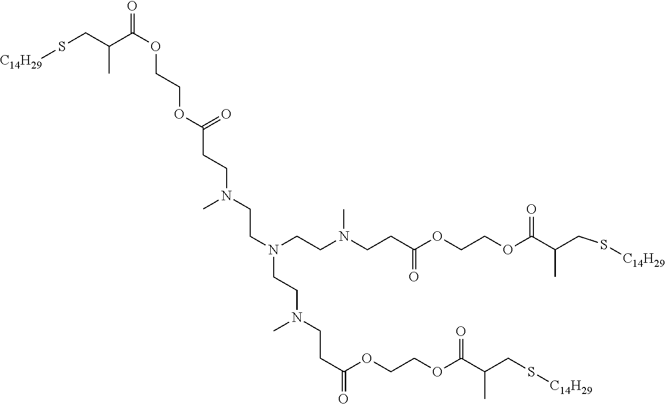 |
| 4A1- SC12 | 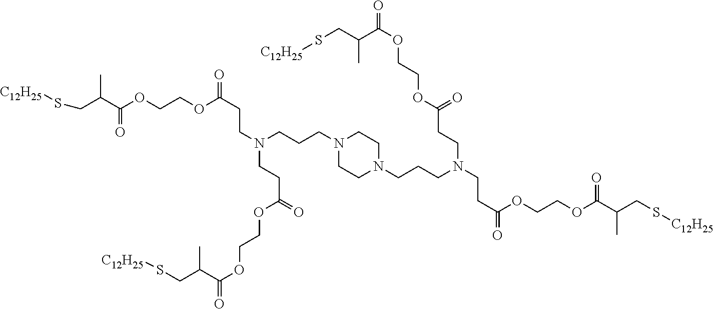 |
| 4A3- SC12 | 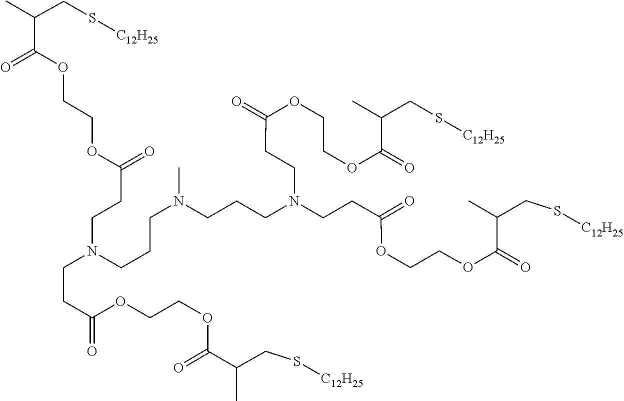 |
| 5A1- SC12 | 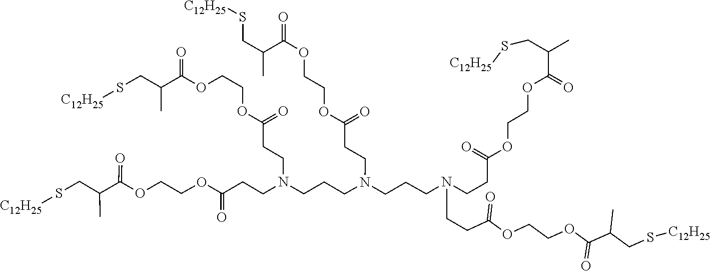 |
| 5A1- SC8 | 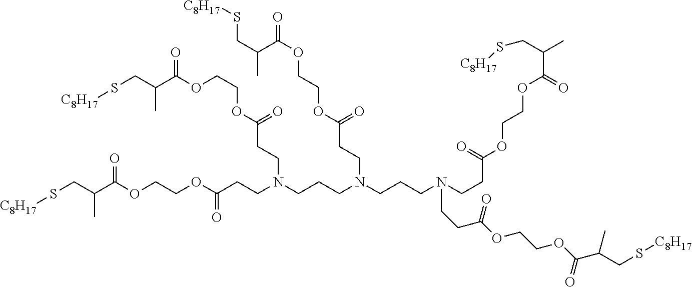 |
| 5A2- 2- SC12 (5- arm) | 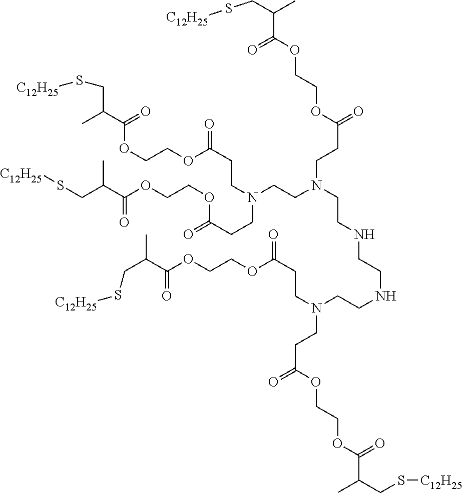 |
| 5A3- 1- SC12 (5 arm) | 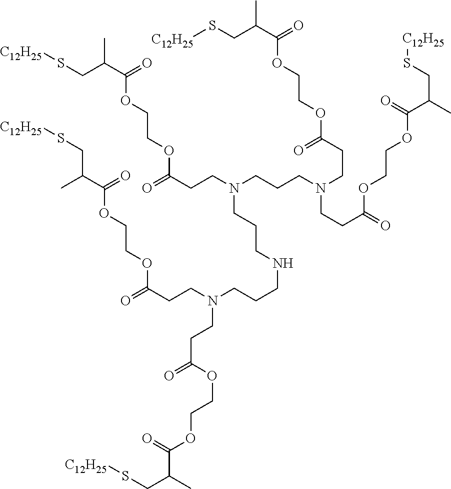 |
| 5A3- 1- SC8 (5- arm) | 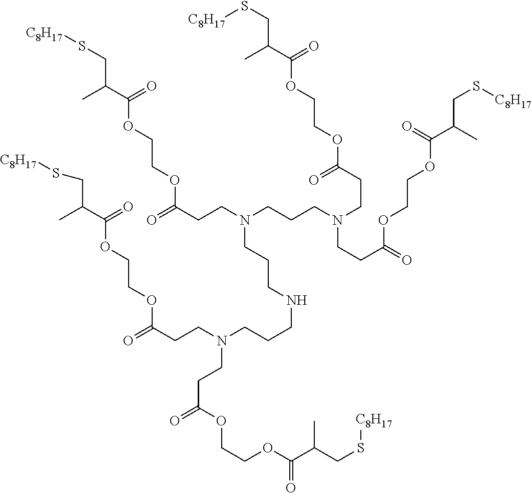 |
| 5A4- 1- SC12 (5- arm) | 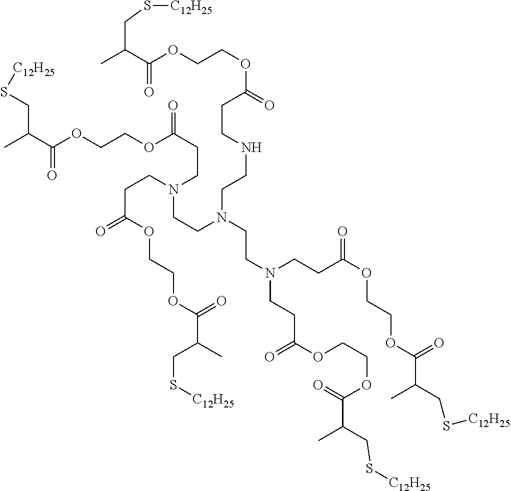 |
| 5A4- 1- SC8 (5- arm) | 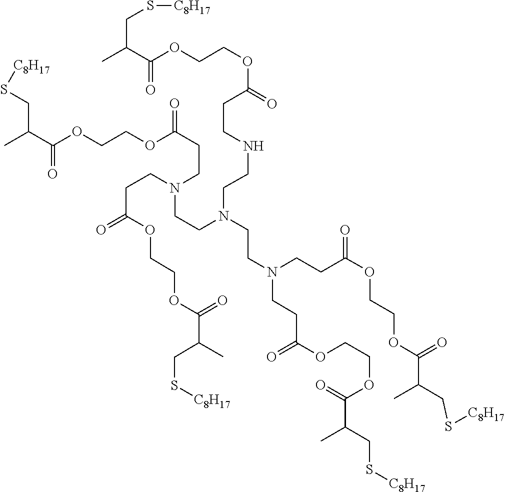 |
| 5A5- SC8 | 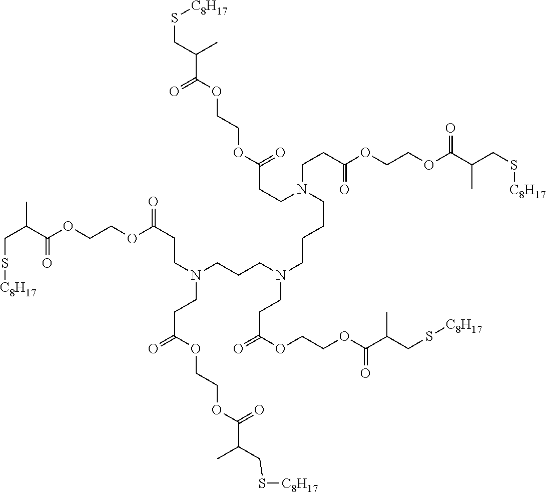 |
| 5A5- SC12 | 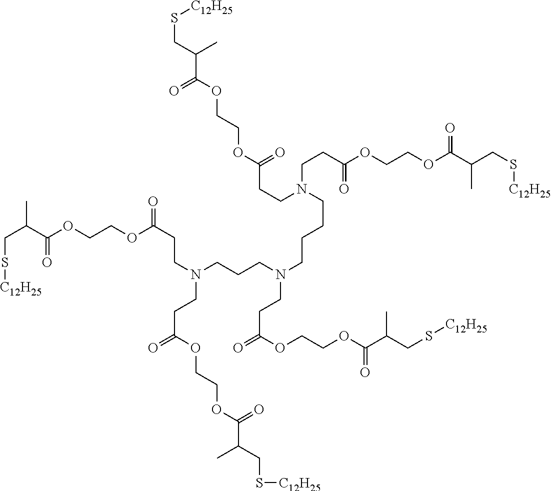 |
| 5A2- 4- SC12 (6- arm) | 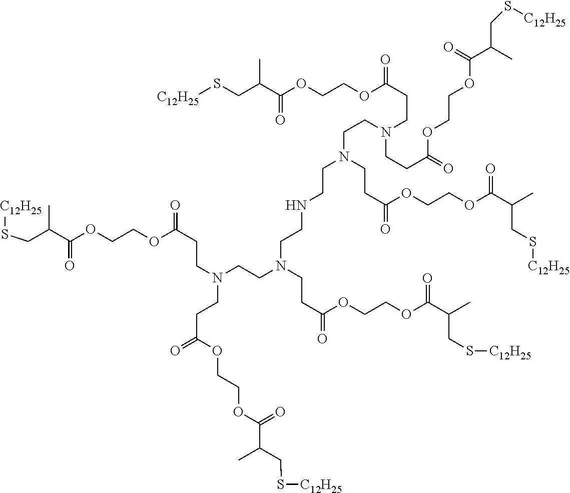 |
| 5A2- 4- SC10 (6- arm) | 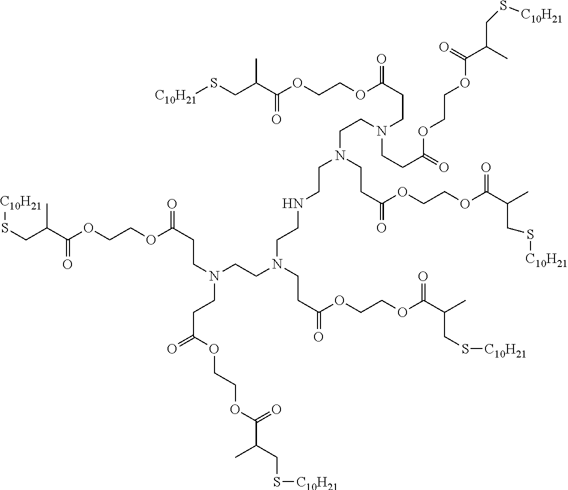 |
| 5A3- 2-- SC8 (6- arm) | 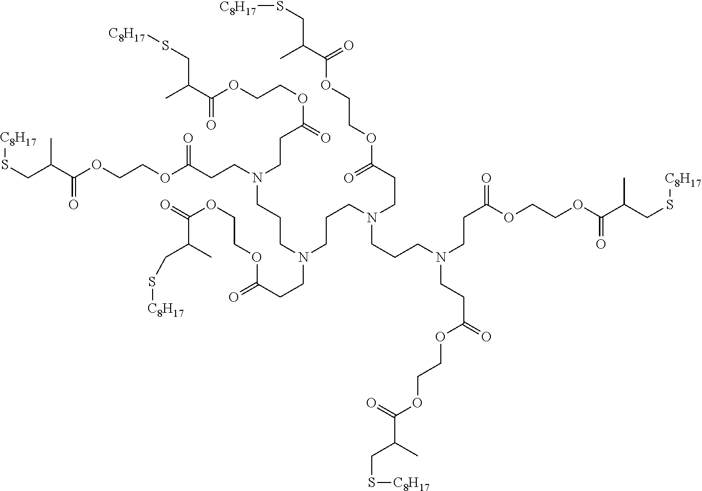 |
| 5A3- 2- SC12 (6- arm) | 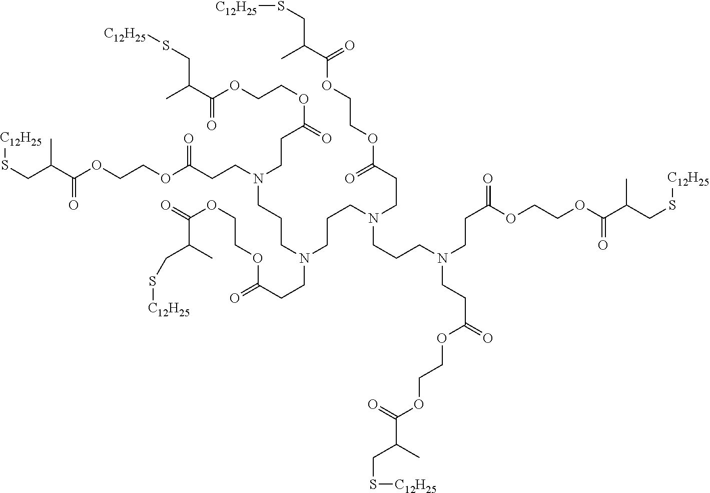 |
| 5A4- 2- SC8 (6- arm) | 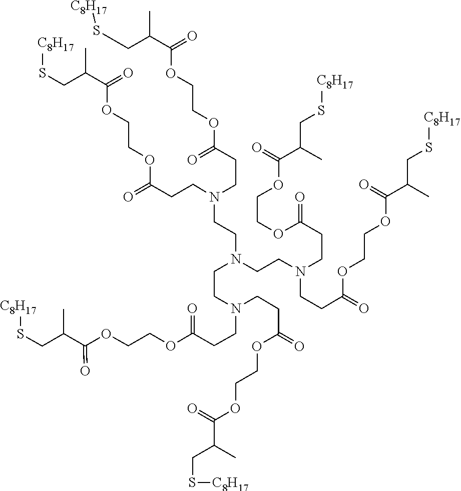 |
| 5A4- 2- SC12 (6- arm) | 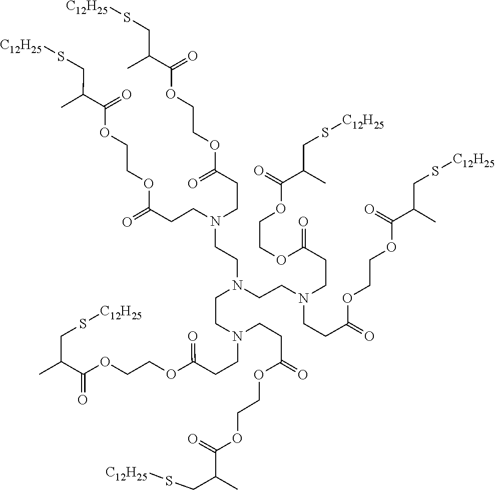 |
| 6A4- SC8 | 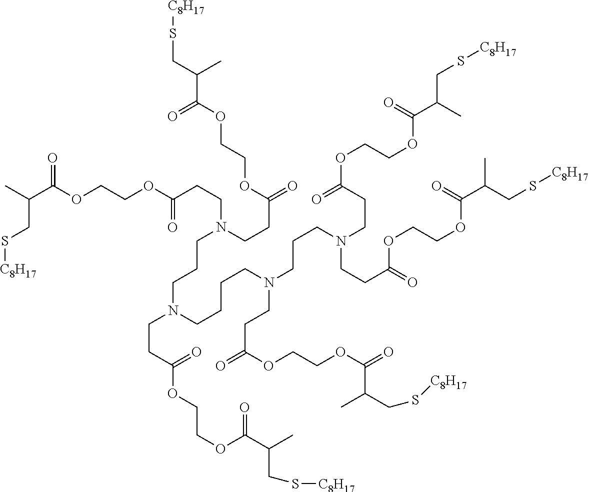 |
| 6A4- SC12 | 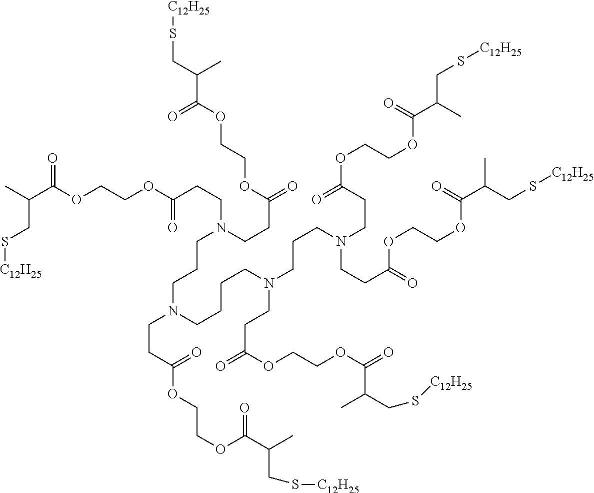 |
| 2A2- g2- SC12 | 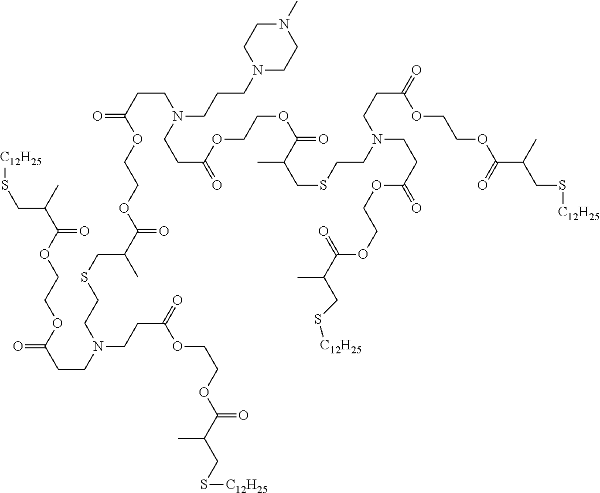 |
| 2A2- g2- SC8 | 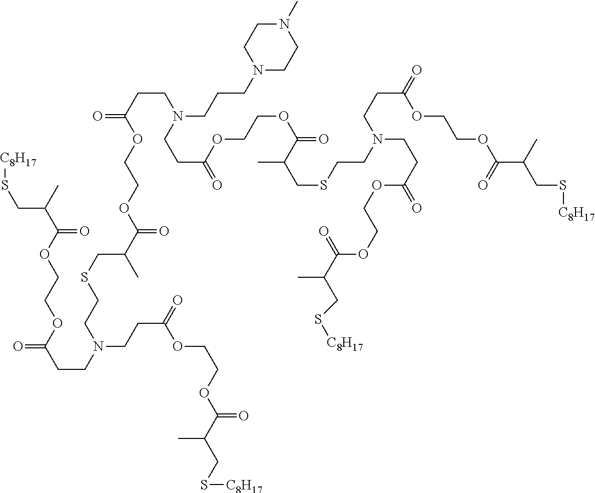 |
| 2A11- g2- SC12 | 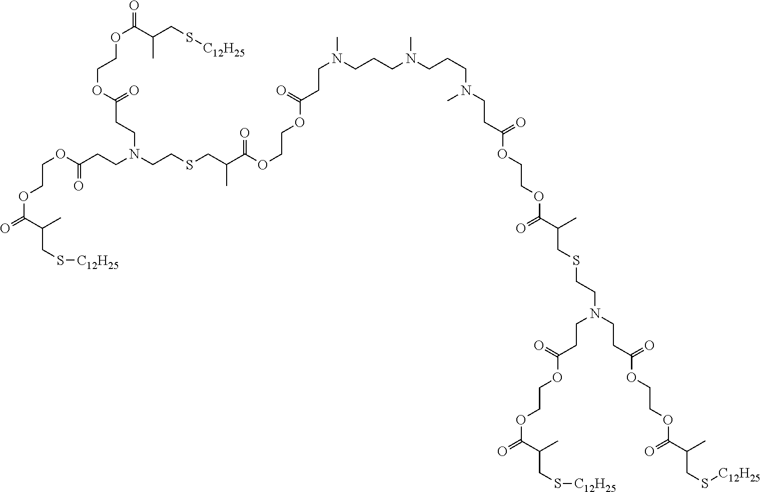 |
| 2A11- g2- SC8 | 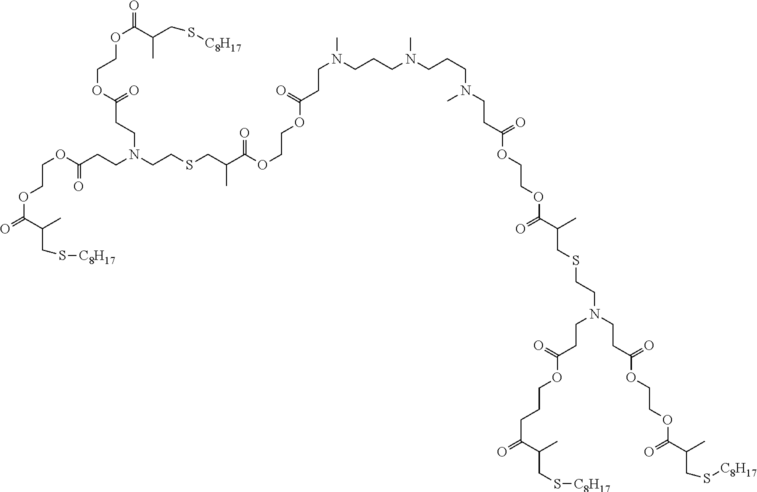 |
| 3A3- g2- SC8 | 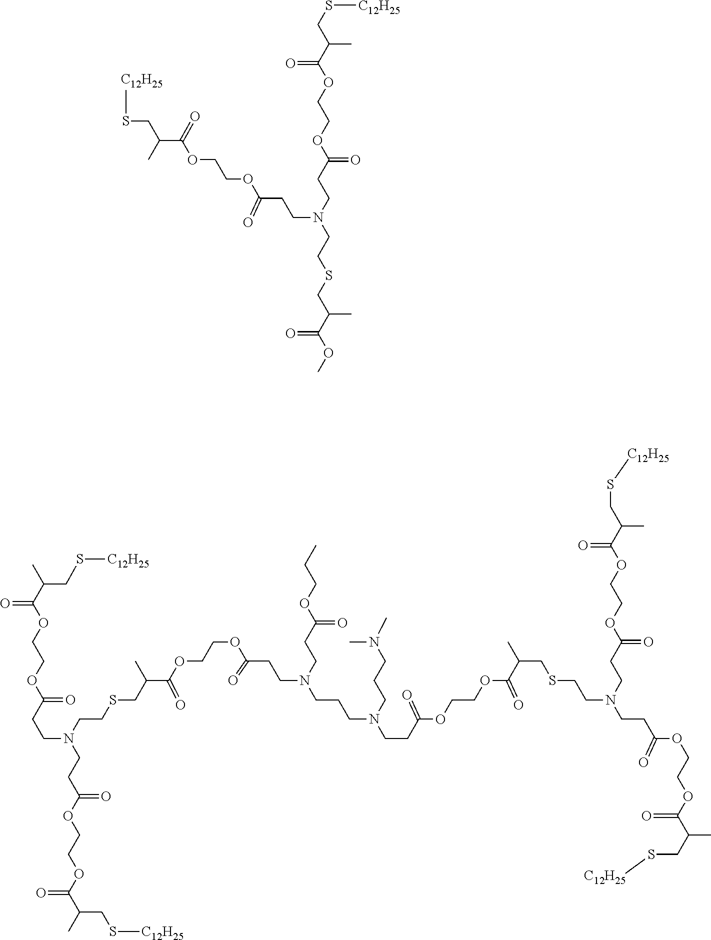 |
| 3A3- g2- SC8 | 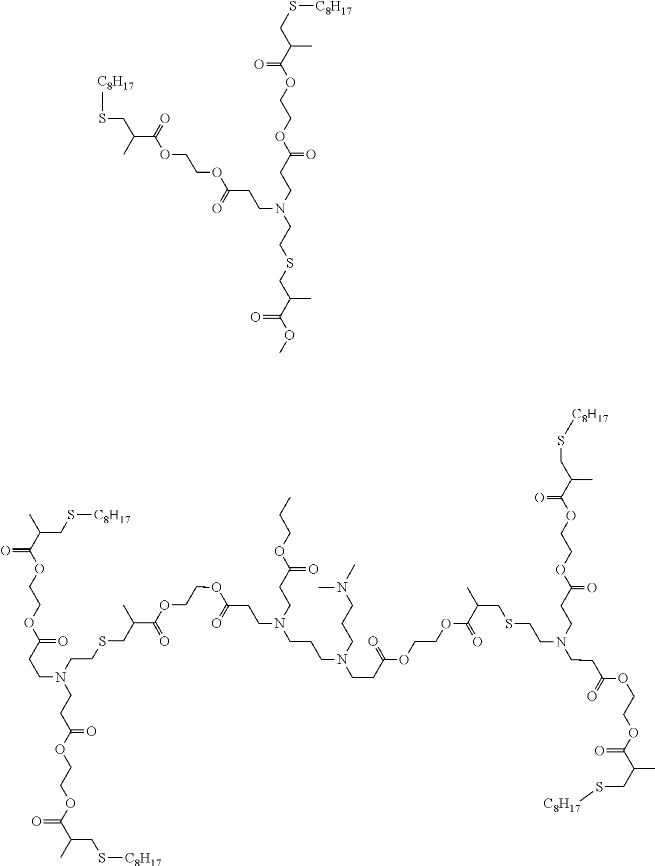 |
| 3A5- g2- SC12 | 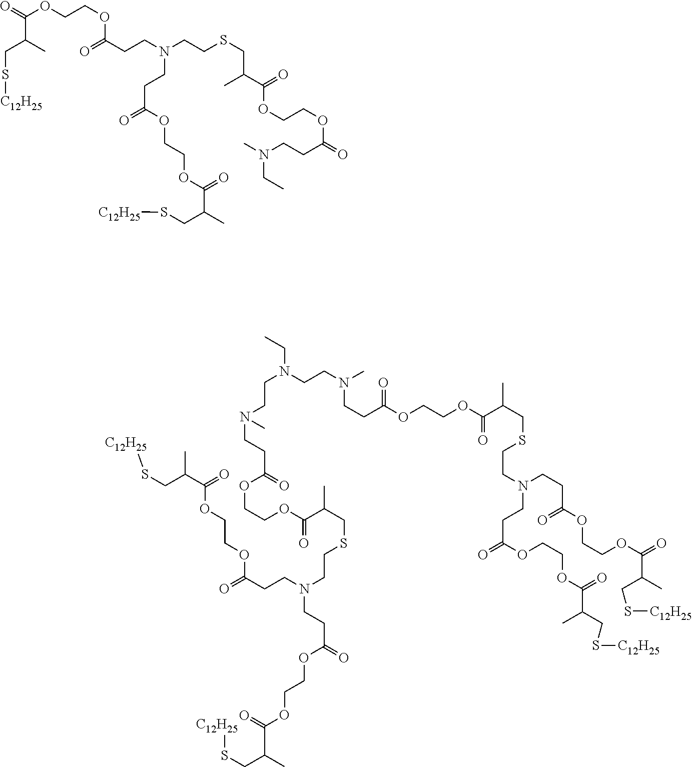 |
| 2A11- g3- SC12 | 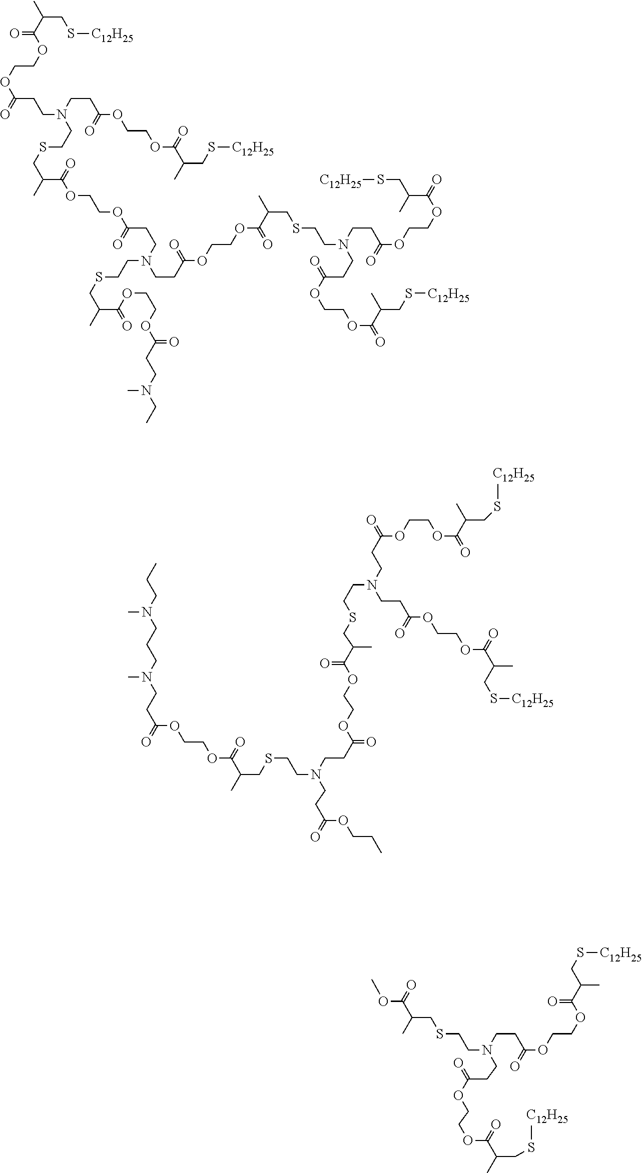 |
| 2A11- g3- SC8 | 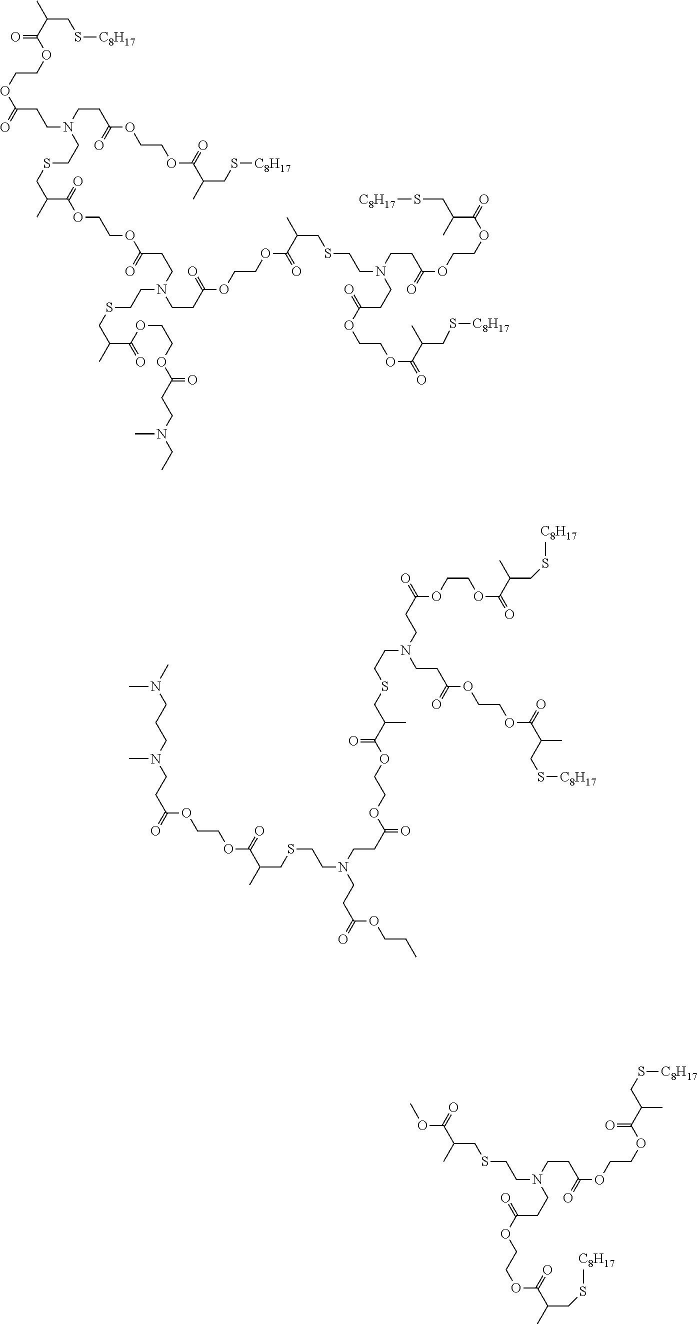 |
| 1A2- g4- SC12 | 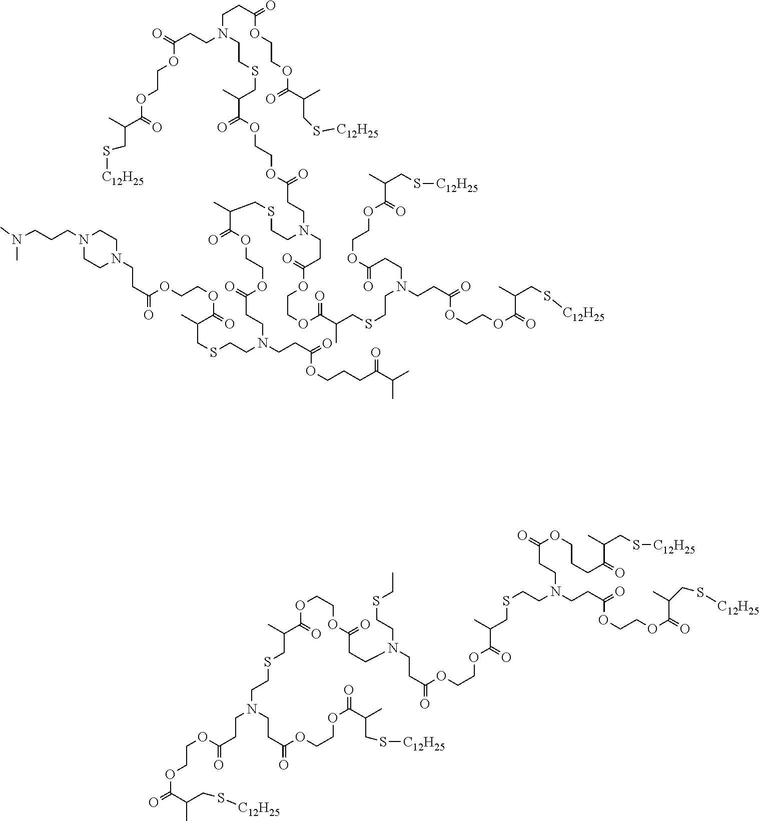 |
| 4A1- g2- SC12 | 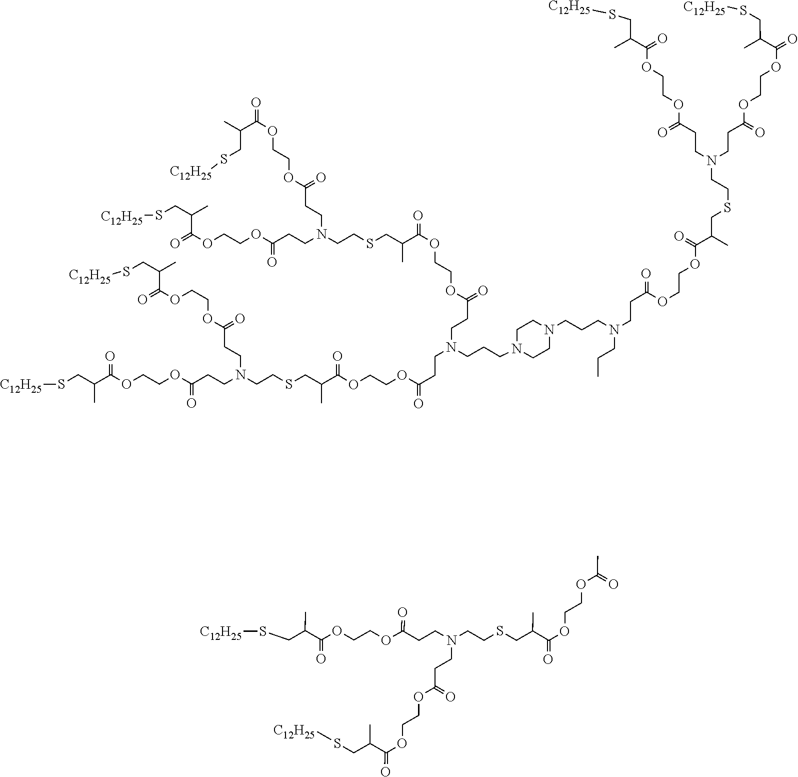 |
| 1A2- g4- SC8 | 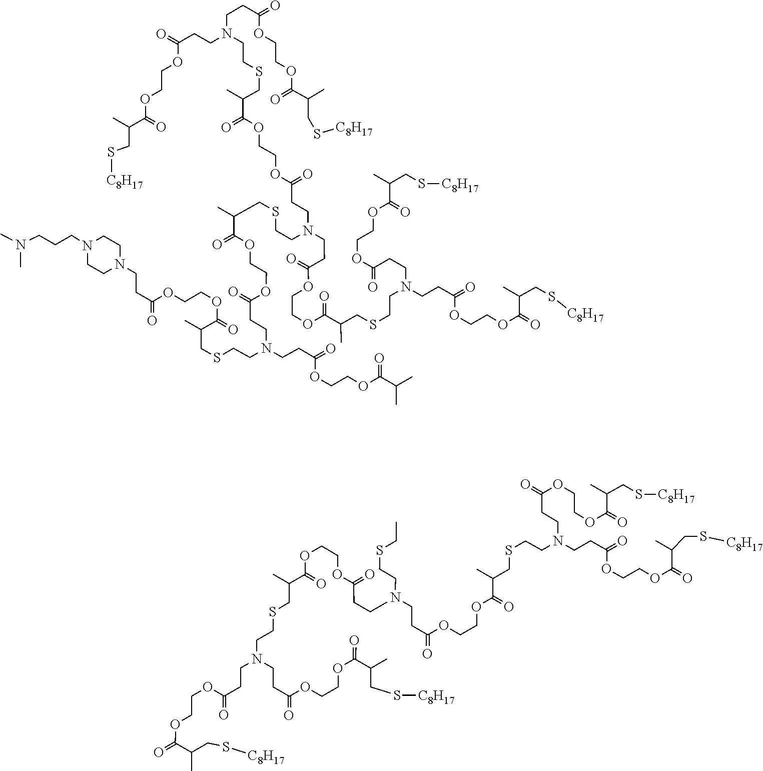 |
| 4A1- g2 SC8 | 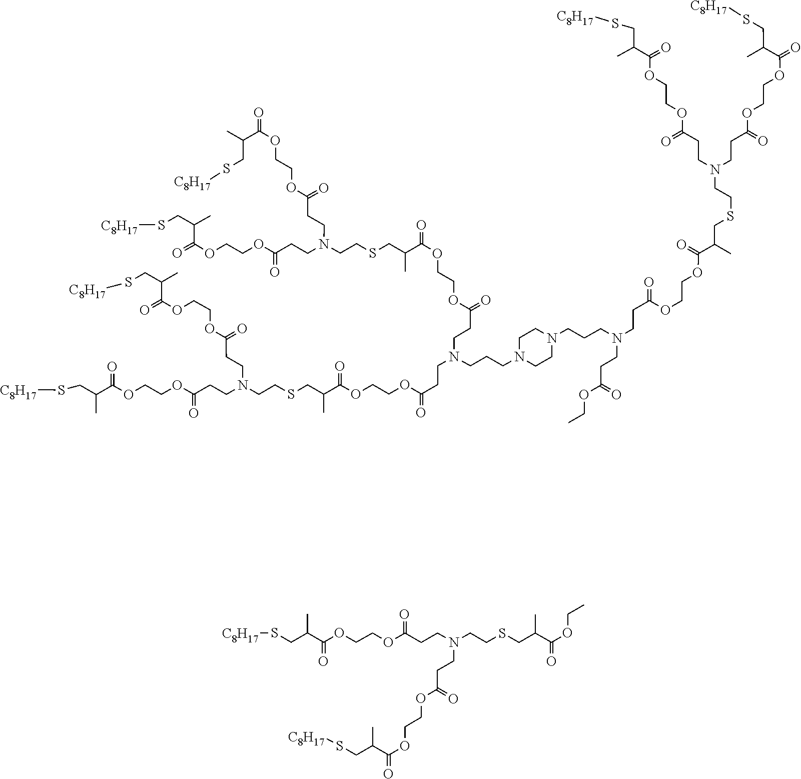 |
| 4A3- g2- SC12 | 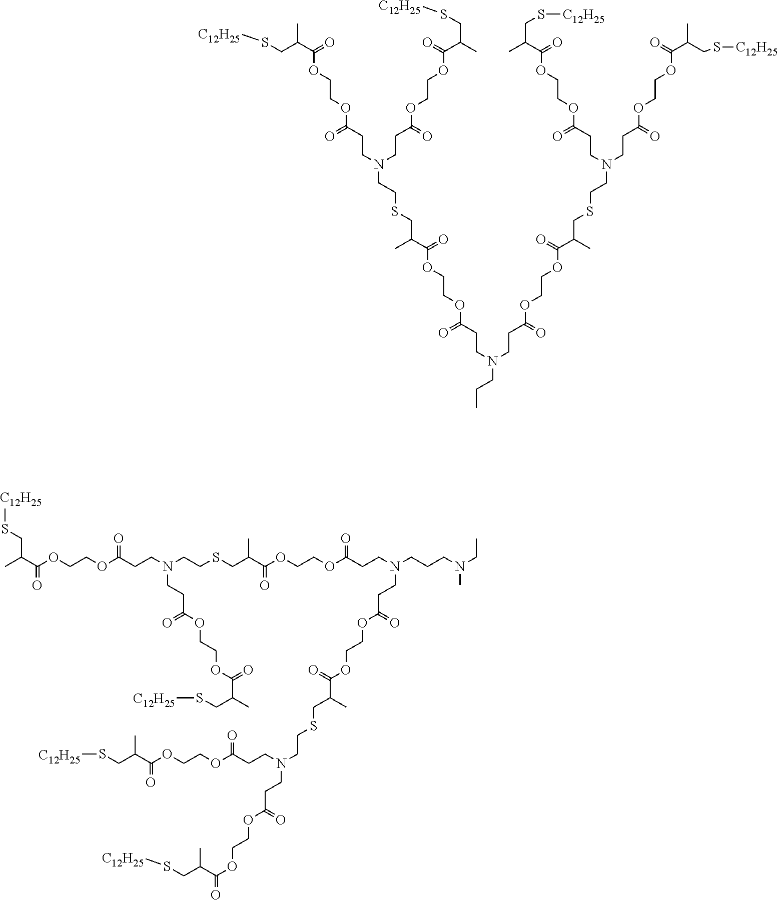 |
| 4A3- g2- SC8 | 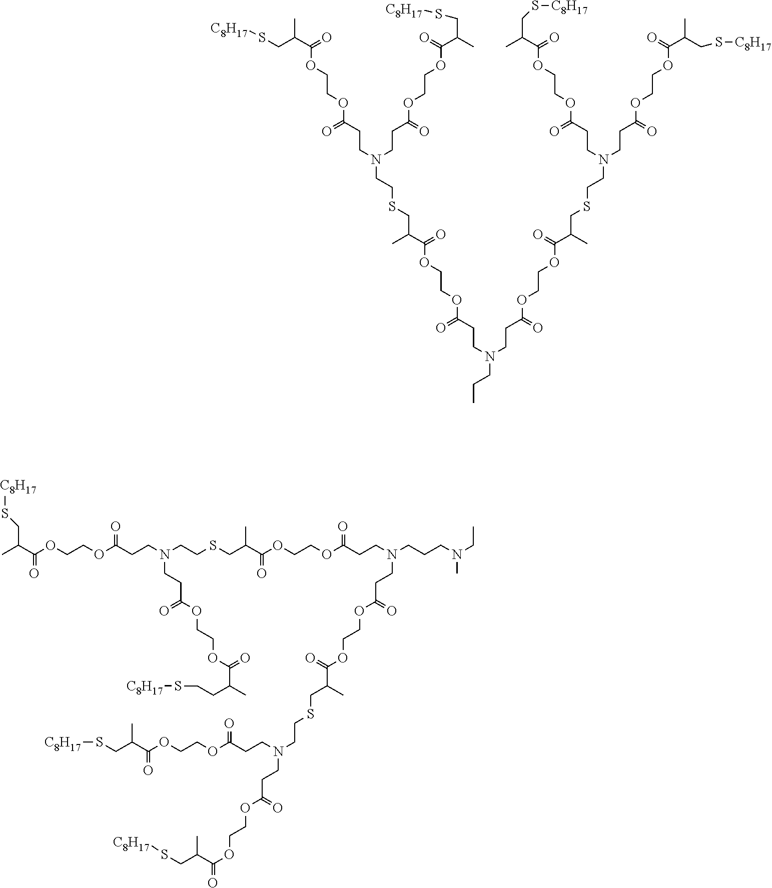 |
| 1A2- g3- SC12 | 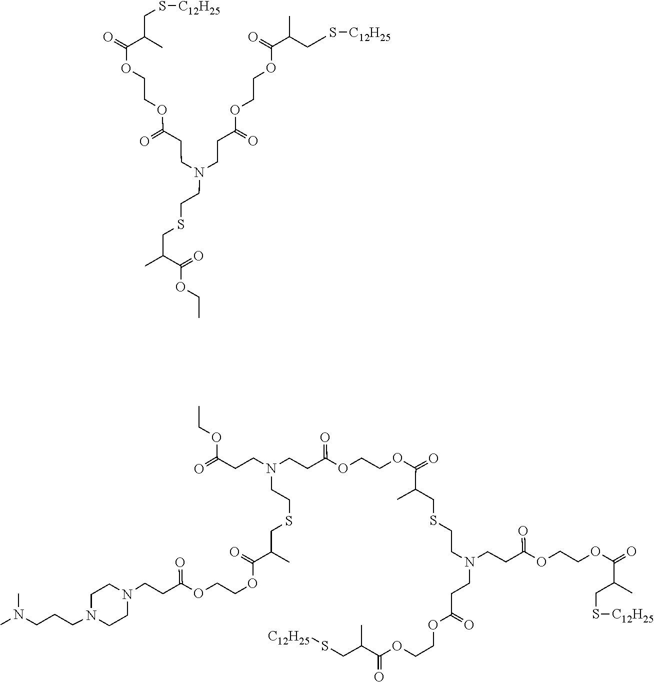 |
| 1A2- g3- SC8 | 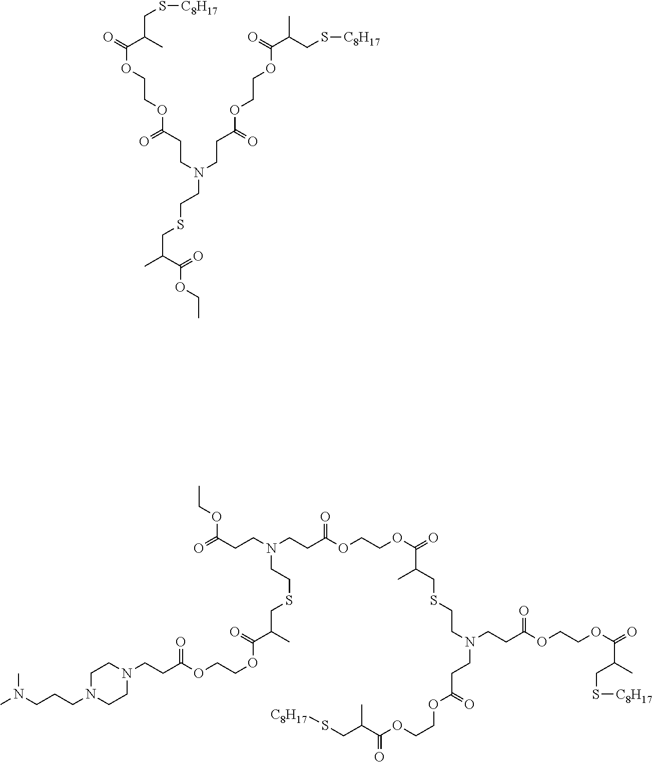 |
| 2A2- g3- SC12 | 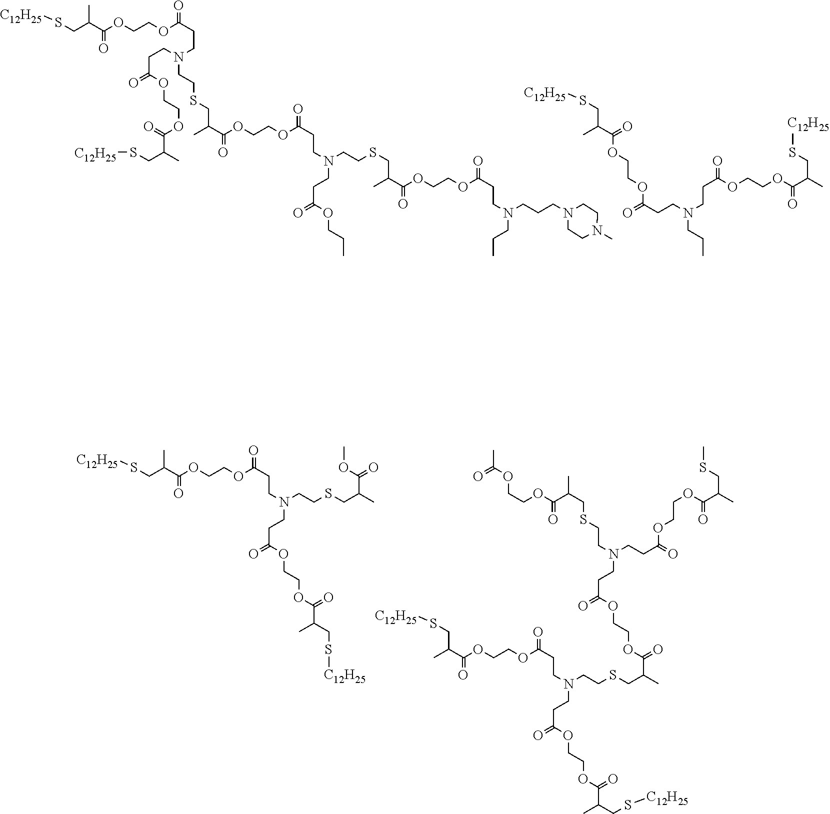 |
| 2A2- g3- SC8 | 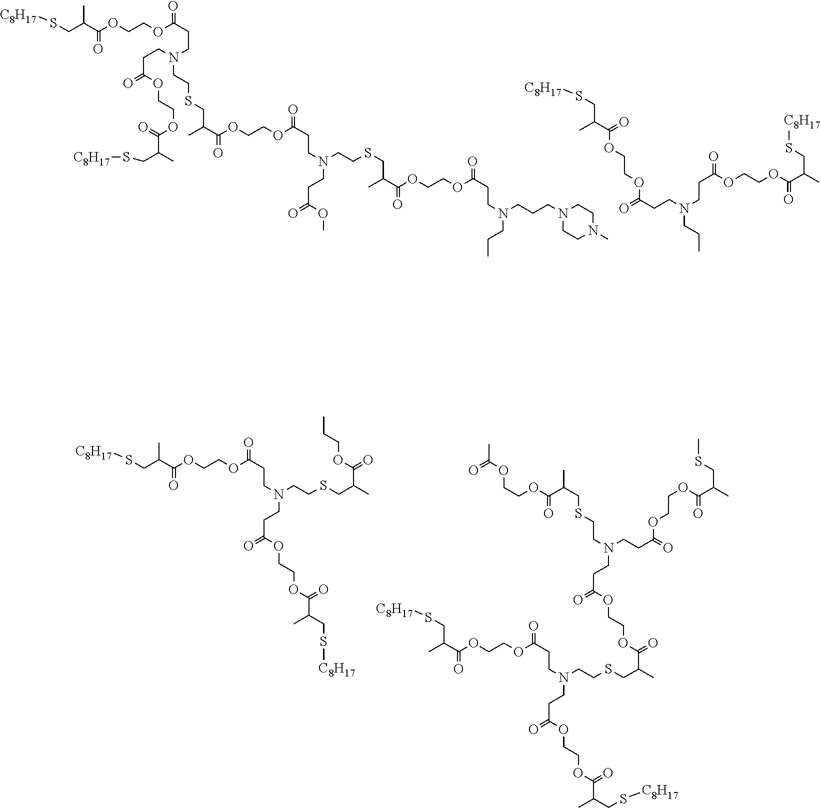 |
| 5A2- 4- SC8 (6- arm) | 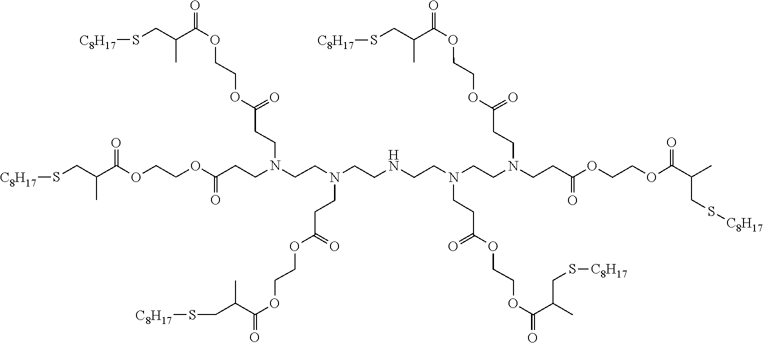 |
| 5A- 5- SC8 (6 arm) | 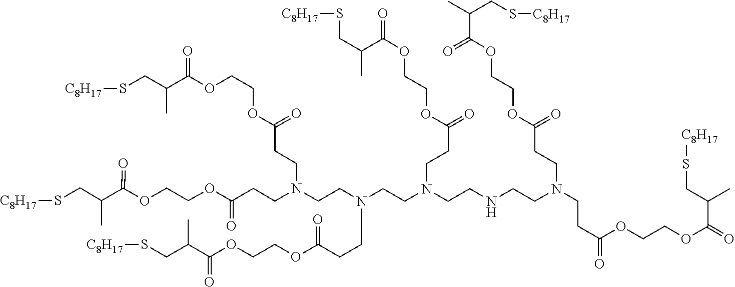 |
| 5A2- 6- SC8 (6 arm) | 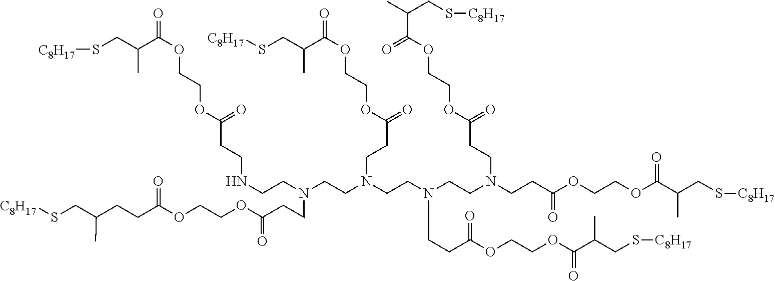 |
| 5A2- 1- SC8 (5- arm) | 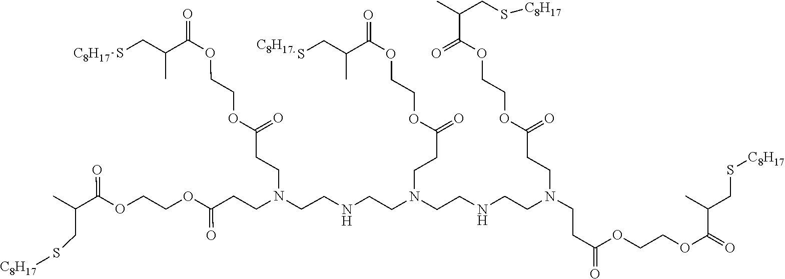 |
| 5A2- 2- SC8 | 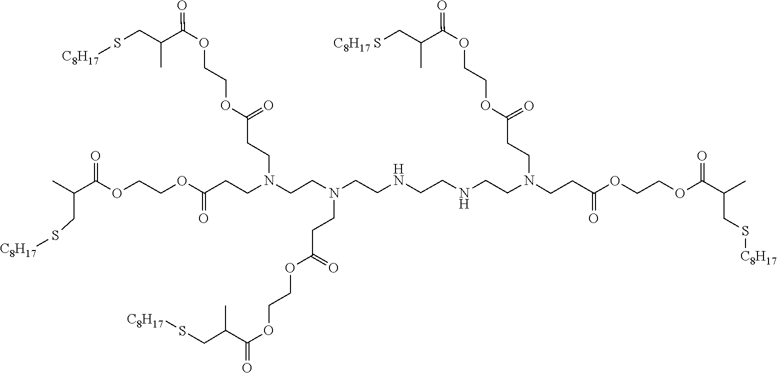 |
| 4A1- SC5 | 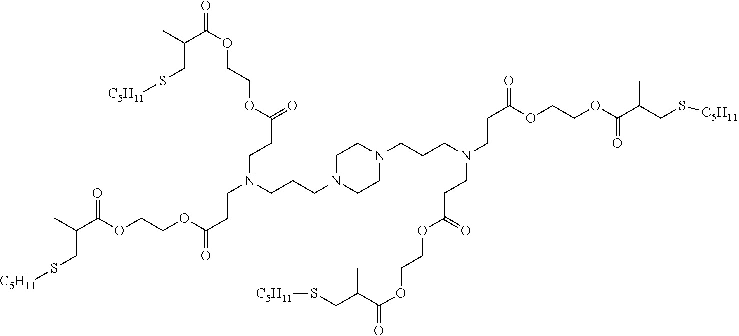 |
| 4A1- SC8 | 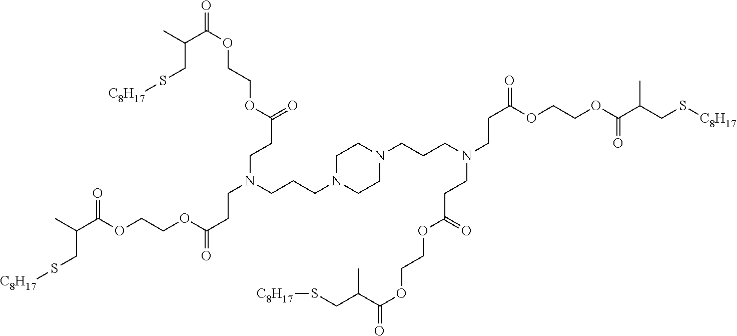 |
| 4A3- SC6 | 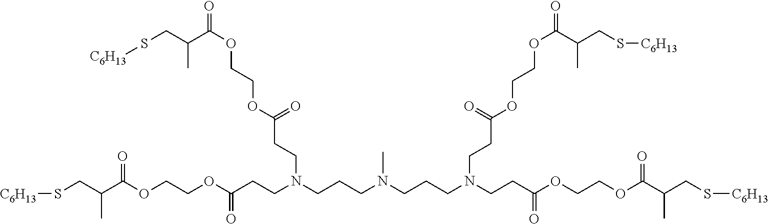 |
| 4A3- SC7 | 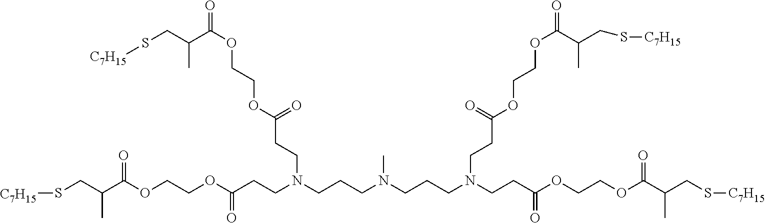 |
| 4A3- SC8 | 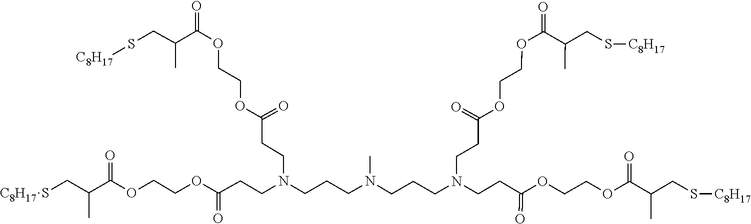 |
| 5A4- 2- SC5 (6 arm) | 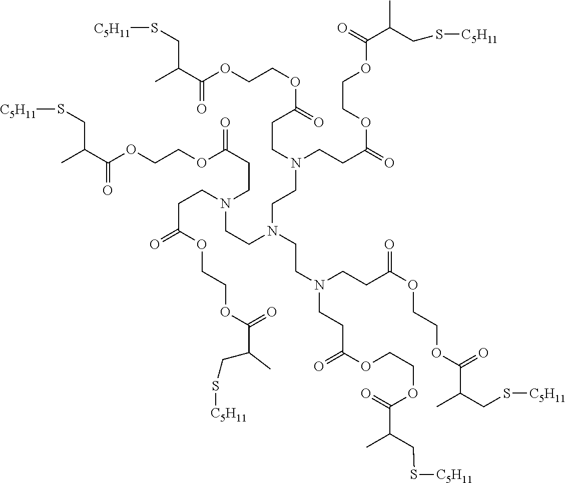 |
| 5A4- 2- SC6 (6 arm) | 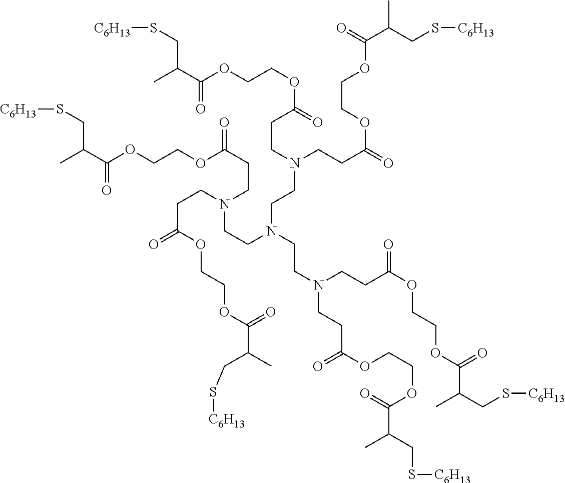 |
| 5A2- 4- SC8 (5- arm) | 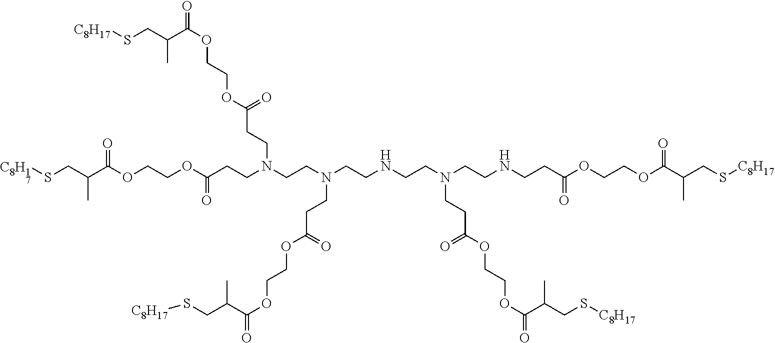 |
| 3A5- g2- SC8 | 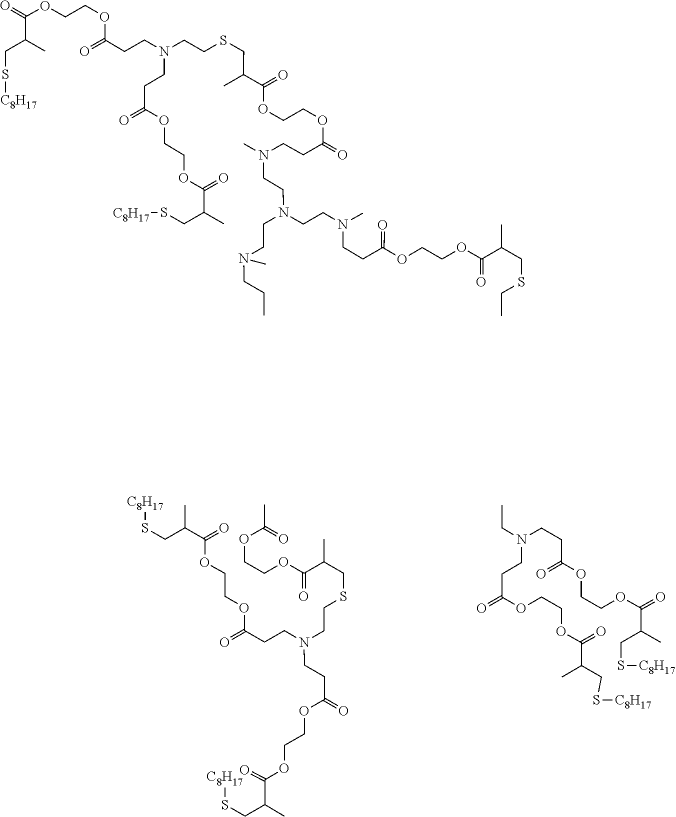 |

### Other Ionizable Cationic Lipids

In some embodiments of the lipid composition, the cationic lipid comprises a structural formula (D-I′):

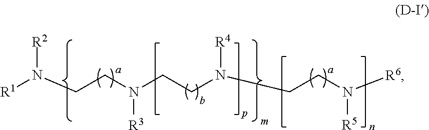

wherein:
   a is 1 and b is 2, 3, or 4; or, alternatively, b is 1 and a is 2, 3, or 4;
   m is 1 and n is 1; or, alternatively, m is 2 and n is 0; or, alternatively, m is 2 and n is 1; and
   R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
   provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
   wherein one or more of the nitrogen atoms indicated in formula (D-I′) may be protonated to provide a cationic lipid.

In some embodiments of the cationic lipid of formula (D-I′), a is 1. In some embodiments of the cationic lipid of formula (D-I′), b is 2. In some embodiments of the cationic lipid of formula (D-I′), m is 1. In some embodiments of the cationic lipid of formula (D-I′), n is 1. In some embodiments of the cationic lipid of formula (D-I′), R¹, R², R³, R⁴, R⁵, and R⁶ are each independently H or —CH₂CH(OH)R⁷. In some embodiments of the cationic lipid of formula (D-I′), R¹, R², R³, R⁴, R⁵, and R⁶ are each independently H or

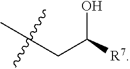

In some embodiments of the cationic lipid of formula (D-I′), R¹, R², R³, R⁴, R⁵, and R⁶ are each independently H or

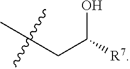

In some embodiments of the cationic lipid of formula (D-I′), R⁷ is C₃-C₁₈ alkyl (e.g., C₆-C₁₂ alkyl).

In some embodiments, the cationic lipid of formula (D-I′) is 13,16,20-tris(2-hydroxydodecyl)-13,16,20,23-tetraazapentatricontane-11,25-diol:

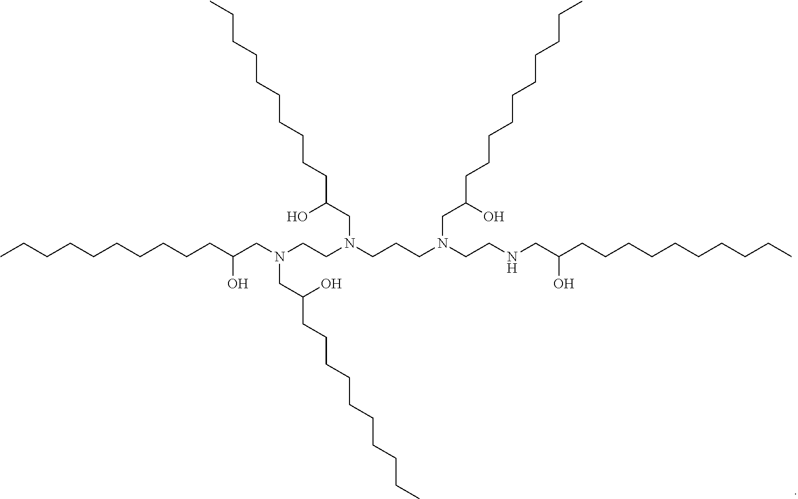 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (D-I′) is (11R,25R)-13,16,20-tris((R)-2-hydroxydodecyl)-13,16,20,23-tetraazapentatricontane-11,25-diol:

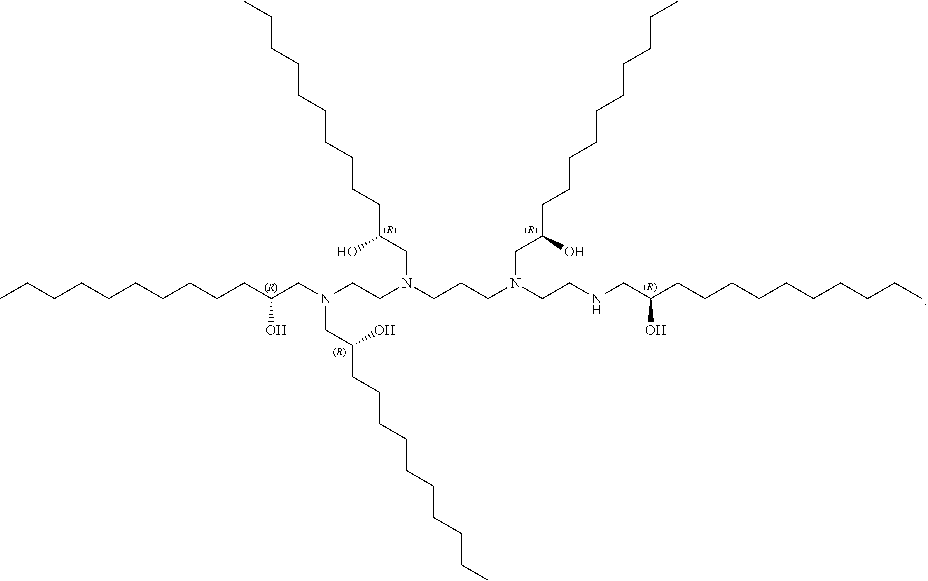

Additional cationic lipids that can be used in the compositions and methods of the present application include those cationic lipids as described in J. McClellan, M. C. King, Cell 2010, 141, 210-217, and International Patent Publication WO 2010/144740, WO 2013/149140, WO 2016/118725, WO 2016/118724, WO 2013/063468, WO 2016/205691, WO 2015/184256, WO 2016/004202, WO 2015/199952, WO 2017/004143, WO 2017/075531, WO 2017/117528, WO 2017/049245, WO 2017/173054 and WO 2015/095340, which are incorporated herein by reference for all purposes. Examples of those ionizable cationic lipids include but are not limited to those as shown in Table 5.

TABLE 5
Example ionizable cationic lipids

| # | Structure of example ionizable cationic lipid |
|---|---|
| 1 | 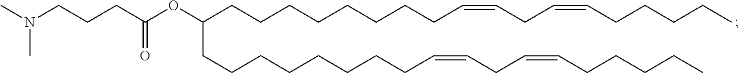 [SMILES: CCCCC/C=C\C/C=C\CCCCCCCCC(CCCCCCCC/C=C\C/C=C\CCCCC)OC(=O)CCCN(C)C] |
| 2 | 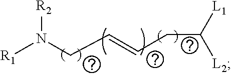 |
| 3 | 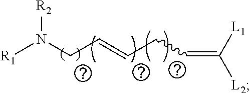 |
| 4 | 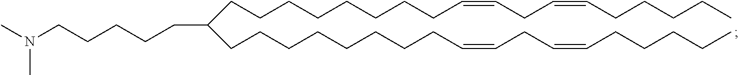 [SMILES: CCCCC/C=C\C/C=C\CCCCCCCCC(CCCCCCCC/C=C\C/C=C\CCCCC)CCCCCN(C)C] |
| 5 | 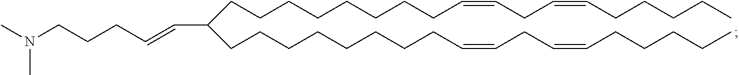 [SMILES: CCCCC/C=C\C/C=C\CCCCCCCCC(/C=C/CCCN(C)C)CCCCCCCC/C=C\C/C=C\CCCCC] |
| 6 | 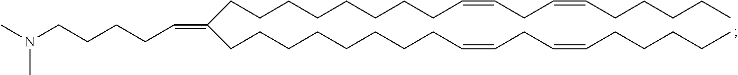 [SMILES: CCCCC/C=C\C/C=C\CCCCCCCCC(=CCCCCN(C)C)CCCCCCCC/C=C\C/C=C\CCCCC] |
| 7 | 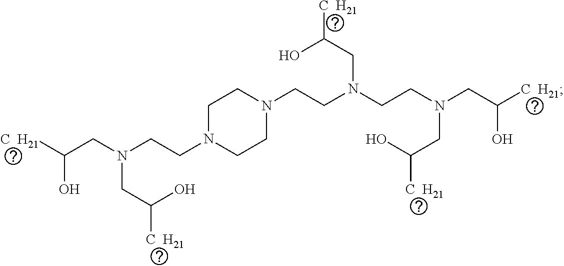 |
| 8 | 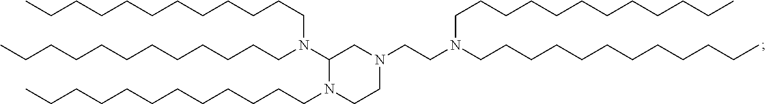 [SMILES: CCCCCCCCCCCCN(CCCCCCCCCCCC)CCN1CCN(CCCCCCCCCCCC)C(N(CCCCCCCCCCCC)CCCCCCCCCCCC)C1] |
| 9 | 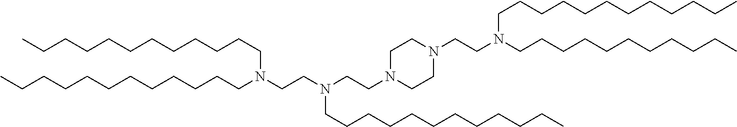 [SMILES: CCCCCCCCCCCCN(CCCCCCCCCCCC)CCN(CCCCCCCCCCCC)CCN1CCN(CCN(CCCCCCCCCCC)CCCCCCCCCCCC)CC1] |
| 10 | 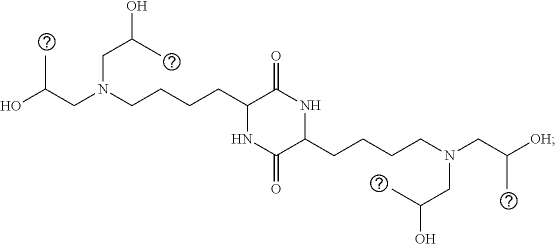 [SMILES: CC(O)CN(CCCCC1NC(=O)C(CCCCN(CC(C)O)CC(C)O)NC1=O)CC(C)O] |
| 11 | 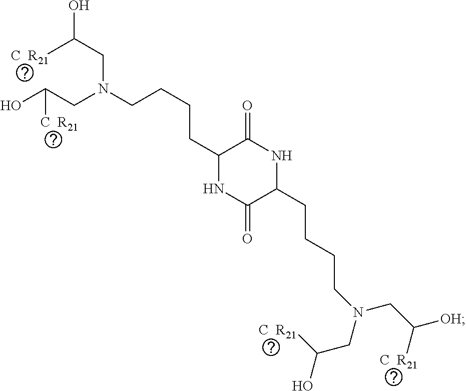 |
| 12 | 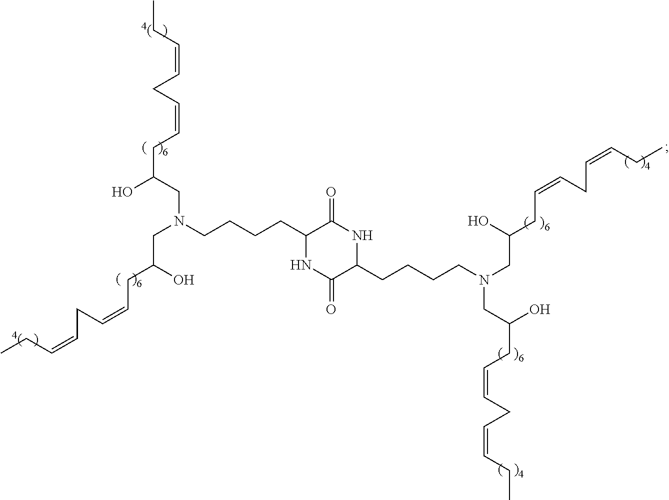 |
| 13 | 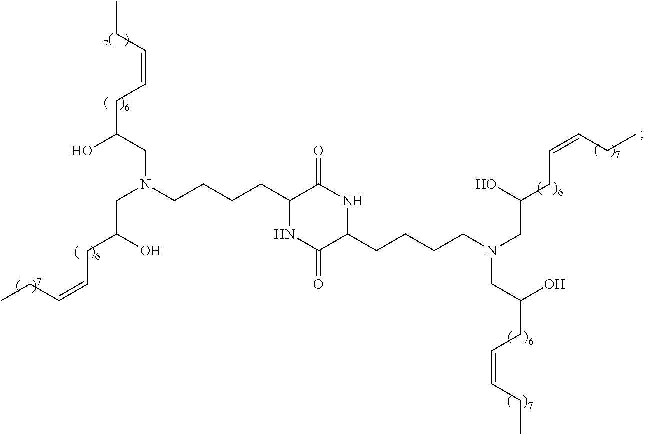 |
| 14 | 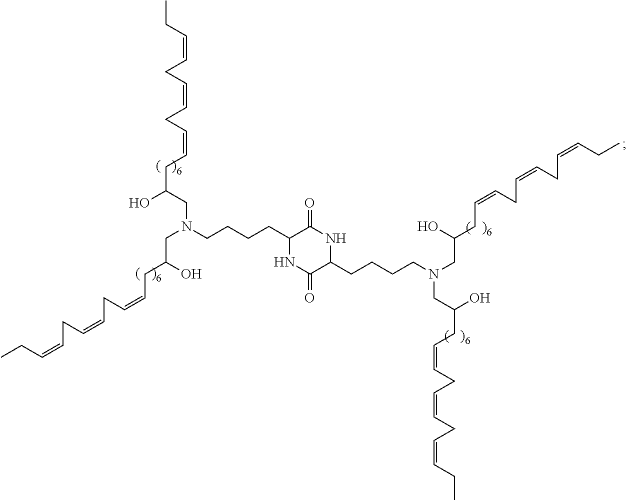 |
| 15 | 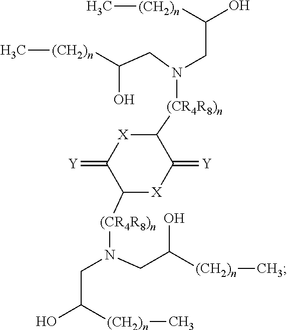 |
| 16 | 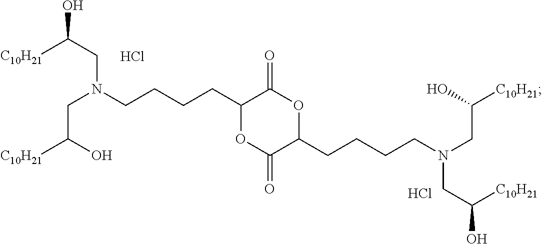 |
| 17 | 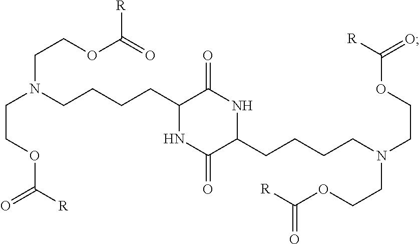 |
| 18 | 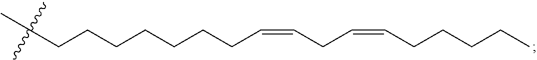 [SMILES: *CCCCCCC/C=C\C/C=C\CCCCC] |
| 19 | 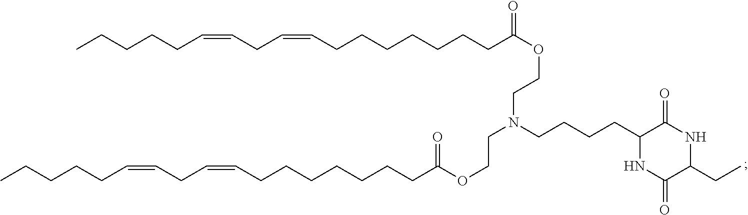 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC] |
| 20 | 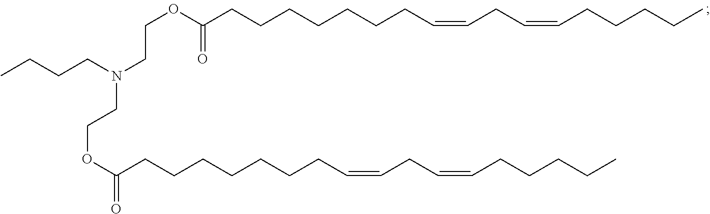 [SMILES: CCCCC/C=C\C/C=C\CCCCCCCC(=O)OCCN(CCCC)CCOC(=O)CCCCCCC/C=C\C/C=C\CCCCC] |
| 21 | 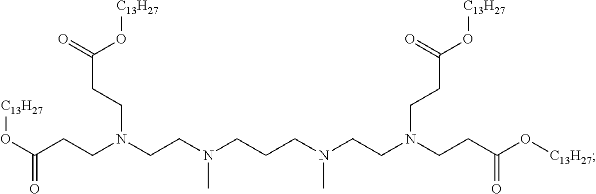 |
| 22 | 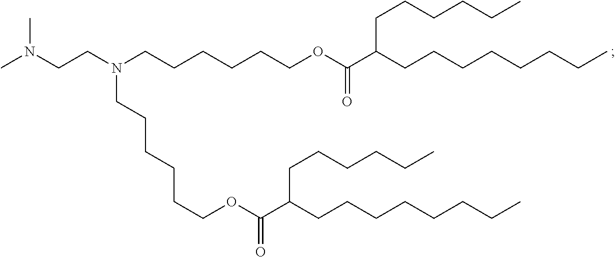 [SMILES: CCCCCCCCC(CCCCCC)C(=O)OCCCCCCN(CCCCCCOC(=O)C(CCCCCC)CCCCCCCC)CCN(C)C] |
| 23 | 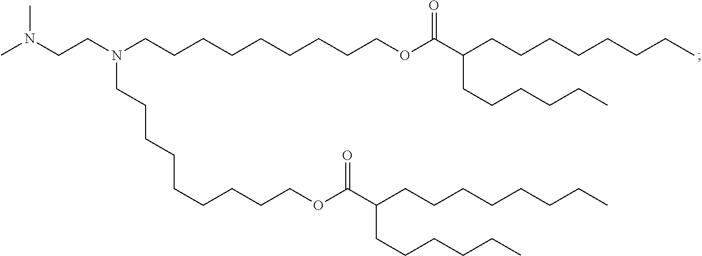 [SMILES: CCCCCCCCC(CCCCCC)C(=O)OCCCCCCCCCN(CCCCCCCCCOC(=O)C(CCCCCC)CCCCCCCC)CCN(C)C] |
| 24 | 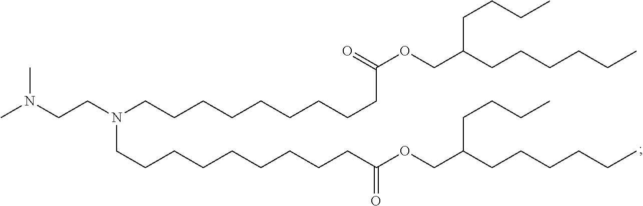 [SMILES: CCCCCCC(CCCC)COC(=O)CCCCCCCCCN(CCCCCCCCCC(=O)OCC(CCCC)CCCCCC)CCN(C)C] |
| 25 | 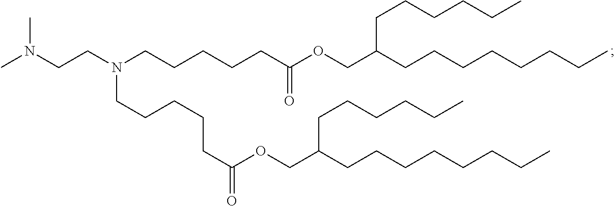 [SMILES: CCCCCCCCC(CCCCCC)COC(=O)CCCCCN(CCCCCC(=O)OCC(CCCCCC)CCCCCCCC)CCN(C)C] |
| 26 | 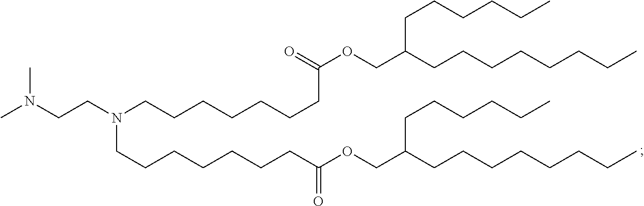 [SMILES: CCCCCCCCC(CCCCCC)COC(=O)CCCCCCCN(CCCCCCCC(=O)OCC(CCCCCC)CCCCCCCC)CCN(C)C] |
| 27 | 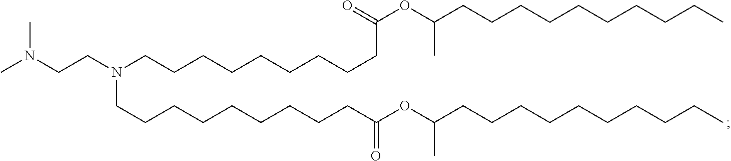 [SMILES: CCCCCCCCCCC(C)OC(=O)CCCCCCCCCN(CCCCCCCCCC(=O)OC(C)CCCCCCCCCC)CCN(C)C] |
| 28 | 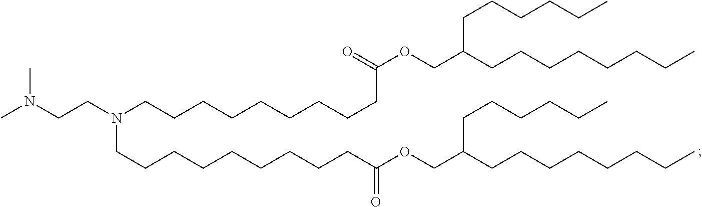 [SMILES: CCCCCCCCC(CCCCCC)COC(=O)CCCCCCCCCN(CCCCCCCCCC(=O)OCC(CCCCCC)CCCCCCCC)CCN(C)C] |
| 29 | 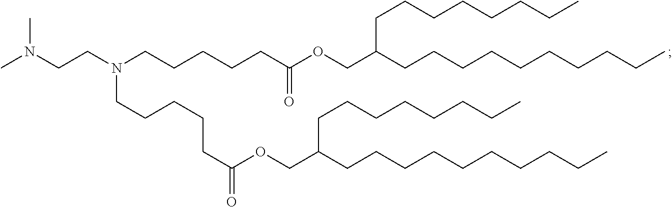 [SMILES: CCCCCCCCCCC(CCCCCCCC)COC(=O)CCCCCN(CCCCCC(=O)OCC(CCCCCCCC)CCCCCCCCCC)CCN(C)C] |
| 30 | 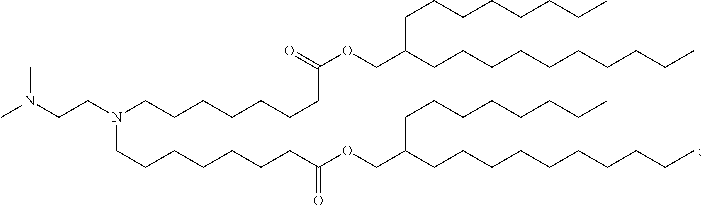 [SMILES: CCCCCCCCCCC(CCCCCCCC)COC(=O)CCCCCCCN(CCCCCCCC(=O)OCC(CCCCCCCC)CCCCCCCCCC)CCN(C)C] |
| 31 | 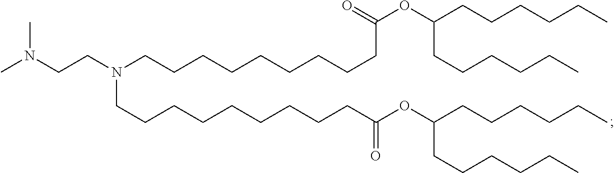 [SMILES: CCCCCCC(CCCCCC)OC(=O)CCCCCCCCCN(CCCCCCCCCC(=O)OC(CCCCCC)CCCCCC)CCN(C)C] |
| 32 | 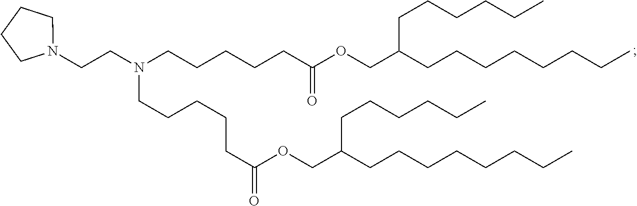 [SMILES: CCCCCCCCC(CCCCCC)COC(=O)CCCCCN(CCCCCC(=O)OCC(CCCCCC)CCCCCCCC)CCN1CCCC1] |
| 33 | 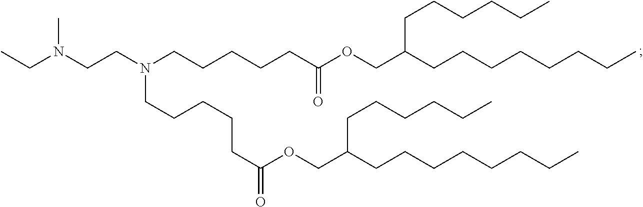 [SMILES: CCCCCCCCC(CCCCCC)COC(=O)CCCCCN(CCCCCC(=O)OCC(CCCCCC)CCCCCCCC)CCN(C)CC] |
| 34 | 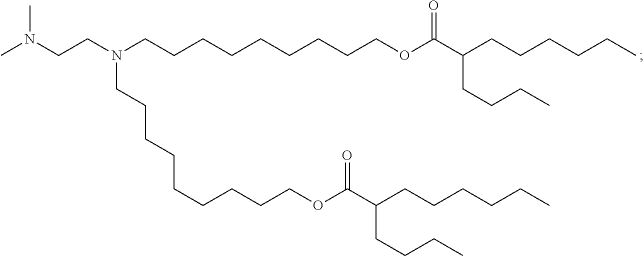 [SMILES: CCCCCCC(CCCC)C(=O)OCCCCCCCCCN(CCCCCCCCCOC(=O)C(CCCC)CCCCCC)CCN(C)C] |
| 35 | 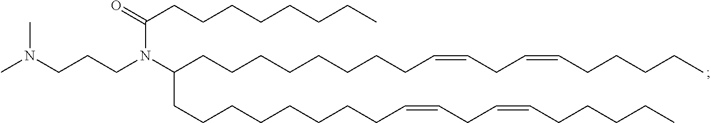 [SMILES: CCCCC/C=C\C/C=C\CCCCCCCCC(CCCCCCCC/C=C\C/C=C\CCCCC)N(CCCN(C)C)C(=O)CCCCCCCC] |
| 36 | 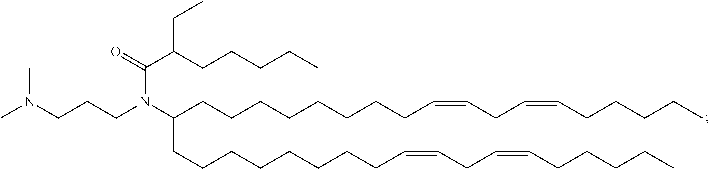 [SMILES: CCCCC/C=C\C/C=C\CCCCCCCCC(CCCCCCCC/C=C\C/C=C\CCCCC)N(CCCN(C)C)C(=O)C(CC)CCCCC] |
| 37 | 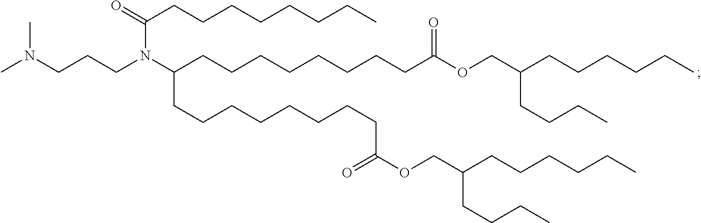 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| 38 | 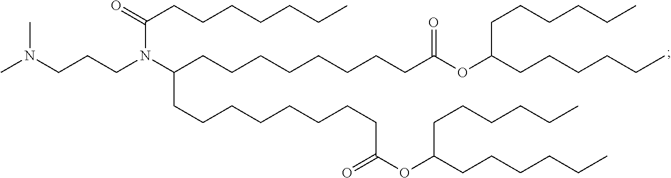 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |
| 39 | 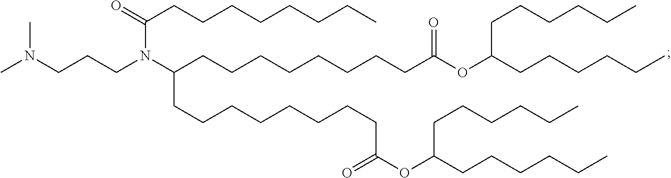 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] |
| 40 | 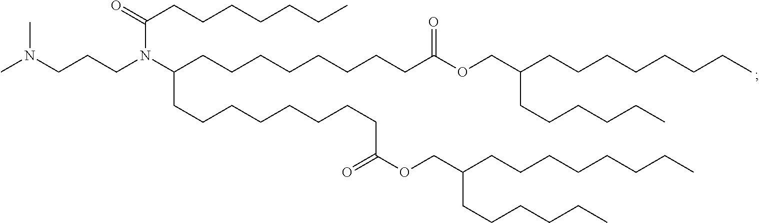 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] |
| 41 | 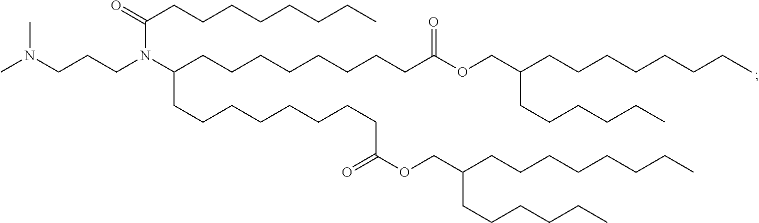 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] |
| 42 | 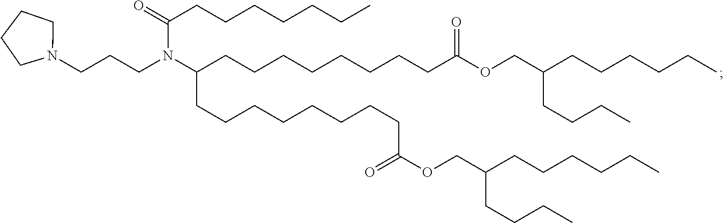 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] |
| 43 | 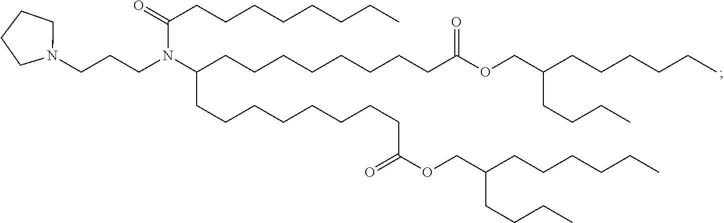 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] |
| 44 | 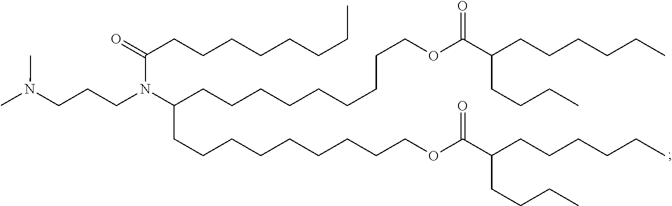 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] |
| 45 | 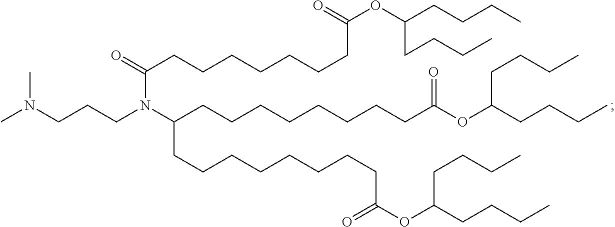 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] |
| 46 | 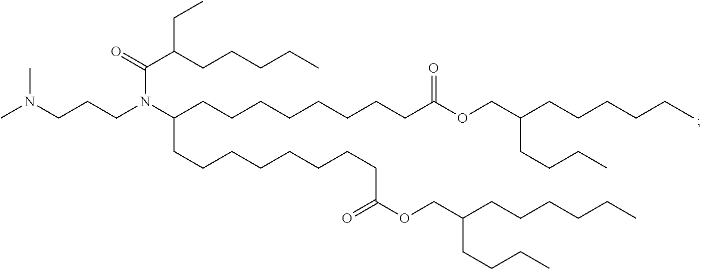 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC] |
| 47 | 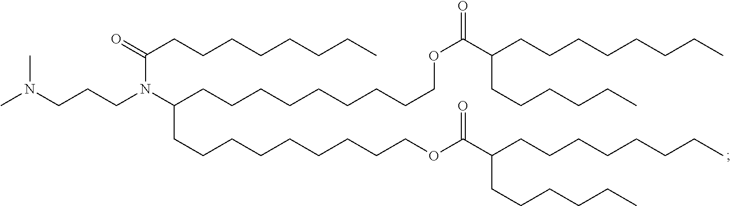 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC] |
| 48 | 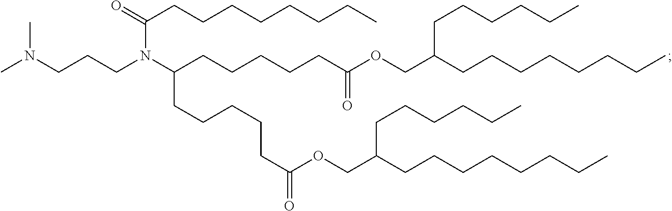 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] |
| 49 | 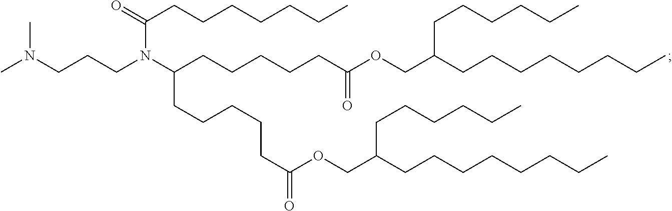 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC] |
| 50 | 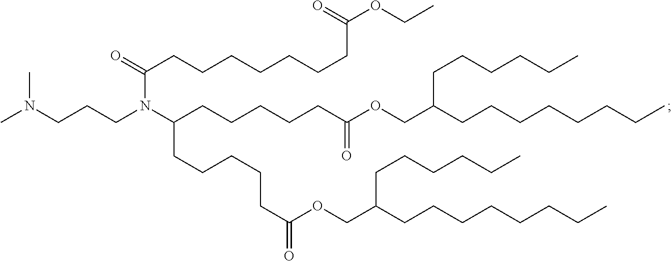 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC] |
| 51 | 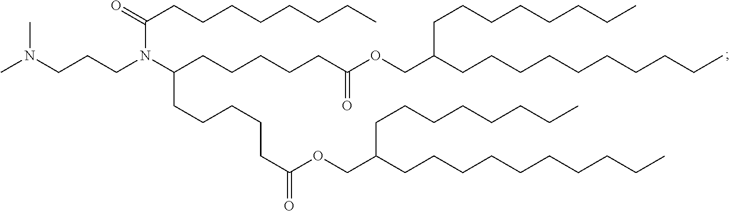 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC] |
| 52 | 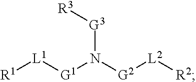 |
| 53 | 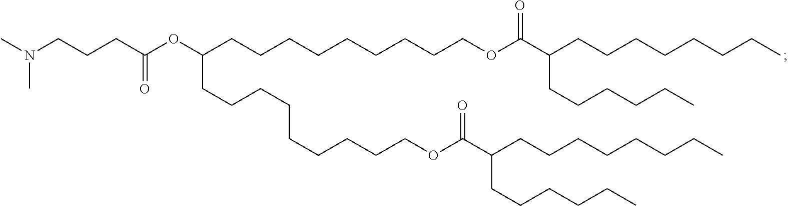 [SMILES: CCCCCCCCC(CCCCCC)C(=O)OCCCCCCCCCC(CCCCCCCCCOC(=O)C(CCCCCC)CCCCCCCC)OC(=O)CCCN(C)C] |
| 54 | 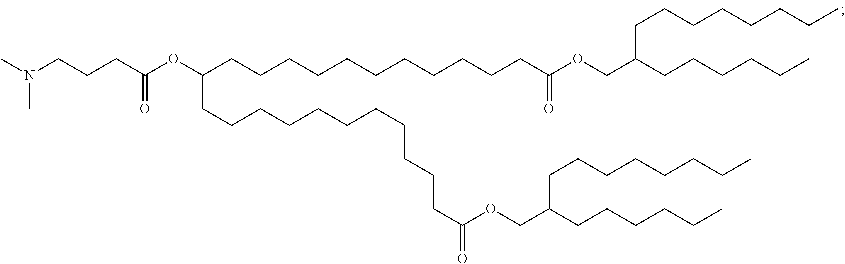 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C] |
| 55 | 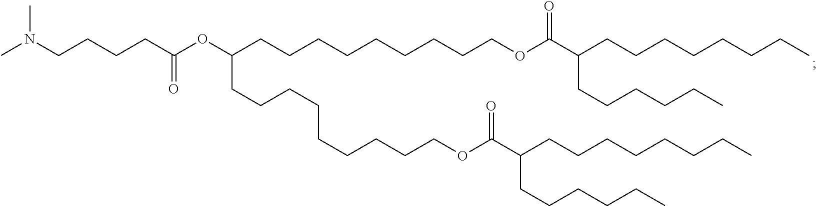 [SMILES: CCCCCCCCC(CCCCCC)C(=O)OCCCCCCCCCC(CCCCCCCCCOC(=O)C(CCCCCC)CCCCCCCC)OC(=O)CCCCN(C)C] |
| 56 | 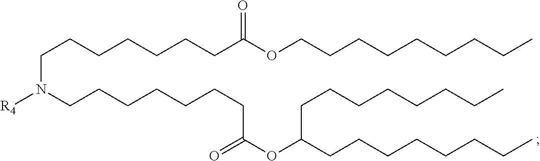 |
| 57 | 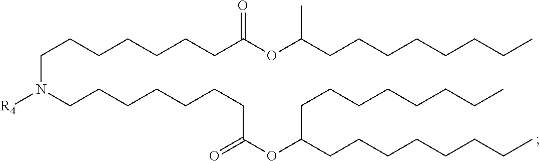 |
| 58 | 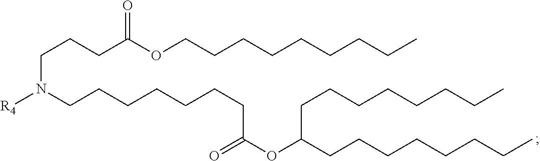 |
| 59 | 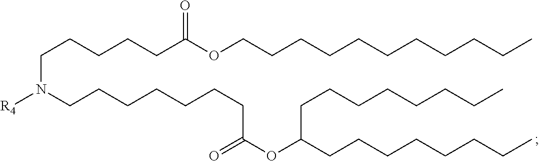 |
| 60 | 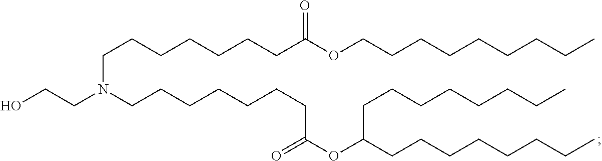 [SMILES: CCCCCCCCCOC(=O)CCCCCCCN(CCO)CCCCCCCC(=O)OC(CCCCCCCC)CCCCCCCC] |
| 61 | 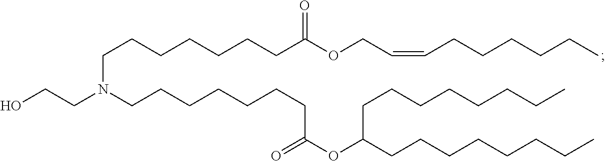 [SMILES: CCCCCC/C=C\COC(=O)CCCCCCCN(CCO)CCCCCCCC(=O)OC(CCCCCCCC)CCCCCCCC] |
| 62 | 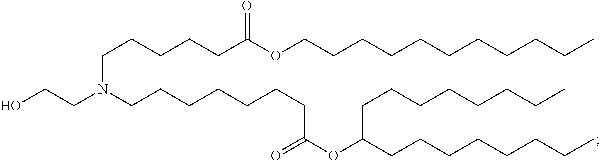 [SMILES: CCCCCCCCCCCOC(=O)CCCCCN(CCO)CCCCCCCC(=O)OC(CCCCCCCC)CCCCCCCC] |
| 63 | 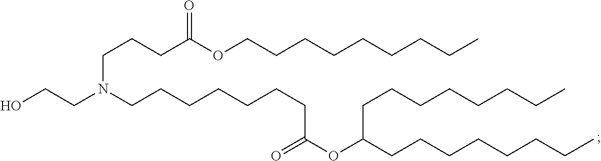 [SMILES: CCCCCCCCCOC(=O)CCCN(CCO)CCCCCCCC(=O)OC(CCCCCCCC)CCCCCCCC] |
| 64 | 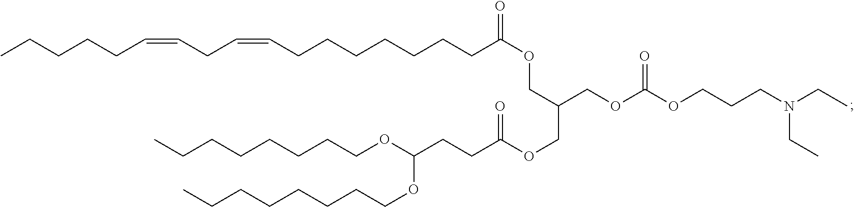 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 65 | 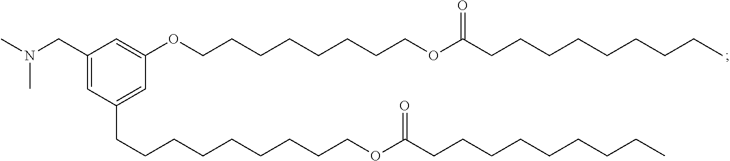 [SMILES: CCCCCCCCCC(=O)OCCCCCCCCCc1cc(CN(C)C)cc(OCCCCCCCCOC(=O)CCCCCCCCC)c1] |
| 66 | 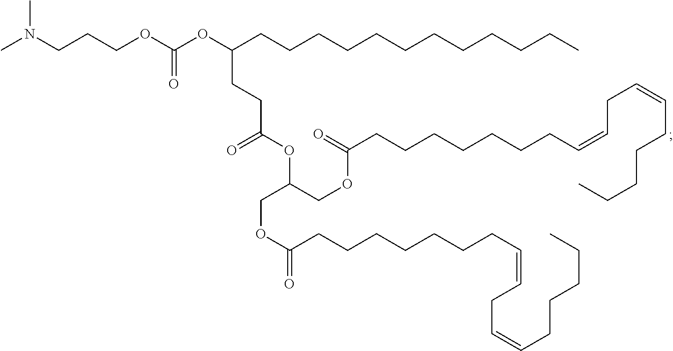 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C] |
| 67 | 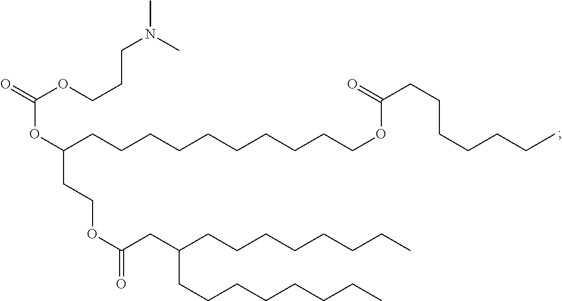 [SMILES: CCCCCCCCC(CCCCCCCC)CC(=O)OCCC(CCCCCCCCCCOC(=O)CCCCCCC)OC(=O)OCCCN(C)C] |
| 68 | 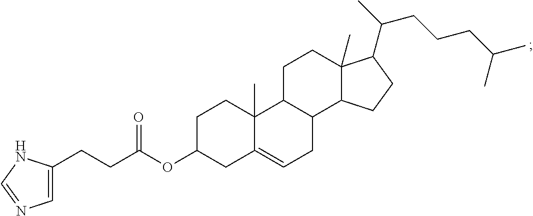 [SMILES: CC(C)CCCC(C)C1CCC2C3CC=C4CC(OC(=O)CCc5cnc[nH]5)CCC4(C)C3CCC12C] |
| 69 | 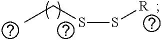 |
| 70 | 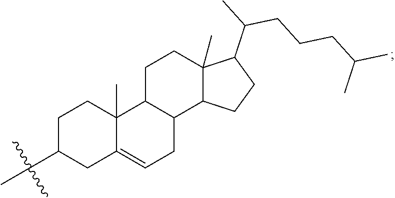 [SMILES: *C1CCC2(C)C(=CCC3C2CCC2(C)C(C(C)CCCC(C)C)CCC32)C1] |
| 71 | 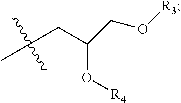 |
| 72 | 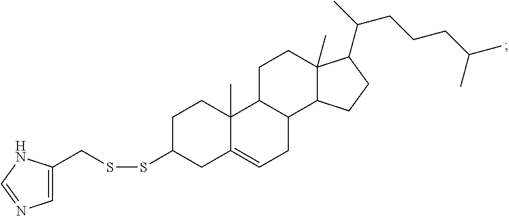 [SMILES: CC(C)CCCC(C)C1CCC2C3CC=C4CC(SSCc5cnc[nH]5)CCC4(C)C3CCC12C] |
| 73 | 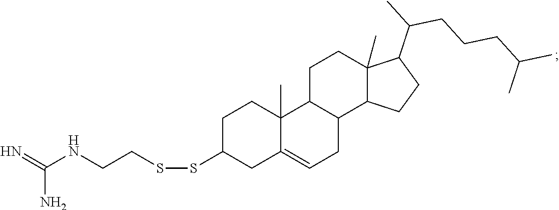 [SMILES: CC(C)CCCC(C)C1CCC2C3CC=C4CC(SSCCNC(=N)N)CCC4(C)C3CCC12C] |
| 74 | 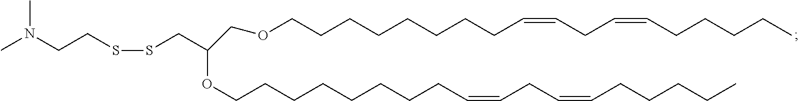 [SMILES: CCCCC/C=C\C/C=C\CCCCCCCCOCC(CSSCCN(C)C)OCCCCCCCC/C=C\C/C=C\CCCCC] |
| 75 | 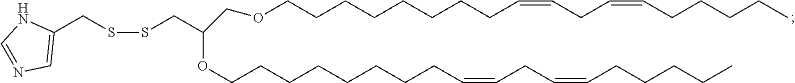 [SMILES: CCCCC/C=C\C/C=C\CCCCCCCCOCC(CSSCc1cnc[nH]1)OCCCCCCCC/C=C\C/C=C\CCCCC] |
| 76 | 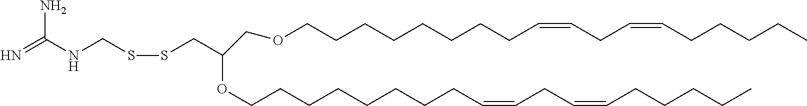 [SMILES: CCCCC/C=C\C/C=C\CCCCCCCCOCC(CSSCNC(=N)N)OCCCCCCCC/C=C\C/C=C\CCCCC] |

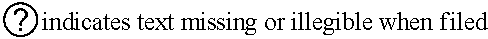

In some embodiments of the lipid composition of the present application, the ionizable cationic lipid is present in an amount from about from about 20 to about 23. In some embodiments, the molar percentage is from about 20, 20.5, 21, 21.5, 22, 22.5, to about 23 or any range derivable therein. In other embodiments, the molar percentage is from about 7.5 to about 20. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the ionizable cationic lipid at a molar percentage from about 5% to about 30%. In some embodiments of the lipid composition of the present application, the lipid composition comprises the ionizable cationic lipid at a molar percentage from about 10% to about 25%. In some embodiments of the lipid composition of the present application, the lipid composition comprises the ionizable cationic lipid at a molar percentage from about 15% to about 20%. In some embodiments of the lipid composition of the present application, the lipid composition comprises the ionizable cationic lipid at a molar percentage from about 10% to about 20%. In some embodiments of the lipid composition of the present application, the lipid composition comprises the ionizable cationic lipid at a molar percentage from about 20% to about 30%. In some embodiments of the lipid composition of the present application, the lipid composition comprises the ionizable cationic lipid at a molar percentage of at least (about) 5%, at least (about) 10%, at least (about) 15%, at least (about) 20%, at least (about) 25%, or at least (about) 30%. In some embodiments of the lipid composition of the present application, the lipid composition comprises the ionizable cationic lipid at a molar percentage of at most (about) 5%, at most (about) 10%, at most (about) 15%, at most (about) 20%, at most (about) 25%, or at most (about) 30%.

### Selective Organ Targeting (SORT) Lipids

In some embodiments of the lipid composition of the present application, the lipid (e.g., nanoparticle) composition is preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments of the lipid composition, the lipid composition comprises one or more selective organ targeting (SORT) lipid which leads to the selective delivery of the composition to a particular organ. In some embodiments, the SORT lipid may have two or more alkyl or alkenyl chains of C₆-C₂₄.

In some embodiments of the lipid compositions, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the lipid compositions, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid may comprise a structural formula:

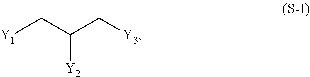

wherein:
Y₁, Y₂, or Y₃ are each independently X₁C(O)R₁ or X₂N⁺R₃R₄R₅;
provided at least one of Y₁, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NRₐ, wherein Rₐ is hydrogen, C₁-C₄ alkyl, or C₁-C₄ substituted alkyl;
X₂ is C₁-C₆ alkanediyl or C₁-C₆ substituted alkanediyl;
R₃, R₄, and R₅ are each independently C₁-C₂₄ alkyl, C₁-C₂₄ substituted alkyl, C₁-C₂₄ alkenyl, C₁-C₂₄ substituted alkenyl; and
A₁ is an anion with a charge equal to the number of X₂N⁺R₃R₄R₅ groups in the compound.

In some embodiments of the SORT lipids, the permanently cationic SORT lipid has a structural formula:

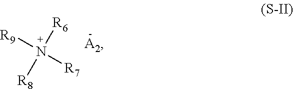

wherein:
R₆-R₉ are each independently C₁-C₂₄ alkyl, C₁-C₂₄ substituted alkyl, C₁-C₂₄ alkenyl, C₁-C₂₄ substituted alkenyl; provided at least one of R₆-R₉ is a group of C₈-C₂₄; and
A₂ is a monovalent anion.

In some embodiments of the lipid compositions, the SORT lipid is ionizable cationic lipid (i.e., comprising one or more hydrophobic components and an ionizable cationic group). The ionizable positively charged moiety may be positively charged at a physiological pH. One ionizable cationic group that may be used in the ionizable cationic lipid is a tertiary ammine group. In some embodiments of the lipid compositions, the SORT lipid has a structural formula:

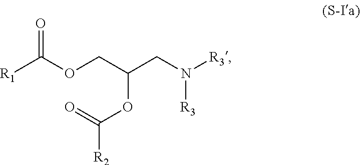

wherein:
   R₁ and R₂ are each independently alkyl_{(C8-C24)}, alkenyl_{(C8-C24)}, or a substituted version of either group; and
   R₃ and R₃′ are each independently alkyl_{(C≤6)} or substituted alkyl_{(C≤6)}.

In some embodiments of the lipid compositions, the SORT lipid comprises a head group of a particular structure. In some embodiments, the SORT lipid comprises a headgroup having a structural formula:

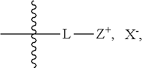

wherein L is a linker; Z⁺ is positively charged moiety and X⁻ is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the lipid compositions, the SORT lipid has a structural formula:

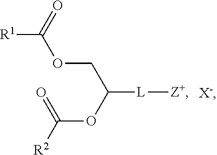

wherein R¹ and R² are each independently an optionally substituted C₆-C₂₄ alkyl, or an optionally substituted C₆-C₂₄ alkenyl.

In some embodiments of the lipid compositions, the SORT lipid has a structural formula:

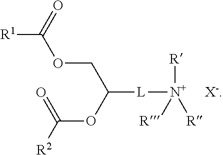

In some embodiments of the lipid compositions, the SORT lipid comprises a Linker (L). In some embodiments, L is

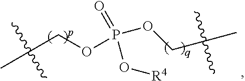

wherein:
p and q are each independently 1, 2, or 3; and
R⁴ is an optionally substituted C₁-C₆ alkyl

In some embodiments of the lipid compositions, the SORT lipid has a structural formula:

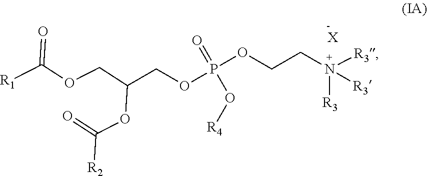

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

In some embodiments of the lipid compositions, the SORT lipid is a phosphotidylcholine (e.g., 14:0 EPC). In some embodiments, the phosphatidylcholine compound is further defined as:

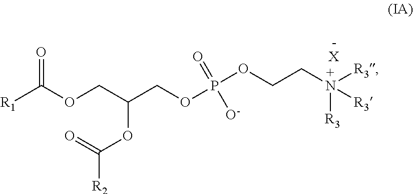

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In some embodiments of the lipid compositions, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the lipid compositions, the SORT lipid has a structural formula:

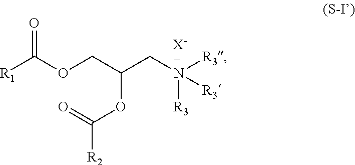

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the lipid compositions, the SORT lipid has a structural formula:

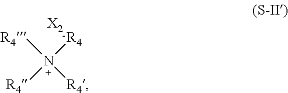

wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid comprises one or more selected from the lipids set forth in Table 6.

TABLE 6
Example SORT lipids

| Lipid Name | Structure |
|---|---|
| 1,2-Dioleoyl-3- dimethylammonium- propane (18:1 DODAP) | 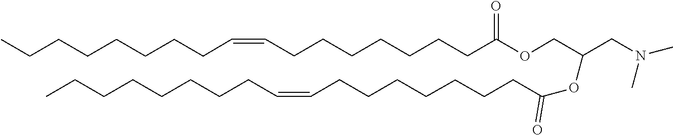 [SMILES: CCCCCCCC/C=C\CCCCCCCC(=O)OCC(CN(C)C)OC(=O)CCCCCCC/C=C\CCCCCCCC] |
| 1,2-Dioleoyl-3- trimethylammonium- propane (18:1 DOTAP) (e.g., chloride salt) | 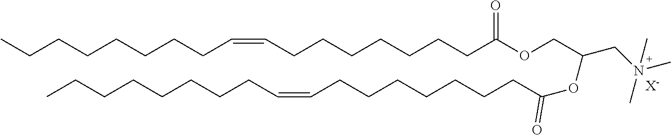 |
| 1,2-Di-O-octadeceny1-3- trimethylammonium propane (DOTMA) (e.g., chloride salt) | 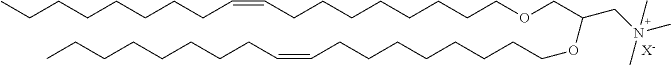 |
| Dimethyldioctadecyl- ammonium (DDAB) (e.g., bromide salt) | 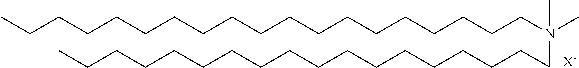 |
| 1,2-Dioleoyl-sn-glycero-3- ethylphosphocholine (14:0 EPC) (e.g., chloride salt) | 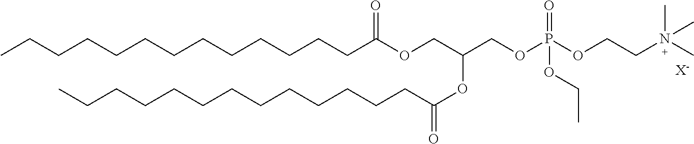 |

X⁻ is a counterion (e.g., Cl⁻, Br⁻, etc.)

In some embodiments of the lipid composition of the present application, the lipid composition comprises the SORT lipid at a molar percentage from about 20% to about 65%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage from about 30% to about 55%. In some embodiments of the lipid composition of the present application, the lipid composition comprises the SORT lipid at a molar percentage from about 20% to about 50%. In some embodiments of the lipid composition of the present application, the lipid composition comprises the SORT lipid at a molar percentage from about 30% to about 60%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage of at least (about) 25%, at least (about) 30%, at least (about) 35%, at least (about) 40%, at least (about) 45%, at least (about) 50%, at least (about) 55%, at least (about) 60%, or at least (about) 65%. In some embodiments of the lipid composition of the present application, the lipid composition comprises the SORT lipid at a molar percentage of at most (about) 25%, at most (about) 30%, at most (about) 35%, at most (about) 40%, at least (about) 45%, at most (about) 50%, at most (about) 55%, at most (about) 60%, or at most (about) 65%. In some embodiments of the lipid composition of the present application, the lipid composition comprises the SORT lipid at a molar percentage of about 20%, 25%, 30%, 35%, 40%, 45%, 50%, 55%, 60%, or 65%, or of a range between (inclusive) any two of the foregoing values.

### Additional Lipids

In some embodiments of the lipid composition of the present application, the lipid composition further comprises an additional lipid including but not limited to a steroid or a steroid derivative, a PEG lipid, and a phospholipid.

### Phospholipids or Other Zwitterionic Lipids

In some embodiments of the lipid composition of the present application, the lipid composition further comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments of the lipid compositions, the phospholipid is not an ethylphosphocholine.

In some embodiments of the lipid composition of the present application, the compositions may further comprise a molar percentage of the phospholipid to the total lipid composition from about 20 to about 23. In some embodiments, the molar percentage is from about 20, 20.5, 21, 21.5, 22, 22.5, to about 23 or any range derivable therein. In other embodiments, the molar percentage is from about 7.5 to about 60. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the phospholipid at a molar percentage from about 8% to about 23%. In some embodiments of the lipid composition of the present application, the lipid composition comprises the phospholipid at a molar percentage from about 10% to about 20%. In some embodiments of the lipid composition of the present application, the lipid composition comprises the phospholipid at a molar percentage from about 15% to about 20%. In some embodiments of the lipid composition of the present application, the lipid composition comprises the phospholipid at a molar percentage from about 8% to about 15%. In some embodiments of the lipid composition of the present application, the lipid composition comprises the phospholipid at a molar percentage from about 10% to about 15%. In some embodiments of the lipid composition of the present application, the lipid composition comprises the phospholipid at a molar percentage from about 12% to about 18%. In some embodiments of the lipid composition of the present application, the lipid composition comprises the phospholipid at a molar percentage of at least (about) 8%, at least (about) 10%, at least (about) 12%, at least (about) 15%, at least (about) 18%, at least (about) 20%, or at least (about) 23%. In some embodiments of the lipid composition of the present application, the lipid composition comprises the phospholipid at a molar percentage of at most (about) 8%, at most (about) 10%, at most (about) 12%, at most (about) 15%, at most (about) 18%, at most (about) 20%, or at most (about) 23%.

### Steroids or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In some embodiments, the ring structure of a steroid comprises three fused cyclohexyl rings and a fused cyclopentyl ring as shown in the formula:

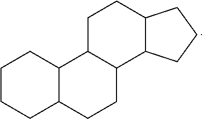 [SMILES: C1CCC2C(C1)CCC1C3CCCC3CCC21]

In some embodiments, a steroid derivative comprises the ring structure above with one or more non-alkyl substitutions. In some embodiments the steroid or steroid derivative is a sterol wherein the formula is further defined as:

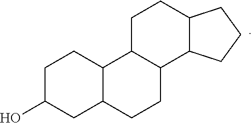 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

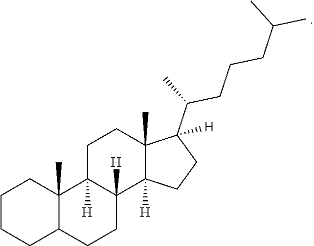

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar percentage of the steroid to the total lipid composition from about 40 to about 46. In some embodiments, the molar percentage is from about 40, 41, 42, 43, 44, 45, to about 46 or any range derivable therein. In other embodiments, the molar percentage of the steroid relative to the total lipid composition is from about 15 to about 40. In some embodiments, the molar percentage is 15, 16, 18, 20, 22, 24, 26, 28, 30, 32, 34, 36, 38, or 40, or any range derivable therein.

In some embodiments of the lipid composition of the present application, the lipid composition comprises the steroid or steroid derivative at a molar percentage from about 15% to about 46%. In some embodiments of the lipid composition of the present application, the lipid composition comprises the steroid or steroid derivative at a molar percentage from about 20% to about 40%. In some embodiments of the lipid composition of the present application, the lipid composition comprises the steroid or steroid derivative at a molar percentage from about 25% to about 35%. In some embodiments of the lipid composition of the present application, the lipid composition comprises the steroid or steroid derivative at a molar percentage from about 30% to about 40%. In some embodiments of the lipid composition of the present application, the lipid composition comprises the steroid or steroid derivative at a molar percentage from about 20% to about 30%. In some embodiments of the lipid composition of the present application, the lipid composition comprises the steroid or steroid derivative at a molar percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, the lipid composition comprises the steroid or steroid derivative at a molar percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain hydrocarbon groups (e.g., C₆-C₂₄ long chain alkyl or alkenyl group or a C₆-C₂₄ fatty acid group) attached to a linker group with a PEG chain. In some embodiments, the alkyl, alkenyl, or fatty acid group is about 6 carbon atoms to about 24 carbon atoms. In some embodiments, the alkyl, alkenyl, or fatty acid group is at least about 6 carbon atoms. In some embodiments, the alkyl, alkenyl, or fatty acid group is at most about 24 carbon atoms. In some embodiments, the alkyl, alkenyl, or fatty acid group is about 6 carbon atoms to about 8 carbon atoms, about 6 carbon atoms to about 10 carbon atoms, about 6 carbon atoms to about 12 carbon atoms, about 6 carbon atoms to about 14 carbon atoms, about 6 carbon atoms to about 16 carbon atoms, about 6 carbon atoms to about 20 carbon atoms, about 6 carbon atoms to about 22 carbon atoms, about 6 carbon atoms to about 24 carbon atoms, about 8 carbon atoms to about 10 carbon atoms, about 8 carbon atoms to about 12 carbon atoms, about 8 carbon atoms to about 14 carbon atoms, about 8 carbon atoms to about 16 carbon atoms, about 8 carbon atoms to about 20 carbon atoms, about 8 carbon atoms to about 22 carbon atoms, about 8 carbon atoms to about 24 carbon atoms, about 10 carbon atoms to about 12 carbon atoms, about 10 carbon atoms to about 14 carbon atoms, about 10 carbon atoms to about 16 carbon atoms, about 10 carbon atoms to about 20 carbon atoms, about 10 carbon atoms to about 22 carbon atoms, about 10 carbon atoms to about 24 carbon atoms, about 12 carbon atoms to about 14 carbon atoms, about 12 carbon atoms to about 16 carbon atoms, about 12 carbon atoms to about 20 carbon atoms, about 12 carbon atoms to about 22 carbon atoms, about 12 carbon atoms to about 24 carbon atoms, about 14 carbon atoms to about 16 carbon atoms, about 14 carbon atoms to about 20 carbon atoms, about 14 carbon atoms to about 22 carbon atoms, about 14 carbon atoms to about 24 carbon atoms, about 16 carbon atoms to about 20 carbon atoms, about 16 carbon atoms to about 22 carbon atoms, about 16 carbon atoms to about 24 carbon atoms, about 20 carbon atoms to about 22 carbon atoms, about 20 carbon atoms to about 24 carbon atoms, or about 22 carbon atoms to about 24 carbon atoms. In some embodiments, the alkyl, alkenyl, or fatty acid group is about 6 carbon atoms, about 8 carbon atoms, about 10 carbon atoms, about 12 carbon atoms, about 14 carbon atoms, about 16 carbon atoms, about 20 carbon atoms, about 22 carbon atoms, or about 24 carbon atoms. In some embodiments, the long chain hydrocarbon group or groups may comprise one or more unsaturated carbon bonds (e.g., double or triple carbon-carbon bonds). In some embodiments, the long chain hydrocarbon groups may comprise at least 1, 2, 3, 4, 5, 6, 7, 8, 9, 10, 11, 12, or more unsaturated carbon bonds. In some embodiments, the hydrocarbon groups may comprise no more than 12, 11, 10, 9, 8, 7, 6, 5, 4, 3, 2, 1, or fewer unsaturated carbon bonds. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. In some embodiments, the polymer-conjugated lipid has a molecular weight from about 500 to about 100,000 Daltons (Da). In some embodiments, the polymer-conjugated lipid has a molecular weight of about 100, 200, 300, 500, 1,000, 2,000, 5,000, 10,000, 20,000, 50,000, 100,000, or more Da. In some embodiments, the polymer-conjugated lipid has a molecular weight of more than about 100, 200, 300, 500, 1,000, 2,000, 5,000, 10,000, 20,000, 50,000, 100,000, or more. In some embodiments, the polymer-conjugated lipid has a molecular weight of no more than 100,000, 50,000, 20,000, 10,000, 5,000, 2,000, 1,000, 500, 300, 200, 100, or less. In some embodiments, the polymer-conjugated lipid has a molecular weight of about 100 Da to about 100,000 Da. In some embodiments, the polymer-conjugated lipid has a molecular weight of at least about 100 Da. In some embodiments, the polymer-conjugated lipid has a molecular weight of at most about 100,000 Da. In some embodiments, the polymer-conjugated lipid has a molecular weight of about 100 Da to about 200 Da, about 100 Da to about 300 Da, about 100 Da to about 500 Da, about 100 Da to about 1,000 Da, about 100 Da to about 2,000 Da, about 100 Da to about 5,000 Da, about 100 Da to about 10,000 Da, about 100 Da to about 20,000 Da, about 100 Da to about 50,000 Da, about 100 Da to about 100,000 Da, about 200 Da to about 300 Da, about 200 Da to about 500 Da, about 200 Da to about 1,000 Da, about 200 Da to about 2,000 Da, about 200 Da to about 5,000 Da, about 200 Da to about 10,000 Da, about 200 Da to about 20,000 Da, about 200 Da to about 50,000 Da, about 200 Da to about 100,000 Da, about 300 Da to about 500 Da, about 300 Da to about 1,000 Da, about 300 Da to about 2,000 Da, about 300 Da to about 5,000 Da, about 300 Da to about 10,000 Da, about 300 Da to about 20,000 Da, about 300 Da to about 50,000 Da, about 300 Da to about 100,000 Da, about 500 Da to about 1,000 Da, about 500 Da to about 2,000 Da, about 500 Da to about 5,000 Da, about 500 Da to about 10,000 Da, about 500 Da to about 20,000 Da, about 500 Da to about 50,000 Da, about 500 Da to about 100,000 Da, about 1,000 Da to about 2,000 Da, about 1,000 Da to about 5,000 Da, about 1,000 Da to about 10,000 Da, about 1,000 Da to about 20,000 Da, about 1,000 Da to about 50,000 Da, about 1,000 Da to about 100,000 Da, about 2,000 Da to about 5,000 Da, about 2,000 Da to about 10,000 Da, about 2,000 Da to about 20,000 Da, about 2,000 Da to about 50,000 Da, about 2,000 Da to about 100,000 Da, about 5,000 Da to about 10,000 Da, about 5,000 Da to about 20,000 Da, about 5,000 Da to about 50,000 Da, about 5,000 Da to about 100,000 Da, about 10,000 Da to about 20,000 Da, about 10,000 Da to about 50,000 Da, about 10,000 Da to about 100,000 Da, about 20,000 Da to about 50,000 Da, about 20,000 Da to about 100,000 Da, or about 50,000 Da to about 100,000 Da. In some embodiments, the polymer-conjugated lipid has a molecular weight of about 100 Da, about 200 Da, about 300 Da, about 500 Da, about 1,000 Da, about 2,000 Da, about 5,000 Da, about 10,000 Da, about 20,000 Da, about 50,000 Da, or about 100,000 Da. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

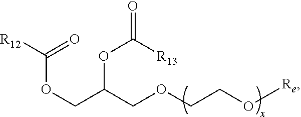

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

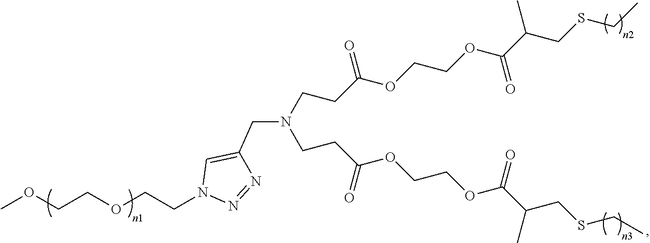

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar percentage of the PEG lipid to the total lipid composition from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

In some embodiments of the lipid composition of the present application, the lipid composition comprises the polymer-conjugated lipid at a molar percentage from about 0.5% to about 10%. In some embodiments of the lipid composition of the present application, the lipid composition comprises the polymer-conjugated lipid at a molar percentage from about 1% to about 8%. In some embodiments of the lipid composition of the present application, the lipid composition comprises the polymer-conjugated lipid at a molar percentage from about 2% to about 7%. In some embodiments of the lipid composition of the present application, the lipid composition comprises the polymer-conjugated lipid at a molar percentage from about 3% to about 5%. In some embodiments of the lipid composition of the present application, the lipid composition comprises the polymer-conjugated lipid at a molar percentage from about 5% to about 10%. In some embodiments of the lipid composition of the present application, the lipid composition comprises the polymer-conjugated lipid at a molar percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, the lipid composition comprises the polymer-conjugated lipid at a molar percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Pharmaceutical Compositions

### Therapeutic or Prophylactic Agents

In some embodiments, provided herein is a pharmaceutical composition comprising a therapeutic agent (or prophylactic agent) assembled to a lipid composition as described herein.

In some embodiments of the pharmaceutical composition, the therapeutic agent (or prophylactic agent) comprises a compound, a polynucleotide, a polypeptide, or a combination thereof.

In some embodiments, the compound, the polynucleotide, the polypeptide, or a combination thereof is exogenous or heterologous to the cell or the subject being treated by the pharmaceutical compositions described herein. In some embodiments, the therapeutic agent (or prophylactic agent) comprises a compound described herein. In some embodiments, the therapeutic agent (or prophylactic agent) comprises a polynucleotide described herein. In some embodiments, the therapeutic agent (or prophylactic agent) comprises a polypeptide described herein. In some embodiments, the therapeutic agent (or prophylactic agent) comprises a compound, a polynucleotide, a polypeptide, or a combination thereof.

In some embodiments, the pharmaceutical composition comprises a therapeutic agent (or prophylactic agent) for treating a lung disease such as asthma, COPD, or lung cancer. In some embodiments, the therapeutic agent (or prophylactic agent) comprises a steroid such as prednisone, hydrocortisone, prednisolone, methylprednisolone, or dexamethasone. In some embodiments, the therapeutic agent (or prophylactic agent) comprises Abraxane, Afatinib Dimaleate, Afinitor, Afinitor Disperz, Alecensa, Alectinib, Alimta, Alunbrig, Atezolizumab, Avastin, Bevacizumab, Brigatinib, Capmatinib Hydrochloride, Carboplatin, Ceritinib, Crizotinib, Cyramza, Dabrafenib Mesylate, Dacomitinib, Docetaxel, Doxorubicin Hydrochloride, Durvalumab, Entrectinib, Erlotinib Hydrochloride, Everolimus, Gavreto, Gefitinib, Gilotrif, Gemcitabine, Ipilimumab, Iressa, Keytruda, Lorbrena, Mekinist, Methotrexate Sodium, Necitumumab, Nivolumab, Osimertinib Mesylate, Paclitaxel, Pembrolizumab, Pemetrexed Disodium, Pralsetinib, Ramucirumab, Retevmo, Selpercatinib, Tabrecta, Tafinlar, Tagrisso, Trametinib Dimethyl Sulfoxide, Vizimpro, Vinorelbine Tartrate, Xalkori, Yervoy, Zirabev, Zykadia, Carboplatin, Gemcitabine-cisplatin, Afinitor, Atezolizumab, Durvalumab, Etopophos, Etoposide, Hycamtin, Imfinzi, Keytruda, Lurbinectedin, Methotrexate Sodium, Nivolumab, Opdivo, Pembrolizumab, Tecentriq, Topotecan Hydrochloride, Trexall, or Zepzelca. Other non-limiting examples of the therapeutic agents (or prophylactic agents) comprising compounds include small molecule selected from 7-Methoxypteridine, 7 Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, or zopiclone.

### Polynucleotides

In some embodiments of the pharmaceutical compositions of the present disclosure, the therapeutic agent (or prophylactic agent) assembled to the lipid composition comprises one or more polynucleotides. The present application is not limited in scope to any particular source, sequence, or type of polynucleotide; however, as one of ordinary skill in the art could readily identify related homologs in various other sources of the polynucleotide including nucleic acids from non-human species (e.g., mouse, rat, rabbit, dog, monkey, gibbon, chimp, ape, baboon, cow, pig, horse, sheep, cat and other species). It is contemplated that the polynucleotide used in the present application can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the polynucleotide comprises nucleic acid sequence that is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

In some embodiments, the polynucleotide used herein may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene; such engineered molecules are sometime referred to as “mini-genes.” At a minimum, these and other nucleic acids of the present application may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the polynucleotide comprises one or more segments comprising a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a or double stranded ribonucleic acid (dsRNA). In some embodiments, the polynucleotide encodes at least one of the therapeutic agent (or prophylactic agent) described herein. In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an endonuclease. Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

In some embodiments, the therapeutic (or prophylactic) agent is a transfer ribonucleic acid (tRNA) that introduces an amino acid into a growing peptide chain of a protein of a target gene.

Some embodiments of the therapeutic agent (or prophylactic agent) provided herein comprise a heterologous polypeptide comprising an actuator moiety. The actuator moiety can be configured to complex with a target polynucleotide corresponding to a target gene. In some embodiments, administration of the therapeutic agent (or prophylactic agent) results in a modified expression or activity of the target gene. The modified expression or activity of the target gene can be detectable, for example, in at least about 1% (e.g., at least about 2%, 5%, 10%, 15%, or 20%) cells (e.g., lung cells, such as lung basal cells) of the subject. The therapeutic agent (or prophylactic agent) may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced shortpalindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Casl, CaslB, Cas2, Cas3, Cas4, Cas5, Cas5e (CasD), Cas6, Cas6e, Cas6f, Cas7, Cas8a, Cas8al, Cas8a2, Cas8b, Cas8c, Csnl, Csxl2, Cas10, Cas10d, CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, or Cul966; any derivative thereof; any variant thereof; or any fragment thereof. In some embodiments, Cas13 can include, but are not limited to, Cas13a, Cas13b, Cas13c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIM5, KYP, SUVR4, Set4, Set1, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. Examples of the genes that may be modified by the guide polynucleotide guided nuclease include CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

In some embodiments, the polynucleotide comprises or encodes at least one mRNA that, upon expression of the mRNA, restores the function of a defective gene in a subject being treated by the pharmaceutical composition described herein. For example, the polynucleotide comprises or encodes an mRNA that expresses a wild type CFTR protein, which may be used to rescue a subject who is afflicted with inborn mutation in CFTR protein. Other examples of mRNA that can be expressed from the polynucleotide includes mRNA that encodes DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. In some embodiments, the at least one chemical modification stabilizes scaffold such as a tRNA scaffold. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₐ), and O—CH₂—C(═O)—N(Rₘ)(Rₐ), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₐ) or O—CH₂—C(═O)—N(Rₘ)(Rₐ), where each Rₘ and Rₐ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rn), O(CH₂)₂O(CH₂)₂N(CH₃)₂, and N-substituted acetamide (O—CH₂—C(═O)—N(Rₘ)(Rₐ) where each Rₘ and Rₐ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof; 4′-C(CH₃)(CH₃)—O-2′ and analogs thereof; 4′-CH₂—N(OCH₃)-2′ and analogs thereof; 4′-CH₂—O—N(CH₃)-2′; 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group; 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In certain embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides polynucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or (S), a or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, the polynucleotides described herein comprise or encode at least one tRNA described herein. In some embodiments, the tRNA expressed from the polynucleotide restores the function of at least one defective tRNA in a subject who is being treated by the pharmaceutical composition described herein. In some embodiments, the at least one tRNA expressed by the polynucleotide described herein may include tRNA that encodes alanine, arginine, asparagine, aspartic acid, cysteine, glutamic acid, glutamine, glycine, histidine, hydroxyproline, isoleucine, leucin, lysine, methionine, phenylaniline, proline, pyroglutamic acid, serine, threonine, tryptophan, tyrosine, or valine. In some embodiments, the at least one tRNA expressed by the polynucleotide described herein may include tRNA that encodes arginine, tryptophan, glutamic acid, glutamine, serine, tyrosine, lysine, leucine, glycine, or cysteine. In some embodiments, the tRNA encoded by the polynucleotide described herein may restore the expression of any one of the genes described herein. In some embodiments, the tRNA encoded by the polynucleotide described herein may restore the expression of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

### Polypeptides

In some embodiments of the pharmaceutical compositions of the present disclosure, the therapeutic agent (or prophylactic agent) assembled to the lipid composition comprises one or more one or more polypeptides. Some polypeptide may include enzymes such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof. In some embodiments, the nuclease enzyme may include Cas proteins such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csxl4, Csx10, Csx16, CsaX, Csx3, Csx1, Csxl5, Csf1, Csf2, Csf3, Csf4, homologs thereof, or modified versions thereof. In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein. For example, the CRISPR enzyme may be directed and cleaved a genomic locus of CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, the present disclosure provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin a, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin a, Darbepoetin a, Follicle-stimulating hormone (FSH), and Factor VIII.

In some embodiments, the polypeptide comprises a peptide sequence that is at least partially identical to any of the therapeutic agent (or prophylactic agent) comprising a peptide sequence. For example, the polypeptide may comprise a peptide sequence that is at least partially identical to an antibody (e.g., a monoclonal antibody) for treating a lung disease such as lung cancer.

In some embodiments, the polypeptide comprises a peptide or protein that restores the function of a defective protein in a subject being treated by the pharmaceutical composition described herein. For example, the polynucleotide comprises a peptide or protein that restores function of cystic fibrosis transmembrane conductance regulator (CFTR) protein, which may be used to rescue a subject who is afflicted with inborn error leading to the expression of the mutated CFTR protein. Other examples of the rescue may include administering to a subject in need thereof a polypeptide comprising a peptide or protein of wild type Dynein axonemal heavy chain 5, Dynein axonemal heavy chain 11,Bone morphogenetic protein receptor type 2,Fumarylacetoacetate hydrolase, Phenylalanine hydroxylase, Alpha-L-iduronidase, Collagen type IV alpha 3 chain, Collagen type IV alpha 4 chain, Collagen type IV alpha 5 chain, Polycystin 1, Polycystin 2, Fibrocystin (or polyductin), Solute carrier family 3 member 1,Solute carrier family 7 member 9,Paired box gene 9,Myosin VIIA, Cadherin related 23, Usherin, Clarin 1, Gap junction beta-2 protein, Gap junction beta-6 protein, Rhodopsin, dystrophia myotonica protein kinase, Dystrophin, Sodium voltage-gated channel alpha subunit 1, Sodium voltage-gated channel beta subunit 1, Coagulation factor VIII, Coagulation factor IX, N-glycanase 1, Tumor protein p53, Palmitoyl-protein thioesterase 1, Tripeptidyl peptidase 1,Kv11.1 (alpha subunit of potassium ion channel), Palmitoyl-protein thioesterase 1, ATM serine/threonine kinase, or Fibrillin 1.

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no more than (about) 20:1, no more than (about) 15:1, no more than (about) 10:1, or no more than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no less than (about) 20:1, no less than (about) 15:1, no less than (about) 10:1, or no less than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 10:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 10:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 15:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1.

In some embodiments of the pharmaceutical composition of the present disclosure, a molar ratio of the therapeutic agent to total lipids of the lipid composition is from about 1:1 to about 1:100. In some embodiments of the pharmaceutical composition of the present application, a molar ratio of the therapeutic agent to total lipids of the lipid composition is from about 1:1 to about 1:50. In some embodiments of the pharmaceutical composition of the present application, a molar ratio of the therapeutic agent to total lipids of the lipid composition is from about 50:1 to about 1:100. In some embodiments of the pharmaceutical composition of the present application, a molar ratio of the therapeutic agent to total lipids of the lipid composition is from about 1:1 to about 1:20. In some embodiments of the pharmaceutical composition of the present application, a molar ratio of the therapeutic agent to total lipids of the lipid composition is from about 20:1 to about 1:50. In some embodiments of the pharmaceutical composition of the present application, a molar ratio of the therapeutic agent to total lipids of the lipid composition is from about 50:1 to about 1:70. In some embodiments of the pharmaceutical composition of the present application, a molar ratio of the therapeutic agent to total lipids of the lipid composition is from about 70:1 to about 1:100. In some embodiments of the pharmaceutical composition of the present application, a molar ratio of the therapeutic agent to total lipids of the lipid composition is no more than (about) 1:1, no more than (about) 1:5, no more than (about) 1:10, no more than (about) 1:15, no more than (about) 1:20, no more than (about) 1:25, no more than (about) 1:30, no more than (about) 1:35, no more than (about) 1:40, no more than (about) 1:45, no more than (about) 1:50, no more than (about) 1:60, no more than (about) 1:70, no more than (about) 1:80, no more than (about) 1:90, or more than (about) 1:100. In some embodiments of the pharmaceutical composition of the present application, a molar ratio of the therapeutic agent to total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100.

In some embodiments of the pharmaceutical composition of the present disclosure, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or (about) 100% of the therapeutic agent is encapsulated in particles of the lipid compositions.

In some embodiments of the pharmaceutical composition of the present disclosure, the lipid composition comprises a plurality of particles characterized by one or more characteristics of the following: (1) a (e.g., average) size of 100 nanometers (nm) or less; (2) a polydispersity index (PDI) of no more than about 0.2; and (3) a zeta potential of −10 millivolts (mV) to 10 mV.

In some embodiments of the pharmaceutical composition of the present disclosure, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present disclosure, the lipid composition comprises a plurality of particles with a (e.g., average) size from about 50 nanometers (nm) to about 80 nanometers (nm). In some embodiments of the pharmaceutical composition of the present disclosure, the lipid composition comprises a plurality of particles with a (e.g., average) size from about 60 nanometers (nm) to about 80 nanometers (nm). In some embodiments of the pharmaceutical composition of the present disclosure, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present disclosure, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments of the pharmaceutical composition of the present disclosure, the lipid composition comprises a plurality of particles with a polydispersity index (PDI) from about 0.05 to about 0.5. In some embodiments of the pharmaceutical composition of the present disclosure, the lipid composition comprises a plurality of particles with a polydispersity index (PDI) from about 0.1 to about 0.5. In some embodiments of the pharmaceutical composition of the present disclosure, the lipid composition comprises a plurality of particles with a polydispersity index (PDI) from about 0.1 to about 0.3. In some embodiments of the pharmaceutical composition of the present disclosure, the lipid composition comprises a plurality of particles with a polydispersity index (PDI) from about 0.2 to about 0.5. In some embodiments of the pharmaceutical composition of the present disclosure, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present disclosure, the lipid composition comprises a plurality of particles with a negative zeta potential of −5 millivolts (mV) or less. In some embodiments of the pharmaceutical composition of the present disclosure, the lipid composition comprises a plurality of particles with a negative zeta potential of −10 millivolts (mV) or less. In some embodiments of the pharmaceutical composition of the present disclosure, the lipid composition comprises a plurality of particles with a negative zeta potential of −15 millivolts (mV) or less. In some embodiments of the pharmaceutical composition of the present disclosure, the lipid composition comprises a plurality of particles with a negative zeta potential of −20 millivolts (mV) or less. In some embodiments of the pharmaceutical composition of the present disclosure, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present disclosure, the lipid composition comprises a plurality of particles with a negative zeta potential of 15 millivolts (mV) or less. In some embodiments of the pharmaceutical composition of the present disclosure, the lipid composition comprises a plurality of particles with a negative zeta potential of 20 millivolts (mV) or less.

In some embodiments of the pharmaceutical composition of the present disclosure, the lipid composition comprises a plurality of particles with a negative zeta potential of −5 millivolts (mV) or more. In some embodiments of the pharmaceutical composition of the present disclosure, the lipid composition comprises a plurality of particles with a negative zeta potential of −10 millivolts (mV) or more In some embodiments of the pharmaceutical composition of the present disclosure, the lipid composition comprises a plurality of particles with a negative zeta potential of −15 millivolts (mV) or more. In some embodiments of the pharmaceutical composition of the present disclosure, the lipid composition comprises a plurality of particles with a negative zeta potential of −20 millivolts (mV) or more. In some embodiments of the pharmaceutical composition of the present disclosure, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present disclosure, the lipid composition comprises a plurality of particles with a negative zeta potential of 15 millivolts (mV) or more. In some embodiments of the pharmaceutical composition of the present disclosure, the lipid composition comprises a plurality of particles with a negative zeta potential of 20 millivolts (mV) or more.

In some embodiments of the pharmaceutical composition of the present disclosure, the lipid composition has an apparent ionization constant (pKa) outside a range of 6 to 7. In some embodiments of the pharmaceutical composition of the present disclosure, the lipid composition has an apparent pKa of about 8 or higher, about 9 or higher, about 10 or higher, about 11 or higher, about 12 or higher, or about 13 or higher. In some embodiments of the pharmaceutical composition of the present disclosure, the lipid composition has an apparent pKa of about 8 to about 13. In some embodiments of the pharmaceutical composition of the present disclosure, the lipid composition has an apparent pKa of about 8 to about 10. In some embodiments of the pharmaceutical composition of the present disclosure, the lipid composition has an apparent pKa of about 9 to about 11. In some embodiments of the pharmaceutical composition of the present disclosure, the lipid composition has an apparent pKa of about 10 to about 13. In some embodiments of the pharmaceutical composition of the present disclosure, the lipid composition has an apparent pKa of about 8 to about 12. In some embodiments of the pharmaceutical composition of the present disclosure, the lipid composition has an apparent pKa of about 10 to about 12.

In some embodiments of the pharmaceutical composition of the present disclosure, the SORT lipid in the pharmaceutical composition effects a delivery of the therapeutic agent characterized by one or more of the following: (a) a greater therapeutic effect in a cell of the subject compared to that achieved with a reference lipid composition; (b) a therapeutic effect in a greater plurality of cells of the subject compared to that achieved with a reference lipid composition; (c) a therapeutic effect in a first plurality of cells of a first cell type and in a greater second plurality of cells of a second cell type; and (d) a greater therapeutic effect in a first cell of a first cell type of the subject compared to that in a second cell of a second cell type of the subject. In some embodiments, the first cell type is different from the second cell type.

In some embodiments of the pharmaceutical composition of the present disclosure, the cell is a lung cell. In some embodiments, the lung cell is a lung airway cell. Example lung airway cells that can be targeted by the delivery of the present disclosure includes but is not limited to basal cell.

In some embodiments of the pharmaceutical composition of the present disclosure, the therapeutic effect is characterized by a therapeutically effective amount of the therapeutic agent, for example, in a lung, a lung cell, a plurality of lung cells, or a lung cell type of the subject. In some embodiments, the therapeutic effect is characterized by an activity of the therapeutic agent, for example, in a lung, a lung cell, a plurality of lung cells, or a lung cell type of the subject. In some embodiments, the therapeutic effect is characterized by an effect of the therapeutic agent, for example, in a lung, a lung cell, a plurality of lung cells, or a lung cell type of the subject. In some embodiments, the greater therapeutic effect is characterized by a greater therapeutic amount of the therapeutic agent. In some embodiments, the greater therapeutic effect is characterized by a greater activity of the therapeutic agent. In some embodiments, the greater therapeutic effect is characterized by a greater effect of the therapeutic agent.

In some embodiments of the pharmaceutical composition of the present disclosure, the SORT lipid in the pharmaceutical composition effects delivery of the therapeutic agent to the cell of the subject characterized by a greater therapeutic effect compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the pharmaceutical composition of the present disclosure, the SORT lipid in the pharmaceutical composition achieves about 1.1-fold to about 20-fold therapeutic effect compared to that achieved with a reference lipid composition. In some embodiments, the SORT lipid achieves about 1.1-fold to about 10-fold therapeutic effect compared to that achieved with a reference lipid composition. In some embodiments, the SORT lipid achieves about 5-fold to about 10-fold therapeutic effect compared to that achieved with a reference lipid composition. In some embodiments, the SORT lipid achieves about 10-fold to about 20-fold therapeutic effect compared to that achieved with a reference lipid composition. In some embodiments, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition.

In some embodiments of the pharmaceutical composition of the present disclosure, the SORT lipid in the pharmaceutical composition achieves about 1.1-fold to about 20-fold therapeutic effect compared to that achieved with a reference lipid composition in basal cell. In some embodiments, the SORT lipid achieves about 1.1-fold to about 10-fold therapeutic effect compared to that achieved with a reference lipid composition in basal cell. In some embodiments, the SORT lipid achieves about 5-fold to about 10-fold therapeutic effect compared to that achieved with a reference lipid composition in basal cell. In some embodiments, the SORT lipid achieves about 10-fold to about 20-fold therapeutic effect compared to that achieved with a reference lipid composition in basal cell. In some embodiments, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition in basal cell.

In some embodiments of the pharmaceutical composition of the present disclosure, the SORT lipid in the pharmaceutical composition effects delivery of the therapeutic agent to cells of the subject characterized by a therapeutic effect in a greater plurality of cells compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the pharmaceutical composition of the present disclosure, the SORT lipid in the pharmaceutical composition achieves therapeutic effect in about 1.1-fold to about 20-fold cells compared to that achieved with a reference lipid composition. In some embodiments, the SORT lipid achieves therapeutic effect in about 1.1-fold to about 10-fold cells compared to that achieved with a reference lipid composition. In some embodiments, the SORT lipid achieves therapeutic effect in about 5-fold to about 10-fold cells compared to that achieved with a reference lipid composition. In some embodiments, the SORT lipid achieves therapeutic effect in about 10-fold to about 20-fold cells compared to that achieved with a reference lipid composition. In some embodiments, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold cells compared to that achieved with a reference lipid composition.

In some embodiments of the pharmaceutical composition of the present disclosure, the SORT lipid in the pharmaceutical composition achieves therapeutic effect in about 1.1-fold to about 20-fold cells compared to that achieved with a reference lipid composition in basal cell. In some embodiments, the SORT lipid achieves therapeutic effect in about 1.1-fold to about 10-fold more cells compared to that achieved with a reference lipid composition in basal cell. In some embodiments, the SORT lipid achieves therapeutic effect in about 5-fold to about 10-fold more cells compared to that achieved with a reference lipid composition in basal cell. In some embodiments, the SORT lipid achieves therapeutic effect in about 10-fold to about 20-fold more cells compared to that achieved with a reference lipid composition in basal cell. In some embodiments, the SORT lipid achieves therapeutic effect in about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold more cells compared to that achieved with a reference lipid composition in basal cell.

In some embodiments of the pharmaceutical composition of the present disclosure, the SORT lipid in the pharmaceutical composition effects delivery of the therapeutic agent to cells of the subject characterized by a therapeutic effect in a first plurality of cells of a first cell type and in a greater therapeutic effect in a second plurality of cells of a second cell type. In some embodiments, the first cell type is different from the second cell type.

In some embodiments of the pharmaceutical composition of the present disclosure, the first cell type is a lung cell. In some embodiments, the first cell type is a lung airway cell. Example lung airway cells that can be targeted by the delivery of the present application includes but is not limited to basal cell.

In some embodiments of the pharmaceutical composition of the present disclosure, the second cell type is a lung cell. In some embodiments, the second cell type is a lung airway cell.

In some embodiments of the pharmaceutical composition of the present disclosure, the SORT lipid in the pharmaceutical composition achieves therapeutic effect in about 1.1-fold to about 20-fold greater second plurality of cells of the second cell type compared to the first plurality of cells of the first cell type. In some embodiments, the SORT lipid achieves therapeutic effect in about 1.1-fold to about 10-fold greater second plurality of cells of the second cell type compared to the first plurality of cells of the first cell type. In some embodiments, the SORT lipid achieves therapeutic effect in about 5-fold to about 10-fold greater second plurality of cells of the second cell type compared to the first plurality of cells of the first cell type. In some embodiments, the SORT lipid achieves therapeutic effect in about 10-fold to about 20-fold greater second plurality of cells of the second cell type compared to the first plurality of cells of the first cell type. In some embodiments, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold greater second plurality of cells of the second cell type compared to the first plurality of cells of the first cell type.

In some embodiments of the pharmaceutical composition of the present disclosure, the SORT lipid in the pharmaceutical composition effects delivery of the therapeutic agent to cells of the subject characterized by a greater therapeutic effect in a first cell of a first cell type compared to that in a second cell of a second cell type. In some embodiments, the first cell type is different from the second cell type.

In some embodiments of the pharmaceutical composition of the present disclosure, the first cell type is a lung cell. In some embodiments, the first cell type is a lung airway cell. Examples of lung airway cells that can be targeted by the delivery of the present application include but is not limited to basal cells.

In some embodiments of the pharmaceutical composition of the present disclosure, the second cell type is a lung cell. In some embodiments, the second cell type is a lung airway cell.

In some embodiments of the pharmaceutical composition of the present disclosure, the SORT lipid in the pharmaceutical composition achieves about 1.1-fold to about 20-fold therapeutic effect in first cell of the first cell type compared to that achieved in the second cell of the second cell type. In some embodiments, the SORT lipid achieves about 1.1-fold to about 10-fold therapeutic effect in first cell of the first cell type compared to that achieved in the second cell of the second cell type. In some embodiments, the SORT lipid achieves about 5-fold to about 10-fold therapeutic effect in first cell of the first cell type compared to that achieved in the second cell of the second cell type. In some embodiments, the SORT lipid achieves about 10-fold to about 20-fold therapeutic effect in first cell of the first cell type compared to that achieved in the second cell of the second cell type. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect in first cell of the first cell type compared to that achieved in the second cell of the second cell type.

In some embodiments, provided herein are (e.g., pharmaceutical) compositions that comprise components that allow for an improved efficacy or outcome based on the delivery of the polynucleotide. The compositions described elsewhere herein may be more effective at delivery to a particular cell, cell type, organ, or bodily region as compared to a reference composition or compound. The compositions described elsewhere herein may be more effective at generating increase expression of a corresponding polypeptide of a delivered polynucleotide. The compositions described elsewhere herein may be more effective at generating a larger number of cells that express a corresponding polypeptide of a delivered polynucleotide. The compositions described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the SORT lipid is present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the SORT lipid is present in an amount in the lipid composition to effect an expression or activity of the polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the SORT lipid is present in an amount in the lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in the lipid composition to effect an uptake of the polynucleotide in a greater amount to a cell compared to that achieved with a reference lipid composition comprising LF92, a phospholipid, cholesterol, and a PEG-lipid.

### Protein Corona Binding

In some embodiments, upon administration, a surface of a pharmaceutical composition as described herein binds to one or more target proteins comprising a protein corona. In some embodiments, the surface of the pharmaceutical composition may bind a first target protein. In some embodiments, the surface of the pharmaceutical composition may bind a first and a second target protein. The surface of the pharmaceutical composition may bind the first target protein and the second target protein at a weight or mass ratio. The weight or mass ratio may be determined, e.g., by an incubation assay.

A surface of a pharmaceutical composition as disclosed herein may comprise a protein corona. The protein corona may comprise one or more (e.g., serum or blood) proteins. The one or more proteins may comprise an apolipoprotein, a complement protein, an immune protein, a coagulation protein, or any other protein. In some embodiments, the one or more proteins comprise target proteins. The one or more target proteins may comprise an apolipoprotein, a complement protein, an immune protein, a coagulation protein, or any other protein. In some embodiments, the one or more target proteins comprise alpha-2-HS-glycoprotein, aomplement C1q subcomponent subunit C, alpha-1-antitrypsin 1-3, Ig alpha chain C region, Ig mu chain C region (fragment), serine protease inhibitor A3K, apolipoprotein C-I, serum albumin, immunoglobulin heavy variable 1-34 (fragment), vitamin K-dependent protein Z, immunoglobulin kappa variable 6-13, Ig gamma-2B chain C region, histone H, beta-2-glycoprotein 1, Ig heavy chain V region X44, protein Z-dependent protease inhibitor, immunoglobulin heavy constant alpha (fragment), C-reactive protein, mannose-binding protein C, immunoglobulin kappa variable 1-110 (fragment), beta-casein, immunoglobulin heavy constant mu, serum paraoxonase/arylesterase, glycosylphosphatidylinositol specific phospholipase D1, inter alpha-trypsin inhibitor, heavy chain, immunoglobulin heavy constant gamma 2C (fragment), complement C3, immunoglobulin kappa variable 17-127 (fragment), Ig heavy chain V region AC38 205.12, complement factor D, serotransferrin, beta-globin, coagulation factor VII, Ig kappa chain V-III region 50S10.1, alpha-S1-casein Inter-alpha-trypsin inhibitor heavy chain H3, apolipoprotein A-IV, protein Igkv12-41 (Fragment), alpha-S2-casein-like A, Ig heavy chain V region 6.96, clusterin, murinoglobulin-1, lactadherin, fibrinogen beta chain. coagulation factor V, Ig kappa chain V-II region 26-10, Ig gamma-1 chain C region secreted form (fragment), Immunoglobulin heavy constant gamma 3 (fragment), platelet factor 4, apolipoprotein A-I, lipopolysaccharide-binding protein, immunoglobulin heavy variable 5-9 (fragment), Ig kappa chain V-V region HP 124E, .histidine-rich glycoprotein, Ig heavy chain V-III region J606, Ig kappa chain V-III region PC 2880/PC 1229, Ig kappa chain V-V region HP 124E1, band 3 anion transport protein, immunoglobulin kappa variable 17-121 (fragment), apolipoprotein N, plasminogen, immunoglobulin kappa chain variable 8-30 (fragment), complement CIs-A subcomponent, hemoglobin subunit beta-2, immunoglobulin kappa variable 1-135 (fragment), vitamin K-dependent protein C, H-2 class I histocompatibility antigen, Q10 alpha chain, alpha globin 1, thrombospondin-1, apolipoprotein D, coagulation factor, fibrinogen gamma chain. immunoglobulin heavy variable 7-1 (fragment), immunoglobulin kappa variable 4-57 (fragment), immunoglobulin heavy variable V1-5, Ig heavy chain V region 914, histone H2B, immunoglobulin heavy constant gamma 3 (fragment), apolipoprotein E, fibrinogen alpha chain, complement C1q subcomponent subunit A, immunoglobulin heavy variable 5-9 (fragment), immunoglobulin kappa constant, apolipoprotein C-III, immunoglobulin heavy constant gamma 2B (fragment), prothrombin, complement C1q subcomponent subunit, carboxypeptidase N catalytic chain, vitronectin, immunoglobulin kappa variable 12-46 (fragment), Ig gamma-2A chain C region, membrane-bound form, or immunoglobulin kappa variable 12-44 (fragment).

In some embodiments, the pharmaceutical composition may bind a first and a second target protein at a certain weight or mass ratio. The weight or mass ratio may range from 1:1 to 20:1 or more. In some embodiments, the weight or mass ration may be 1:1, 2:1, 3:1, 4:1, 5:1, 6:1, 7:1, 8:1. 9:1, 10:1, 11:1, 12:1, 13:1, 14:1, 15:1, 16:1, 17:1, 18:1, 19:1, 20:1, or more. In some embodiments, the weight or mass ratio may be 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1 or less.

In some embodiments, the composition of a protein corona may determine an organ and/or cell type tropism (e.g., targeting) of a pharmaceutical composition as described herein. The organ and/or cell type targeting may be determined by the presence or absence of a certain target protein, the weight or mass ratio between one target protein and another target protein or proteins, or some combination thereof. The presence of absence of a protein and/or a weight or mass ratio between one protein or another or among a group of proteins may be determined by an incubation assay. In some embodiments, the incubation assay may comprise incubating a lipid composition in a serum or plasma sample from an organism (e.g., mouse). Protein bound to the lipid composition may be isolated and/or purified and quantified by any suitable technique known in the art such as Bradford or other colorimetric assays, UV-vis spectroscopy, biuret assays, and fluorescent assays. Proteins isolated from such samples may be further characterized or identified by mass spectrometry, gel electrophoresis (e.g., native gel, SDS PAGE gel), or other suitable techniques known in the art.

The targeting of a composition comprising a given protein corona may be determined by measuring (directly, indirectly, and/or relatively) the amount of the composition or portion thereof (e.g., payload, therapeutic agent) delivered to one or more organs or cell types. For example, measuring the targeting of the composition comprising the protein corona may comprise measuring the amount, expression, or activity of a therapeutic payload contained in the composition in a target organ or cell type versus a reference organ or cell type. In some embodiments, the amount, expression, or activity of a therapeutic agent delivered by the composition comprising the protein corona is at least about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold or higher. In some embodiments, the amount, expression, or activity of a therapeutic agent delivered by the composition comprising the protein corona is less than about 20-fold, about 19-fold, about 18-fold, about 17-fold, about 16-fold, about 15-fold, about 14-fold, about 13-fold, about 12-fold, about 11-fold, about 10-fold, about 9-fold, about 8-fold, about 7-fold, about 6-fold, about 5-fold, about 4-fold, about 3-fold, about 2-fold, or less.

Assays to determine the targeting of a composition comprising a protein corona may comprise any suitable quantification procedure or functional assay known in the art. By way of nonlimiting example, such assays may comprise quantification of luminescence of a fluorescent payload or transcription/translation product thereof, immunofluorescence assays targeting a payload or translation product thereof, and the like.

In some embodiments, the protein corona comprises apolipoprotein E (Apo E) and serum albumin. In such cases, the composition comprising the protein corona may target a liver or liver cell. In some embodiments, the Apo E is present a weight or mass ratio or no more than about 6:1, about 5:1, about 4:1, or about 3:1 to the serum albumin. In some embodiments, the protein corona further comprises complement C1q subcomponent subunit A, immunoglobulin heavy constant mu, complement C1q subcomponent subunit B, immunoglobulin kappa constant, immunoglobulin heavy constant gamma 2B, beta-globin, immunoglobulin (Ig) gamma-2A chain C region, complement C1q subcomponent subunit C, immunoglobulin heavy constant alpha, fibrinogen beta chain, fibrinogen gamma chain, immunoglobulin kappa variable 17-127, alpha globin 1, fibrinogen alpha chain, clusterin, another protein, or any combination thereof as determined by an incubation assay. In some embodiments, the protein corona comprises at least one, at least two, or at least three protein(s) listed in Table 10 (e.g., those not listed in or listed different from Table 9).

In some embodiments, the protein corona comprises Apo E in a lesser amount than an endogenous protein which is not Apo E, as determined by an incubation assay. In some embodiments, the endogenous protein is beta-2 glycoprotein 1 (β2-GP1) or apolipoprotein H (Apo H), immunoglobulin kappa constant, complement C1q subcomponent subunit A, vitronectin, and serum paraoxonase/arylesterase 1, clusterin another protein, or combinations thereof. In some cases, the protein corona further comprises apolipoprotein C (Apo C). In such cases, the protein corona may comprise less Apo C than Apo E, as determined by an incubation assay.

In some embodiments, the protein corona comprises vitronectin and clusterin. In such cases, the composition comprising the protein corona may target a lung or a lung cell. In some embodiments, the vitronectin is present at a weight or mass ratio of no more than about 6:1 or about 5:1 to the clusterin, as determined by an incubation assay. In some embodiments, the protein corona further comprises serum paraoxonase/arylesterase 1, apolipoprotein E (Apo E), serum albumin, immunoglobulin kappa constant, prothrombin, complement C1q subcomponent subunit A, fibrinogen beta chain, beta-2 glycoprotein 1 (β2-GP1) or apolipoprotein H (Apo H), immunoglobulin (Ig) mu chain C region, alpha-S1-casein, immunoglobulin heavy constant gamma 2B, fibrinogen gamma chain, fibrinogen alpha chain, vitamin K-dependent protein Z, alpha-1-antitrypsin 1-3, plasminogen, apolipoprotein C-III, complement C1q subcomponent subunit B, thrombospondin-1, coagulation factor X, apolipoprotein A-I, immunoglobulin heavy constant alpha, immunoglobulin (Ig) gamma-2A chain C region, beta-globin, complement C1q subcomponent subunit C, protein Z-dependent protease inhibitor, clusterin, another protein, or any combination thereof as determined by an incubation assay. In some embodiments, the protein corona comprises at least one, at least two, or at least three protein(s) listed in Table 12 (e.g., those not listed in or listed different from Table 9).

In some embodiments, the protein corona comprises beta-2 glycoprotein 1 (β2-GP1) or apolipoprotein H (Apo H) and a second target protein that is different from beta-2 glycoprotein 1 or Apo H. The beta-2 glycoprotein 1 or Apo H may be present at a weight or mass ratio of no more than about 20:1, 15:1, of 10:1 to the second target protein. In such cases, the pharmaceutical composition may target the spleen, bone marrow, or a lymph node or a cell therein. In some embodiments, the cell is a spleen cell or a microphase. In some embodiments, the second target protein comprises immunoglobulin kappa constant, complement C1q subcomponent subunit A, apolipoprotein E (Apo E), immunoglobulin heavy constant gamma 2B, complement C1q subcomponent subunit B, vitronectin, complement C1q subcomponent subunit C, apolipoprotein C-I, immunoglobulin (Ig) gamma-2A chain C region, immunoglobulin (Ig) mu chain C region, serum albumin, serum paraoxonase/arylesterase 1, immunoglobulin heavy constant alpha, clusterin, and immunoglobulin kappa variable 6-13, another protein, or a combination thereof. In some embodiments, the protein corona comprises at least one, at least two, or at least three protein(s) listed in Table 11 (e.g., those not listed in or listed different from Table 9). In some embodiments, the protein corona does not comprise beta-2 glycoprotein 1 or Apo H. In such cases, the composition may target an organ that is not a spleen bone marrow, or lymph node or a cell therein. The composition may not target a spleen cell or macrophage.

### Methods for Targeted Delivery

In some embodiments, provided herein is a method for targeted delivery of a therapeutic agent to an organ or a cell therein in a subject in need thereof. The method may comprise administering to the subject the therapeutic agent assembled with a lipid composition such as those described herein. In some embodiments, the lipid composition comprises: an ionizable cationic lipid; a polymer-conjugated lipid; and a selective organ targeting (SORT) lipid separate from the ionizable cationic lipid and the polymer-conjugated lipid. In some embodiments, the administering comprises intravenously administering. In some embodiments, a bodily fluid (e.g., plasma or serum) of the subject comprises the plurality of target proteins. In some embodiments, the plurality of target proteins is a plurality of endogenous proteins of the subject.

In some embodiments of the method described herein, upon the administering, a surface of the lipid composition binds to a plurality of target proteins as determined by an incubation assay, which plurality of target proteins comprises a first target protein at a weight or mass ratio of no more than about 20:1, 15:1, or 10:1 to a second target protein that is different from the first target protein, thereby delivering the therapeutic agent to the target organ or the target cell in the subject. In some embodiments, the method provides a (e.g., at least about 2-fold) greater amount, expression or activity of the therapeutic agent in the organ or the cell therein in the subject as compared to that achieved with a corresponding reference lipid composition (e.g., absent binding to the plurality of target proteins). In some embodiments, the method provides a (e.g., at least about 2-fold) greater amount, expression or activity of the therapeutic agent in the organ or the cell therein in the subject as compared to that achieved absent the polymer-conjugated lipid. In some embodiments, in the method provides a (e.g., at least about 2-fold) greater amount, expression or activity of the therapeutic agent in the organ or the cell therein in the subject as compared to that achieved in a reference organ or a reference cell.

In some embodiments, the method described herein is for targeted delivery of a therapeutic agent to liver or a liver cell therein in a subject in need thereof. In some embodiments, upon the administering, a surface of the lipid composition binds to a plurality of target proteins as determined by an incubation assay, which plurality of target proteins comprises apolipoprotein E (Apo E) and serum albumin, thereby delivering the therapeutic agent to the liver or the liver cell in the subject. In some embodiments, the Apo E is present at a weight or mass ratio of no more than about 6:1, 5:1, 4:1, or 3:1 to the serum albumin in the plurality of target proteins as determined by an incubation assay. In some embodiments, the plurality of target proteins further comprise complement C1q subcomponent subunit A, immunoglobulin heavy constant mu, complement C1q subcomponent subunit B, immunoglobulin kappa constant, immunoglobulin heavy constant gamma 2B, beta-globin, immunoglobulin (Ig) gamma-2A chain C region, complement C1q subcomponent subunit C, immunoglobulin heavy constant alpha, fibrinogen beta chain, fibrinogen gamma chain, immunoglobulin kappa variable 17-127, alpha globin 1, fibrinogen alpha chain, or any combination thereof as determined by an incubation assay. In some embodiments, the plurality of target proteins comprises at least one, at least two, or at least three protein(s) listed in Table 10 (e.g., those not listed in or listed different from Table 9). In some embodiments, the SORT lipid comprises an ionizable cationic moiety (e.g., a tertiary amine moiety). In some embodiments, the SORT lipid is an ionizable cationic lipid. In some embodiments, the lipid composition comprises the SORT lipid at a molar percentage from about 5% to about 65%. In some embodiments, the method provides a (e.g., at least about 2-, 3-, 4-, 5-, or 6-fold) greater amount, expression or activity of the therapeutic agent in the liver or the liver cell in the subject as compared to that achieved with a corresponding reference lipid composition (e.g., absent the binding to the plurality of target proteins).

In some embodiments, the method described herein is for targeted delivery of a therapeutic agent to a non-liver organ or a non-liver cell therein in a subject in need thereof. In some embodiments, upon the administering, wherein, upon the administering, a surface of the SORT lipid composition interacts with apolipoprotein E (Apo E) to a lesser degree than with an endogenous protein that is not Apo E in the subject as determined by an incubation assay, which endogenous protein that is not Apo E is selected from beta-2 glycoprotein 1 (β2-GP1) or apolipoprotein H (Apo H), immunoglobulin kappa constant, complement C1q subcomponent subunit A, vitronectin, and serum paraoxonase/arylesterase 1, thereby delivering the therapeutic agent to the non-liver organ or the non-liver cell in the subject. In some embodiments, the non-liver organ comprises a lung, spleen, bone marrow, or a lymph node. In some embodiments, non-liver cell comprises a lung cell, a spleen cell, or a macrophage. In some embodiments, apolipoprotein E (Apo E) is not the most abundant protein in the plurality of target proteins. In some embodiments, upon the administering, a surface of the lipid composition interacts with apolipoprotein C (Apo C) to a lesser degree than with apolipoprotein E (Apo E) in the subject as determined by an incubation assay. In some embodiments, the method provides a lesser amount or activity of the therapeutic agent in liver or a cell therein in the subject as compared to that achieved absent the polymer-conjugated lipid. In some embodiments, the SORT lipid is a permanent cationic lipid, an ionizable cationic lipid, a zwitterionic lipid, or an anionic lipid. In some embodiments, the lipid composition comprises the SORT lipid at a molar percentage from about 5% to about 65%.

In some embodiments, the method described herein is for targeted delivery of a therapeutic agent to a lung or a lung cell therein in a subject in need thereof. In some embodiments, upon the administering, a surface of the lipid composition binds to a plurality of target proteins as determined by an incubation assay, which plurality of target proteins comprises vitronectin (Vtn) and clusterin, thereby delivering the therapeutic agent to the lung or the lung cell in the subject. In some embodiments, the vitronectin is present at a weight or mass ratio of no more than about 6:1, or 5:1 to the clusterin in the plurality of target proteins as determined by an incubation assay. In some embodiments, the plurality of target proteins further comprise serum paraoxonase/arylesterase 1, apolipoprotein E (Apo E), serum albumin, immunoglobulin kappa constant, prothrombin, complement C1q subcomponent subunit A, fibrinogen beta chain, beta-2 glycoprotein 1 (β2-GP1) or apolipoprotein H (Apo H), immunoglobulin (Ig) mu chain C region, alpha-S1-casein, immunoglobulin heavy constant gamma 2B, fibrinogen gamma chain, fibrinogen alpha chain, vitamin K-dependent protein Z, alpha-1-antitrypsin 1-3, plasminogen, apolipoprotein C-III, complement C1q subcomponent subunit B, thrombospondin-1, coagulation factor X, apolipoprotein A-I, immunoglobulin heavy constant alpha, immunoglobulin (Ig) gamma-2A chain C region, beta-globin, complement C1q subcomponent subunit C, protein Z-dependent protease inhibitor, or any combination thereof as determined by an incubation assay. In some embodiments, the plurality of target proteins comprises at least one, at least two, or at least three protein(s) listed in Table 12 (e.g., those not listed in or listed different from Table 9). In some embodiments, the SORT lipid is a cationic lipid. In some embodiments, the SORT lipid is a permanent cationic lipid. In some embodiments, the SORT lipid is an ionizable cationic lipid. In some embodiments, the lipid composition comprises the SORT lipid at a molar percentage from about 5% to about 65%. In some embodiments, the method provides a (e.g., at least about 2-, 5-, 10-, 11-, 12-, 13-, 14-, 15-, 16-, 17-, 18-, 19-, or 20-fold) greater amount, expression or activity of the therapeutic agent in the lung or the lung cell in the subject as compared to that achieved with a corresponding reference lipid composition (e.g., absent binding to the plurality of target proteins).

In some embodiments, the method described herein is for targeted delivery of a therapeutic agent to spleen, bone marrow, or a lymph node or a cell therein in a subject in need thereof. In some embodiments, upon the administering, a surface of the lipid composition binds to a plurality of target proteins as determined by an incubation assay, which plurality of target proteins comprises beta-2 glycoprotein 1 (β2-GP1) or apolipoprotein H (Apo H) at a weight or mass ratio of no more than about 20:1, 15:1, or 10:1 to a second target protein that is different from the beta-2 glycoprotein 1 (β2-GP1) or apolipoprotein H (Apo H), thereby delivering the therapeutic agent to the spleen, bone marrow, or a lymph node or the cell in the subject. In some embodiments, the cell comprises a spleen cell, or a macrophage. In some embodiments, the second target protein is selected from: immunoglobulin kappa constant, complement C1q subcomponent subunit A, apolipoprotein E (Apo E), immunoglobulin heavy constant gamma 2B, complement C1q subcomponent subunit B, vitronectin, complement C1q subcomponent subunit C, apolipoprotein C-I, immunoglobulin (Ig) gamma-2A chain C region, immunoglobulin (Ig) mu chain C region, serum albumin, serum paraoxonase/arylesterase 1, immunoglobulin heavy constant alpha, and immunoglobulin kappa variable 6-13. In some embodiments, the SORT lipid is a permanent cationic lipid, or an anionic lipid. In some embodiments, the plurality of target proteins comprises at least one, at least two, or at least three protein(s) listed in Table 11 (e.g., those not listed in or listed different from Table 9). In some embodiments, the SORT lipid is a permanent cationic lipid. In some embodiments, the SORT lipid is an anionic lipid. In some embodiments, the lipid composition comprises the SORT lipid at a molar percentage from about 5% to about 65%. In some embodiments, the method provides a (e.g., at least about 2-fold) greater amount, expression or activity of the therapeutic agent in the lung or the lung cell in the subject as compared to that achieved with a corresponding reference lipid composition (e.g., absent binding to the plurality of target proteins).

In some embodiments, the method described herein is for targeted delivery of a therapeutic agent to a non-spleen organ or a non-spleen cell therein in a subject in need thereof. In some embodiments, upon the administering, a surface of the lipid composition binds to a plurality of target proteins as determined by an incubation assay, which plurality of target proteins comprises a first target protein at a weight or mass ratio of no more than about 20:1, 15:1, or 10:1 to a second target protein that is different from the first target protein, thereby delivering the therapeutic agent to the non-spleen organ or the non-spleen cell in the subject. In some embodiments, the non-spleen organ is not spleen, bone marrow, or a lymph node. In some embodiments, the non-spleen cell is not a spleen cell, or a macrophage. In some embodiments, beta-2 glycoprotein 1 (β2-GP1) or apolipoprotein H (Apo H) is not the most abundant protein in the plurality of target proteins. In some embodiments, the plurality of target proteins comprises clusterin. In some embodiments, the SORT lipid is a permanent cationic lipid, an ionizable cationic lipid, a zwitterionic lipid, or an anionic lipid. In some embodiments, the lipid composition comprises the SORT lipid at a molar percentage from about 5% to about 65%.

In some instances, in the methods described herein, the therapeutic agent may comprise a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

### Formulations

In some embodiments, in any of the methods or compositions provided herein, the therapeutic agents provided herein may be present in intravenous compositions. In some embodiments, the therapeutic agents provided herein may be present in aerosol compositions. In some embodiments, the lipid composition may be formulated as an aerosol. In some embodiments, the compositions provided herein may be formulated as an aerosol dosage form. In other embodiments, the compositions provided herein are formulated as intravenous dosage forms. In some embodiments, the lipid composition may be formulated as a nebulizer. In some embodiments, the compositions described herein are dispensed via a nebulizer. In some embodiments, the lipid composition may be dispensed as an aerosol. In some embodiments, the compositions described herein may be stored at or below −70° C.

In some embodiments, the compositions described herein may be formulated as a dispersion. In some embodiments, the concentration of the dispersion is about 0.5 mg/mL to about 5 mg/mL. In some embodiments, the concentration of the dispersion is about 0.5 mg/mL to about 1 mg/mL. In some embodiments, the concentration of the dispersion is about 0.5 mg/mL to about 2 mg/mL. In some embodiments, the concentration of the dispersion is about 0.5 mg/mL to about 3 mg/mL. In some embodiments, the concentration of the dispersion is about 2 mg/mL to about 3 mg/mL. In some embodiments, the concentration of the dispersion is about 2 mg/mL to about 4 mg/mL. In some embodiments, the concentration of the dispersion is no more than 5 mg/mL. In some embodiments, the concentration of the dispersion is 1 mg/mL. In some embodiments, the compositions described herein are dispersed at pH 7.5.

In some embodiments, the compositions provided herein are administered to a human. In some embodiments, the compositions provided herein are administered to an adult. In other embodiments, the compositions provided herein are administered to a child. In some embodiments, the compositions provided herein are administered to a patient with a body mass index of 18 to 35 kg/m². In other embodiments, the compositions provided herein are administered to a patient with a total body weight of ≥50 kg. In some embodiments, the compositions described herein are administered intravenously. In some embodiments, the compositions described herein are delivered via inhalation. In some embodiments, the compositions described herein may comprise administration by nebulization. In some embodiments, the compositions described herein may comprise administration to a lung by nebulization. In some embodiments, the compositions described herein are administered at least once a week. In some embodiments, the compositions described herein are administered at least twice a week.

In any of the compositions or methods provided herein, the compositions are administered in any suitable dose. In some embodiments, the dose refers to the amount of the composition. In some embodiments the dose refers to the amount of the therapeutic agent. In some embodiments, the administered dose is about 1 mg to about 30 mg. In some embodiments, the administered dose is about 1 mg to about 2.5 mg, about 1 mg to about 5 mg, about 1 mg to about 7.5 mg, about 1 mg to about 10 mg, about 1 mg to about 15 mg, about 1 mg to about 20 mg, about 1 mg to about 25 mg, about 1 mg to about 30 mg, about 2.5 mg to about 5 mg, about 2.5 mg to about 7.5 mg, about 2.5 mg to about 10 mg, about 2.5 mg to about 15 mg, about 2.5 mg to about 20 mg, about 2.5 mg to about 25 mg, about 2.5 mg to about 30 mg, about 5 mg to about 7.5 mg, about 5 mg to about 10 mg, about 5 mg to about 15 mg, about 5 mg to about 20 mg, about 5 mg to about 25 mg, about 5 mg to about 30 mg, about 7.5 mg to about 10 mg, about 7.5 mg to about 15 mg, about 7.5 mg to about 20 mg, about 7.5 mg to about 25 mg, about 7.5 mg to about 30 mg, about 10 mg to about 15 mg, about 10 mg to about 20 mg, about 10 mg to about 25 mg, about 10 mg to about 30 mg, about 15 mg to about 20 mg, about 15 mg to about 25 mg, about 15 mg to about 30 mg, about 20 mg to about 25 mg, about 20 mg to about 30 mg, or about 25 mg to about 30 mg. In some embodiments, the administered dose is about 1 mg, about 2.5 mg, about 5 mg, about 7.5 mg, about 10 mg, about 15 mg, about 20 mg, about 25 mg, or about 30 mg. In some embodiments, the administered dose is at least about 1 mg, about 2.5 mg, about 5 mg, about 7.5 mg, about 10 mg, about 15 mg, about 20 mg, or about 25 mg. In some embodiments, the administered dose is at most about 2.5 mg, about 5 mg, about 7.5 mg, about 10 mg, about 15 mg, about 20 mg, about 25 mg, or about 30 mg. In some embodiments, the administered dose is about 2.5 mg. In some embodiments, the administered dose is about 5.0 mg. In some embodiments, the adminstered dose is about 10.0 mg. In some embodiments, the administered dose is about 20.0 mg.

In any of the compositions or methods provided herein, the dose may be determined in reference to body weight. In any of these compositions or methods, any suitable dose may be used. In some embodiments, the dose is about 0.01 mg/kg body weight to about 1 mg/kg body weight. In some embodiments, the dose is about 0.01 mg/kg body weight to about 0.05 mg/kg body weight, about 0.01 mg/kg body weight to about 0.1 mg/kg body weight, about 0.01 mg/kg body weight to about 0.5 mg/kg body weight, about 0.01 mg/kg body weight to about 0.8 mg/kg body weight, about 0.01 mg/kg body weight to about 1 mg/kg body weight, about 0.05 mg/kg body weight to about 0.1 mg/kg body weight, about 0.05 mg/kg body weight to about 0.5 mg/kg body weight, about 0.05 mg/kg body weight to about 0.8 mg/kg body weight, about 0.05 mg/kg body weight to about 1 mg/kg body weight, about 0.1 mg/kg body weight to about 0.5 mg/kg body weight, about 0.1 mg/kg body weight to about 0.8 mg/kg body weight, about 0.1 mg/kg body weight to about 1 mg/kg body weight, about 0.5 mg/kg body weight to about 0.8 mg/kg body weight, about 0.5 mg/kg body weight to about 1 mg/kg body weight, or about 0.8 mg/kg body weight to about 1 mg/kg body weight. In some embodiments, the dose is about 0.01 mg/kg body weight, about 0.05 mg/kg body weight, about 0.1 mg/kg body weight, about 0.5 mg/kg body weight, about 0.8 mg/kg body weight, or about 1 mg/kg body weight. In some embodiments, the dose is at least about 0.01 mg/kg body weight, about 0.05 mg/kg body weight, about 0.1 mg/kg body weight, about 0.5 mg/kg body weight, or about 0.8 mg/kg body weight. In some embodiments, the dose is at most about 0.05 mg/kg body weight, about 0.1 mg/kg body weight, about 0.5 mg/kg body weight, about 0.8 mg/kg body weight, or about 1 mg/kg body weight. In some embodiments, a dose comprises no more than about 1.0, 0.5, 0.1, 0.05, or 0.01 mg/kg body weight.

In any of the compositions or methods provided herein, the dose may be determined in reference to lung weight. In any of these compositions or methods, any suitable dose may be used. In some embodiments, the administered dose is about 0.002 mg/g lung weight to about 0.03 mg/g lung weight. In some embodiments, the administered dose is about 0.002 mg/g lung weight to about 0.005 mg/g lung weight, about 0.002 mg/g lung weight to about 0.008 mg/g lung weight, about 0.002 mg/g lung weight to about 0.01 mg/g lung weight, about 0.002 mg/g lung weight to about 0.015 mg/g lung weight, about 0.002 mg/g lung weight to about 0.02 mg/g lung weight, about 0.002 mg/g lung weight to about 0.025 mg/g lung weight, about 0.002 mg/g lung weight to about 0.03 mg/g lung weight, about 0.005 mg/g lung weight to about 0.008 mg/g lung weight, about 0.005 mg/g lung weight to about 0.01 mg/g lung weight, about 0.005 mg/g lung weight to about 0.015 mg/g lung weight, about 0.005 mg/g lung weight to about 0.02 mg/g lung weight, about 0.005 mg/g lung weight to about 0.025 mg/g lung weight, about 0.005 mg/g lung weight to about 0.03 mg/g lung weight, about 0.008 mg/g lung weight to about 0.01 mg/g lung weight, about 0.008 mg/g lung weight to about 0.015 mg/g lung weight, about 0.008 mg/g lung weight to about 0.02 mg/g lung weight, about 0.008 mg/g lung weight to about 0.025 mg/g lung weight, about 0.008 mg/g lung weight to about 0.03 mg/g lung weight, about 0.01 mg/g lung weight to about 0.015 mg/g lung weight, about 0.01 mg/g lung weight to about 0.02 mg/g lung weight, about 0.01 mg/g lung weight to about 0.025 mg/g lung weight, about 0.01 mg/g lung weight to about 0.03 mg/g lung weight, about 0.015 mg/g lung weight to about 0.02 mg/g lung weight, about 0.015 mg/g lung weight to about 0.025 mg/g lung weight, about 0.015 mg/g lung weight to about 0.03 mg/g lung weight, about 0.02 mg/g lung weight to about 0.025 mg/g lung weight, about 0.02 mg/g lung weight to about 0.03 mg/g lung weight, or about 0.025 mg/g lung weight to about 0.03 mg/g lung weight. In some embodiments, the administered dose is about 0.002 mg/g lung weight, about 0.005 mg/g lung weight, about 0.008 mg/g lung weight, about 0.01 mg/g lung weight, about 0.015 mg/g lung weight, about 0.02 mg/g lung weight, about 0.025 mg/g lung weight, or about 0.03 mg/g lung weight.

The following are examples of compositions and evaluations of compositions of the disclosure. It is understood that various other embodiments may be practiced, given the general description provided above.

### EXAMPLES

### Example 1: Preparation of DOTAP or DODAP Modified Lipid Nanoparticles

Lipid nanoparticles (LNPs) are the most efficacious carrier class for in vivo nucleic acid delivery. Historically, effective LNPs are composed of 4 components: an ionizable cationic lipid, zwitterionic phospholipid, cholesterol, and lipid poly(ethylene glycol) (PEG). However, these LNPs result in only general delivery of nucleic acids, rather than organ or tissue targeted delivery. LNPs typically delivery RNAs only to the liver. Therefore, new formulations of LNPs were sought in an effort to provide targeted nucleic acid delivery.

The four canonical types of lipids were mixed in a 15:15:30:3 molar ratio, with or without the addition of a permanently cationic lipid. Briefly, LNPs were prepared by mixing a dendrimer or dendron lipid (ionizable cationic), DOPE (zwitterionic), cholesterol, DMG-PEG, and DOTAP (permanently cationic). Alternatively, DOTAP can be substituted for DODAP to generate an LNP comprising DODAP.

For preparation of the LNP formulation, a dendrimer or dendron lipid, DOPE, Cholesterol and DMG-PEG were dissolved in ethanol at desired molar ratios. The mRNA was dissolved in citrate buffer (10 mM, pH 4.0). The mRNA was then diluted into the lipids solution to achieve a weight ratio of 40:1 (total lipids:mRNA) by rapidly mixing the mRNA into the lipid solution at a volume ratio of 3:1 (mRNA:lipids, v/v). This solution was then incubated for 10 min at room temperature. For formation of DOTAP modified LNP formulations, mRNA was dissolved in 1×PBS or citrate buffer (10 mM, pH 4.0), and mixed rapidly into ethanol containing 5A2-SC8, DOPE, Cholesterol, DMG-PEG and DOTAP, fixing the weight ratio of 40:1 (total lipids:mRNA) and volume ratio of 3:1 (mRNA:lipids). Formulations are named X % DOTAP Y (or X % DODAP Y) where X represents the DOTAP (or DODAP) molar percentage in total lipids, and Y represents the type of dendrimer or dendron lipid. Alternatively, formulation may be named Y X % DOTAP or Y X % DODAP where X represents the DOTAP (or DODAP) molar percentage in total lipids, and Y represents the type of dendrimer or dendron lipid.

### Example 2: SORT LNP Stability

Lipid (LNP) compositions are tested for stability. Lipid compositions as described herein, such as those comprising a dendrimer or dendron (e.g., 5A2-SC8), as an ionizable cationic lipid, and a selective organ-targeting (SORT) lipid (e.g., DODAP), e.g., at a molar percentage in total lipids from 20% to 50%, are generated using either a microfluidic mixing method or a cross/tee mixing method. Size, polydispersity index (PDI) and zeta-potential of different LNP formulations are characterized by dynamic light scattering (DLS) (3 separate times for each formulation).

Encapsulation efficiency of the LNPs is tested using a Ribogreen RNA assay (Zhao et al., 2016). Briefly, mRNA is encapsulated with >90% (e.g., >95%) efficiency in LNPs when the mRNA is dissolved in acidic buffer (e.g., 10 mM citrate, pH 4). The characteristics are observed over 28 days for the tested LNPs, e.g., over the course of 28 days.

In addition, stability of the lipid compositions as described herein (LNPs) in solution and resulting mRNA expression are observed in mice. Briefly, mice are injected intravenously with less than 1 mg/kg and observed in vivo. Luciferin is added 5 hours after injection and visualized. SORT (e.g., lung-SORT) LNP generated tissue specific radiance in the lungs remain highly detectable even after 14 day with a slight decay in signal by the 21^{st} and 28^{th} day. Images of organs of the tested mice at specific time periods after treated with example SORT LNP are taken.

### Example 3A: SORT Molecules Alter LNP Biodistribution

Messenger RNA therapeutics must overcome multiple barriers for intracellular delivery as they do not readily diffuse through anionic cellular membranes and are susceptible to degradation by RNases in the blood. Ideally, LNPs will encapsulate and protect mRNA from enzymatic degradation, enable accumulation in the target organ, facilitate receptor-mediated endocytosis into cells, and release mRNA from endosomes into the cytosol to undergo translation into a functional protein. Through the development of SORT LNPs, we showed that the chemical identity and amount of the incorporated SORT molecule systematically alters tissue-specific protein expression following mRNA delivery. Inclusion of ionizable cationic lipids enhanced liver targeting, anionic lipids resulted in retargeting of delivery to the spleen, and permanently cationic lipids bearing a quaternary ammonium headgroup retargeted delivery to the lungs (FIG. 1A). These experiments revealed how the choice of SORT molecule served as a design parameter to predictably dictate where mRNA would be translated to functional proteins in vivo, but did not elucidate the organs where example SORT LNPs accumulated and the mechanism of how SORT molecules function. Biodistribution to the target organ should, in principle, be a requisite step for targeted delivery.

We first investigated the biodistribution of mRNA encapsulated in example SORT LNPs to determine whether there was a correlation between the organs in which the SORT LNPs accumulated and their tissue-specific mRNA activity. We formulated example liver, lung, and spleen SORT LNPs encapsulating Cy5-labelled mRNA following the identical protocols and formulation parameters used in our previous publications. We administered each LNP IV to C57BL/6 mice and tracked the in vivo biodistribution of the Cy5-mRNA using fluorescence imaging. Six hours post-injection, organs were excised and imaged ex vivo (FIG. 1B). The average fluorescence produced by the liver, spleen, and lungs was quantified (FIG. 1C). The incorporation of the ionizable cationic lipid DODAP to reference mDLNP resulted in an increase in average Cy5-mRNA signal in the liver and a decrease of average Cy5-mRNA signal in the spleen, with optimal liver biodistribution at 20% inclusion of DODAP (FIGS. 1B-1C). Meanwhile, increasing the percentage of anionic SORT molecule 18PA added to the LNP increased average Cy5-mRNA signal in the spleen (FIGS. 1B-1C). Similarly, as the permanently cationic SORT molecule DOTAP was included in the LNP in increasing proportions, there was a progressive increase in Cy5-mRNA signal in the lungs (FIGS. 1B-1C). Importantly, these tracking studies quantify where the example SORT LNPs biodistribute but not where mRNA is translated to protein (functional delivery). These studies demonstrate that inclusion of a SORT molecule in reference mDLNP facilitates mRNA biodistribution to the target organ but is not sufficient to explain tissue-specific mRNA activity.

### Example 3B: SORT Molecules Alter Apparent LNP pKₐ

The apparent pKa of 67 different example SORT LNPs (Table 7) prepared as described in Example 1, all of which were efficacious for tissue-specific mRNA delivery in vivo, was analyzed using the 6-(p-toluidino)-2-naphthalenesulfonic acid (TNS) assay. Briefly, mRNA formulations (60 μM total lipids) and the TNS probe (2 μM) were incubated for 5 min with a series of buffers, containing 10 mM HEPES, 10 mM MES (4-morpholineethanesulfonic acid), 10 mM ammonium acetate and 130 mM NaCl (the pH ranged from 2.5 to 11). The mean fluorescence intensity of each well (black bottom 96-well plate) was measured by a Tecan plate reader with excitation wavelength (λ_{Ex})=321 nm and emission wavelength (λ_{Em})=445 nm, and data were normalized to the value of pH=2.5. Typically, the apparent pKₐ is defined by the pH at half-maximum fluorescence. Although this method was useful for estimating LNP global/apparent pKₐ for most all LNPs, it could not be used for example SORT LNPs containing >40% permanently cationic lipid because these LNPs are always charged. Therefore, the relative pKₐ was instead estimated compared to base LNP formulation (no added SORT lipid) when 50% fluorescence normalized to the lowest fluorescence measurement was produced. This alternative calculation did not change the pKₐ for most LNPs but did allow estimation of the pKₐ of example SORT LNPs with >40% cationic lipid that agreed with experimental results for tissue selective RNA delivery. Representative results of the assay are shown in FIG. 1D. Full results of the assay for the 67 different example SORT LNPs are shown in FIG. 6.

TABLE 7
Molecular composition of example SORT LNPs studied in the TNS assay.

Molar Ratio
(Molar Percentage)

mDLNP-based SORT LNPs

| | | | | | SORT |
|---|---|---|---|---|---|
| Name | 5A2-SC8 | DOPE | Cholesterol | DMG-PEG2K | Molecule |
| mDLNP | 15 | 15 | 30 | 3 | n/a |
| (reference) | (23.81) | (23.81) | (47.62) | (4.76) | (0) |

Cationic Lipid SORT Molecule

| | | | | | |
|---|---|---|---|---|---|
| 5% DOTAP | 15 | 15 | 30 | 3 | 3.315 |
| | (22.62) | (22.62) | (45.24) | (4.52) | (5) |
| 10% DOTAP | 15 | 15 | 30 | 3 | 7 |
| | (21.43) | (21.43) | (42.86) | (4.29) | (10) |
| 15% DOTAP | 15 | 15 | 30 | 3 | 11.12 |
| | (20.24) | (20.24) | (40.47) | (4.05) | (15) |
| 20% DOTAP | 15 | 15 | 30 | 3 | 15.75 |
| | (19.05) | (19.05) | (38.10) | (3.81) | (20) |
| 30% DOTAP | 15 | 15 | 30 | 3 | 27 |
| | (16.67) | (16.67) | (33.33) | (3.33) | (30) |
| 40% DOTAP | 15 | 15 | 30 | 3 | 42 |
| | (14.29) | (14.29) | (28.57) | (2.86) | (40) |
| 50% DOTAP | 15 | 15 | 30 | 3 | 63 |
| (Lung SORT) | (11.90) | (11.90) | (23.81) | (2.38) | (50) |
| 60% DOTAP | 15 | 15 | 30 | 3 | 94.5 |
| | (9.52) | (9.52) | (19.05) | (1.90) | (60) |
| 70% DOTAP | 15 | 15 | 30 | 3 | 147 |
| | (7.14) | (7.14) | (14.29) | (1.43) | (70) |
| 80% DOTAP | 15 | 15 | 30 | 3 | 252 |
| | (4.76) | (4.76) | (9.52) | (0.95) | (80) |
| 90% DOTAP | 15 | 15 | 30 | 3 | 567 |
| | (2.38) | (2.38) | (4.76) | (0.48) | (90) |
| 5% DDAB | 15 | 15 | 30 | 3 | 3.315 |
| | (22.62) | (22.62) | (45.24) | (4.52) | (5) |
| 15% DDAB | 15 | 15 | 30 | 3 | 11.12 |
| | (20.24) | (20.24) | (40.47) | (4.05) | (15) |
| 40% DDAB | 15 | 15 | 30 | 3 | 42 |
| | (14.29) | (14.29) | (28.57) | (2.86) | (40) |
| 50% DDAB | 15 | 15 | 30 | 3 | 63 |
| | (11.90) | (11.90) | (23.81) | (2.38) | (50) |
| 5% 14EPC | 15 | 15 | 30 | 3 | 3.315 |
| | (22.62) | (22.62) | (45.24) | (4.52) | (5) |
| 15% 14EPC | 15 | 15 | 30 | 3 | 11.12 |
| | (20.24) | (20.24) | (40.47) | (4.05) | (15) |
| 40% 14EPC | 15 | 15 | 30 | 3 | 42 |
| | (14.29) | (14.29) | (28.57) | (2.86) | (40) |
| 50% 14EPC | 15 | 15 | 30 | 3 | 63 |
| | (11.90) | (11.90) | (23.81) | (2.38) | (50) |

Anionic Lipid SORT Molecule

| | | | | | |
|---|---|---|---|---|---|
| 5% 18PA | 15 | 15 | 30 | 3 | 3.315 |
| | (22.62) | (22.62) | (45.24) | (4.52) | (5) |
| 10% 18PA | 15 | 15 | 30 | 3 | 7 |
| | (21.43) | (21.43) | (42.86) | (4.29) | (10) |
| 20% 18PA | 15 | 15 | 30 | 3 | 15.75 |
| | (19.05) | (19.05) | (38.10) | (3.81) | (20) |
| 30% 18PA | 15 | 15 | 30 | 3 | 27 |
| (Spleen SORT) | (16.67) | (16.67) | (33.33) | (3.33) | (30) |
| 40% 18PA | 15 | 15 | 30 | 3 | 42 |
| | (14.29) | (14.29) | (28.57) | (2.86) | (40) |
| 5% 14PA | 15 | 15 | 30 | 3 | 3.315 |
| | (22.62) | (22.62) | (45.24) | (4.52) | (5) |
| 10% 14PA | 15 | 15 | 30 | 3 | 7 |
| | (21.43) | (21.43) | (42.86) | (4.29) | (10) |
| 20% 14PA | 15 | 15 | 30 | 3 | 15.75 |
| | (19.05) | (19.05) | (38.10) | (3.81) | (20) |
| 30% 14PA | 15 | 15 | 30 | 3 | 27 |
| | (16.67) | (16.67) | (33.33) | (3.33) | (30) |
| 5% 18BMP | 15 | 15 | 30 | 3 | 3.315 |
| | (22.62) | (22.62) | (45.24) | (4.52) | (5) |
| 10% 18BMP | 15 | 15 | 30 | 3 | 7 |
| | (21.43) | (21.43) | (42.86) | (4.29) | (10) |
| 20% 18BMP | 15 | 15 | 30 | 3 | 15.75 |
| | (19.05) | (19.05) | (38.10) | (3.81) | (20) |
| 30% 18BMP | 15 | 15 | 30 | 3 | 27 |
| | (16.67) | (16.67) | (33.33) | (3.33) | (30) |

Ionizable Cationic Lipid SORT Molecule

| | | | | | |
|---|---|---|---|---|---|
| 10% DODAP | 15 | 15 | 30 | 3 | 7 |
| | (21.43) | (21.43) | (42.86) | (4.29) | (10) |
| 20% DODAP | 15 | 15 | 30 | 3 | 15.75 |
| (Liver SORT) | (19.05) | (19.05) | (38.10) | (3.81) | (20) |
| 50% DODAP | 15 | 15 | 30 | 3 | 63 |
| | (11.90) | (11.90) | (23.81) | (2.38) | (50) |
| 80% DODAP | 15 | 15 | 30 | 3 | 252 |
| | (4.76) | (4.76) | (9.52) | (0.95) | (80) |
| 10% C12-200 | 15 | 15 | 30 | 3 | 7 |
| | (21.43) | (21.43) | (42.86) | (4.29) | (10) |
| 20% C12-200 | 15 | 15 | 30 | 3 | 15.75 |
| | (19.05) | (19.05) | (38.10) | (3.81) | (20) |
| 50% C12-200 | 15 | 15 | 30 | 3 | 63 |
| | (11.90) | (11.90) | (23.81) | (2.38) | (50) |
| 10% 5A2-SC8 | 15 | 15 | 30 | 3 | 7 |
| | (21.43) | (21.43) | (42.86) | (4.29) | (10) |
| 15% 5A2-SC8 | 15 | 15 | 30 | 3 | 11.12 |
| | (20.24) | (20.24) | (40.47) | (4.05) | (15) |
| 20% 5A2-SC8 | 15 | 15 | 30 | 3 | 15.75 |
| | (19.05) | (19.05) | (38.10) | (3.81) | (20) |
| 25% 5A2-SC8 | 15 | 15 | 30 | 3 | 21 |
| | (17.86) | (17.86) | (35.71) | (3.57) | (25) |
| 30% 5A2-SC8 | 15 | 15 | 30 | 3 | 27 |
| | (16.67) | (16.67) | (33.33) | (3.33) | (30) |

Zwitterionic Lipid SORT Molecule

| | | | | | |
|---|---|---|---|---|---|
| 20% DSPC | 15 | 15 | 30 | 3 | 15.75 |
| | (19.05) | (19.05) | (38.10) | (3.81) | (20) |
| 50% DSPC | 15 | 15 | 30 | 3 | 63 |
| | (11.90) | (11.90) | (23.81) | (2.38) | (50) |
| 80% DSPC | 15 | 15 | 30 | 3 | 252 |
| | (4.76) | (4.76) | (9.52) | (0.95) | (80) |
| 20% DOCPe | 15 | 15 | 30 | 3 | 15.75 |
| | (19.05) | (19.05) | (38.10) | (3.81) | (20) |
| 50% DOCPe | 15 | 15 | 30 | 3 | 63 |
| | (11.90) | (11.90) | (23.81) | (2.38) | (50) |

DLin-MC3-DMA-based LNPs

| | DLin-MC3- | | | | SORT |
|---|---|---|---|---|---|
| Name | DMA | DOPE | Cholesterol | DMG-PEG2K | molecule |
| MC3-LNP | 15 | 15 | 30 | 3 | n/a |
| | (23.81) | (23.81) | (47.62) | (4.76) | (0) |

Cationic Lipid SORT Molecule

| | | | | | |
|---|---|---|---|---|---|
| 15% DOTAP | 15 | 15 | 30 | 3 | 11.12 |
| MC3 | (20.24) | (20.24) | (40.47) | (4.05) | (15) |
| 50% DOTAP | 15 | 15 | 30 | 3 | 63 |
| MC3 | (11.90) | (11.90) | (23.81) | (2.38) | (50) |

Anionic Lipid SORT Molecule

| | | | | | |
|---|---|---|---|---|---|
| 10% 18PA | 15 | 15 | 30 | 3 | 7 |
| MC3 | (21.43) | (21.43) | (42.86) | (4.29) | (10) |
| 20% 18PA | 15 | 15 | 30 | 3 | 15.75 |
| MC3 | (19.05) | (19.05) | (38.10) | (3.81) | (20) |
| 30% 18PA | 15 | 15 | 30 | 3 | 27 |
| MC3 | (16.67) | (16.67) | (33.33) | (3.33) | (30) |

C12-200-based LNPs

| | | | | | SORT |
|---|---|---|---|---|---|
| Name | C12-200 | DOPE | Cholesterol | DMG-PEG2K | molecule |
| C12-200-LNP | 15 | 15 | 30 | 3 | n/a |
| | (23.81) | (23.81) | (47.62) | (4.76) | (0) |

Cationic Lipid SORT Molecule

| | | | | | |
|---|---|---|---|---|---|
| 15% DOTAP | 15 | 15 | 30 | 3 | 11.12 |
| C12-200 | (20.24) | (20.24) | (40.47) | (4.05) | (15) |
| 50% DOTAP | 15 | 15 | 30 | 3 | 63 |
| C12-200 | (11.90) | (11.90) | (23.81) | (2.38) | (50) |

Anionic Lipid SORT Molecule

| | | | | | |
|---|---|---|---|---|---|
| 10% 18PA | 15 | 15 | 30 | 3 | 7 |
| C12-200 | (21.43) | (21.43) | (42.86) | (4.29) | (10) |
| 20% 18PA | 15 | 15 | 30 | 3 | 15.75 |
| C12-200 | (19.05) | (19.05) | (38.10) | (3.81) | (20) |
| 30% 18PA | 15 | 15 | 30 | 3 | 27 |
| C12-200 | (16.67) | (16.67) | (33.33) | (3.33) | (30) |

C1-based LNPs

| | | | | | |
|---|---|---|---|---|---|
| | | | | | SORT |
| Name | 5A2-SC8 | DOPE | Cholesterol | DMG-PEG2K | molecule |

Ionizable Cationic Lipid SORT Molecule

| | | | | | |
|---|---|---|---|---|---|
| 20% DODAP | 15 | 15 | 30 | 3 | 15.75 |
| C1 | (19.05) | (19.05) | (38.10) | (3.81) | (20) |
| 30% DODAP | 15 | 15 | 30 | 3 | 27 |
| C1 | (16.67) | (16.67) | (33.33) | (3.33) | (30) |

Cationic Lipid SORT Molecule

| | | | | | |
|---|---|---|---|---|---|
| 10% DOTAP | 15 | 15 | 30 | 3 | 7 |
| C1 | (21.43) | (21.43) | (42.86) | (4.29) | (10) |
| 20% DOTAP | 15 | 15 | 30 | 3 | 15.75 |
| C1 | (19.05) | (19.05) | (38.10) | (3.81) | (20) |
| 30% DOTAP | 15 | 15 | 30 | 3 | 27 |
| C1 | (16.67) | (16.67) | (33.33) | (3.33) | (30) |

FIG. 1E shows a plot of relative pKₐ with respect to tissue-specific activity. Example SORT LNPs targeting the liver (score=1) had apparent pKₐ in the 6-7 range. All lung-targeting (score=3) SORT LNPs had a higher apparent pKₐ (e.g., >9) while spleen targeting (score=2) LNPs had a lower pKₐ, by way of example(s).

It is established that global/apparent pKa plays an important role in determining the potency of RNA delivery by LNPs. Indeed, siRNA delivery to liver hepatocytes strongly depends on LNP pKa, with a pKa of 6.2-6.4 being optimal for gene silencing; LNPs with a pKa outside of that narrow range were unable to functionally deliver siRNA to the liver. Based on the link between pKa and cellular delivery, we analyzed the apparent pKa of 67 different example SORT LNPs (Table 7), all of which were efficacious for tissue-specific mRNA delivery in vivo, using the 6-(p-toluidino)-2-naphthalenesulfonic acid (TNS) assay (FIGS. 1D-IE and 6-7). Because SORT involves inclusion of additional charged lipids, the resulting TNS titration curves capture the ionization behavior of more complex mixed species LNPs. As the lower limit TNS fluorescence of 0% was not measured for some SORT LNPs over the pH range of buffers used in the TNS assay, we defined the apparent pKa as the pH at which 50% normalized TNS fluorescence signal is measured (FIG. 1D).

When plotting relative pKa with respect to tissue-specific activity, the example SORT LNPs grouped into defined ranges based on their organ-targeting properties (FIG. 1E). Confirming the literature precedent, all liver-targeting SORT LNPs had an apparent pKa within the well-established 6-7 range (FIG. 1E). Surprisingly, all lung-targeting SORT LNPs had a higher apparent pKa (e.g., greater than 9) (FIG. 1E), while spleen-targeting LNPs had a lower pKa, between 2 and 6 (FIG. 1E). This result contrasts with the dogma for conventional LNP delivery to the liver and contributes to the understanding of why SORT LNPs are unconventional and can enable extrahepatic delivery. It is important to note that the SORT LNPs tested here possess a similar, near neutral zeta potential surface charge (see Table 8). Thus, the addition of a SORT molecule to a multi-component LNP alters its overall pKa that directly relates to the organ-targeting properties of the LNP.

### Example 4: PEG-Lipid Desorption Contributes to Effective mRNA Delivery In Vivo in Mice

LNPs incorporate a PEG-lipid on the surface to promote colloidal stability. Since PEG-lipid molecules are non-covalently incorporated into the LNP, they desorb at a rate inversely proportional to the length of their corresponding lipid anchor. Further, PEG-lipid on an LNP surface is known to impair serum or plasma protein adsorption. Shedding of PEG-lipid would be expected to expose the underlying SORT molecules for recognition by serum or plasma proteins, promoting their binding of the example SORT LNP in the blood.

To characterize the consequences of PEG-lipid desorption on mRNA delivery, the effects of substituting an mPEG glyceride with a shorter alkyl tail (e.g., DMG-PEG2000 (C14-PEG2K) with a 14-carbon alkyl tail) versus with an mPEG glyceride with a longer alkyl tail (e.g., DSG-PEG2000 (C18-PEG2K), with an 18-carbon long alkyl tail), on the in vivo potency of example SORT LNPs were measured. An mPEG with a shorter alkyl tail is expected to be less-sheddable from the LNP compared to an mPEG with a longer alkyl tail. The effects of increasing the PEG-lipid anchor length on the delivery of luciferase mRNA was measured by injecting C57BL/6 mice IV at a dosage of 0.1 mg/kg mRNA and imaging organ luminescence ex vivo at a timepoint of 6 hours post-injection. Imaging results are shown in FIG. 2A. Average fluorescence of the liver, lung, and spleen were measured using Living Image Software (Perkin Elmer) by drawing regions of interest around each organ. The relative fluorescence for each organ was calculated as:

$${F}_{relative , organ} = \frac{{F}_{average , organ}}{\sum {F}_{average , organ}} \times 100.$$

As shown in FIG. 2B, total luminescence produced by each organ was reduced when less-sheddable PEG-lipid as used, suggesting PEG-lipid desorption is a key process for efficacious mRNA delivery by example SORT LNPs. FIG. 2C shows quantification of luciferase mRNA (as determined by total luminescence) in target organs of treated mice.

To further characterize the consequences of PEG-lipid desorption on mRNA delivery, human erythropoietin (hEPO) mRNA was delivered to target tissues in vivo using example Liver, Spleen, or Lung SORT LNPs incorporating either shorter (e.g., C14-PEG2K) or longer (e.g, C18-PEG2K) alkyl chain PEG-lipid. Since hEPO is a secreted protein, delivery efficacy was quantified by measuring hEPO levels in the serum or plasma of treated mice. Serum or plasma hEPO concentration was lower in mice treated with C18-PEG2K SORT LNPs compared to those treated with C14-PEG2K SORT LNPs, as shown in FIG. 2D. Thus, SORT LNPs which incorporate a PEG-lipid with a longer hydrophobic anchor were less effective for mRNA delivery to target organs and further suggested that PEG-lipid desorption is a necessary process for effective mRNA delivery to target tissues by LNPs.

Conventional four-component LNPs are known to enable functional RNA delivery into liver hepatocytes via an endogenous mechanism where the desorption of PEG-lipid from the LNP surface enables apolipoprotein E (ApoE) to bind the LNP; this subsequently enables receptor-mediated binding and uptake of the LNPs by the low-density lipoprotein receptor (LDL-R). We hypothesized that example SORT LNPs might operate by a similar mechanism, where (1) PEG-lipid desorbs from the LNP to expose underlying SORT molecules, (2) distinct serum proteins recognize the exposed SORT molecules and adsorb to the LNP surface, and (3) these surface-adsorbed proteins interact with cognate receptors which mediate uptake of the LNPs by cells in target tissues (FIG. 2A). First, we established the role of the PEG-lipid component on mRNA delivery efficacy in vivo. LNPs incorporate a PEG-lipid on the surface to promote colloidal stability. Because the PEG-lipid molecules are non-covalently incorporated into the LNP, they spontaneously desorb at a rate inversely proportional to the length of the PEG-lipid's hydrophobic anchor. PEG-lipid on the LNP surface can impair serum protein adsorption, resulting in reduced cellular targeting and delivery efficacy. Shedding of PEG-lipid would be expected to expose the underlying SORT molecules for recognition by serum proteins, promoting their binding of the example SORT LNP in the blood.

To functionally characterize the consequences of PEG-lipid desorption on mRNA delivery, we measured the effects of substituting DMG-PEG2000 (C14-PEG2K), a mPEG glyceride with a 14-carbon long alkyl tail, with DSG-PEG2000 (C18-PEG2K), a mPEG glyceride with an 18-carbon long alkyl tail, on the in vivo potency of example SORT LNPs. C18-PEG2K is expected to be less-sheddable from the LNP compared to C14-PEG2K due to its longer hydrophobic anchor. First, we measured the effects of increasing the PEG-lipid anchor length on the delivery of luciferase mRNA by injecting C57BL/6 mice IV at a dosage of 0.1 mg/kg mRNA and imaging organ luminescence ex vivo at a timepoint of 6 hours post-injection (FIG. 2B). Switching to C18-PEG2K significantly reduced the total luminescence produced by luciferase mRNA translated into a functional protein in target organs when compared to C14-PEG2K for all example SORT LNPs (FIG. 2C). To confirm these findings, we delivered human erythropoietin (hEPO) mRNA to target tissues in vivo using an example Liver, Spleen, or Lung SORT LNPs incorporating either C14-PEG2K or C18-PEG2K. Because hEPO is a secreted protein, delivery efficacy can be readily quantified by measuring hEPO levels in the serum. We verified that serum hEPO concentration was lower in mice treated with example C18-PEG2K SORT LNPs compared to those treated with example C14-PEG2K SORT LNPs (FIG. 2D). Thus, we quantitatively confirmed that example SORT LNPs which incorporate a PEG-lipid with a longer hydrophobic anchor are less effective for mRNA delivery to target organs. These studies suggest that PEG-lipid desorption is a necessary process for efficacious mRNA delivery to target tissues by example SORT LNPs.

### Example 5: SORT Molecule Choice Influences LNP-Protein Interactions in the Serum

The plasma proteins which bind a conventional four-component LNP formulation (mDLNP), and three different example SORT LNP formulations as described herein (Liver SORT, comprising an ionizable cationic lipid (e.g., DODAP); Lung SORT, comprising a cationic lipid (e.g., DOTAP); Spleen SORT, comprising an anionic lipid (e.g., 18PA)) were isolated using differential centrifugation following ex vivo incubation with mouse plasma. Details of the SORT formulations are described in Table 8. Briefly, mouse plasma was added to a solution of each LNP (prepared as in Example 1 and diluted with 1×PBS 1 g/L concentration of lipid) at a 1:1 volume ratio and incubated for 15 minutes at 37° C. A 0.7 M sucrose solution was prepared by dissolving solid sucrose in MilliQ water. The LNP/Plasma mixture was loaded onto a 0.7 M sucrose cushion of equal volume to the mixture and centrifuged at 15,300 g and 4° C. for 1 hour. The supernatant was removed, and the pellet was washed with 1×PBS. Next the pellet was centrifuged at 15,300 g and 4° C. for 5 minutes, and the supernatant was removed. Washing was performed twice more for a total of three washes. Following the final wash, the pellet was resuspended in 2 wt % SDS. Excess lipids were removed from each sample by following the protocol provided with the ReadyPrep 2-D Cleanup (BioRad). The resulting pellet from the cleanup step was resuspended in 2× Laemmli buffer. The concentration of protein in each sample was quantified by Bradford Assay using the Pierce 660 nm Protein Assay Reagent mixed with Ionic Detergent Compatibility Reagent. Treating 4-component mDLNP reference as the base liver-targeting LNP composition, an SDS-PAGE gel was run to qualitatively study how the choice of SORT molecule impacted which proteins adsorbed to 5-component SORT LNPs. As demonstrated in FIG. 2E, while the set of plasma proteins that adsorbed to Liver SORT was qualitatively similar to that of mDLNP (ref), the plasma proteins that bound SORT LNPs targeting extrahepatic organs were markedly distinct. In particular, as shown in FIG. 2E, a key band was highly enriched for Spleen SORT LNPs at 54 kDa while a band near 65 kDa was highly enriched for Lung SORT LNPs.

TABLE 8
Composition and physiochemical characterization of LNP formulations
Lipid Nanoparticle Composition and Properties

| | Lipid | Molar Percentage | Size | | ζ-potential |
|---|---|---|---|---|---|
| Name | Composition | (Molar Ratio) | (nm) | PDI | (mV) |
| mDLNP | 5A2-SC8:DOPE:Cholesterol:DMG- | 23.8:23.8:47.6:4.8 | 144.0 | 0.135 | −3.59 |
| (reference) | PEG2K | (15:15:30:3) | | | |
| Example | 5A2-SC8:DOPE:Cholesterol:DMG- | 19:19:38:4:20 | 154.6 | 0.115 | −3.82 |
| Liver SORT | PEG2K:DODAP | (15:15:30:3:15.75) | | | |
| Example | 5A2-SC8:DOPE:Cholesterol:DMG- | 16.7:16.7:33.3:3.3:30 | 167.8 | 0.144 | −1.84 |
| Spleen SORT | PEG2K:18PA | (15:15:30:3:27) | | | |
| Example | 5A2-SC8:DOPE:Cholesterol:DMG- | 11.9:11.9:23.8:2.4:50 | 114.8 | 0.159 | −0.89 |
| Lung SORT | PEG2K:DOTAP | (15:15:30:3:63) | | | |

Unbiased mass spectrometry was used for identification and quantification of which proteins bound SORT LNPs in the plasma. Plasma proteins were classified into the physiological classes of apolipoproteins, coagulation proteins, complement proteins, immune proteins, and other proteins. Proteomic signatures corresponding to conventional four-component and SORT LNPs are shown in FIG. 2F. The proportions of proteins in each class varied depending on choice of SORT molecule.

The isoelectric point (pI) of the major proteins that bound mDLNP (reference) and each SORT LNP was determined from a database (Expasy). As shown in the top two panels of FIG. 2G, inclusion of an ionizable cationic lipid (e.g., DODAP) in mDLNPs did not greatly alter the pI distribution of the major proteins which bound the LNP. In contrast, the inclusion of a lipid with an anionic headgroup (e.g., 18PA) in mDLNPs promoted the adsorption of plasma proteins with a pI greater than physiological pH (as shown in the third panel of FIG. 2G) while the inclusion of a lipid with a cationic head group (e.g., DOTAP) favored the enrichment of proteins with a pI below physiological pH (as shown in the bottom panel of FIG. 2G).

LNPs were further characterized based on the 5 most abundant proteins in their respective coronas. FIG. 2H shows that the most highly enriched protein in each corona was found to depend on the identity of SORT molecule. The top left and right panel of FIG. 2H shows ApoE was found to be the most highly enriched serum or plasma protein for mDLNP (reference) and Lung SORT LNPs, on average composing 13.9% of mDLNP's protein corona and 13.3% for Lung SORT. In contrast, Spleen SORT LNPs were most highly enriched in β2-glycoprotein I (β2-GPI), at an average abundance of 20.1% as shown in the bottom left of FIG. 2H, while Lung SORT LNPs were most highly enriched in vitronectin (Vtn), at an average abundance of 12.2%, as shown in the bottom right of FIG. 2H. FIG. 2H thus demonstrates that different SORT molecules may result in unique protein corona signatures. Average abundance, isoelectronic point, and physiological function of the proteins which constitute 80% of the protein corona for each LNP formulation are listed in Tables 9-12.

TABLE 9
Average abundance, isoelectric point, and physiological function of
the proteins which constitute 80% of reference mDLNP's protein corona

| Protein | CD1¹ | C57BL/6² | pI | Functional Class |
|---|---|---|---|---|
| Apolipoprotein E | 13.95% | 33.82% | 5.56 | Apolipoprotein |
| Complement C1q subcomponent | 7.79% | 2.79% | 9.25 | Complement |
| subunit A | | | | Protein |
| Immunoglobulin kappa constant | 7.74% | 4.51% | 5.6 | Immune Protein |
| Immunoglobulin heavy constant | 4.86% | 1.36% | 6.1 | Immune Protein |
| gamma 2B (Fragment) | | | | |
| Complement C1q subcomponent | 4.78% | 4.71% | 8.3 | Complement |
| subunit B | | | | Protein |
| Vitronectin | 4.33% | 0.99% | 5.67 | Other |
| Ig gamma-2A chain C region, | 3.96% | 0.04% | 5.88 | Immune Protein |
| membrane-bound form | | | | |
| Complement C1q subcomponent | 3.93% | 2.52% | 8.84 | Complement |
| subunit C | | | | Protein |
| Ig mu chain C region (Fragment) | 3.77% | n.d.³ | 6.6 | Immune Protein |
| Serum albumin | 2.27% | 5.98% | 5.75 | Other |
| Immunoglobulin kappa variable | 1.50% | 0.09% | 6.24 | Immune Protein |
| 6-13 | | | | |
| Beta-2-glycoprotein 1 | 1.26% | 0.25% | 8.59 | Other |
| Immunoglobulin heavy constant | 1.22% | 0.45% | 5.12 | Immune Protein |
| alpha (Fragment) | | | | |
| Immunoglobulin kappa variable | 1.15% | n.d. | 9.06 | Immune Protein |
| 1-110 (Fragment) | | | | |
| Serum paraoxonase/arylesterase 1 | 1.09% | 0.09% | 5.07 | Other |
| Immunoglobulin heavy constant | 1.04% | 0.57% | 6.49 | Immune Protein |
| gamma 2C (Fragment) | | | | |
| Ig heavy chain V region AC38 | 0.94% | n.d. | 6.85 | Immune Protein |
| 205.12 | | | | |
| Beta-globin | 0.83% | 0.09% | 7.13 | Other |
| Alpha-S1-casein | 0.81% | 5.16% | 6.08 | Other |
| Protein Igkv12-41 (Fragment) | 0.73% | n.d. | n/a | Immune Protein |
| Clusterin | 0.68% | 0.11% | 5.45 | Other |
| Fibrinogen beta chain | 0.66% | 1.31% | 6.68 | Coagulation |
| | | | | Protein |
| Ig gamma-1 chain C region | 0.57% | 0.11% | 7.2 | Immune Protein |
| secreted form (Fragment) | | | | |
| Apolipoprotein A-I | 0.57% | 0.49% | 5.51 | Apolipoprotein |
| Ig kappa chain V-V region HP | 0.56% | n.d. | 8.64 | Immune Protein |
| 124E1 | | | | |
| Ig kappa chain V-III region PC | 0.55% | n.d. | 5.18 | Immune Protein |
| 2880/PC 1229 | | | | |
| Immunoglobulin kappa variable | 0.55% | 2.11% | 4.17 | Immune Protein |
| 17-121 (Fragment) | | | | |
| Immunoglobulin kappa chain | 0.54% | 0.18% | 6.09 | Immune Protein |
| variable 8-30 (Fragment) | | | | |
| Immunoglobulin kappa variable | 0.51% | 0.23% | 6.54 | Immune Protein |
| 1-135 (Fragment) | | | | |
| Alpha globin 1 | 0.50% | 0.46% | 7.97 | Other |
| Apolipoprotein D | 0.49% | 1.84% | 4.82 | Apolipoprotein |
| Fibrinogen gamma chain | 0.49% | 1.44% | 5.38 | Coagulation |
| | | | | Protein |
| Immunoglobulin kappa variable | 0.47% | 0.08% | 9.06 | Immune Protein |
| 4-57 (Fragment) | | | | |
| Ig heavy chain V region 914 | 0.47% | 0.005% | 9.27 | Immune Protein |
| Immunoglobulin heavy constant | 0.46% | 0.45% | 6.77 | Immune Protein |
| gamma 3 (Fragment) | | | | |
| Fibrinogen alpha chain | 0.45% | 0.86% | 5.78 | Coagulation |
| | | | | Protein |
| Immunoglobulin heavy variable | 0.39% | 0.02% | 9.1 | Immune Protein |
| 5-9 (Fragment) | | | | |
| Apolipoprotein C-III | 0.38% | 0.02% | 5.43 | Apolipoprotein |
| Prothrombin | 0.37% | 0.05% | 6.04 | Coagulation |
| | | | | Protein |
| Carboxypeptidase N catalytic | 0.35% | 0.63% | 8.42 | Other |
| chain | | | | |
| Immunoglobulin kappa variable | 0.35% | 0.02% | 5.59 | Immune Protein |
| 12-46 (Fragment) | | | | |
| Immunoglobulin kappa variable | 0.34% | 0.11% | 7.71 | Immune Protein |
| 12-44 (Fragment) | | | | |
| Alpha-1-antitrypsin 1-3 | 0.34% | 0.41% | 5.25 | Other |
| Serine protease inhibitor A3K | 0.33% | 0.25% | 5.05 | Other |
| Immunoglobulin heavy variable | 0.33% | n.d. | 8.85 | Immune Protein |
| 1-34 (Fragment) | | | | |
| Ig gamma-2B chain C region | 0.32% | 0.01% | 6.10 | Immune Protein |
| Ig heavy chain V region X44 | 0.32% | 0.005% | 8.00 | Immune Protein |

¹Samples run using a Thermo Fusion Lumos mass spectrometer.
²Samples run using a Thermo QExactive HF mass spectrometer.
³n.d., not detected.

TABLE 10
Average abundance, isoelectric point, and physiological function of the
proteins which constitute 80% of example Liver SORT's protein corona.

| Protein | CD1¹ | C57BL/6² | pI | Functional Class |
|---|---|---|---|---|
| Apolipoprotein E | 13.34% | 23.81% | 5.56 | Apolipoprotein |
| Complement C1q subcomponent subunit A | 8.20% | 2.89% | 9.25 | Complement Protein |
| Immunoglobulin heavy constant mu | 6.74% | 8.31% | 6.56 | Immune Protein |
| Complement C1q subcomponent subunit B | 6.01% | 4.65% | 8.3 | Complement Protein |
| Serum albumin | 5.68% | 7.06% | 5.75 | Other |
| Immunoglobulin kappa constant | 4.92% | 3.48% | 5.6 | Immune Protein |
| Immunoglobulin heavy constant gamma 2B | 2.93% | 1.81% | 6.1 | Immune Protein |
| (Fragment) | | | | |
| Beta-globin | 2.67% | 0.19% | 7.13 | Other |
| Ig gamma-2A chain C region, membrane-bound | 2.39% | 0.04% | 5.88 | Immune Protein |
| form | | | | |
| Complement C1q subcomponent subunit C | 2.28% | 2.67% | 8.84 | Complement Protein |
| Immunoglobulin heavy constant alpha | 2.01% | 0.67% | 5.12 | Immune Protein |
| (Fragment) | | | | |
| Fibrinogen beta chain | 1.81% | 3.30% | 6.68 | Coagulation Protein |
| Fibrinogen gamma chain | 1.55% | 3.02% | 5.38 | Coagulation Protein |
| Immunoglobulin kappa variable 17-127 | 1.53% | n.d.³ | 4.17 | Immune Protein |
| (Fragment) | | | | |
| Alpha globin 1 | 1.26% | 0.38% | 7.97 | Other |
| Fibrinogen alpha chain | 1.15% | 1.69% | 5.78 | Coagulation Protein |
| Apolipoprotein A-I | 0.98% | 0.41% | 5.51 | Apolipoprotein |
| Vitronectin | 0.92% | 1.38% | 5.67 | Other |
| Ig heavy chain V region AC38 205.12 | 0.90% | n.d. | 6.85 | Immune Protein |
| Alpha-2-HS-glycoprotein | 0.90% | 0.08% | 6.04 | Other |
| Ig alpha chain C region | 0.79% | n.d. | 4.97 | Immune Protein |
| Serine protease inhibitor A3K | 0.78% | n.d. | 5.05 | Other |
| Ig gamma-1 chain C region secreted form | 0.64% | 0.24% | 7.2 | Immune Protein |
| (Fragment) | | | | |
| Histone H4 | 0.62% | 0.03% | 11.36 | Other |
| Immunoglobulin heavy constant gamma 2C | 0.57% | 1.81% | 6.49 | Immune Protein |
| (Fragment) | | | | |
| Ig kappa chain V-V region HP 124E1 | 0.51% | n.d. | 8.64 | Immune Protein |
| Immunoglobulin kappa variable 1-110 | 0.49% | n.d. | 9.06 | Immune Protein |
| (Fragment) | | | | |
| Inter alpha-trypsin inhibitor, heavy chain 4 | 0.47% | 0.001% | 5.99 | Other |
| Immunoglobulin heavy constant gamma 3 | 0.46% | 0.46% | 6.77 | Immune Protein |
| (Fragment) | | | | |
| Serotransferrin | 0.46% | 0.37% | 6.94 | Other |
| Ig kappa chain V-III region 50S10.1 | 0.45% | 0.02% | 4.9 | Immune Protein |
| Apolipoprotein A-IV | 0.44% | 0.09% | 5.34 | Apolipoprotein |
| Ig heavy chain V region 6.96 | 0.42% | 0.08% | 8 | Immune Protein |
| Lactadherin | 0.41% | 0.40% | 6.1 | Other |
| Ig kappa chain V-II region 26-10 | 0.40% | 0.91% | 9.04 | Immune Protein |
| Platelet factor 4 | 0.39% | 0.002% | 9.42 | Other |
| Alpha-1-antitrypsin 1-3 | 0.38% | n.d. | 5.25 | Other |
| Ig heavy chain V-III region J606 | 0.38% | n.d. | 6.86 | Immune Protein |
| Band 3 anion transport protein | 0.36% | 0.05% | 5.31 | Other |
| Apolipoprotein D | 0.35% | 2.30% | 4.82 | Apolipoprotein |
| Hemoglobin subunit beta-2 | 0.34% | n.d. | 7.85 | Other |
| H-2 class I histocompatibility antigen, Q10 alpha | 0.34% | 0.18% | 5.13 | Immune Protein |
| chain | | | | |
| Immunoglobulin kappa variable 6-13 | 0.33% | 0.07% | 6.24 | Immune Protein |
| Alpha-S1-casein | 0.32% | 2.52% | 6.08 | Other |
| Immunoglobulin heavy variable 7-1 (Fragment) | 0.31% | 0.55% | 8.49 | Immune Protein |
| Immunoglobulin heavy variable V1-5 | 0.31% | n.d. | 9.18 | Immune Protein |
| Histone H2B | 0.31% | n.d. | 10.37 | Other |

¹Samples run using a Thermo Fusion Lumos mass spectrometer.
²Samples run using a Thermo QExactive HF mass spectrometer.
³n.d., not detected.

TABLE 11
Average abundance, isoelectric point, and physiological function of the
proteins which constitute 80% of example Spleen SORT's protein corona.

| Protein | CD1¹ | C57BL/6² | pI | Functional Class |
|---|---|---|---|---|
| Beta-2-glycoprotein 1 | 20.10% | 42.02% | 8.59 | Other |
| Immunoglobulin kappa constant | 7.38% | 2.96% | 5.6 | Immune Protein |
| Complement C1q subcomponent subunit A | 6.86% | 1.92% | 9.25 | Complement Protein |
| Apolipoprotein E | 6.20% | 5.52% | 5.56 | Apolipoprotein |
| Immunoglobulin heavy constant gamma 2B | 3.65% | 0.54% | 6.1 | Immune Protein |
| (Fragment) | | | | |
| Complement C1q subcomponent subunit B | 3.61% | 3.85% | 8.3 | Complement Protein |
| Vitronectin | 3.52% | 1.26% | 5.67 | Other |
| Complement C1q subcomponent subunit C | 2.74% | 2.28% | 8.84 | Complement Protein |
| Apolipoprotein C-I | 2.58% | 0.59% | 9.05 | Apolipoprotein |
| Ig gamma-2A chain C region, membrane-bound | 2.57% | 0.02% | 5.88 | Immune Protein |
| form | | | | |
| Ig mu chain C region (Fragment) | 2.36% | n.d. | 6.6 | Immune Protein |
| Serum albumin | 1.80% | 4.69% | 5.75 | Other |
| Serum paraoxonase/arylesterase 1 | 1.22% | 0.15% | 5.07 | Other |
| Immunoglobulin heavy constant alpha | 1.17% | 0.11% | 5.12 | Immune Protein |
| (Fragment) | | | | |
| Immunoglobulin kappa variable 6-13 | 1.09% | 0.06% | 6.24 | Immune Protein |
| Immunoglobulin heavy constant gamma 2C | 0.87% | 0.39% | 6.49 | Immune Protein |
| (Fragment) | | | | |
| Fibrinogen beta chain | 0.81% | 1.62% | 6.68 | Coagulation Protein |
| Alpha-S1-casein | 0.80% | 2.02% | 6.08 | Other |
| Ig heavy chain V region AC38 205.12 | 0.80% | n.d. | 6.85 | Immune Protein |
| Clusterin | 0.73% | 0.15% | 5.45 | Other |
| Immunoglobulin kappa variable 1-110 | 0.66% | n.d. | 9.06 | Immune Protein |
| (Fragment) | | | | |
| Protein Igkv12-41 (Fragment) | 0.63% | n.d. | n/a | Immune Protein |
| Beta-globin | 0.61% | 0.06% | 7.13 | Other |
| Fibrinogen alpha chain | 0.57% | 1.09% | 5.76 | Coagulation Protein |
| Apolipoprotein A-I | 0.54% | 0.58% | 5.51 | Apolipoprotein |
| Fibrinogen gamma chain | 0.52% | 1.69% | 5.38 | Coagulation Protein |
| Lipopolysaccharide-binding protein | 0.52% | 0.61% | 8.64 | Other |
| Histidine-rich glycoprotein | 0.51% | 0.13% | 7.26 | Other |
| Ig kappa chain V-V region HP 124E1 | 0.48% | n.d. | 8.64 | Immune Protein |
| Ig kappa chain V-III region PC 2880/PC 1229 | 0.46% | n.d. | 5.18 | Immune Protein |
| Ig gamma-1 chain C region secreted form | 0.44% | 0.03% | 7.2 | Immune Protein |
| (Fragment) | | | | |
| Immunoglobulin kappa chain variable 8-30 | 0.42% | 0.14% | 6.09 | Immune Protein |
| (Fragment) | | | | |
| Apolipoprotein C-III | 0.39% | 0.001% | 5.43 | Apolipoprotein |
| Alpha globin 1 | 0.38% | 0.71% | 7.97 | Other |
| Immunoglobulin heavy variable 5-9 (Fragment) | 0.37% | 0.47% | 9.1 | Immune Protein |
| Complement C3 | 0.36% | 0.24% | 6.29 | Complement Protein |
| Coagulation factor V | 0.36% | 0.17% | 5.68 | Coagulation Protein |
| Alpha-1-antitrypsin 1-3 | 0.35% | 0.30% | 5.25 | Other |
| Carboxypeptidase N catalytic chain | 0.35% | 0.29% | 8.42 | Other |
| Immunoglobulin heavy constant gamma 3 | 0.34% | 0.32% | 6.77 | Immune Protein |
| (Fragment) | | | | |

¹Samples run using a Thermo Fusion Lumos mass spectrometer.
²Samples run using a Thermo QExactive HF mass spectrometer.
³n.d., not detected.

TABLE 12
Average abundance, isoelectric point, and physiological function of the
proteins which constitute 80% of example Lung SORT's protein corona.

| Protein | CD1¹ | C57BL/6² | pI | Functional Class |
|---|---|---|---|---|
| Vitronectin | 12.23% | 22.08% | 5.67 | Other |
| Serum paraoxonase/arylesterase 1 | 6.43% | 9.26% | 5.07 | Other |
| Apolipoprotein E | 6.07% | 1.43% | 5.56 | Apolipoprotein |
| Serum albumin | 5.38% | 6.47% | 5.75 | Other |
| Clusterin | 4.01% | 4.66% | 5.45 | Other |
| Immunoglobulin kappa constant | 3.37% | 0.84% | 5.6 | Immune Protein |
| Prothrombin | 2.26% | 7.12% | 6.04 | Coagulation Protein |
| Complement C1q subcomponent subunit A | 2.12% | 0.38% | 9.25 | Complement Protein |
| Fibrinogen beta chain | 2.11% | 6.96% | 6.68 | Coagulation Protein |
| Beta-2-glycoprotein 1 | 1.96% | 0.07% | 8.59 | Apolipoprotein |
| Ig mu chain C region (Fragment) | 1.70% | n.d.³ | 6.6 | Immune Protein |
| Alpha-S1-casein | 1.68% | 1.96% | 6.08 | Other |
| Immunoglobulin heavy constant gamma 2B | 1.55% | 0.47% | 6.1 | Immune Protein |
| (Fragment) | | | | |
| Fibrinogen gamma chain | 1.50% | 6.00% | 5.38 | Coagulation Protein |
| Fibrinogen alpha chain | 1.41% | 1.64% | 5.78 | Coagulation Protein |
| Vitamin K-dependent protein Z | 1.33% | 0.73% | 5.6 | Coagulation Protein |
| Alpha-1-antitrypsin 1-3 | 1.33% | 0.02% | 5.25 | Other |
| Plasminogen | 1.31% | 1.26% | 6.21 | Coagulation Protein |
| Apolipoprotein C-III | 1.30% | 0.15% | 5.43 | Apolipoprotein |
| Complement C1q subcomponent subunit B | 1.27% | 0.78% | 8.3 | Complement Protein |
| Thrombospondin-1 | 1.26% | 0.26% | 4.72 | Coagulation Protein |
| Coagulation factor X | 1.18% | 0.16% | 5.48 | Coagulation Protein |
| Apolipoprotein A-I | 1.17% | 0.10% | 5.51 | Apolipoprotein |
| Immunoglobulin heavy constant alpha | 1.17% | 0.20% | 5.12 | Immune Protein |
| (Fragment) | | | | |
| Ig gamma-2A chain C region, membrane-bound | 1.11% | 0.02% | 5.88 | Immune Protein |
| form | | | | |
| Beta-globin | 1.04% | 0.07% | 7.13 | Other |
| Complement C1q subcomponent subunit C | 0.99% | 0.42% | 8.84 | Complement Protein |
| Protein Z-dependent protease inhibitor | 0.96% | 0.75% | 5.49 | Coagulation Protein |
| Mannose-binding protein C | 0.85% | 0.15% | 4.94 | Complement Protein |
| Serine protease inhibitor A3K | 0.77% | 0.001% | 5.05 | Other |
| Vitamin K-dependent protein C | 0.76% | 0.62% | 5.94 | Coagulation Protein |
| Immunoglobulin kappa variable 6-13 | 0.73% | 0.03% | 6.24 | Immune Protein |
| Carboxypeptidase N catalytic chain | 0.68% | 0.34% | 8.42 | Other |
| Apolipoprotein N | 0.67% | 0.67% | 5.56 | Apolipoprotein |
| H-2 class I histocompatibility antigen, Q10 alpha | 0.62% | 0.64% | 5.13 | Immune Protein |
| chain | | | | |
| Complement C1s-A subcomponent | 0.62% | n.d. | 4.96 | Complement Protein |
| Alpha globin 1 | 0.60% | 0.29% | 7.97 | Other |
| C-reactive protein | 0.60% | 0.01% | 5.82 | Other |
| Beta-casein | 0.59% | 0.24% | 5.82 | Other |
| Glycosylphosphatidylinositol specific | 0.58% | 0.21% | 6.32 | Other |
| phospholipase D1 | | | | |
| Complement C3 | 0.55% | 0.34% | 6.29 | Complement Protein |
| Complement factor D | 0.50% | 0.74% | 6.18 | Complement Protein |
| Coagulation factor VII | 0.48% | 0.53% | 6.19 | Coagulation Protein |
| Inter-alpha-trypsin inhibitor heavy chain H3 | 0.44% | 0.16% | 5.70 | Other |
| Alpha-S2-casein-like A | 0.43% | 0.01% | 6.20 | Other |
| Murinoglobulin-1 | 0.40% | n.d. | 6.00 | Other |

¹Samples run using a Thermo Fusion Lumos mass spectrometer.
²Samples run using a Thermo QExactive HF mass spectrometer.
³n.d., not detected.

The second step of the hypothesized endogenous targeting mechanism for example SORT LNPs involves the adsorption of distinct proteins to the LNP surface to form unique protein coronas (FIG. 2A). Following PEG-lipid desorption, serum proteins readily adsorb to the surface of intravenously-administered LNPs, forming an interfacial layer known as the “protein corona” that defines their biological identity. Adsorption of ApoE has been shown to drive liver targeting of conventional four-component LNPs through binding of the LDL-R highly expressed on hepatocytes. Given the strikingly different organ-targeting capabilities of example Lung and Spleen SORT LNPs, we hypothesized that the set of serum proteins that bind the LNPs would be distinct. We isolated the plasma proteins which bind mDLNP, Liver SORT, Spleen SORT, and Lung SORT using differential centrifugation following ex vivo incubation with mouse plasma. Treating 4-component mDLNP as the base liver-targeting LNP composition, we used SDS-PAGE to qualitatively study how the choice of SORT molecule impacted which proteins adsorbed to example 5-component SORT LNPs (FIG. 2E). While the set of plasma proteins that adsorb to Liver SORT looks qualitatively similar to that of the reference mDLNP (mRNA optimized dendrimer lipid nanoparticle (formulation)), the plasma proteins that bind example SORT LNPs targeting extrahepatic organs are markedly distinct (FIG. 2E). In particular, a key band is highly enriched for Spleen SORT at a Mw of 54 kDa while a band near 65 kDa is highly enriched for example Lung SORT (FIG. 2E). Thus, adding a SORT molecule to mDLNP alters the composition of the protein corona in accordance with the SORT molecule's chemical structure.

Unbiased mass spectrometry proteomics enabled identification and quantification of which proteins bind example SORT LNPs in the plasma. We found that over 900 different proteins adsorbed to example SORT LNPs, but nearly 98% of these proteins are present at an abundance of less than 0.1%. We anticipate that the most abundant proteins are the ones most likely to be functionally important, thus we focused our subsequent analysis on the proteins that constitute the majority of the protein corona (80% abundance) for each LNP (Tables 9-12). First, we assessed the biological function of these highly-abundant proteins. By clustering plasma proteins into the physiological classes of apolipoproteins, coagulation proteins, complement proteins, immune proteins, and other proteins, we discovered that the proportion of proteins in each class changed based on the choice of SORT molecule, suggesting that the inclusion of a SORT molecule results in large-scale differences in the ensemble of proteins which bind an LNP (FIG. 2F). The distinct functions of these proteins may play a role in shaping an LNP's endogenous identity and subsequent fate in vivo.

To better understand what drives these large-scale differences in the functional composition of the protein corona, we studied how a SORT molecule's chemical structure might affect which proteins bind to LNPs. While each SORT molecule shares a common hydrophobic scaffold that enables the molecule to self-assemble into the LNP, they are distinguished by the chemical structure and charge state of the headgroup. These molecular features may play a role in differential enrichment of proteins with distinct characteristics, possibly through electrostatic forces which bring specific proteins into proximity of the example SORT LNP to facilitate further protein-LNP interactions. A protein's isoelectric point (pI) is defined as the pH at which the protein molecule bears no net charge, providing an estimate of a protein's charge state in the physiological milieu. We determined the pI of the major proteins that bound mDLNP and each example SORT LNP using the bioinformatics resource ExPASy. The inclusion of DODAP, an ionizable cationic lipid, to mDLNP did not greatly alter the pI distribution of the major proteins which bound the LNP (FIG. 2G). In contrast, the inclusion of 18PA, a lipid with an anionic headgroup, to mDLNP promoted the adsorption of plasma proteins with a pI greater than physiological pH (FIG. 2G) while the inclusion of DOTAP, a lipid with a cationic quaternary ammonium headgroup, in mDLNP favored the enrichment of proteins with a pI below physiological pH (FIG. 2G). Although all example SORT LNPs bound proteins across a broad range of pIs, the nature of the headgroup of a chosen SORT molecule does affect which proteins adsorb to the LNP.

We further analyzed the 5 most abundant proteins in the protein corona of each LNP. It was discovered that the most highly enriched protein in each protein corona was unique based on the choice of SORT molecule (FIG. 2H). ApoE was the most highly enriched plasma protein for mDLNP, a conventional four-component LNP which targets the liver, on average composing 13.9% of mDLNP's protein corona (FIG. 2H). This result agrees with the established role of ApoE in RNA delivery to the liver, supporting the validity of the experimental approach. Additionally, Liver SORT most avidly bound ApoE at an average abundance of 13.3%, representing a 55-fold enrichment compared to native mouse plasma (FIG. 2H). In contrast, Spleen SORT was most highly enriched in β2-glycoprotein I (β2-GPI), at an average abundance of 20.1% (125-fold higher than native mouse plasma) (FIG. 2H), while Lung SORT was most highly enriched in vitronectin (Vtn), at an average abundance of 12.2% (108-fold higher than native mouse plasma) (FIG. 2H). Thus, example SORT LNPs for extrahepatic mRNA most avidly bind proteins distinct from ApoE compared to mDLNP. Furthermore, there were additional distinctions in the top 5 plasma proteins bound to example SORT LNPs, in terms of individual molecular species as well as their physiological function (FIG. 2H). To further validate our findings, we examined the protein coronas formed around example SORT LNPs in the plasma of an additional mouse strain and identified the same key proteins highly enriched in the protein coronas of example SORT LNPs (Tables 9-12). Ultimately, example SORT LNPs bind low abundance serum proteins to form unique protein coronas with quantitatively distinct composition, based on the nature of the incorporated SORT molecule, which may impact tissue-specific mRNA delivery.

### Example 6: Key Serum Proteins Define SORT LNP Cellular Targeting

As illustrated in FIG. 3A, SORT LNPs were incubated with either ApoE, β2-GPI, or Vtn, the most highly enriched serum or plasma proteins to associate with SORT LNPs as described in Example 5 hereinabove, and the effects of these proteins on LNP uptake and mRNA delivery efficiency in vitro were investigated. SORT LNPs encapsulating Cy5-mRNA were incubated with ApoE, and cellular uptake in HuH-7 and Hep G2 cells, two cell lines which highly express ApoE's receptor, LDL-R, was measured.

Briefly, cells (e.g., HuH-7, Hep G2, A-498, U-87 MG cells) were seeded into 12 well plates at a density of 1×105 cells per well and incubated at 37° C. overnight while undifferentiated THP-1 cells were seeded into 12 well plates at a density of 2×105 cells per well and treated with 100 nM of Phorbol 12-myristate 13-acetate overnight (to enable differentiation into macrophages). All cells were cultured in complete medium, as recommended by the suppliers. Then, the old media was refreshed and cells treated with 250 ng of Cyanine 5 FLuc mRNA encapsulated in SORT LNPs. LNPs were either uncoated, pre-incubated with 1.0 g protein/g total lipid of ApoE, 1.0 g protein/g total lipid β2-GPI, or pre-incubated with 0.25 g protein/g total lipid of Vtn. Ninety minutes after treatment, cells were washed two times with 1×PBS and stained with Hoechst 33342 (0.1 mg mL⁻¹) for 10 min at 37° C. Cells were washed twice more with PBS, then imaged by fluorescence microscopy (Keyence BZ-X800). Images were captured using a 10× magnification. All image settings were kept consistent for a single experiment. Cy5 fluorescence intensity was quantified using ImageJ software version 1.53c (NIH). Each treatment group was performed in duplicates and five images were taken for each well.

Intracellular accumulation of Cy5-mRNA was increased in HuH-7 and Hep G2 cells by a factor of 2.4, as shown in the top row of each of FIGS. 3B-3C, and the 6.5, respectively, when Liver SORT LNPs were incubated with ApoE. THP-1 macrophages, a cell line known to interact with β2-GPI-bound particles containing anionic phospholipids, was incubated with β2-GPI enhanced Cy5-mRNA SORT LNPs and showed increased update by a factor of 2.1 as shown by representative middle row of each of FIGS. 3B-3C and second panel of FIG. 3D. A-498 and U-87 MG cells, which highly express Vtn's receptor αvβ3 integrin were examined. Incubation with Vtn increased cellular uptake of Lung SORT LNPs at a factor of 23.2 (as shown by representative bottom row of each of FIGS. 3B-3C and bottom panel of FIG. 3D) and 4.2 (as shown by FIG. 8) times greater than the uncoated LNPs.

Further, the activity luciferase enzyme translated from mRNA delivered by uncoated and coated SORT LNPs in vitro was measured. Luciferase activity was enhanced in HuH-7 and Hep G2 cells by ApoE-coated Liver SORT LNPs whereas luciferase activity in these cells was not improved by Spleen or Lung SORT LNPs pre-incubated with ApoE), as shown in the top panel of FIG. 3D, suggesting that Spleen and Lung SORT LNPs do not efficiently bind ApoE and enter LDL-R expressing cells. Thus, ApoE exclusively enhanced functional mRNA delivery by Liver SORT LNPs in LDL-R expressing cells. Similarly, incubating Spleen SORT LNPs with β2-GPI exclusively enhanced functional mRNA delivery to THP-1 macrophages as illustrated in the middle panel of FIG. 4C, while incubating Lung SORT LNPs with Vtn exclusively enhanced functional mRNA delivery to A-498 and U-87 MG cells, as shown in the bottom panel of FIG. 3D. Collectively, these studies revealed that unique interactions between specific SORT molecules and individual plasma proteins selectively promote SORT LNP targeting to distinct cell types by enhancing cellular uptake.

One mechanism by which surface-adsorbed proteins can endogenously target specific tissues is through interactions with cognate cellular receptors, resulting in receptor-mediated endocytosis of the LNP (FIG. 2A). Having identified the serum proteins which most avidly bind example SORT LNPs, we functionally characterized how these single proteins affect intracellular delivery of mRNA by example SORT LNPs. We incubated all example SORT LNPs with either ApoE, β2-GPI, or Vtn, the most highly enriched serum proteins discovered to associate with SORT LNPs in our proteomics studies and measured the effects of these single proteins on LNP uptake and mRNA delivery efficacy in vitro (FIG. 3A). First, we incubated example SORT LNPs encapsulating Cy5-mRNA with ApoE and measured cellular uptake in HuH-7 and Hep G2 cells, two cell lines which highly express ApoE's receptor, LDL-R. Intracellular accumulation of Cy5-mRNA was increased in HuH-7 and Hep G2 cells by a factor of 2.4 (FIGS. 3B-3C) and 6.5 (FIG. 7), respectively, when example Liver SORT LNPs were incubated with ApoE. Next, we examined THP-1 macrophages, a cell line known to interact with β2-GPI-bound particles containing anionic phospholipids, and found that incubation with β2-GPI enhanced Cy5-mRNA uptake of example Spleen SORT LNPs by a factor of 2.1 (FIGS. 3B-3C). Finally, we examined A-498 and U-87 MG cells, which highly express Vtn's receptor αvβ3 integrin. Incubation with Vtn increased cellular uptake of example Lung SORT LNPs at a factor of 23.2 (FIGS. 3B-3C) and 4.2 (FIG. 8) times greater than the uncoated LNPs. Thus, the choice of SORT molecule impacts which plasma proteins adsorb to the LNP surface and these proteins likely interact with cognate receptors expressed by target cells to enhance cellular uptake promote intracellular mRNA delivery.

Having determined that key proteins can enhance cellular uptake by distinct receptors, we verified that these proteins improve functional mRNA delivery by measuring the activity of luciferase enzyme translated from mRNA delivered by uncoated and coated SORT LNPs in vitro. Additionally, we aimed to determine whether single protein coatings specifically enhanced the delivery of the individual formulations which most avidly bound those proteins. Cells were treated with example Liver, Spleen, and Lung SORT LNPs, encapsulating luciferase mRNA, that were incubated with increasing amounts of ApoE, β2-GPI, or Vtn (FIG. 3D). Luciferase activity was enhanced in HuH-7 and Hep G2 cells by ApoE-coated Liver SORT LNPs whereas luciferase activity in these cells was not improved by Spleen or Lung SORT LNPs pre-incubated with ApoE (FIG. 3D, FIG. 7), suggesting that example Spleen and Lung SORT LNPs do not efficiently bind ApoE and enter LDL-R expressing cells. Thus, ApoE exclusively enhanced functional mRNA delivery by example Liver SORT LNPs in LDL-R expressing cells. Similarly, incubating example Spleen SORT LNPs with β2-GPI exclusively enhanced functional mRNA delivery to THP-1 macrophages (FIG. 3D), while incubating Lung SORT LNPs with Vtn exclusively enhanced functional mRNA delivery to A-498 and U-87 MG cells (FIG. 3D, FIG. 8). Interestingly, incubating example Lung SORT LNPs at a ratio of 1.0 g vitronectin/g total lipid resulted in diminished luciferase activity, suggesting that excess (free in media) vitronectin may be inhibiting αvβ3 integrin to limit mRNA delivery. Collectively, these studies reveal that unique interactions between specific example SORT molecules and individual serum proteins selectively promote example SORT LNP targeting to distinct cell types by enhancing cellular uptake.

### Example 7: Extrahepatic mRNA Delivery by SORT LNPs Occurs by an ApoE-Independent Mechanism

The addition of a SORT molecule (e.g., DOTAP, PA18) to a conventional four-component LNP was studied to determine how it impacts the functional role of ApoE on tissue-specific mRNA delivery using knockout mice. SORT LNPs were prepared according to Example 1. B6.129P2-Apoe^{tm/Unc}/J mice with a weight of 18-20 g, were IV injected with mDLNP (reference) and Liver, Lung, and Spleen SORT LNPs at a dosage of 0.1 mg/kg FLuc mRNA (n=3). As a comparison, C57BL/6 mice with weight of 18-20 g, were IV injected mDLNP and Liver, Lung, and Spleen SORT LNPs at a dosage of 0.1 mg/kg FLuc mRNA (n=3). After 6 hours, mice were injected with D-Luciferin (150 mg/kg, intraperitoneal (IP)) and imaged by an IVIS Lumina system (Perkin Elmer). Total luminescence of target organs was quantified using Living Image Software (Perkin Elmer). The delivery of luciferase mRNA by SORT LNPs to target organs in B6.129P2-Apoetm1Unc/J (ApoE−/−) mice, a genetic knockout model lacking ApoE expression, was compared to that in wild type (WT) C57BL/6 mice, containing normal levels of ApoE in the serum or plasma. Luminescence was quantified to measure functional luciferase mRNA delivery to target organs. Representative images are shown in FIGS. 4A-4B. By eliminating ApoE from the serum or plasma, mDLNPs exhibited significantly reduced mRNA delivery to the liver, with an average reduction of luciferase activity by 78%, compared to WT C57BL/6 mice, as shown in the top row of FIGS. 4A-4B. Liver SORT LNPs, which also bind ApoE selectively from the serum or plasma, maintained this dependence on ApoE for mRNA delivery to the liver: knockout of ApoE significantly impaired liver targeting, with the second row of FIGS. 4A-4B showing an average reduction in luciferase activity by 87% compared to WT C57BL/6 mice. Thus, ApoE is indispensable for efficacious targeting of the liver by both mDLNPs and Liver SORT LNPs. In contrast, mRNA delivery by Spleen SORT to the spleen was enhanced by a factor of 2.2 in ApoE−/− mice (third row of FIGS. 4A-4B), suggesting that ApoE plays an antagonistic role in efficacious mRNA delivery to the spleen. Tissue-specific mRNA delivery by Lung SORT LNPs, as shown and measured in the bottom row of FIGS. 4A-4B, was not significantly different in ApoE−/− mice compared to WT mice, indicating that this serum or plasma protein is not functionally important for modulating lung targeting. Together, these results indicate that mRNA delivery is no longer an ApoE-dependent process upon inclusion of either an anionic lipid or cationic lipid to mDLNP (ref). Rather, Spleen SORT and Lung SORT LNPs can enable extrahepatic mRNA delivery via an ApoE-independent mechanism. Interactions between ApoE and LDL-R drive hepatic accumulation of endogenous lipoproteins during colestoral metabolism and may explain why ApoE adsorption is a hallmark of Liver SORT. Analogously, Vtn could bind its cognate receptor, αᵥβ₃ integrin, which is highly expressed by the pulmonary endothelium but not by liver cells or other vascular beds providing a plausible explanation for why Vtn promotes lung-specificity. Finally, β2-GPI can bind phosphatidyl serine, an anionic lipid exposed by senescent red blood cells to promote their filtration from the circulation in the s[leen, suggesting how β2-GPI could be implicated in Spleen SORT LNP targeting. These findings support the functional role of individual proteins on the organ-targeting properties of SORT LNPs. Other highly enriched proteins in enhancing organ targeting, such as albumin for Liver SORT may play a role in organ targeting as well. For example, clusterin, highly enriched in Lung SORT, could play a role in evading the mononuclear phagocyte system to promote lung targeting. Additionally, based on the key role of ApoE in driving liver targeting, the reduced binding of ApoE to Spleen and Lung SORT LNPs could promote extrahepatic mRNA delivery, possibly due to displacement of ApoE by apolipoprotein C, which is present in the protein coronas of Spleen and Lung SORT LNPs but not Liver SORT.

Adsorption of ApoE to the surface of conventional four-component LNPs is a crucial process necessary for highly efficacious RNA delivery to liver hepatocytes. mDLNP, which is a four-component LNP for mRNA delivery to the liver, avidly binds to ApoE in the serum. However, the addition of a SORT molecule to mDLNP alters which serum proteins adsorb to the LNP surface. The set of proteins which bind the LNP might play a role in defining the organ-targeting properties of example SORT LNPs via an endogenous mechanism, whereby surface-adsorbed proteins interact with cognate receptors expressed by cells in the target organ. Since example Lung and Spleen SORT LNPs include molecules (DOTAP (1,2-dioleoyl-3-trimethylammonium-propane) and 18PA (1,2-dioleoyl-sn-glycero-3-phosphate), respectively) that are not common to conventional LNPs, we reasoned that differences in organ targeting could be driven by differences in protein corona composition. Genetically modified mice can be used to deplete key proteins from the serum, preventing their adsorption to the LNP surface and possibly impairing expected organ targeting. To evaluate the plausibility of an endogenous targeting mechanism in vivo, we investigated how the addition of a SORT molecule to a conventional four-component LNP impacts the functional role of ApoE on tissue-specific mRNA delivery using knockout mice.

We compared the delivery of luciferase mRNA by example SORT LNPs to target organs in B6.129P2-Apoetm1Unc/J (ApoE−/−) mice, a genetic knockout model lacking ApoE expression, to wild type (WT) C57BL/6 mice, containing normal levels of ApoE in the serum. Luminescence was quantified to measure functional luciferase mRNA delivery to the target organs of each example SORT LNP (FIGS. 4A-4C). By eliminating ApoE from the serum, mDLNP exhibited significantly reduced mRNA delivery to the liver, with an average reduction of luciferase activity by 78%, compared to WT C57BL/6 mice (FIGS. 4A-4C). Example liver SORT LNPs, which also bind ApoE selectively from the serum, maintain this dependence on ApoE for mRNA delivery to the liver: knockout of ApoE significantly impaired liver targeting, with an average reduction in luciferase activity by 87% compared to WT C57BL/6 mice (FIGS. 4A-4C). Thus, ApoE is indispensable for efficacious targeting of the liver by both mDLNP reference and example Liver SORT LNPs. In stark contrast, mRNA delivery by example Spleen SORT to the spleen was enhanced by a factor of 2.2 in ApoE−/− mice, suggesting that ApoE plays an antagonistic role in efficacious mRNA delivery to the spleen (FIGS. 4A-4C). Tissue-specific mRNA delivery by example Lung SORT LNPs was not significantly different in ApoE−/− mice compared to WT mice, indicating that this serum protein is not functionally important for modulating lung targeting (FIGS. 4A-4C). Together, these results indicate that mRNA delivery is no longer an ApoE-dependent process upon inclusion of either an anionic lipid or cationic lipid to mDLNP. Rather, example Spleen SORT and Lung SORT LNPs can enable extrahepatic mRNA delivery via an ApoE-independent mechanism. It is plausible that other serum proteins, such as those identified in the proteomics study, may be responsible for endogenous targeting to the spleen and lungs in vivo.

### DISCUSSION

Clinical applications of genetic medicines are limited by the availability of efficacious carriers for the intracellular delivery of nucleic acid biomolecules to target tissues. Since intravenously administered LNPs have been limited to targeting liver hepatocytes to date, there is great need to develop drug delivery systems capable of extrahepatic targeting. Here, we identified mechanistic factors which underly the tissue-targeting properties of example SORT LNPs for extrahepatic mRNA delivery. We found that a SORT molecule's chemical structure uniquely impacts an LNP's biodistribution, apparent pKa, and serum protein interactions. Furthermore, we provide evidence for a plausible endogenous targeting mechanism involving PEG-lipid desorption, serum protein adsorption, and receptor binding, followed by cellular uptake, to possibly explain the organ-targeting profiles of example SORT LNPs (FIG. 2A). Preferential uptake and activity of example SORT LNPs likely occurs in the organs which highly express the relevant cellular receptors that favorably interact with serum proteins enriched at the surface of example SORT LNPs. Although we cannot rule out additional mechanisms, we have identified multiple key factors which define the organ-targeting properties of example SORT LNPs.

The proposed mechanism may have similarity with that of lipoproteins, which can be considered as a natural class of nanoparticles for physiological cholesterol transport through the blood. Acquisition of ApoE in the serum results in uptake of lipoproteins by liver hepatocytes through receptor-mediated endocytosis by LDL-R. This physiological function of ApoE is leveraged by DLin-MC3-DMA LNPs for siRNA delivery to the liver; by binding ApoE in the serum, these nanoparticles can endogenously target LDL-R highly expressed by liver hepatocytes. The hypothesized mechanism we provide evidence for herein builds upon and extends the scope of this endogenous targeting concept. Importantly. example SORT LNPs include classes of “out of the box” supplemental molecules that can tune an LNP's molecular composition to promote the binding of distinct protein species to the LNP, which are not typically observed in the protein corona, and enable mRNA delivery to cells and organs beyond liver hepatocytes. Because example SORT LNPs include additional classes of molecules that have not been included in LNPs before, the SORT platform expands the toolbox of molecules available to control the protein corona without loss of efficacy. Although a static ex vivo incubation does not fully recapitulate the dynamic flow environment in which the protein corona is formed in vivo, studies have not identified significant differences in the relative abundance of the key proteins we discovered to associate with SORT LNPs during a static incubation versus a dynamic incubation.

It is known that the composition of the protein corona is influenced by a nanoparticle's surface chemistry. Since SORT LNPs incorporate SORT molecules with distinct chemical structures, SORT LNPs likely have different surface chemistries underneath the PEG layer. Desorption of PEG-lipid from the LNP unveils this surface, enabling specific plasma protein adsorption. It was recently shown that binding of ApoE induces rearrangement of lipids in DLin-MC3-DMA LNPs, promoting migration of certain lipids from the core to the shell. These observations suggest that protein adsorption can alter the surface composition of SORT LNPs, possibly generating unique nano-domains which further promote the adsorption of distinct proteins to the LNP surface. The distinct surface chemistry of example SORT LNPs can explain which specific proteins bind to the LNP. β2-GPI is known to interact with anionic phospholipids, including 18PA, and Vtn has been associated with DOTAP. Further, ApoE, which facilitates liver targeting of LNPs, is highly enriched in Liver SORT LNPs. These results further support the proteomics findings.

Understanding the molecular interactions of these individual proteins can illuminate why their enrichment might result in the observed organ-targeting properties of SORT LNPs. Interactions between ApoE and LDL-R drive hepatic accumulation of endogenous lipoproteins during cholesterol metabolism, possibly explaining why ApoE adsorption is a hallmark of many liver-targeting LNPs, including Liver SORT. In a similar manner, Vtn could bind its cognate receptor, αvβ3 integrin, which is highly expressed by the pulmonary endothelium but not by liver cells or other vascular beds, providing a plausible explanation for why Vtn promotes lung-specificity. Finally, β2-GPI can bind phosphatidyl serine, an anionic lipid exposed by senescent red blood cells to promote their filtration from the circulation in the spleen, suggesting how β2-GPI could be implicated in Spleen SORT LNP targeting. These findings support the functional role of individual proteins on the organ-targeting properties of SORT LNPs. We acknowledge that the role of other highly enriched proteins in enhancing organ targeting, such as albumin for Liver SORT, should not be discounted. Clusterin, highly enriched in Lung SORT, could potentially play a role in evading the mononuclear phagocyte system to promote lung targeting. Additionally, based on the key role of ApoE in driving liver targeting, the reduced binding of ApoE to Spleen and Lung SORT LNPs could promote extrahepatic mRNA delivery, possibly due to displacement of ApoE by apolipoprotein C, which is present in the protein coronas of Spleen and Lung SORT LNPs but not Liver SORT (Tables 10-12).

Our proposed mechanism suggests that tuning the molecular composition of an LNP serves as a simple and effective method for manipulating the endogenous ligands which bind the nanoparticle and govern its subsequent biological fate. Our work clarifies that endogenous targeting via serum proteins acts as a targeting mechanism that can be generalized to other LNP systems besides those currently utilized for nucleic acid delivery to liver hepatocytes. With the key parameters for organ targeting identified, Spleen and Lung SORT LNPs may be further optimized for more efficacious tissue-specific delivery. Moving forward, the function of highly enriched serum proteins with respect to extrahepatic targeting in vivo should be further elucidated. Additionally, identifying SORT molecules that bind serum proteins distinct from those detailed within this manuscript may serve as a worthwhile strategy for the discovery of new LNPs which target other organs beyond the liver, spleen, and lungs. We view endogenous targeting, that is engineering nanoparticle composition to facilitate interactions with distinct serum proteins and thereby tissue-specific delivery, as an effective and broad paradigm for designing nanoparticles of various material compositions to overcome liver accumulation and target extrahepatic organs.

While the disclosure has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A composition comprising a therapeutic agent assembled with a lipid composition, which lipid composition comprises:
an ionizable cationic lipid;
a polymer-conjugated lipid comprising one or more hydrocarbon chains that each comprise about 8 to about 20 carbon atoms; and
a selective organ targeting (SORT) lipid separate from said ionizable cationic lipid and said polymer-conjugated lipid.
wherein said lipid composition is characterized by an apparent ionization constant (pKa) from about 6 to about 7 as determined by a 2-(p-toluidino)-6-naphthalenesulfonic acid (TNS) titration assay.

2. A composition comprising a therapeutic agent assembled with a lipid composition, which lipid composition comprises:
an ionizable cationic lipid;
a polymer-conjugated lipid comprising one or more hydrocarbon chains that each comprise about 8 to about 20 carbon atoms; and
a selective organ targeting (SORT) lipid separate from said ionizable cationic lipid and said polymer-conjugated lipid.
wherein said lipid composition is characterized by an apparent ionization constant (pKa) outside a range of about 6 to about 7 as determined by a 2-(p-toluidino)-6-naphthalenesulfonic acid (TNS) titration assay.

3. The composition of claim 2, wherein said lipid composition is characterized by an apparent ionization constant (pKa) of about 6 or lower as determined by a 2-(p-toluidino)-6-naphthalenesulfonic acid (TNS) titration assay.

4. The composition of claim 3, wherein said lipid composition is characterized by an apparent ionization constant (pKa) of about 3 to about 6 as determined by a 2-(p-toluidino)-6-naphthalenesulfonic acid (TNS) titration assay.

5. A composition comprising a therapeutic agent assembled with a lipid composition, which lipid composition comprises:
an ionizable cationic lipid;
a polymer-conjugated lipid comprising one or more hydrocarbon chains that each comprise about 8 to about 20 carbon atoms; and
a selective organ targeting (SORT) lipid separate from said ionizable cationic lipid and said polymer-conjugated lipid.
wherein said lipid composition is characterized by an apparent ionization constant (pKa) of greater than about 6 as determined by a 2-(p-toluidino)-6-naphthalenesulfonic acid (TNS) titration assay.

6. The composition of claim 2 or 5, wherein said lipid composition is characterized by an apparent ionization constant (pKa) of about 8 or greater as determined by a 2-(p-toluidino)-6-naphthalenesulfonic acid (TNS) titration assay.

7. The composition of claim 6, wherein said lipid composition is characterized by an apparent ionization constant (pKa) of about 8 to about 13 as determined by a 2-(p-toluidino)-6-naphthalenesulfonic acid (TNS) titration assay.

8. The composition of claim 6, wherein said lipid composition is characterized by an apparent ionization constant (pKa) of about 9 or greater as determined by a 2-(p-toluidino)-6-naphthalenesulfonic acid (TNS) titration assay.

9. The composition of claim 8, wherein said lipid composition is characterized by an apparent ionization constant (pKa) of about 9 to about 13 as determined by a 2-(p-toluidino)-6-naphthalenesulfonic acid (TNS) titration assay.

10. The composition of any one of claims 1-9, wherein said lipid composition is characterized by a zeta (ζ) potential of said lipid composition of about −10 millivolts (mV) to about 10 mV as determined by dynamic light scattering (DLS).

11. The composition of claim 10, wherein said lipid composition is characterized by a zeta (ζ) potential of said lipid composition of about 0 millivolt (mV) to about 10 mV as determined by dynamic light scattering (DLS).

12. The composition of any one of claims 1-11, wherein said polymer-conjugated lipid is a polyethylene glycol (PEG)-conjugated lipid.

13. The composition of any one of claims 1-12, wherein said one or more hydrocarbon chains each comprise about 8 to about 18 carbon atoms.

14. The composition of any one of claims 1-12, wherein said one or more hydrocarbon chains each comprise about 8 to about 16 carbon atoms.

15. The composition of any one of claims 1-12, wherein said one or more hydrocarbon chains each comprise about 8 to about 14 carbon atoms.

16. The composition of any one of claims 1-15, wherein a hydrocarbon chain of said one or more hydrocarbon chains of said polymer-conjugated lipid comprises no more than 3 unsaturated carbon-carbon bonds.

17. The composition of any one of claims 1-15, wherein a hydrocarbon chain of said one or more hydrocarbon chains of said polymer-conjugated lipid comprises no more than 2 unsaturated carbon-carbon bonds.

18. The composition of any one of claims 1-17, wherein said polymer-conjugated lipid comprises a polymer having a molecular weight of about 100 Daltons (Da) to about 100,000 Da.

19. The composition of any one of claims 1-17, wherein said polymer-conjugated lipid comprises a polymer having a molecular weight of about 500 Da to about 100,000 Da.

20. The composition of any one of claims 1-19, wherein said lipid composition comprises said polymer-conjugated lipid at a molar percentage from about 0.5% to about 20%.

21. The composition of any one of claims 1-19, wherein said lipid composition comprises said polymer-conjugated lipid at a molar percentage from about 0.5% to about 15%.

22. The composition of any one of claims 1-19, wherein said lipid composition comprises said polymer-conjugated lipid at a molar percentage from about 0.5% to about 10%.

23. The composition of any one of claims 1-22, wherein said cationic ionizable lipid is comprises a dendron or dendrimer comprising one or more branches, wherein said one or more branches each comprise two or more degradable functional groups.

24. The composition of any one of claims 1-23, wherein said cationic ionizable lipid is a dendron or dendrimer that comprises one or more diacyl groups.

25. The composition of any one of claims 1-24, wherein the ionizable cationic lipid is a dendrimer or dendron of a generation (g) having a structural formula: 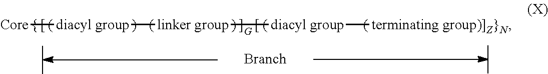
or a pharmaceutically acceptable salt thereof, wherein:
(a) the core comprises a structural formula (X_{Core}): 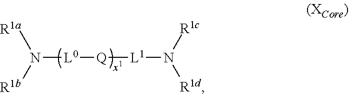
wherein:
Q is independently at each occurrence a covalent bond, —O—, —S—, —NR²—, or —CR^{3a}R^{3b}—;
R² is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene](e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}): 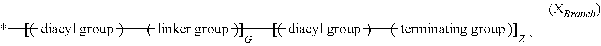
wherein:
* indicates a point of attachment of the branch to the core;
g is 1, 2, 3, or 4;
Z=2^{(g-1)};
G=0, when g=1; or
$$G = {\sum }_{i = 0}^{i = g - 2} {2}^{i} ,$$
when g≠1;
(c) each diacyl group independently comprises a structural formula 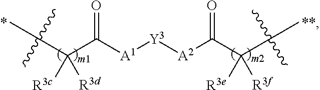
wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein:
R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula 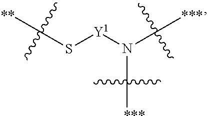
wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

26. The composition of claim 25, wherein x¹ is 0, 1, 2, or 3.

27. The composition of claim 25 or 26, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl 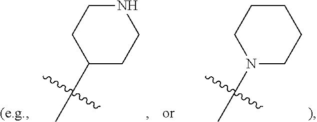 [SMILES: *C1CCNCC1 ; *N1CCCCC1]
N—(C₁-C₃ alkyl)-piperidinyl 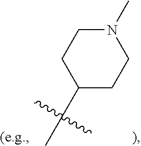 [SMILES: *C1CCN(C)CC1]
piperazinyl 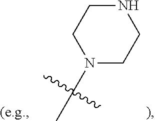 [SMILES: *N1CCNCC1]
N—(C₁-C₃ alkyl)-piperadizinyl 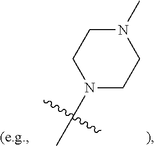 [SMILES: *N1CCN(C)CC1]
morpholinyl 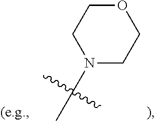 [SMILES: *N1CCOCC1]
N-pyrrolidinyl 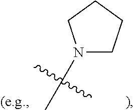 [SMILES: *N1CCCC1]
pyrrolidinyl 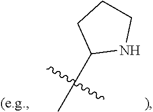 [SMILES: *C1CCCN1]
or N—(C₁-C₃ alkyl)-pyrrolidinyl 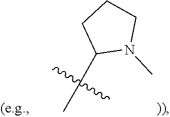 [SMILES: *C1CCCN1C]
(e.g., C₆-C₁₀) aryl, and C₃-C₅ heteroaryl (e.g., imidazolyl 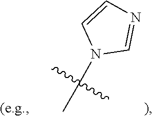 [SMILES: *n1ccnc1]
or pyridinyl

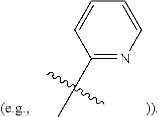 [SMILES: *c1ccccn1]

28. The composition of any one of claims 25-27, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

29. The composition of any one of claims 25-28, wherein R^{3a} and R^{3b} are each independently at each occurrence hydrogen.

30. The composition of any one of claims 25-29, wherein the plurality (N) of branches comprises at least 3 (e.g., at least 4, or at least 5) branches.

31. The composition of any one of claims 25-30, wherein g=1; G=0; and Z=1.

32. The composition of claim 31, wherein each branch of the plurality of branches comprises a structural formula

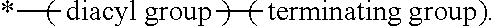

33. The composition of any one of claims 25-30, wherein g=2; G=1; and Z=2.

34. The composition of claim 33, wherein each branch of the plurality of branches comprises a structural formula

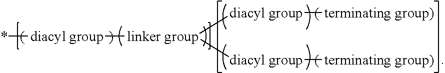

35. The composition of any one of claims 25-30, wherein g=3; G=3; and Z=4.

36. The composition of claim 35, wherein each branch of the plurality of branches comprises a structural formula

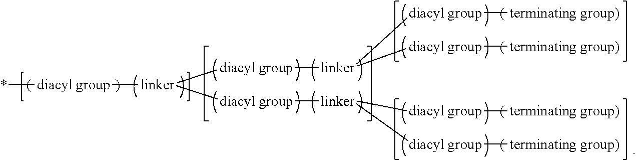

37. The composition of any one of claims 25-30, wherein g=4; G=7; and Z=8.

38. The composition of claim 37, wherein each branch of the plurality of branches comprises a structural formula

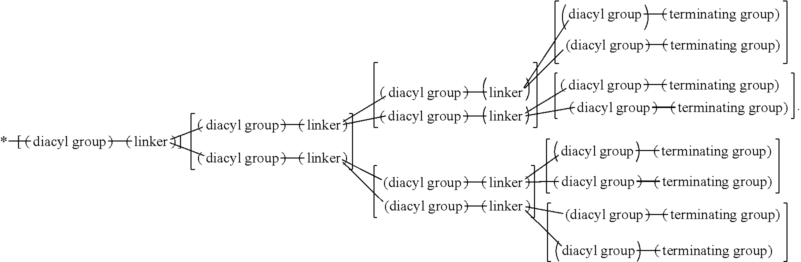

39. The composition of any one of claims 25-38, wherein the core comprises a structural formula:

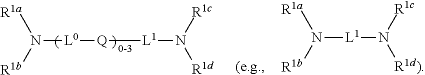

40. The composition of any one of claims 25-38, wherein the core comprises a structural formula:

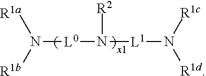

41. The composition of any one of claims 25-38, wherein the core comprises a structural formula:

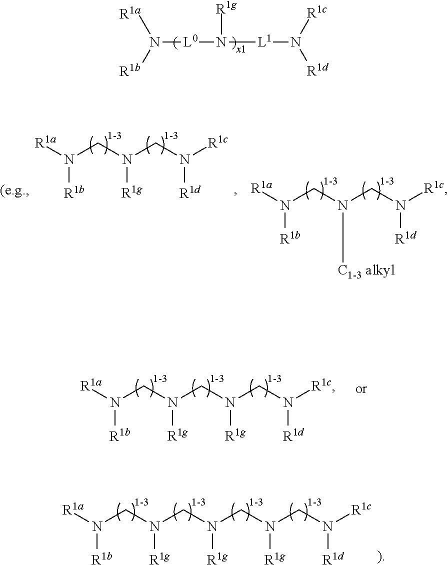

42. The composition of any one of claims 25-38, wherein the core comprises a structural formula:

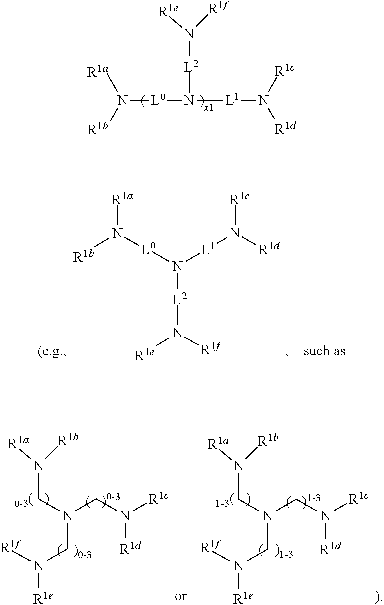

43. The composition of any one of claims 25-38, wherein the core comprises a structural formula: 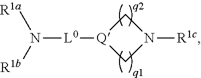
wherein Q′ is —NR²— or —CR^{3a}R^{3b}—; q¹ and q² are each independently 1 or 2.

44. The composition of any one of claims 25-38, wherein the core comprises a structural formula:

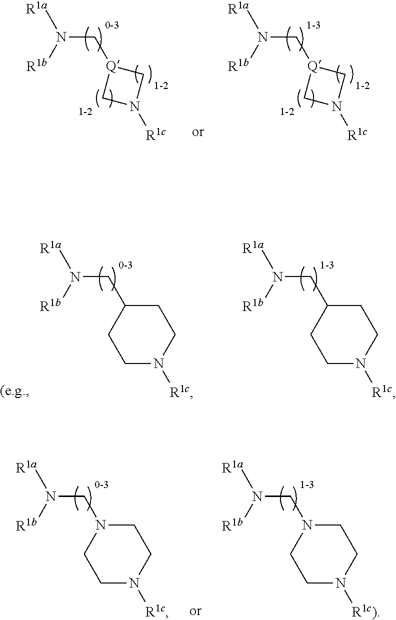

45. The composition of any one of claims 25-38, wherein the core comprises a structural formula 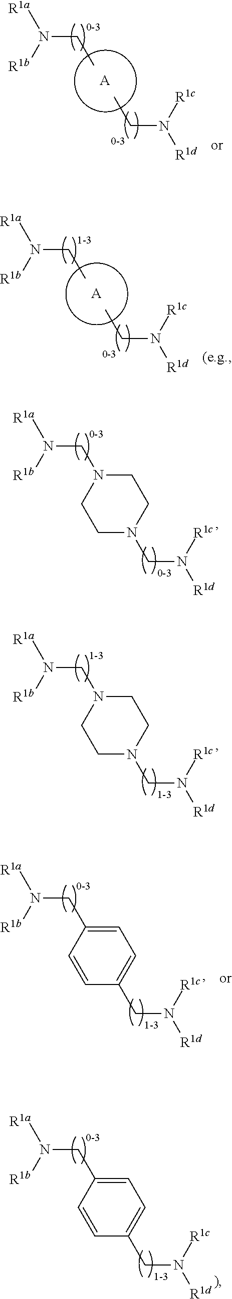
wherein ring A is an optionally substituted aryl or an optionally substituted (e.g., C₃-C₁₂, such as C₃-C₅) heteroaryl.

46. The composition of any one of claims 25-38, wherein the core comprises has a structural formula

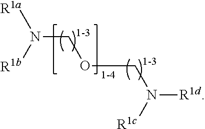

47. The composition of any one of claims 25-38, wherein the core comprises a structural formula selected from the group consisting of: 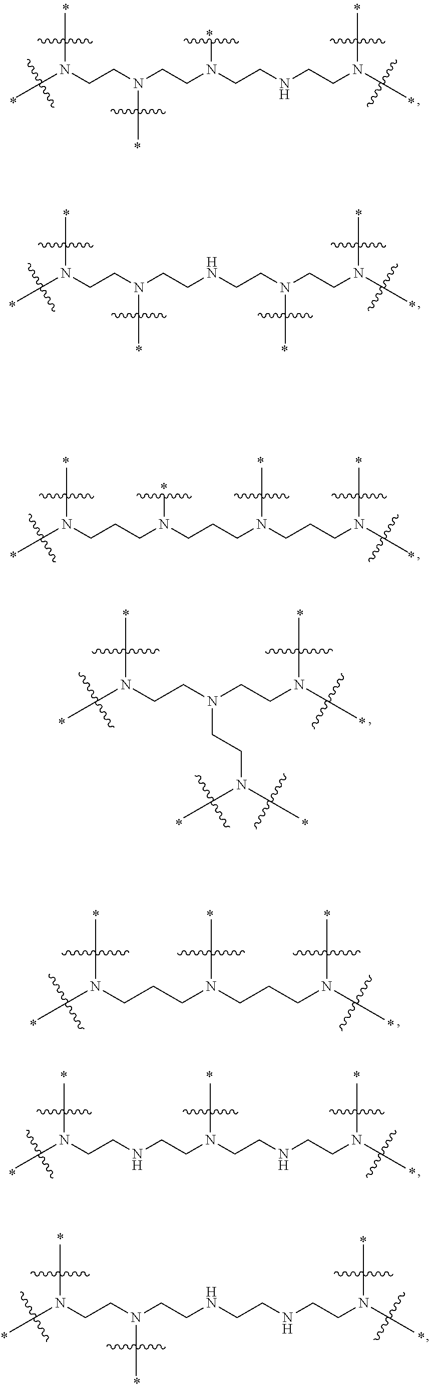 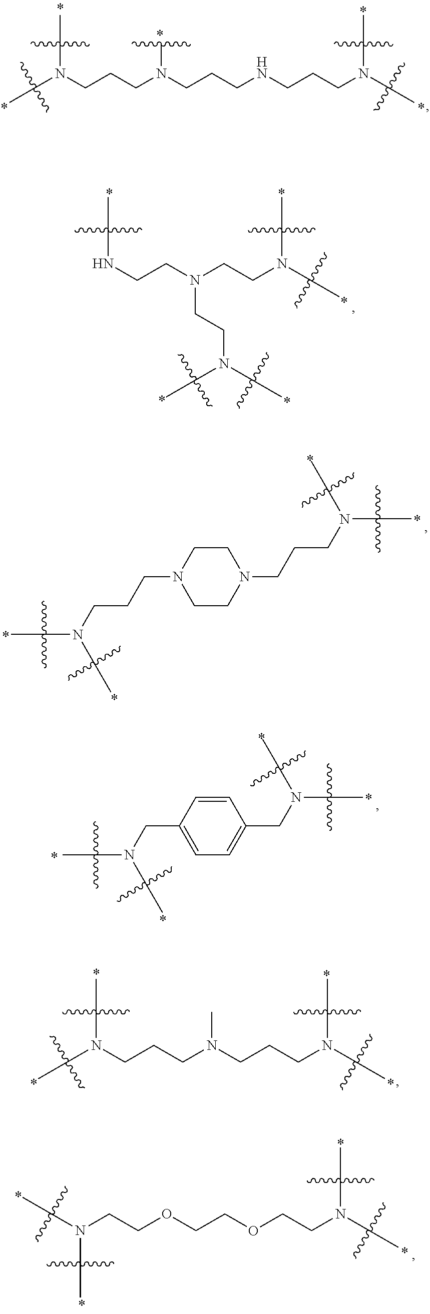 [SMILES: *N(*)CCCNCCCN(*)CCCN(*)* ; *NCCN(CCN(*)*)CCN(*)* ; *N(*)CCCN1CCN(CCCN(*)*)CC1 ; *N(*)Cc1ccc(CN(*)*)cc1 ; *N(*)CCCN(C)CCCN(*)* ; *N(*)CCOCCOCCN(*)*] 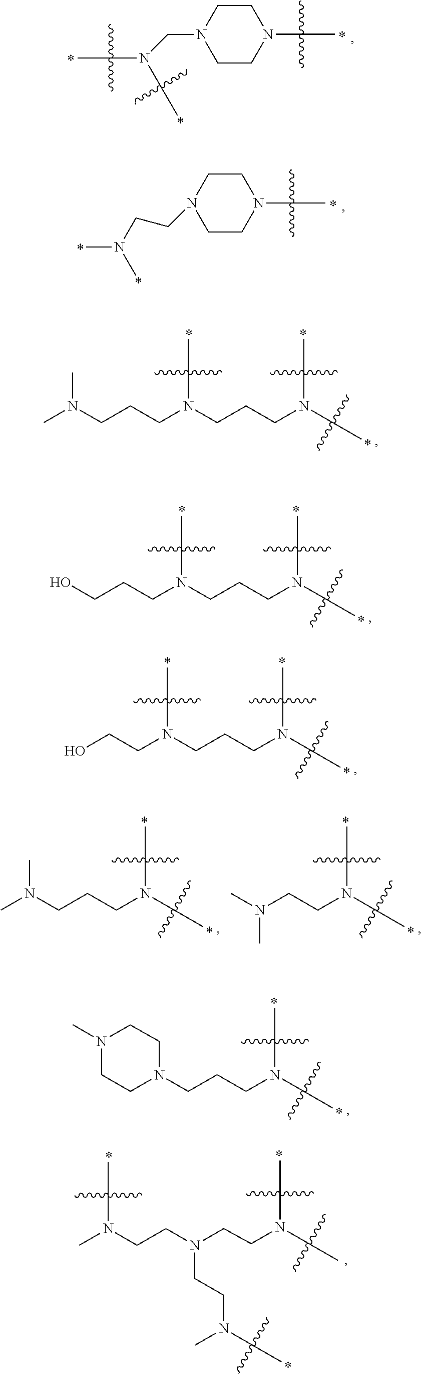
and pharmaceutically acceptable salts thereof, wherein * indicates a point of attachment of the core to a branch of the plurality of branches.

48. The composition of any one of claims 25-47, wherein A¹ is —O— or —NH—.

49. The composition of any one of claims 25-48, wherein A² is —O— or —NH—.

50. The composition of any one of claims 25-49, wherein Y³ is C₁-C₁₂ (e.g., C₁-C₆, such as C₁-C₃) alkylene.

51. The composition of any one of claims 25-50, wherein the diacyl group independently at each occurrence comprises a structural formula 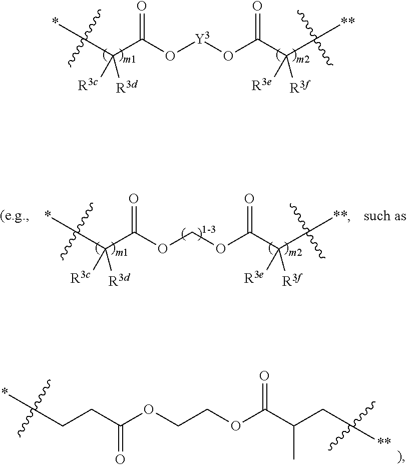
optionally wherein R^{3c}, R^{3d}, R^{3e}, and R^{3f} are each independently at each occurrence hydrogen or C₁-C₃ alkyl.

52. The composition of any one of claims 25-51, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene] 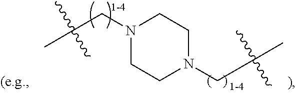
and [(C₁-C₄) alkylene]-1-4 phenylene-[(C₁-C₄) alkylene]

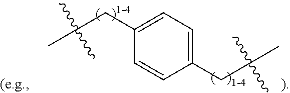

53. The composition of any one of claims 25-51, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

54. The composition of any one of claims 25-51, wherein L⁰, L¹, and L² are each independently at each occurrence C₁-C₆ alkylene (e.g., C₁-C₃ alkylene).

55. The composition of any one of claims 25-51, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

56. The composition of any one of claims 25-51, wherein L⁰, L¹, and L² are each independently at each occurrence selected from [(C₁-C₄) alkylene]-[(C₄-C₆) heterocycloalkyl]-[(C₁-C₄) alkylene](e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene](e.g., —(C₁-C₃ alkylene)-piperazinyl-(C₁-C₃ alkylene)-).

57. The composition of any one of claims 25-56, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino 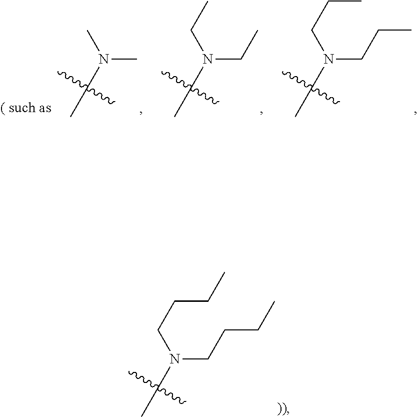
C₄-C₆ N-heterocycloalkyl 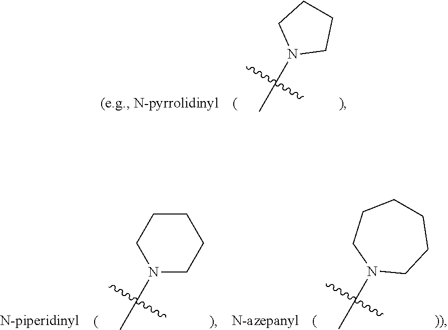 [SMILES: *N1CCCC1 ; *N1CCCCC1 ; *N1CCCCCC1]
—OH, —C(O)OH, —C(O)N(C₁-C₃ alkyl)-(C₁-C₆ alkylene)-(C₁-C₁₂ alkylamino (e.g., mono- or di-alkylamino)) 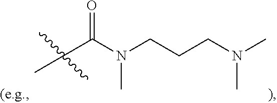 [SMILES: *C(=O)N(C)CCCN(C)C]
—C(O)N(C₁-C₃ alkyl)-(C₁-C₆ alkylene)-(C₄-C₆ N-heterocycloalkyl) 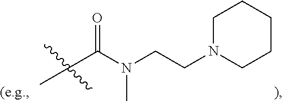 [SMILES: *C(=O)N(C)CCN1CCCCC1]
—C(O)—(C₁-C₁₂ alkylamino (e.g., mono- or di-alkylamino)), and —C(O)—(C₄-C₆ N-heterocycloalkyl) 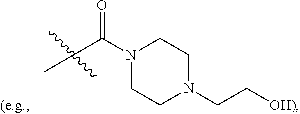
wherein the C₄-C₆ N-heterocycloalkyl moiety of any of the preceding substituents is optionally substituted with C₁-C₃ alkyl or C₁-C₃ hydroxyalkyl.

58. The composition of any one of claims 25-56, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino 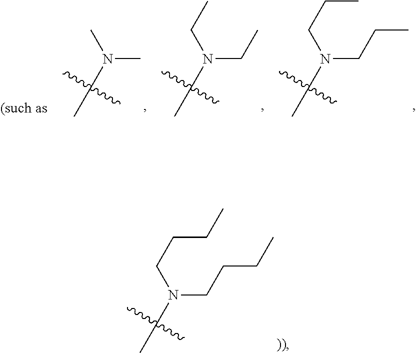
C₄-C₆ N-heterocycloalkyl 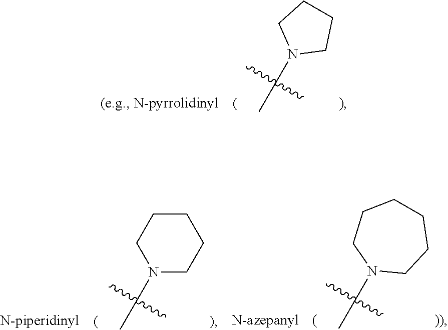 [SMILES: *N1CCCC1 ; *N1CCCCC1 ; *N1CCCCCC1]
—OH, —C(O)OH, —C(O)N(C₁-C₃ alkyl)-(C₁-C₆ alkylene)-(C₁-C₁₂ alkylamino (e.g., mono- or di-alkylamino)) 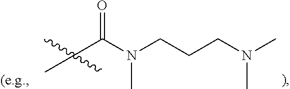 [SMILES: *C(=O)N(C)CCCN(C)C]
—C(O)N(C₁-C₃ alkyl)-(C₁-C₆ alkylene)-(C₄-C₆ N-heterocycloalkyl) 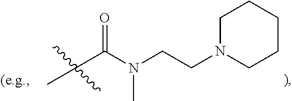 [SMILES: *C(=O)N(C)CCN1CCCCC1]
and —C(O)—(C₄-C₆ N-heterocycloalkyl) 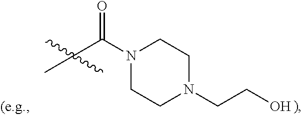 [SMILES: *C(=O)N1CCN(CCO)CC1]
wherein the C₄-C₆ N-heterocycloalkyl moiety of any of the preceding substituents is optionally substituted with C₁-C₃ alkyl or C₁-C₃ hydroxyalkyl.

59. The composition of any one of claims 25-56, wherein each terminating group is independently C₁-C₁₈ (e.g., C₄-C₁₈) alkylthiol, wherein the alkyl moiety is optionally substituted with one substituent —OH.

60. The composition of any one of claims 25-56, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino 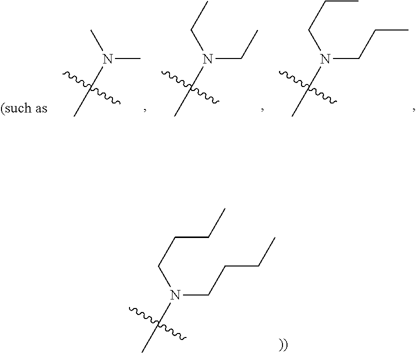
and C₄-C₆ N-heterocycloalkyl

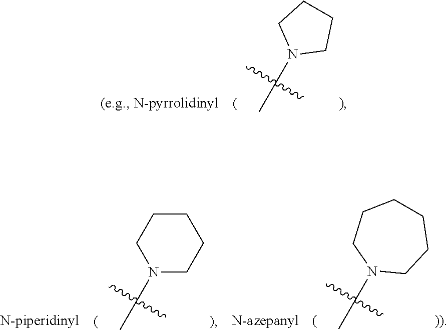 [SMILES: *N1CCCC1 ; *N1CCCCC1 ; *N1CCCCCC1]

61. The composition of any one of claims 25-56, wherein each terminating group is independently C₁-C₁₈ (e.g., C₄-C₁₈) alkenylthiol or C₁-C₁₈ (e.g., C₄-C₁₈) alkylthiol.

62. The composition of any one of claims 25-56, wherein each terminating group is independently C₁-C₁₈ (e.g., C₄-C₁₈) alkylthiol.

63. The composition of any one of claims 25-56, wherein each terminating group is independently selected from the group consisting of:

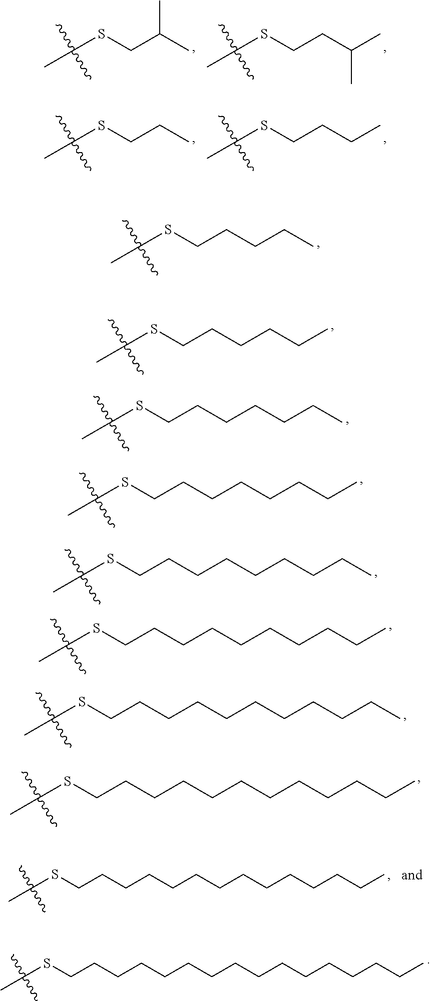

64. The composition of claim 25, wherein the dendrimer or dendron is selected from the group consisting of 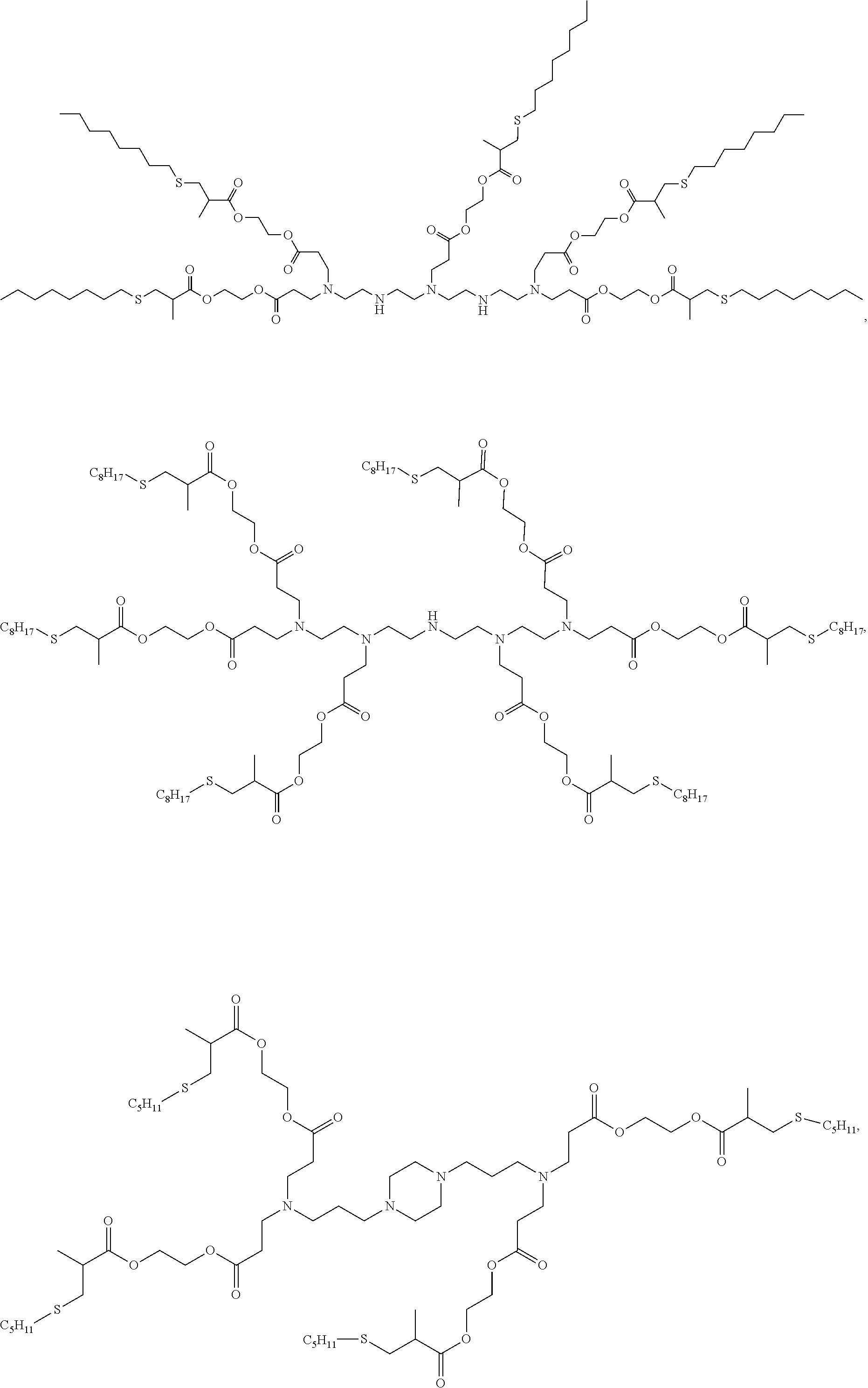 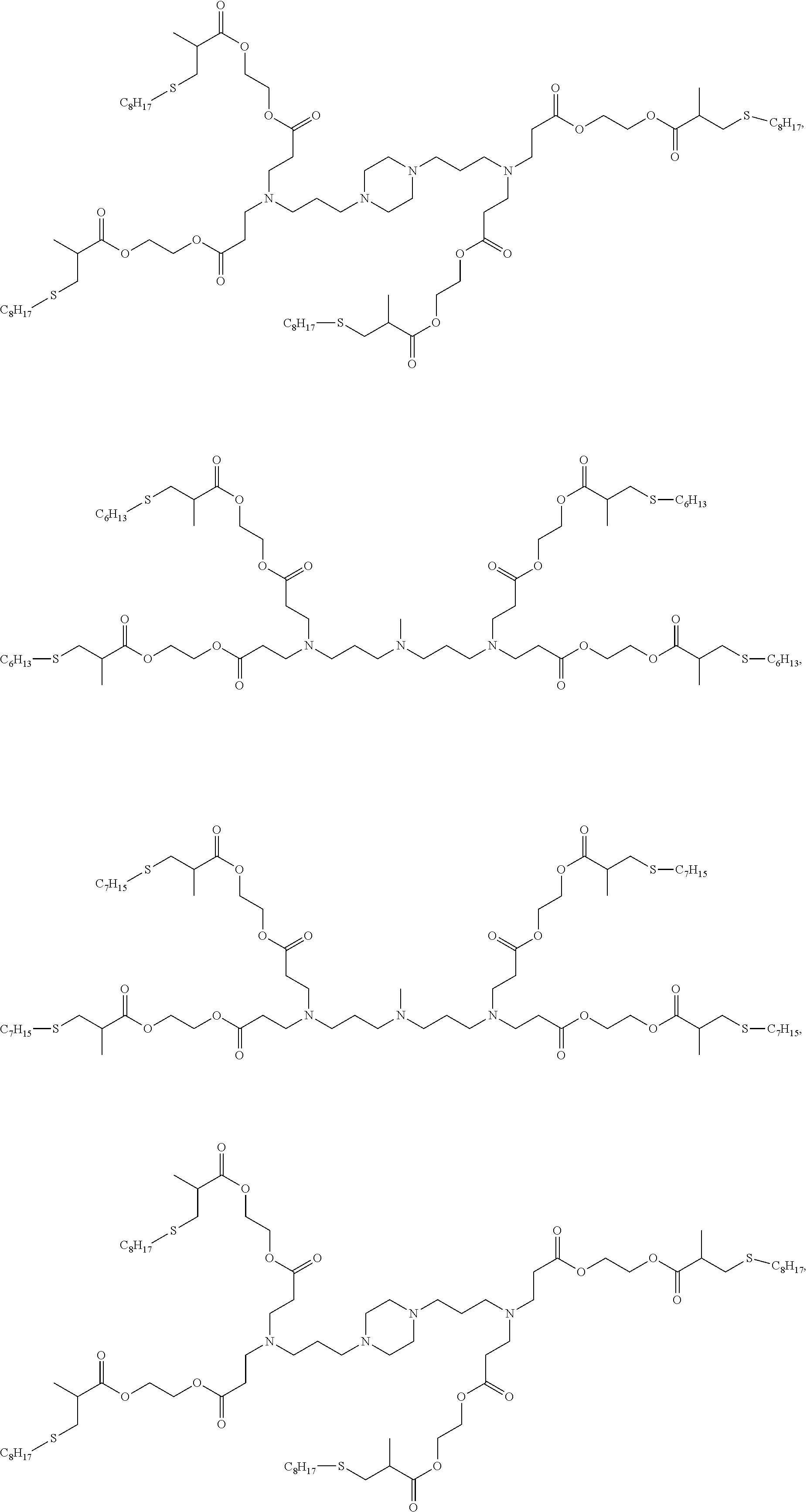 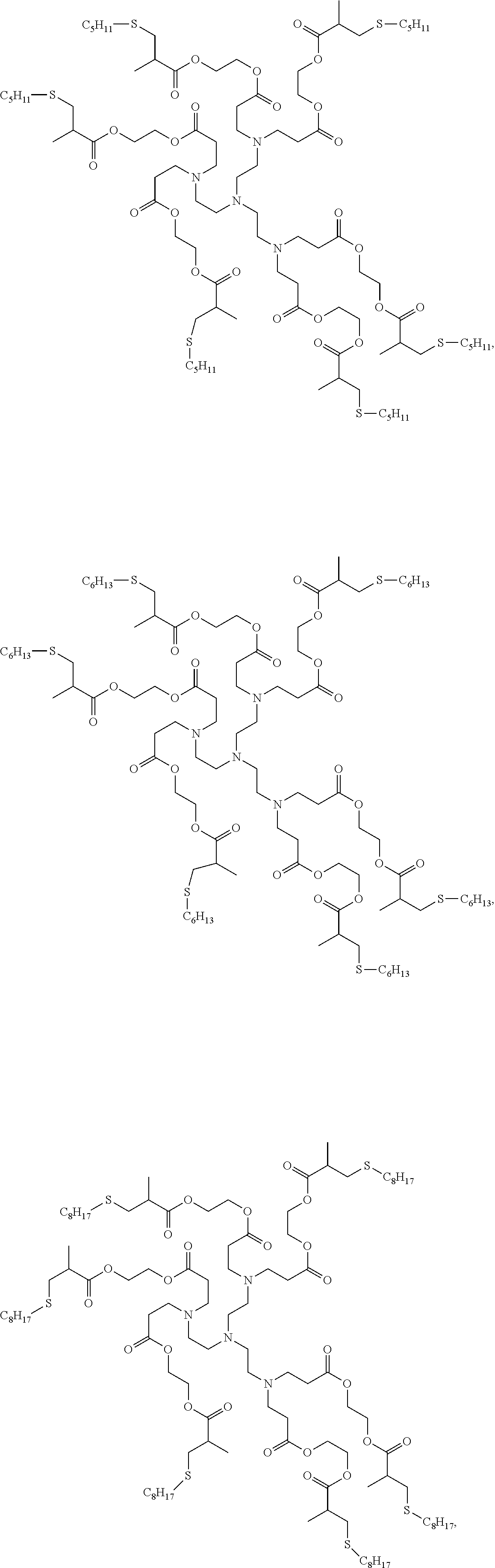
and pharmaceutically acceptable salts thereof.

65. The composition of any one of claims 1-64, wherein said lipid composition comprises said ionizable cationic lipid at a molar percentage from about 5% to about 30%.

66. The composition of any one of claims 1-65, wherein said lipid composition further comprises a phospholipid.

67. The composition of claim 66, wherein said lipid composition comprises said phospholipid at a molar percentage from about 5% to about 30%.

68. The composition of claim 66, wherein said lipid composition comprises said phospholipid at a molar percentage from about 8% to about 23%.

69. The composition of any one of claims 66-68, wherein said phospholipid is not an ethylphosphocholine.

70. The composition of any one of claims 1-69, wherein said lipid composition further comprises a steroid or steroid derivative.

71. The composition of claim 70, wherein said lipid composition comprises said steroid or steroid derivative at a molar percentage from about 15% to about 46%.

72. The composition of claim 70 or 71, wherein said steroid or steroid derivative is cholesterol.

73. The composition of any one of claims 1-72, wherein said SORT lipid is cationic.

74. The composition of any one of claims 1-73, wherein said SORT lipid comprises an ionizable cationic moiety (e.g., a tertiary amine moiety).

75. The composition of claim 74, wherein said SORT lipid has a structural formula: 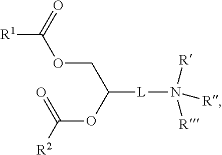
wherein:
L is a bond or a (e.g., biodegradable) linker;
R₁ and R₂ are each independently alkyl_{(C8-C24)}, alkenyl_{(C8-C24)}, or a substituted version of either group; and
R′, R″ and R′″ are each independently alkyl_{(C≤6)} or substituted alkyl_{(C≤6)}.

76. The composition of claim 74, wherein said SORT lipid has a structural formula: 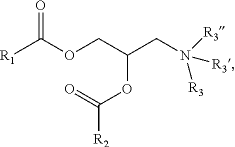
wherein:
R₁ and R₂ are each independently alkyl_{(C8-C24)}, alkenyl_{(C8-C24)}, or a substituted version of either group; and
R₃, R₃′, and R₃″ are each independently alkyl_{(C≤6)} or substituted alkyl_{(C≤6)}.

77. The composition of any one of claims 1-73, wherein said SORT lipid comprises a permanent cationic moiety (e.g., a quaternary ammonium ion).

78. The composition of claim 77, wherein said SORT lipid comprises a counterion to said permanent cationic moiety.

79. The composition of claim 77 or 78, wherein said SORT lipid is an alkylated phosphocholine (e.g., ethylphosphocholine).

80. The composition of claim 77 or 78, wherein said SORT lipid comprises a headgroup having a structural formula: 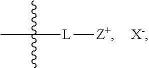
wherein L is a bond or a (e.g., biodegradable) linker; Z⁺ is positively charged moiety (e.g., a quaternary ammonium ion); and X⁻ is a counterion.

81. The composition of claim 80, wherein said SORT lipid has a structural formula: 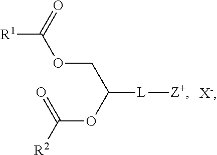
wherein R¹ and R² are each independently an optionally substituted C₆-C₂₄ alkyl, or an optionally substituted C₆-C₂₄ alkenyl.

82. The composition of claim 80, wherein said SORT lipid has a structural formula: 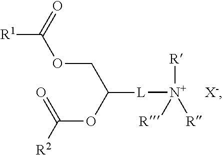
wherein:
R₁ and R₂ are each independently alkyl_{(C8-C24)}, alkenyl_{(C8-C24)}, or a substituted version of either group;
R′, R″ and R′″ are each independently alkyl_{(C≤6)} or substituted alkyl_{(C≤6)}; and
X⁻ is a monovalent anion.

83. The composition of any one of claims 80-82, wherein L is 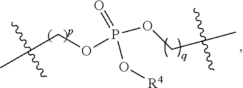
wherein:
p and q are each independently 1, 2, or 3; and
R⁴ is an optionally substituted C₁-C₆ alkyl.

84. The composition of claim 77 or 78, wherein said SORT lipid has a structural formula: 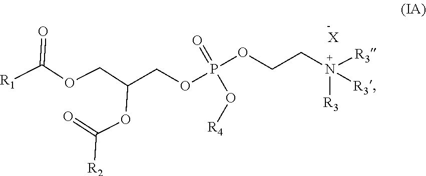
wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

85. The composition of claim 77 or 78, wherein said SORT lipid has a structural formula: 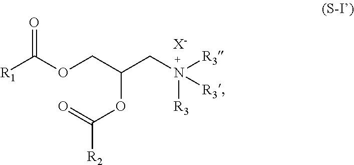
wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
X⁻ is a monovalent anion.

86. The composition of claim 77 or 78, wherein said SORT lipid has a structural formula: 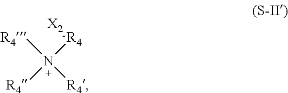
wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂ is a monovalent anion.

87. The composition of any one of claims 1-86, wherein said lipid composition comprises said SORT lipid at a molar percentage from about 20% to about 65%.

88. The composition of any one of claims 2-4 and 10-72, wherein said SORT lipid is zwitterionic.

89. The composition of claim 88, wherein said SORT lipid comprises a hydrophobically modified phosphate anion, a sulfonate anion, or a carboxylate anion.

90. The composition of any one of claims 2-4 and 10-72, wherein said SORT lipid is anionic.

91. The composition of claim 90, wherein said SORT lipid has a structural formula: 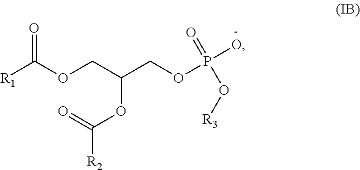
wherein:
R₁ and R₂ are each independently alkyl_{(C8-C24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
Y₁ is alkanediyl_{(C≤6)} or substituted alkanediyl_{(C≤6)}; and
R₄ is acyloxy_{(C≤8-24)} or substituted acyloxy_{(C≤8-24)}.

92. The composition of any one of claims 1-91, wherein said lipid composition is characterized by an average diameter of about 200 nanometers (nm) or less as determined by dynamic light scattering (DLS).

93. The composition of any one of claims 1-91, wherein said lipid composition is characterized by an average diameter of about 150 nanometers (nm) or less as determined by dynamic light scattering (DLS).

94. The composition of any one of claims 1-91, wherein said lipid composition is characterized by an average diameter of about 100 nanometers (nm) or less as determined by dynamic light scattering (DLS).

95. The composition of any one of claims 1-94, wherein said lipid composition is characterized by a polydispersity index (PDI) of about 0.2 or less as determined by dynamic light scattering (DLS).

96. The composition of any one of claims 1-95, wherein said lipid composition is characterized by a lipid fusion percentage of at least about 5%, 6%, 7%, 8%, 9%, or 10% as determined by a fluorescence resonance energy transfer (FRET)-based assay.

97. The composition of any one of claims 1-96, wherein said therapeutic agent comprises a compound, a polynucleotide, a polypeptide, a protein, or a combination thereof.

98. The composition of any one of claims 1-96, wherein said therapeutic agent comprises a polypeptide or a protein.

99. The composition of any one of claims 1-96, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

100. The composition of any one of claims 1-96, wherein said therapeutic agent comprises a polynucleotide; and wherein a molar ratio of nitrogen in said lipid composition to phosphate in said polynucleotide (N/P ratio) is no more than about 20:1.

101. The composition of claim 100, wherein said N/P ratio is from about 5:1 to about 20:1.

102. The composition of claim 100 or 101, wherein said therapeutic agent comprises two or more polynucleotides that comprises said polynucleotide.

103. The composition of any one of claims 1-102, wherein a molar ratio of said therapeutic agent to total lipids of said lipid composition is no more than about 1:1, 1:10, 1:50, or 1:100.

104. The composition of any one of claims 1-103, wherein at least about 85% of said therapeutic agent is encapsulated in particles of said lipid compositions.

105. The composition of any one of claims 1-104, wherein said SORT lipid is present in said composition in an amount sufficient to achieve a therapeutic effect at a dose of said therapeutic agent (e.g., at least about 1.1- or 10-fold) lower than that required with a reference lipid composition.

106. The composition of any one of claims 1-105, wherein said therapeutic agent (e.g., heterologous polynucleotide) is present in said composition at a dose of no more than about 2 milligram per kilogram (mg/kg, or mpk) body weight.

107. The composition of any one of claims 1-106, wherein said therapeutic agent (e.g., heterologous polynucleotide) is present in said intravenous composition at a dose of no more than about 1.0, 0.5, 0.1, 0.05, or 0.01 mg/kg body weight.

108. The composition of any one of claims 1-106, wherein the therapeutic agent is present in an aerosol composition at a dose of no more than 1.0, 0.5, 0.1, 0.05, or 0.01 mg/kg body weight.

109. The composition of any one of claims 1-107, wherein said therapeutic agent (e.g., heterologous polynucleotide) is present in said intravenous dosage form at a concentration of no more than about 5 or 2 milligram per milliliter (mg/mL).

110. A method for targeted delivery of a therapeutic agent to an organ or a cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, a surface of said lipid composition binds to a plurality of target proteins as determined by an incubation assay, which plurality of target proteins comprises a first target protein at a weight or mass ratio of no more than about 20:1, 15:1, or 10:1 to a second target protein that is different from said first target protein, thereby delivering said therapeutic agent to said target organ or said target cell in said subject.

111. The method of claim 110, wherein the composition is according to any one of claims 1-109.

112. The method of claim 110 or 111, wherein the method provides a (e.g., at least about 2-fold) greater amount, expression or activity of said therapeutic agent in said organ or said cell therein in said subject as compared to that achieved with a corresponding reference lipid composition (e.g., absent binding to said plurality of target proteins).

113. The method of any one of claims 110-112, wherein the method provides a (e.g., at least about 2-fold) greater amount, expression or activity of said therapeutic agent in said organ or said cell therein in said subject as compared to that achieved absent said polymer-conjugated lipid.

114. The method of any one of claims 110-113, wherein the method provides a (e.g., at least about 2-fold) greater amount, expression or activity of said therapeutic agent in said organ or said cell therein in said subject as compared to that achieved in a reference organ or a reference cell.

115. The method of any one of claims 110-114, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

116. A method for targeted delivery of a therapeutic agent to liver or a liver cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, a surface of said lipid composition binds to a plurality of target proteins as determined by an incubation assay, which plurality of target proteins comprises apolipoprotein E (Apo E) and serum albumin, thereby delivering said therapeutic agent to said liver or said liver cell in said subject.

117. The method of claim 116, wherein said Apo E is present at a weight or mass ratio of no more than about 6:1, 5:1, 4:1, or 3:1 to said serum albumin in said plurality of target proteins as determined by an incubation assay.

118. The method of claim 116 or 117, wherein said plurality of target proteins further comprise complement C1q subcomponent subunit A, immunoglobulin heavy constant mu, complement C1q subcomponent subunit B, immunoglobulin kappa constant, immunoglobulin heavy constant gamma 2B, beta-globin, immunoglobulin (Ig) gamma-2A chain C region, complement C1q subcomponent subunit C, immunoglobulin heavy constant alpha, fibrinogen beta chain, fibrinogen gamma chain, immunoglobulin kappa variable 17-127, alpha globin 1, fibrinogen alpha chain, or any combination thereof as determined by an incubation assay.

119. The method of any one of claims 116-118, wherein said SORT lipid comprises an ionizable cationic moiety (e.g., a tertiary amine moiety).

120. The method of any one of claims 116-118, wherein said SORT lipid is an ionizable cationic lipid.

121. The method of any one of claims 116-120, wherein said lipid composition comprises said SORT lipid at a molar percentage from about 5% to about 65%.

122. The method of any one of claims 116-121, wherein said lipid composition is according to any one of claim 1, 10-76 and 92-109.

123. The method of any one of claims 116-122, wherein the method provides a (e.g., at least about 2-, 3-, 4-, 5-, or 6-fold) greater amount, expression or activity of said therapeutic agent in said liver or said liver cell in said subject as compared to that achieved with a corresponding reference lipid composition (e.g., absent said binding to said plurality of target proteins).

124. A method for targeted delivery of a therapeutic agent to a non-liver organ or a non-liver cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, wherein, upon said administering, a surface of said SORT lipid composition interacts with apolipoprotein E (Apo E) to a lesser degree than with an endogenous protein that is not Apo E in said subject as determined by an incubation assay, which endogenous protein that is not Apo E is selected from beta-2 glycoprotein 1 (β2-GP1) or apolipoprotein H (Apo H), immunoglobulin kappa constant, complement C1q subcomponent subunit A, vitronectin, and serum paraoxonase/arylesterase 1, thereby delivering said therapeutic agent to said non-liver organ or said non-liver cell in said subject.

125. The method of claim 124, wherein said non-liver organ comprises a lung, spleen, bone marrow, or a lymph node.

126. The method of claim 124 or 125, wherein said non-liver cell comprises a lung cell, a spleen cell, or a macrophage.

127. The method of any one of claims 124-126, wherein apolipoprotein E (Apo E) is not the most abundant protein in said plurality of target proteins.

128. The method of any one of claims 124-127, wherein, upon said administering, a surface of said lipid composition interacts with apolipoprotein C (Apo C) to a lesser degree than with apolipoprotein E (Apo E) in said subject as determined by an incubation assay.

129. The method of any one of claims 124-128, wherein the method provides a lesser amount or activity of said therapeutic agent in liver or a cell therein in said subject as compared to that achieved absent said polymer-conjugated lipid.

130. The method of any one of claims 124-129, wherein said SORT lipid is a permanent cationic lipid, an ionizable cationic lipid, a zwitterionic lipid, or an anionic lipid.

131. The method of any one of claims 124-130, wherein said lipid composition comprises said SORT lipid at a molar percentage from about 5% to about 65%.

132. The method of any one of claims 124-131, wherein said lipid composition is according to any one of claims 2-109.

133. A method for targeted delivery of a therapeutic agent to a lung or a lung cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, a surface of said lipid composition binds to a plurality of target proteins as determined by an incubation assay, which plurality of target proteins comprises vitronectin (Vtn) and clusterin, thereby delivering said therapeutic agent to said lung or said lung cell in said subject.

134. The method of claim 133, wherein said vitronectin is present at a weight or mass ratio of no more than about 6:1, or 5:1 to said clusterin in said plurality of target proteins as determined by an incubation assay.

135. The method of claim 133 or 134, wherein said plurality of target proteins further comprise serum paraoxonase/arylesterase 1, apolipoprotein E (Apo E), serum albumin, immunoglobulin kappa constant, prothrombin, complement C1q subcomponent subunit A, fibrinogen beta chain, beta-2 glycoprotein 1 (β2-GP1) or apolipoprotein H (Apo H), immunoglobulin (Ig) mu chain C region, alpha-S1-casein, immunoglobulin heavy constant gamma 2B, fibrinogen gamma chain, fibrinogen alpha chain, vitamin K-dependent protein Z, alpha-I-antitrypsin 1-3, plasminogen, apolipoprotein C-III, complement C1q subcomponent subunit B, thrombospondin-1, coagulation factor X, apolipoprotein A-I, immunoglobulin heavy constant alpha, immunoglobulin (Ig) gamma-2A chain C region, beta-globin, complement C1q subcomponent subunit C, protein Z-dependent protease inhibitor, or any combination thereof as determined by an incubation assay.

136. The method of any one of claims 133-135, wherein said SORT lipid is a cationic lipid.

137. The method of claim 136, wherein said SORT lipid is a permanent cationic lipid.

138. The method of claim 136, wherein said SORT lipid is an ionizable cationic lipid.

139. The method of any one of claims 133-138, wherein said lipid composition comprises said SORT lipid at a molar percentage from about 5% to about 65%.

140. The method of any one of claims 133-139, wherein said lipid composition is according to any one of claims 2 and 5-109.

141. The method of any one of claims 133-140, wherein the method provides a (e.g., at least about 2-, 5-, 10-, 11-, 12-, 13-, 14-, 15-, 16-, 17-, 18-, 19-, or 20-fold) greater amount, expression or activity of said therapeutic agent in said lung or said lung cell in said subject as compared to that achieved with a corresponding reference lipid composition (e.g., absent binding to said plurality of target proteins).

142. The method of any one of claims 133-141, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

143. A method for targeted delivery of a therapeutic agent to spleen, bone marrow, or a lymph node or a cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, a surface of said lipid composition binds to a plurality of target proteins as determined by an incubation assay, which plurality of target proteins comprises beta-2 glycoprotein 1 (β2-GP1) or apolipoprotein H (Apo H) at a weight or mass ratio of no more than about 20:1, 15:1, or 10:1 to a second target protein that is different from said beta-2 glycoprotein 1 (β2-GP1) or apolipoprotein H (Apo H), thereby delivering said therapeutic agent to said spleen, bone marrow, or a lymph node or said cell in said subject.

144. The method of claim 143, wherein said cell comprises a spleen cell, or a macrophage.

145. The method of claim 143 or 144, wherein said second target protein is selected from: immunoglobulin kappa constant, complement C1q subcomponent subunit A, apolipoprotein E (Apo E), immunoglobulin heavy constant gamma 2B, complement C1q subcomponent subunit B, vitronectin, complement C1q subcomponent subunit C, apolipoprotein C-I, immunoglobulin (Ig) gamma-2A chain C region, immunoglobulin (Ig) mu chain C region, serum albumin, serum paraoxonase/arylesterase 1, immunoglobulin heavy constant alpha, and immunoglobulin kappa variable 6-13.

146. The method of any one of claims 143-145, wherein said SORT lipid is a permanent cationic lipid, or an anionic lipid.

147. The method of claim 146, wherein said SORT lipid is a permanent cationic lipid.

148. The method of claim 146, wherein said SORT lipid is an anionic lipid.

149. The method of any one of claims 143-148, wherein said lipid composition comprises said SORT lipid at a molar percentage from about 5% to about 65%.

150. The method of any one of claims 143-149, wherein said lipid composition is according to any one of claims 3-4 and 10-109.

151. The method of any one of claims 143-150, wherein the method provides a (e.g., at least about 2-fold) greater amount, expression or activity of said therapeutic agent in said lung or said lung cell in said subject as compared to that achieved with a corresponding reference lipid composition (e.g., absent binding to said plurality of target proteins).

152. A method for targeted delivery of a therapeutic agent to a non-spleen organ or a non-spleen cell therein in a subject in need thereof, the method comprising administering to said subject said therapeutic agent assembled with a lipid composition, which lipid composition comprises:
an ionizable cationic lipid;
a polymer-conjugated lipid; and
a selective organ targeting (SORT) lipid separate from said ionizable cationic lipid and said polymer-conjugated lipid,
wherein, upon said administering, a surface of said lipid composition binds to a plurality of target proteins as determined by an incubation assay, which plurality of target proteins comprises a first target protein at a weight or mass ratio of no more than about 20:1, 15:1, or 10:1 to a second target protein that is different from said first target protein, thereby delivering said therapeutic agent to said non-spleen organ or said non-spleen cell in said subject.

153. The method of claim 152, wherein said non-spleen organ is not spleen, bone marrow, or a lymph node.

154. The method of claim 152 or 153, wherein said non-spleen cell is not a spleen cell, or a macrophage.

155. The method of any one of claims 152-154, wherein beta-2 glycoprotein 1 (β2-GP1) or apolipoprotein H (Apo H) is not the most abundant protein in said plurality of target proteins.

156. The method of any one of claims 152-155, wherein said plurality of target proteins comprise clusterin.

157. The method of any one of claims 152-156, wherein said SORT lipid is a permanent cationic lipid, an ionizable cationic lipid, a zwitterionic lipid, or an anionic lipid.

158. The method of any one of claims 152-157, wherein said lipid composition comprises said SORT lipid at a molar percentage from about 5% to about 65%.

159. The method of any one of claims 152-158, wherein said lipid composition is according to any one of claims 1-2 and 5-109.

160. The method of any one of claims 152-159, wherein said therapeutic agent comprises a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a double stranded ribonucleic acid (dsRNA), a CRISPR-associated (Cas) protein, or a combination thereof.

161. The method of any one of claims 110-159, wherein said polymer-conjugated lipid is a polyethylene glycol (PEG)-conjugated lipid.

162. The method of any one of claims 110-161, wherein said one or more hydrocarbon chains each comprise about 8 to about 20 carbon atoms.

163. The method of any one of claims 110-161, wherein said one or more hydrocarbon chains each comprise about 8 to about 18 carbon atoms.

164. The method of any one of claims 110-161, wherein said one or more hydrocarbon chains each comprise about 8 to about 16 carbon atoms.

165. The method of any one of claims 110-161, wherein said one or more hydrocarbon chains each comprise about 8 to about 14 carbon atoms.

166. The method of any one of claims 110-165, wherein a hydrocarbon chain of said one or more hydrocarbon chains of said polymer-conjugated lipid comprises no more than 3 unsaturated carbon-carbon bonds.

167. The method of any one of claims 110-165, wherein a hydrocarbon chain of said one or more hydrocarbon chains of said polymer-conjugated lipid comprises no more than 2 unsaturated carbon-carbon bonds.

168. The method of any one of claims 110-167, wherein said polymer-conjugated lipid comprises a polymer having a molecular weight of about 100 Daltons (Da) to about 100,000 Da.

169. The method of any one of claims 110-167, wherein said polymer-conjugated lipid comprises a polymer having a molecular weight of about 500 Da to about 100,000 Da.

170. The method of any one of claims 110-169, wherein said lipid composition comprises said polymer-conjugated lipid at a molar percentage from about 0.5% to about 20%.

171. The method of any one of claims 110-169, wherein said lipid composition comprises said polymer-conjugated lipid at a molar percentage from about 0.5% to about 15%.

172. The method of any one of claims 110-169, wherein said lipid composition comprises said polymer-conjugated lipid at a molar percentage from about 0.5% to about 10%.

173. The method of any one of claims 110-172, wherein said administering comprises intravenously administering.

174. The method of any one of claims 110-173, wherein a bodily fluid (e.g., plasma or serum) of said subject comprises said plurality of target proteins.

175. The method of any one of claims 110-174, wherein said plurality of target proteins are a plurality of endogenous proteins of said subject.
